(19) United States
(12) Patent Application Publication
(10) Pub. No.: US 2009/0253669 A1
(43) Pub. Date: Oct. 8, 2009

(54) Substituted Sulfonamide Compounds

(75) Inventors: Stefan OBERBOERSCH, Aachen (DE); Melanie REICH, Aachen (DE); Bernd SUNDERMANN, Friedrichsdorf (DE); Werner ENGLBERGER, Stolberg (DE); Sabine HEES, Aachen (DE); Ruth JOSTOCK, Stolberg (DE); Stefan SCHUNK, Aachen (DE); Edward BIJSTERVELD, GE Nijmegen (NL); Fritz THEIL, Berlin (DE)

Correspondence Address: CROWELL & MORING LLP;INTELLECTUAL PROPERTY GROUP, P.O. BOX 14300, WASHINGTON, DC, 20044-4300, US

(73) Assignee: GRUENENTHAL GmbH, Aachen (DE)

(21) Appl. No.: 12/404,876

(22) Filed: Mar. 16, 2009

Related U.S. Application Data
(60) Provisional application No. 61/037,189, filed on Mar. 17, 2008.

(30) Foreign Application Priority Data
Mar. 17, 2008 (EP) 08004922.4

(51) Int. Cl. A61K 31/397; A61K 31/40; C07D 207/48; A61K 31/18; C07C 311/18; C07D 205/04

(52) U.S. Cl. 514/210.01; 514/425; 548/542; 514/604; 564/92; 548/950

(57) ABSTRACT

Substituted sulfonamide compounds corresponding to the formula I:

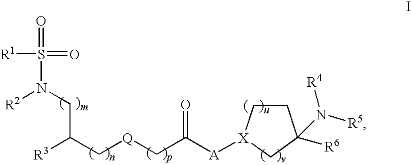

processes for the preparation thereof, pharmaceutical composition containing these compounds and the use of substituted sulfonamide compounds for the preparation of pharmaceutical compositions.

### CROSS REFERENCE TO RELATED APPLICATIONS

This application claims priority from U.S. provisional patent application No. 61/037,189 and European patent application no. EP 08004922.4, both filed Mar. 17, 2008, the entire disclosures of which are incorporated herein by reference.

### BACKGROUND OF THE INVENTION

The present invention relates to substituted sulfonamide compounds, processes for the preparation thereof, pharmaceutical compositions containing these compounds and the use of substituted sulfonamide compounds for the preparation of pharmaceutical compositions.

In contrast to the constitutive expression of the bradykinin 2 receptor (B2R), in most tissues the bradykinin 1 receptor (B1R) is not expressed or expressed only weakly. Nevertheless, expression of B1R can be induced on various cells. For example, in the course of inflammation reactions a rapid and pronounced induction of B1R takes place on neuronal cells, but also various peripheral cells, such as fibroblasts, endothelial cells, granulocytes, macrophages and lymphocytes. In the course of inflammation reactions, a switch from a B2R to a B1R dominance thus occurs on the cells involved. The cytokines interleukin-1 (IL-1) and tumour necrosis factor alpha (TNFα) are involved to a considerable degree in this upwards regulation of B1R (Passos et al. J. Immunol. 2004, 172, 1839-1847). After activation with specific ligands, B1R-expressing cells then themselves can secrete inflammation-promoting cytokines such as IL-6 and IL-8 (Hayashi et al., Eur. Respir. J. 2000, 16, 452-458). This leads to inwards migration of further inflammation cells, e.g. neutrophilic granulocytes (Pesquero et al., PNAS 2000, 97, 8140-8145). The bradykinin B1R system can contribute towards chronification of diseases via these mechanisms. This is demonstrated by a large number of animal studies (overviews in Leeb-Lundberg et al., Pharmacol. Rev. 2005, 57, 27-77 and Pesquero et al., Biol. Chem. 2006, 387, 119-126). On humans too, an enhanced expression of B1R, e.g. on enterocytes and macrophages, in the affected tissue of patients with inflammatory intestinal diseases (Stadnicki et al., Am. J. Physiol. Gastrointest. Liver Physiol. 2005, 289, G361-366) or on T lymphocytes of patients with multiple sclerosis (Prat et al., Neurology. 1999; 53, 2087-2092) or an activation of the bradykinin B2R-B1R system in the course of infections with Staphylococcus aureus (Bengtson et al., Blood 2006, 108, 2055-2063) is found. Infections with Staphylococcus aureus are responsible for syndromes such as superficial infections of the skin up to septic shock.

Based on the pathophysiological relationships described, there is a great therapeutic potential for the use of B1R antagonists on acute and, in particular, chronic inflammatory diseases. These include diseases of the respiratory tract (bronchial asthma, allergies, COPD/chronic obstructive pulmonary disease, cystic fibrosis etc.), inflammatory intestinal diseases (ulcerative colitis, CD/Crohn's disease etc.), neurological diseases (multiple sclerosis, neurodegeneration etc.), inflammations of the skin (atopic dermatitis, psoriasis, bacterial infections etc.) and mucous membranes (Behcet's disease, pelvitis, prostatitis etc.), rheumatic diseases (rheumatoid arthritis, osteoarthritis etc.), septic shock and reperfusion syndrome (following cardiac infarction, stroke).

The bradykinin (receptor) system is moreover also involved in regulation of angiogenesis (potential as an angiogenesis inhibitor in cancer cases and macular degeneration on the eye), and B1R knockout mice are protected from induction of obesity by a particularly fat-rich diet (Pesquero et al., Biol. Chem. 2006, 387, 119-126). B1R antagonists are therefore also suitable for treatment of obesity.

B1R antagonists are particularly suitable for treating pain, especially inflammation pain and neuropathic pain (Calixto et al., Br. J. Pharmacol. 2004, 1-16), and here in particular diabetic neuropathy (Gabra et al., Biol. Chem. 2006, 387, 127-143). They are furthermore suitable for treatment of migraine.

In the development of B1R modulators, however, there is the problem that the human and the rat B1R receptor differ so widely that many compounds which are good B1R modulators on the human receptor have only a poor or no affinity for the rat receptor. This makes pharmacological studies on animals considerably difficult, since many studies are usually conducted on the rat. However, if no activity exists on the rat receptor, neither the action nor side effects can be investigated on the rat. This has already led to transgenic animals with human B1 receptors being produced for pharmacological studies on animals (Hess et al., Biol. Chem. 2006; 387(2):195-201). Working with transgenic animals, however, is more expensive than working with the unmodified animals. Since in the development of pharmaceutical compositions, however, precisely long-term toxicity studies on the rat belong to the standard studies, but this is inappropriate in the event of an absence of activity on the receptor, an important established instrument for checking safety is lacking for the development of such compounds. There is therefore a need for novel B1R modulators, B1R modulators which bind both to the rat receptor and to the human receptor offering particular advantages.

### SUMMARY OF THE INVENTION

One object of the present invention was therefore to provide novel compounds which are suitable in particular as pharmacologically active compounds in pharmaceutical compositions, preferably pharmaceutical compositions for treatment of disorders or diseases which are at least partly mediated by B1R receptors.

This and other objects have been achieved by the substituted sulfonamide compounds according to the invention.

The invention therefore provides substituted sulfonamide compounds corresponding to formula I

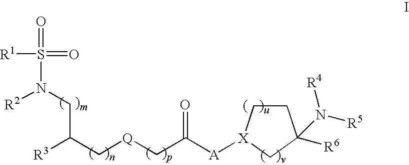

wherein
m represents 0 or 1;
n and p each independently represent 0, 1 or 2;
u and v each independently represent 0, 1, 2, 3 or 4, with the proviso that u+v=1, 2, 3 or 4;
Q represents a single bond, —CH₂— or —O—;
A represents a single bond and X represents N, or
A represents —N(R⁷)—(CH₂)₀₋₅— and X represents CH;
R¹ represents aryl, heteroaryl or an aryl or heteroaryl bonded via a C₁₋₃-alkylene group;
R² and R³ are defined as described under (i) or (ii):
(i) R² represents H, C₁₋₆-alkyl, C₃₋₈-cycloalkyl, aryl or heteroaryl; or denotes a C₃₋₈-cycloalkyl, aryl or heteroaryl bonded via a C₁₋₆-alkylene group, C₂₋₆-alkenylene group or C₂₋₆-alkynylene group; and
R³ represents H, C₁₋₆-alkyl, aryl or heteroaryl; or denotes an aryl or heteroaryl bonded via a C₁₋₆-alkylene group, C₂₋₆-alkenylene group or C₂₋₆-alkynylene group; or
(ii) R² and R³ together with the —N—(CH₂)ₘ—CH— group joining them form a heterocyclic ring, which can be fused with an aryl or heteroaryl ring, wherein the heterocyclic ring may be saturated or mono- or polyunsaturated, but not aromatic, is 4-, 5-, 6- or 7-membered, can contain, in addition to the N hetero atom to which R² is bonded, at least one further hetero atom or a hetero atom group selected from the group consisting of N, NR⁸, O, S, S═O or S(═O)₂; wherein R⁸ denotes H, C₁₋₆-alkyl, —C(═O)—R⁹, C₃₋₈-cycloalkyl, aryl, heteroaryl or a C₃₋₈-cycloalkyl, aryl or heteroaryl bonded via a C₁₋₃-alkylene group, and R⁹ denotes C₁₋₆-alkyl, C₃₋₈-cycloalkyl, aryl, heteroaryl or a C₃₋₈-cycloalkyl, aryl or heteroaryl bonded via a C₁₋₃-alkylene group;
R⁴ and R⁵ are defined as described under (iii) or (iv):
(iii) R⁴ and R⁵ each independently denote H, C₁₋₆-alkyl, C₂₋₆-alkenyl, C₃₋₈-cycloalkyl, 3- to 8-membered heterocycloalkyl, aryl or heteroaryl or a C₃₋₈-cycloalkyl, 3- to 8-membered heterocycloalkyl, aryl or heteroaryl bonded via a C₁₋₃-alkylene group; or
(iv) R⁴ and R⁵ together with the nitrogen atom joining them form an unsubstituted or mono- or polysubstituted heterocyclic ring, which can be fused with a saturated, or mono- or polyunsaturated or aromatic, unsubstituted or mono- or polysubstituted ring system,
   wherein the heterocyclic ring may be saturated or mono- or polyunsaturated, but not aromatic, is 4-, 5-, 6- or 7-membered, can contain, in addition to the N hetero atom to which R⁴ and R⁵ are bonded, at least one further hetero atom or a hetero atom group selected from the group consisting of N, NR¹⁰, O, S, S═O and S(═O)₂,
   the ring system is 4-, 5-, 6- or 7-membered, can contain at least one hetero atom or a hetero atom group selected from the group consisting of N, NR¹¹, O, S, S═O and S(═O)₂,
   R¹⁰ represents a radical selected from the group consisting of H, C₁₋₆-alkyl, C₃₋₈-cycloalkyl, aryl, heteroaryl or an aryl, heteroaryl or C₃₋₈-cycloalkyl bonded via a C₁₋₃-alkylene group and
   R¹¹ represents a radical selected from the group consisting of H, C₁₋₆-alkyl, C₃₋₈-cycloalkyl, aryl, heteroaryl or an aryl, heteroaryl or C₃₋₈-cycloalkyl bonded via a C₁₋₃-alkylene group;
R⁶ represents an aryl, heteroaryl or an aryl or heteroaryl bonded via a C₁₋₆-alkylene group;
R⁷ represents H, C₁₋₆-alkyl, C₃₋₈-cycloalkyl or a C₃₋₈-cycloalkyl bonded via a C₁₋₃-alkylene group;
   wherein the abovementioned C₁₋₆-alkyl, C₂₋₆-alkenyl, C₁₋₃-alkylene, C₁₋₆-alkylene, C₂₋₆-alkenylene, C₂₋₆-alkynylene, C₃₋₈-cycloalkyl, heterocycloalkyl, aryl and heteroaryl groups can in each case be unsubstituted or substituted one or more times by identical or different substituents and the abovementioned C₁₋₆-alkyl, C₂₋₆-alkenyl, C₁₋₃-alkylene, C₁₋₆-alkylene, C₂₋₆-alkenylene and C₂₋₆-alkynylene groups can in each case be branched or unbranched;
   optionally in the form of an individual enantiomer or of an individual diastereomer, of the racemate, of the enantiomers, of the diastereomers, mixtures of the enantiomers and/or diastereomers, and in each case in the form of their bases and/or physiologically acceptable salts.

In the context of the present invention, the term “halogen” preferably represents F, Cl, Br and I, particularly preferably F, Cl and Br.

In the context of this invention, the expression “C₁₋₆-alkyl” includes acyclic saturated hydrocarbon groups having 1, 2, 3, 4, 5 or 6 C atoms, which can be branched- or straight-chain (unbranched) and unsubstituted or substituted one or more times, for example 2, 3, 4 or 5 times, by identical or different substituents. The alkyl groups can preferably be selected from the group consisting of methyl, ethyl, n-propyl, iso-propyl, n-butyl, iso-butyl, sec-butyl, tert-butyl, n-pentyl, iso-pentyl, neo-pentyl and hexyl. Particularly preferred alkyl groups can be selected from the group consisting of methyl, ethyl, n-propyl, iso-propyl, n-butyl, sec-butyl, iso-butyl and tert-butyl.

In the context of this invention, the expression “C₂₋₆-alkenyl” includes acyclic unsaturated hydrocarbon groups having 2, 3, 4, 5 or 6 C atoms, which can be branched or straight-chain (unbranched) and unsubstituted or substituted one or more times, for example 2, 3, 4 or 5 times, by identical or different substituents. In this context, the alkenyl radicals contain at least one C═C double bond. Alkenyl radicals can preferably be selected from the group consisting of vinyl, prop-1-enyl, allyl, 2-methylprop-1-enyl, but-1-enyl, but-2-enyl, but-3-enyl, but-1,3-dienyl, 2-methylprop-1-enyl, but-2-en-2-yl, but-1-en-2-yl, pentenyl and hexenyl. Particularly preferred alkenyl radicals can be selected from the group consisting of vinyl, prop-1-enyl, allyl, 2-methylprop-1-enyl, but-1-enyl, but-2-enyl, but-3-enyl, but-1,3-dienyl, 2-methylprop-1-enyl, but-2-en-2-yl and but-1-en-2-yl.

In the context of this invention, the expression “C₃₋₈-cycloalkyl” denotes cyclic saturated hydrocarbon groups having 3, 4, 5, 6, 7 or 8 carbon atoms, which can be unsubstituted or substituted one or more times, for example by 2, 3, 4 or 5 identical or different substituents, on one or more ring members. C₃₋₈-Cycloalkyl can preferably be selected from the group consisting of cyclopropyl, cyclobutyl, cyclopentyl, cyclohexyl, cycloheptyl and cyclooctyl.

The expression “3- to 8-membered heterocycloalkyl” designates saturated heterocyclic rings which can contain as ring members, selected independently of one another, 1, 2, 3, 4 or 5 identical or different hetero atoms, preferably from the group N, O or S. In the case where the heterocycloalkyl is bonded to a hetero atom, for example N, bonding to the heterocycloalkyl is preferably via one of the carbon ring members of the heterocycloalkyl. 3- to 8-membered heterocycloalkyls can, in particular, be 4-, 5- or 6-membered. Examples of 3- to 8-membered heterocycloalkyls include azetidinyl, pyrrolidinyl, piperidinyl, piperazinyl, morpholinyl, tetrahydropyranyl, dioxanyl and dioxolanyl, which can optionally be substituted as explained below.

In the context of this invention, the expression “aryl” denotes aromatic hydrocarbons, in particular phenyls and naphthyls. The aryl groups can also be condensed with further saturated, (partially) unsaturated or aromatic ring systems. Each aryl group can be unsubstituted or substituted one or more times, for example 2, 3, 4 or 5 times, wherein the substituents on the aryl can be identical or different and can be in any desired and possible position of the aryl. Aryl can advantageously be selected from the group consisting of phenyl, 1-naphthyl and 2-naphthyl, which can in each case be unsubstituted or substituted one or more times, for example by 2, 3, 4 or 5 substituents.

In the context of the present invention, the expression “heteroaryl” represents a 5-, 6- or 7-membered cyclic aromatic group which contains at least 1, optionally also 2, 3, 4 or 5 hetero atoms, wherein the hetero atoms can be identical or different and the heteroaryl can be unsubstituted or substituted one or more times, for example 2, 3, 4 or 5 times, by identical or different substituents. The substituents can be bonded in any desired and possible position of the heteroaryl. The heterocyclic ring can also be part of a bi- or polycyclic, in particular a mono-, bi- or tricyclic system, which can then be more than 7-membered in total, preferably up to 14-membered. Preferred hetero atoms are selected from the group consisting of N, O and S. The heteroaryl group can preferably be selected from the group consisting of pyrrolyl, indolyl, furyl (furanyl), benzofuranyl, thienyl (thiophenyl), benzothienyl, benzothiadiazolyl, benzothiazolyl, benzotriazolyl, benzodioxolanyl, benzodioxanyl, benzoxazolyl, benzoxadiazolyl, imidazothiazolyl, dibenzofuranyl, dibenzothienyl, phthalazinyl, pyrazolyl, imidazolyl, thiazolyl, oxadiazolyl, isoxazoyl, pyridinyl, pyridazinyl, pyrimidinyl, pyrazinyl, pyranyl, indazolyl, purinyl, indolizinyl, quinolinyl, isoquinolinyl, quinazolinyl, quinoxalinyl, carbazolyl, phenazinyl, phenothiazinyl and oxadiazolyl, wherein bonding to the general structure I can be via any desired and possible ring member of the heteroaryl radical. The heteroaryl radical can be particularly preferably selected from the group consisting of furyl, thienyl and pyridinyl.

In the context of the present invention, the expression “C₁₋₃-alkylene group” or “C₁₋₆-alkylene group” includes acyclic saturated hydrocarbon groups having 1, 2 or 3 or, respectively, 1, 2, 3, 4, 5 or 6 C atoms, which can be branched- or straight-chain (unbranched) and unsubstituted or substituted one or more times, for example 2, 3, 4 or 5 times, by identical or different substituents and which link a corresponding group to the main general structure. The alkylene groups can preferably be selected from the group consisting of —CH₂—, —CH₂—CH₂—, —CH(CH₃)—, —CH₂—CH₂—CH₂—, —CH(CH₃)—CH₂—, —CH(CH₂CH₃)—, —CH₂—(CH₂)₂—CH₂—, —CH(CH₃)—CH₂—CH₂—, —CH₂—CH(CH₃)—CH₂—, —CH(CH₃)—CH(CH₃)—, —CH(CH₂CH₃)—CH₂—, —C(CH₃)₂—CH₂—, —CH(CH₂CH₂CH₃)—, —C(CH₃)(CH₂CH₃)—, —CH₂—(CH₂)₃—CH₂—, —CH(CH₃)—CH₂—CH₂—CH₂—, —CH₂—CH(CH₃)—CH₂—CH₂—, —CH(CH₃)—CH₂—CH(CH₃)—, —CH(CH₃)—CH(CH₃)—CH₂—, —C(CH₃)₂—CH₂—CH₂—, —CH₂—C(CH₃)₂—CH₂—, —CH(CH₂CH₃)—CH₂—CH₂—, —CH₂—CH(CH₂CH₃)—CH₂—, —C(CH₃)₂—CH(CH₃)—, —CH(CH₂CH₃)—CH(CH₃)—, —C(CH₃)(CH₂CH₃)—CH₂—, —CH(CH₂CH₂CH₃)—CH₂—, —C(CH₂CH₂CH₃)—CH₂—, —CH(CH₂CH₂CH₂CH₃)—, —C(CH₃)(CH₂CH₂CH₃)—, —C(CH₂CH₃)₂— and —CH₂—(CH₂)₄—CH₂—. The alkylene groups can be particularly preferably selected from the group consisting of —CH₂—, —CH₂—CH₂— and —CH₂—CH₂—CH₂—.

In the context of the present invention, the expression “C₂₋₆-alkenylene group” includes acyclic hydrocarbon groups having 2, 3, 4, 5 or 6 C atoms, which are unsaturated one or more times, for example 2, 3 or 4 times, and can be branched- or straight-chain (unbranched) and unsubstituted or substituted one or more times, for example 2, 3, 4 or 5 times, by identical or different substituents and which link a corresponding group to the main general structure. In this context, the alkenylene groups contain at least one C═C double bond. The alkenylene groups can preferably be selected from the group consisting of —CH═CH—, —CH═CH—CH₂—, —C(CH₃)═CH₂—, —CH═CH—CH₂—CH₂—, —CH₂—CH═CH—CH₂—, —CH═CH—CH═CH—, —C(CH₃)═CH—CH₂—, —CH═C(CH₃)—CH₂—, —C(CH₃)═C(CH₃)—, —C(CH₂CH₃)═CH—, —CH═CH—CH₂—CH₂—CH₂—, —CH₂—CH═CH₂—CH₂—CH₂—, —CH₂—CH═CH—CH₂—CH₂— and —CH═CH₂—CH—CH═CH₂—.

In the context of the invention, the expression “C₂₋₆-alkynylene group” includes acyclic hydrocarbon groups having 2, 3, 4, 5 or 6 C atoms, which are unsaturated one or more times, for example 2, 3 or 4 times, and can be branched- or straight-chain (unbranched) and unsubstituted or substituted one or more times, for example 2, 3, 4 or 5 times, by identical or different substituents and which link a corresponding group to the main general structure. In this context, the alkynylene groups contain at least one C≡C triple bond. The alkynylene groups can preferably be selected from the group consisting of —C≡C—, —C≡C—CH₂—, —C≡C—CH₂—CH₂—, —C≡C—CH(CH₃)—, —CH₂—C≡C—CH₂—, —C≡C—C≡C—, —C≡C—C(CH₃)₂—, —C≡C—CH₂—CH₂—CH₂—, —CH₂—C≡C—CH₂—CH₂—, —C≡C—C≡C—CH₂— and —C≡C—CH₂—C≡C—.

In the context of the present invention, the expression “aryl or heteroaryl bonded via a C₁₋₃-alkylene group, a C₁₋₆-alkylene group, C₂₋₆-alkenylene group or C₂₋₆-alkynylene group” means that the C₁₋₃-alkylene groups, C₁₋₆-alkylene groups, C₂₋₆-alkenylene groups, C₂₋₆-alkynylene groups and aryl or heteroaryl have the meanings defined above and the aryl or heteroaryl is bonded to the main general structure via a C₁₋₃-alkylene group, C₁₋₆-alkylene group, C₂₋₆-alkenylene group or C₂₋₆-alkynylene group. Examples of such groups include benzyl, phenethyl and phenylpropyl.

In the context of the present invention, the expression “C₃₋₈-cycloalkyl and heterocycloalkyl bonded via a C₁₋₃-alkylene group, C₁₋₆-alkylene group, C₂₋₆-alkenylene group or C₂₋₆-alkynylene group” means that the C₁₋₃-alkylene, C₁₋₆-alkylene group, C₂₋₆-alkenylene group, C₂₋₆-alkynylene group, C₃₋₈-cycloalkyl and heterocycloalkyl have the meanings defined above and C₃₋₈-cycloalkyl and heterocycloalkyl are bonded to the main general structure via a C₁₋₃-alkylene group, C₁₋₆-alkylene group, C₂₋₆-alkenylene group or C₂₋₆-alkynylene group.

In connection with “alkyl”, “alkenyl”, “alkylene”, alkenylene”, “alkynylene” and “cycloalkyl”, in the context of this invention the term “substituted” is understood as meaning replacement of a hydrogen by F, Cl, Br, I, CN, NH₂, NH—C₁₋₆-alkyl, NH—C₁₋₆-alkylene-OH, C₁₋₆-alkyl, N(C₁₋₆-alkyl)₂, N(C₁₋₆-alkylene-OH)₂, NO₂, SH, S—C₁₋₆-alkyl, S-benzyl, O—C₁₋₆-alkyl, OH, O—C₁₋₆-alkylene-OH, ═O, O-benzyl, C(═O)C₁₋₆-alkyl, CO₂H, CO₂—C₁₋₆-alkyl or benzyl, where polysubstituted groups are to be understood as meaning those groups which are substituted several times, for example two or three times, either on different or on the same atoms, for example three times on the same carbon atom, as in the case of CF₃ or CH₂CF₃, or at different places, as in the case of CH(Cl)—CH═CH—CHCl₂. Substitution several times can be by identical or different substituents, such as, for example, in the case of CH(OH)—CH═CH—CHCl₂.

With respect to “aryl” and “heteroaryl”, in the context of this invention “substituted” is understood as meaning replacement one or more times, for example 2, 3, 4 or 5 times, of one or more hydrogen atoms on the corresponding ring system by F, Cl, Br, I, CN, NH₂, NH—C₁₋₆-alkyl, NH—C₁₋₆-alkylene-OH, N(C₁₋₆-alkyl)₂, N(C₁₋₆-alkylene-OH)₂, NH-aryl¹, N(aryl¹)₂, N(C₁₋₆-alkyl)aryl¹, pyrrolinyl, piperazinyl, morpholinyl, NO₂, SH, S—C₁₋₆-alkyl, OH, O—C₁₋₆-alkyl, O—C₁₋₆-alkyl-OH, C(═O)C₁₋₆-alkyl, NHSO₂C₁₋₆-alkyl, NHCOC₁₋₆-alkyl, CO₂H, CH₂SO₂-phenyl, CO₂—C₁₋₆-alkyl, OCF₃, CF₃, —O—CH₂—O—, —O—CH₂—CH₂—O—, —O—C(CH₃)₂—CH₂—, unsubstituted C₁₋₆-alkyl, pyrrolidinyl, imidazolyl, piperidinyl, benzyloxy, phenoxy, phenyl, naphthyl, pyridinyl, —C₁₋₃-alkylene-aryl¹, benzyl, thienyl, furyl, wherein aryl¹ represents phenyl, furyl, thienyl or pyridinyl, on one or various atoms, wherein the abovementioned substituents—unless stated otherwise—can optionally be substituted in turn by the aforementioned substituents. Substitution of aryl and heteroaryl several times can be by identical or different substituents. Preferred substituents for aryl and heteroaryl can be selected from the group consisting of —O—C₁₋₃-alkyl, unsubstituted C₁₋₆-alkyl, F, Cl, Br, I, CF₃, OCF₃, OH, SH, phenyl, naphthyl, furyl, thienyl and pyridinyl, in particular from the group consisting of F, Cl, Br, CF₃, CH₃ and OCH₃.

In connection with “3- to 8-membered heterocycloalkyl”, the term “substituted” means replacement of a hydrogen on one or more ring members by F, Cl, Br, I, —CN, NH₂, NH—C₁₋₆-alkyl, NH—C₁₋₆-alkylene-OH, C₁₋₆-alkyl, N(C₁₋₆-alkyl)₂, N(C₁₋₆-alkylene-OH)₂, pyrrolinyl, piperazinyl, morpholinyl, NO₂, SH, S—C₁₋₆-alkyl, S-benzyl, O—C₁₋₆-alkyl, OH, O—C₁₋₆-alkylene-OH, ═O, O-benzyl, C(═O)C₁₋₆-alkyl, CO₂H, CO₂—C₁₋₆-alkyl or benzyl. Substitution several times can be by identical or different substituents. A hydrogen bonded to an N ring member can be replaced by a C₁₋₆-alkyl, C₃₋₈-cycloalkyl, aryl, heteroaryl or a C₃₋₈-cycloalkyl, aryl or heteroaryl bonded via a C₁₋₃-alkylene group, wherein these alkyl, cycloalkyl, alkylene and aryl and heteroaryl groups can be unsubstituted or substituted as defined above. Examples of substituted 3- to 8-membered heterocycloalkyl groups are 1-methylpiperidin-4-yl, 1-phenylpiperidin-4-yl, 1-benzylpiperidin-4-yl, 1-methylpyrrolidin-3-yl, 1-phenylpyrrolidin-3-yl, 1-benzyl-pyrrolin-3-yl, 1-methylazetidin-3-yl, 1-phenyl-azetidin-3-yl or 1-benzylazetidin-3-yl.

In connection with “heterocyclic ring”, in the context of this invention the term “substituted” is understood as meaning replacement of a hydrogen bonded to a carbon ring atom by F, Cl, Br, I, CN, NH₂, NH—C₁₋₆-alkyl, NH—C₁₋₆-alkylene-OH, C₁₋₆-alkyl, N(C₁₋₆-alkyl)₂, N(C₁₋₆-alkylene-OH)₂, NO₂, SH, S—C₁₋₆-alkyl, S-benzyl, O—C₁₋₆-alkyl, OH, O—C₁₋₆-alkylene-OH, ═O, O-benzyl, C(═O)C₁₋₆-alkyl, CO₂H, CO₂—C₁₋₆-alkyl or benzyl. If a heterocyclic ring is substituted several times, the substituents can be on one and/or more carbon ring atoms. In preferred embodiments, one or more hydrogens on one or more carbon ring atoms are exchanged for F.

In connection with the “saturated or at least partly unsaturated ring system” which is fused with the heterocyclic ring formed by R⁴ and R⁵, in the context of this invention the term “substituted” means replacement of a hydrogen bonded to a carbon ring atom by F, Cl, Br, I, CN, NH₂, NH—C₁₋₆-alkyl, NH—C₁₋₆-alkylene-OH, C₁₋₆-alkyl, N(C₁₋₆-alkyl)₂, N(C₁₋₆-alkylene-OH)₂, NO₂, SH, S—C₁₋₆-alkyl, S-benzyl, O—C₁₋₆-alkyl, OH, O—C₁₋₆-alkylene-OH, ═O, O-benzyl, C(═O)C₁₋₆-alkyl, CO₂H, CO₂—C₁₋₆-alkyl or benzyl. If the ring system is substituted several times, the substituents can be on one and/or more carbon ring atoms. In connection with the “aromatic ring system”, which is fused with the heterocyclic ring formed by R⁴ and R⁵, in the context of this invention the term “substituted” in understood as meaning the corresponding substitution as defined for aryl and heteroaryl.

In the context of the present description the symbol

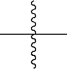

used in the formulas designates a linking of a corresponding group to the particular main general structure.

Those skilled in the art understand that if R² and R³ together with the —N—(CH₂)ₘ—CH— group joining them form a 4-, 5-, 6- or 7-membered heterocyclic ring which has no further hetero atoms and m=0, the following partial structure

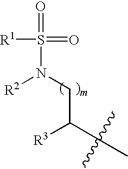

can assume the following forms:

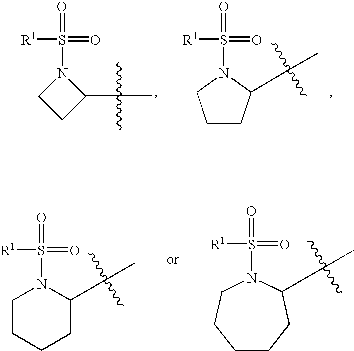

If R² and R³ together with the —N—(CH₂)ₘ—CH— group joining them form a 4-, 5-, 6- or 7-membered heterocyclic ring which has no further hetero atoms and m=1, the following forms result:

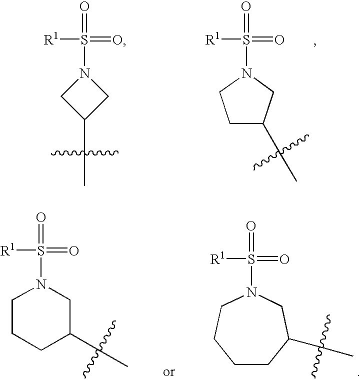

The abovementioned nitrogen-containing heterocyclic rings can furthermore be fused with one or optionally more, in particular with one or two, 5- or 6-membered ring(s). This is illustrated by way of example by the following partial structures:

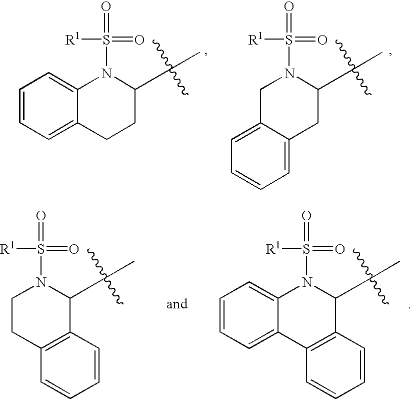

Substituents R² and R³ together with the —N—(CH₂)ₘ—CH— group joining them may also form a 4-, 5-, 6- or 7-membered heterocyclic ring which contains further hetero atoms as stated above. Such a heterocyclic ring may then also be fused with one or optionally more, in particular with one or two, 5- or 6-membered ring(s). This is illustrated by way of example by the following partial structure:

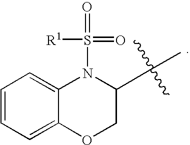

In the context of this invention, the term “physiologically acceptable salt” is understood as meaning preferably salts of the compounds according to the invention with inorganic or organic acids, which are physiologically acceptable—in particular when used on humans and/or mammals. Examples of suitable acids include hydrochloric acid, hydrobromic acid, sulfuric acid, methanesulfonic acid, formic acid, acetic acid, oxalic acid, succinic acid, tartaric acid, mandelic acid, fumaric acid, maleic acid, lactic acid, citric acid, glutamic acid, 1,1-dioxo-1,2-dihydro1λ⁶-benzo[d]isothiazol-3-one (saccharic acid), monomethylsebacic acid, 5-oxo-proline, hexane-1-sulfonic acid, nicotinic acid, 2-, 3- or 4-aminobenzoic acid, 2,4,6-trimethylbenzoic acid, α-liponic acid, acetylglycine, hippuric acid, phosphoric acid and/or aspartic acid. The salts of hydrochloric acid (hydrochlorides) and of citric acid (citrates) are particularly preferred.

In a preferred embodiment of the present invention, in the substituted sulfonamide compounds according to the invention R¹ represents phenyl, naphthyl, Indolyl, benzofuranyl, benzothiophenyl (benzothienyl); benzoxazolyl, benzoxadiazolyl, pyrrolyl, furanyl, thienyl, pyridinyl, pyridazinyl, pyrimidinyl, pyrazinyl, imidazothiazolyl, carbazolyl, dibenzofuranyl, dibenzothiophenyl (dibenzothienyl), benzyl or 2-phenylethyl, preferably phenyl, naphthyl, benzothiophenyl, benzoxadiazolyl, thiophenyl, pyridinyl, imidazothiazolyl or dibenzofuranyl, particularly preferably phenyl or naphthyl, in each case unsubstituted or substituted one or more times by identical or different substituents, wherein the substituents are preferably selected independently of one another from the group consisting of —O—C₁₋₃-alkyl, C₁₋₆-alkyl, —F, —Cl, —Br, —I, —CF₃, —OCF₃, —OH, —SH, phenyl, naphthyl, furyl, thienyl and pyridinyl.

In a further preferred embodiment of the present invention, in the substituted sulfonamide compounds according to the invention R¹ represents phenyl or naphthyl, wherein the phenyl or naphthyl is unsubstituted or substituted one or more times, for example 2, 3, 4 or 5 times, by identical or different substituents selected from the group consisting of methyl, methoxy, CF₃, OCF₃, F, Cl and Br.

In a further preferred embodiment, R¹ in the sulfonamide compounds according to the invention is selected from the group consisting of 4-methoxy-2,3,6-trimethyl-phenyl, 4-methoxy-2,6-dimethylphenyl, 4-methoxy-2,3,5-trimethylphenyl, 2,4,6-trimethylphenyl, 2-chloro-6-methylphenyl, 2,4,6-trichlorophenyl, 2-chloro-6-(trifluoro-methyl)phenyl, 2,6-dichloro-4-methoxyphenyl, 2,4-dichloro-6-methylphenyl, 2-methyl-naphthyl, 2-chloronaphthyl, 2-fluoronaphthyl, 2-chloro-4-(trifluoromethoxy)phenyl, 4-chloro-2,5-dimethylphenyl, 2,3-dichlorophenyl, 2,4-dichlorophenyl, 3,4-dichlorophenyl, 2,6-dichlorophenyl, 2-(trifluoromethyl)phenyl, 3-(trifluoromethyl)phenyl, 4-(trifluoromethyl)phenyl, 2-methoxyphenyl, 3-methoxyphenyl, 4-methoxyphenyl, 1-naphthyl and 2-naphthyl.

In a further preferred embodiment, R¹ in the sulfonamide compounds according to the invention is selected from the group consisting of 3,4-dichlorophenyl, 4-methoxyphenyl, 4-methoxy-2,6-dimethylphenyl, 4-methoxy-2,3,6-trimethylphenyl, 2.6-dichlorophenyl, 2,4-dichlorophenyl, 2,4,6-trichlorophenyl, 2-chloro-6-methylphenyl, 2,4,6-trimethylphenyl, 2-(trifluoromethyl)phenyl, 3-(trifluoromethyl)phenyl, 1-naphthyl, 2-naphthyl, 2,4-dichloro-6-methylphenyl and 4-chloro-2,5-dimethylphenyl, more preferably R¹ is selected from the group consisting of 3,4-dichlorophenyl, 4-methoxyphenyl, 4-methoxy-2,6-dimethylphenyl, 4-methoxy-2,3,6-trimethylphenyl, 2.6-dichlorophenyl, 2,4-dichlorophenyl, 2,4,6-trichlorophenyl, 2,4,6-trimethylphenyl, 3-(trifluoromethyl)phenyl, 2-naphthyl, 2,4-dichloro-6-methylphenyl and 4-chloro-2,5-dimethylphenyl.

In a further preferred embodiment, R¹ in the sulfonamide compounds according to the invention is 4-methoxy-2,6-dimethylphenyl.

In a further preferred embodiment of the present invention, in the substituted sulfonamide compounds according to the invention R² represents H, C₁₋₆-alkyl, C₃₋₆-cycloalkyl or aryl; or a C₃₋₆-cycloalkyl or aryl bonded via a C₁₋₆-alkylene group, C₂₋₆-alkenylene group or C₂₋₆-alkynylene group, wherein the radicals C₁₋₆-alkyl, C₃₋₆-cycloalkyl, C₁₋₆-alkylene, C₂₋₆-alkenylene, C₂₋₆-alkynylene and aryl are in each case unsubstituted or substituted one or more times, wherein aryl in particular is substituted one or more times by identical or different substituents which are selected independently of one another from the group consisting of C₁₋₆-alkyl, C₁₋₆-alkyl-O—, F, Cl, Br, I, CF₃, OCF₃, OH and SH.

In a further preferred embodiment of the present invention, in the substituted sulfonamide compounds according to the invention R² represents H, C₁₋₆-alkyl, cyclopropyl or phenyl; or a phenyl bonded via a C₁₋₆-alkylene group, wherein the phenyl is each case unsubstituted or substituted one or more times by identical or different substituents, wherein the substituents are selected independently of one another from the group consisting of methyl, ethyl, n-propyl, iso-propyl, n-butyl, iso-butyl, sec-butyl, tert-butyl, methoxy, F, Cl, Br, I, CF₃, OCF₃ and OH.

In a further preferred embodiment of the present invention, in the substituted sulfonamide compounds according to the invention R² represents H, methyl, ethyl, n-propyl, iso-propyl, n-butyl, iso-butyl, sec-butyl, tert-butyl, phenyl or benzyl. Preferably R² represents H, methyl, ethyl, n-propyl, iso-propyl, n-butyl, iso-butyl, sec-butyl or tert-butyl.

In a further preferred embodiment of the present invention, in the substituted sulfonamide compounds according to the invention R² represents H, methyl, ethyl, phenyl or benzyl. Preferably R² represents H, methyl or ethyl.

Preferably, R³ in the sulfonamide compounds according to the invention can represent H, C₁₋₆-alkyl or aryl; wherein the groups C₁₋₆-alkyl and aryl are in each case unsubstituted or substituted one or more times, wherein the aryl in particular is unsubstituted or substituted one or more times by identical or different substituents selected independently of one another from the group consisting of C₁₋₆-alkyl, C₁₋₆-alkyl-O—, F, Cl, Br, I, CF₃, OCF₃, OH and SH.

In a further preferred embodiment of the sulfonamide compounds according to the invention, R³ represents H or phenyl, wherein the phenyl is each case unsubstituted or substituted one or more times by identical or different substituents, wherein the substituents are selected independently of one another from the group consisting of methyl, ethyl, n-propyl, iso-propyl, n-butyl, iso-butyl, sec-butyl, tert-butyl, methoxy, F, Cl, Br, I, CF₃, OCF₃ and OH.

In a further preferred embodiment of the sulfonamide compounds according to the invention, R³ represents H or unsubstituted phenyl.

In a further preferred embodiment of the sulfonamide compounds according to the invention, R² and R³ together with the —N—(CH₂)ₘ—CH— group joining them form a 4-, 5-, 6- or 7-membered, preferably 5-, 6- or 7-membered heterocyclic ring, which can be fused with one or two 6-membered aromatic ring(s) (benzo group), wherein the heterocyclic ring is saturated or at least monounsaturated, but not aromatic, and can contain, in addition to the N hetero atom to which R² is bonded, at least one oxygen atom.

In yet a further preferred embodiment of the sulfonamide compounds according to the invention, R² and R³ together with the —N—(CH₂)ₘ—CH— group joining them form a 4-, 5-, 6- or 7-membered, preferably 5-, 6- or 7-membered heterocyclic ring, which can be fused with one or two 6-membered aromatic ring(s) (benzo group).

In a further preferred embodiment of the sulfonamide compounds according to the invention, R² and R³ together with the —N—(CH₂)ₘ—CH— group joining them form a 5- or 6-membered heterocyclic ring which can be fused with a 6-membered aromatic ring (benzo group), wherein the heterocyclic ring is saturated or at least monounsaturated, but not aromatic, and can contain, in addition to the N hetero atom to which R² is bonded, at least one oxygen atom.

In yet a further preferred embodiment of the sulfonamide compounds according to the invention, R² and R³ together with the —N—(CH₂)ₘ—CH— group joining them form a 5- or 6-membered heterocyclic ring which can be fused with a 6-membered aromatic ring (benzo group).

In a further preferred embodiment of the sulfonamide compounds according to the invention, A represents a single bond and X represents N, or A represents a group selected from the group consisting of —N(R⁷)—, —N(R⁷)—(CH₂)—, N(R⁷)—(CH₂)₂— and N(R⁷)—(CH₂)₃— and X represents CH. Preferably, in the cases where A represents a nitrogen-containing group, this is in each case linked to the adjacent carbonyl group via the nitrogen atom.

In a further preferred embodiment of the sulfonamide compounds according to the invention, R⁴ and R⁵ each independently represent H, or substituted or unsubstituted C₁₋₆-alkyl; or the group —NR⁴R⁵ represents a heterocylic ring corresponding to the following formula IIa:

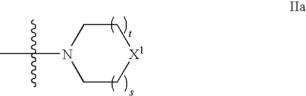

wherein
X¹ represents O, S, NR¹², CH₂ or C(halogen)₂, wherein R¹² represents H; C₁₋₆-alkyl, in particular methyl, ethyl, n-propyl, iso-propyl, n-butyl, iso-butyl, sec-butyl, tert-butyl, or aryl, preferably phenyl or naphthyl; or heteroaryl, preferably a 5- to 6-membered heteroaryl having 1 or 2 N hetero atoms, in particular 2-, 3- or 4-pyridinyl; or R¹² represents an aryl, preferably phenyl or naphthyl, bonded via a C₁₋₃-alkylene group; or a heteroaryl, preferably a 5- to 6-membered heteroaryl having 1 or 2 N hetero atoms, in particular 2-, 3- or 4-pyridinyl, bonded via a C₁₋₃-alkylene group. In the group C(halogen)₂, halogen preferably represents F, Cl, Br or I, particularly preferably F. In the structure according to the general formula IIa, s and t each independently represent 0, 1 or 2, with the proviso that s+t=0, 1, 2 or 3. Preferably, s and t are each not 0 if X¹ represent the group NR¹². The radicals C₁₋₆-alkyl, C₁₋₃-alkylene, aryl and heteroaryl mentioned above in connection with R¹² can in each case be unsubstituted or substituted one or more times by identical or different substituents. For example, the aryl or heteroaryl can in each case be unsubstituted or substituted one or more times, for example 2, 3, 4 or 5 times, by identical or different substituents which are selected independently of one another from the group consisting of O—C₁₋₃-alkyl, unsubstituted C₁₋₆-alkyl, F, Cl, Br, I, CF₃, OCF₃, OH and SH.

In particular, the ring corresponding to formula IIa can be selected from the group consisting of:

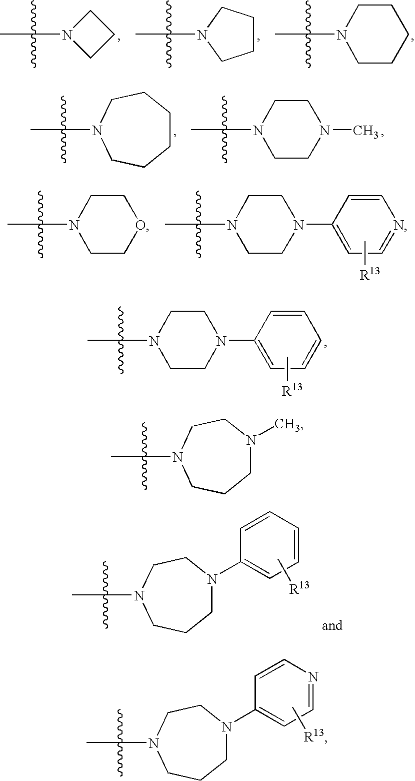

wherein R¹³ in each case represents one or more, optionally 1, 2, 3, 4 or 5 substituents which can be selected independently of one another from the group consisting of H, F and Cl.

The group —NR⁴R⁵ in the substituted sulfonamide compounds according to the invention can furthermore represent a ring corresponding to the following formula lib:

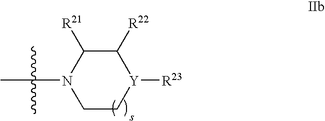

wherein s can be 0 or 1, Y represents CH or N, under the condition that if s=0, Y does not represent N, and two adjacent groups R²¹, R²² and R²³ together form a fused-on group corresponding to any of the following partial formulas:

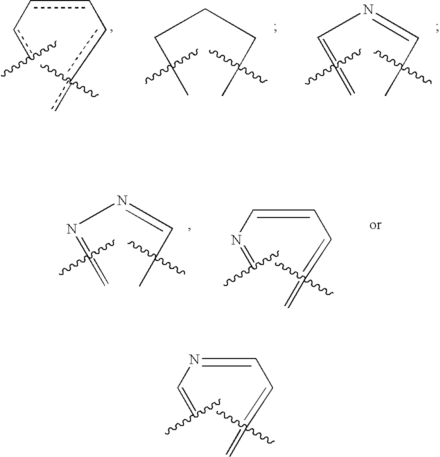

and the respective third group from R²¹, R²² and R²³ denotes H, and [symbol] denotes a single or double bond.

Those skilled in the art furthermore will understand that if two adjacent groups from R²¹, R²² and R²³ form a fused-on ring which is aromatic, the two carbon atoms to which these two adjacent groups are bonded can no longer carry a hydrogen.

For example, —NR⁴R⁵ can represent one of the following groups:

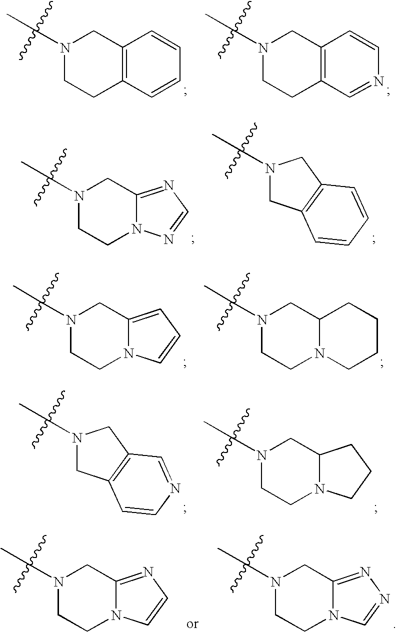

In a further preferred embodiment of the sulfonamide compounds according to the invention, R⁴ and R⁵ each independently represent H, or C₁₋₆-alkyl, in particular H, methyl, ethyl, n-propyl, iso-propyl, n-butyl, iso-butyl, sec-butyl or tert-butyl, or the group —NR⁴R⁵ represents a heterocylic ring corresponding to the following formula IIa:

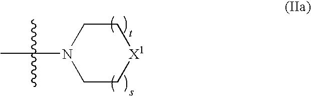

wherein
X¹ represents O, S, NR¹², CH₂ or C(halogen)₂, wherein halogen preferably denotes F, Cl or Br, R¹² represents H; C₁₋₆-alkyl, phenyl, naphthyl or pyridinyl;
s and t each independently represent 0, 1 or 2, with the proviso that s+t=0, 1, 2 or 3,
   wherein if X¹ denotes O, S or NR¹², s and t preferably each represent 1.

In a further preferred embodiment of the sulfonamide compounds according to the invention, R⁴ and R⁵ each independently represent a group selected from the group consisting of H, methyl, ethyl, n-propyl, iso-propyl, n-butyl, iso-butyl, sec-butyl and tert-butyl, preferably each represent H or methyl, or R⁴ and R⁵ together with the nitrogen atom joining them form a heterocyclic ring which is selected from the group consisting of

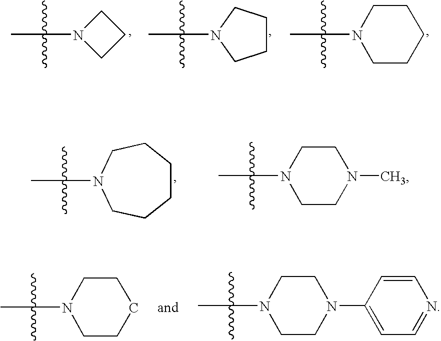

In a further preferred embodiment of the sulfonamide compounds according to the invention, R⁶ represents phenyl, naphthyl, furyl, thienyl or pyridinyl or a phenyl, naphthyl, furyl, thienyl or pyridinyl bonded via a C₁₋₃-alkylene group, wherein the phenyl, naphthyl, furyl, thienyl and pyridinyl are in each case unsubstituted or substituted one or more times by identical or different substituents selected independently of one another from the group consisting of C₁₋₄-alkyl, O—C₁₋₄-alkyl, F, Cl, Br, I, CF₃, OCF₃, OH, —NO₂ and —CN.

In a further preferred embodiment of the sulfonamide compounds according to the invention, R⁶ represents phenyl or pyridinyl or a phenyl or pyridinyl bonded via —(CH₂)—, —(CH₂)₂— or —(CH₂)₃—, wherein the phenyl or pyridinyl is in each case unsubstituted or substituted one or more times by identical or different substituents selected independently of one another from the group consisting of methyl, ethyl, methoxy, ethoxy, F, Cl, Br, I, CN, CF₃, OCF₃ and OH.

In a further preferred embodiment of the sulfonamide compounds according to the invention, R⁷ represents a group selected from the group consisting of H, methyl, ethyl, n-propyl, iso-propyl, n-butyl, iso-butyl, sec-butyl and tert-butyl, preferably H or methyl.

In a further preferred embodiment of the sulfonamide compounds according to the invention, n, p and Q in the partial structure

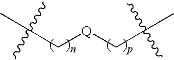

are selected such that this partial structure is selected from the group consisting of a single bond, —(CH₂)—; —(CH₂)₂—; —(CH₂)₃—; —(CH₂)—O—(CH₂)—; —(CH₂)₂—O—(CH₂); —(CH₂)—O—(CH₂)₂; —(CH₂)₂—O—(CH₂)₂; —O—(CH₂) and —(CH₂)—O—, preferably from the group consisting of a single bond, —(CH₂)—; —(CH₂)₂—; —(CH₂)—O—(CH₂)—; —(CH₂)₂—O—(CH₂); —(CH₂)—O—(CH₂)₂; —(CH₂)₂—O—(CH₂)₂; —O—(CH₂) and —(CH₂)—O—.

In a further preferred embodiment of the sulfonamide compounds according to the invention, u and v each independently represent 0, 1, 2 or 3, with the proviso that u+v=2 or 3.

In a further preferred embodiment of the sulfonamide compounds according to the invention, u=1 and v=1 or u=0 and v=2 or u=1 and v=2.

In a further preferred embodiment of the sulfonamide compounds according to the invention, m represents 0 if R² and R³ are defined as under (i).

Substituted sulfonamide compounds corresponding to formula I according to the invention which are likewise preferred are those wherein
m represents 0 or 1;
n and p each independently represent 0, 1 or 2;
u and v each independently represent 0, 1, 2, 3 or 4, with the proviso that u+v=1, 2, 3 or 4;
Q represents a single bond, —CH₂— or —O—;
R¹ represents phenyl, naphthyl, indolyl, benzofuranyl, benzothiophenyl (benzothienyl); benzoxazolyl, benzoxadiazolyl, pyrrolyl, furanyl, thienyl, pyridinyl, pyridazinyl, pyrimidinyl, pyrazinyl, imidazothiazolyl, carbazolyl, dibenzofuranyl or dibenzothiophenyl (dibenzothienyl), in each case unsubstituted or substituted one or more times, wherein the substituents are selected independently of one another from the group consisting of —O—C₁₋₃-alkyl, C₁₋₆-alkyl, —F, —Cl, —Br, —I, —CF₃, —OCF₃, —OH, —SH, phenyl, naphthyl, furyl, thienyl and pyridinyl;
R² represents H, C₁₋₄-alkyl, phenyl or benzyl; preferably R² represents H or C₁₋₄-alkyl;
R³ represents H, C₁₋₆-alkyl or aryl; or denotes an aryl bonded via a C₁₋₆-alkylene group, wherein the aryl is in each case unsubstituted or substituted one or more times by identical or different substituents, wherein the substituents are selected independently of one another from the group consisting of C₁₋₆-alkyl, C₁₋₆-alkyl-O—, F, Cl, Br, I, CF₃, OCF₃, OH and SH; or
R² and R³ together with the —N—(CH₂)ₘ—CH— group joining them form a 4-, 5-, 6- or 7-membered heterocyclic ring, which can be fused with one or two 6-membered aromatic ring(s) (benzo group); wherein the heterocyclic ring is saturated or at least monounsaturated, but not aromatic, and can contain, in addition to the N hetero atom to which R² is bonded, at least one oxygen atom, preferably R² and R³ together with the —N—(CH₂)ₘ—CH— group joining them form a 4-, 5-, 6- or 7-membered heterocyclic ring, which can be fused with one or two 6-membered aromatic ring(s) (benzo group);
A represents a single bond and X represents N, or
A represents —N(R⁷)—(CH₂)_{0, 1, 2 or 3}— and X represents CH;
R⁴ and R⁵ each independently represent H or C₁₋₆-alkyl, or the group —NR⁴R⁵ represents a heterocylic ring corresponding to the formula IIa:

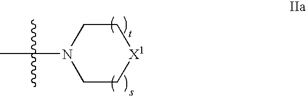

wherein
X¹ represents O, S, NR¹², CH₂ or C(halogen)₂, wherein halogen preferably denotes F, Cl or Br, and R¹² represents H; C₁₋₆-alkyl, phenyl, naphthyl or pyridinyl;
s and t each independently represent 0, 1 or 2, with the proviso that s+t=0, 1, 2 or 3,
wherein if X¹ denotes O, S or NR¹², s and t preferably each represent 1;
R⁶ represents phenyl, naphthyl, furyl, thienyl and pyridinyl or a phenyl, naphthyl, furyl, thienyl and pyridinyl bonded via a C₁₋₃-alkylene group, wherein the phenyl, naphthyl, furyl, thienyl and pyridinyl are in each case unsubstituted or substituted one or more times by identical or different substituents selected independently of one another from the group consisting of C₁₋₄-alkyl, O—C₁₋₄-alkyl, F, Cl, Br, I, CF₃, OCF₃, OH, —NO₂ and —CN;
R⁷ represents H, methyl, ethyl, n-propyl, iso-propyl, n-butyl, iso-butyl, sec-butyl, tert-butyl or cyclopropyl;
   optionally in the form of an individual enantiomer or of an individual diastereomer, of the racemate, of the enantiomers, of the diastereomers, mixtures of the enantiomers and/or diastereomers, and in each case in the form of their bases and/or physiologically acceptable salts.

Substituted sulfonamide compounds corresponding to formula I according to the invention which are likewise preferred are those wherein
m represents 0 or 1;
n and p each independently represent 0, 1 or 2;
u and v each independently represent 0, 1, 2, 3 or 4, with the proviso that u+v=1, 2, 3 or 4;
Q represents a single bond, —CH₂— or —O—;
R¹ represents phenyl or naphthyl, in each case unsubstituted or substituted one or more times by identical or different substituents, wherein the substituents are selected independently of one another from the group consisting of methyl, methoxy, CF₃, F, Cl and Br;
R² represents H, methyl, ethyl, n-propyl, iso-propyl, n-butyl, iso-butyl, sec-butyl, tert-butyl, phenyl or benzyl, preferably R² represents H, methyl, ethyl, n-propyl, iso-propyl, n-butyl, iso-butyl, sec-butyl or tert-butyl, R³ represents H or phenyl, or
R² and R³ together with the —N—(CH₂)ₘ—CH— group joining them form a 5-, 6- or 7-membered heterocyclic ring, which can be fused with one or two 6-membered aromatic ring(s) (benzo group); wherein the heterocyclic ring is saturated or at least monounsaturated, but not aromatic, and can contain, in addition to the N hetero atom to which R² is bonded, at least one oxygen atom, preferably R² and R³ together with the —N—(CH₂)ₘ—CH— group joining them form a 5-, 6- or 7-membered heterocyclic ring, which can be fused with one or two 6-membered aromatic ring(s) (benzo group);
A represents a single bond and X represents N, or
A represents —N(R⁷)—(CH₂)_{0, 1, 2 or 3}— and X represents CH;
R⁴ and R⁵ each independently represent H or C₁₋₆-alkyl, or
the group —NR⁴R⁵ represents a heterocylic ring corresponding to formula IIa:

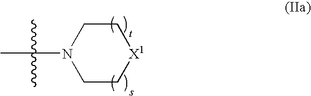

wherein
X¹ represents O, S, NR¹², CH₂ or C(halogen)₂, wherein halogen preferably denotes F, Cl or Br, R¹² represents H; C₁₋₆-alkyl, phenyl, naphthyl or pyridinyl;
s and t each independently represent 0, 1 or 2, with the proviso that s+t=0, 1, 2 or 3,
wherein if X¹ denotes O, S or NR¹², s and t preferably each represent 1;
R⁶ represents phenyl, naphthyl, furyl, thienyl or pyridinyl or a phenyl, naphthyl, furyl, thienyl or pyridinyl bonded via a C₁₋₃-alkylene group, wherein the phenyl, naphthyl, furyl, thienyl and pyridinyl are in each case unsubstituted or substituted one or more times by identical or different substituents selected independently of one another from the group consisting of C₁₋₄-alkyl, O—C₁₋₄-alkyl, F, Cl, Br, I, CF₃, OCF₃, OH, —NO₂ and —CN;
R⁷ represents H, methyl, ethyl, n-propyl, iso-propyl, n-butyl, iso-butyl, sec-butyl, tert-butyl or cyclopropyl;
   optionally in the form of an individual enantiomer or of an individual diastereomer, of the racemate, of the enantiomers, of the diastereomers, mixtures of the enantiomers and/or diastereomers, and in each case in the form of their bases and/or physiologically acceptable salts.

Substituted sulfonamide compounds corresponding to formula I according to the invention which are also preferred are those wherein
m represents 0 or 1;
n and p each independently represent 0, 1 or 2;
u and v each independently represent 0, 1, 2, 3 or 4, with the proviso that u+v=1, 2, 3 or 4;
Q represents a single bond, —CH₂— or —O—;
R¹ represents 3,4-dichlorophenyl, 4-methoxyphenyl, 4-methoxy-2,6-dimethylphenyl, 4-methoxy-2,3,6-trimethylphenyl, 2.6-dichlorophenyl, 2,4-dichlorophenyl, 2,4,6-trichlorophenyl, 2-chloro-6-methylphenyl, 2,4,6-trimethylphenyl, 2-(trifluoromethyl)-phenyl, 3-(trifluoromethyl)phenyl, 1-naphthyl, 2-naphthyl, 2,4-dichloro-6-methylphenyl or 4-chloro-2,5-dimethylphenyl; preferably R¹ represents 3,4-dichlorophenyl, 4-methoxyphenyl, 4-methoxy-2,6-dimethylphenyl, 4-methoxy-2,3,6-trimethylphenyl, 2.6-dichlorophenyl, 2,4-dichlorophenyl, 2,4,6-trichlorophenyl, 2,4,6-trimethylphenyl, 3-(trifluoromethyl)phenyl, 2-naphthyl, 2,4-dichloro-6-methylphenyl or 4-chloro-2,5-dimethylphenyl;
R² represents H, methyl, ethyl, phenyl or benzyl, preferably R² represents H, methyl or ethyl;
R³ represents H or phenyl, or
R² and R³ together with the —N—(CH₂)ₘ—CH— group joining them form a 5- or 6-membered heterocyclic ring, which can be fused with a 6-membered aromatic ring (benzo group); wherein the heterocyclic ring is saturated or at least monounsaturated, but not aromatic, and can contain, in addition to the N hetero atom to which R² is bonded, at least one oxygen atom, preferably R² and R³ together with the —N—(CH₂)ₘ—CH— group joining them form a 5- or 6-membered heterocyclic ring, which can be fused with a 6-membered aromatic ring (benzo group);
A represents a single bond and X represents N, or
A represents —N(R⁷)—(CH₂)_{0, 1, 2 or 3}— and X represents CH;
R⁴ and R⁵ each independently represent H or methyl, or
R⁴ and R⁵ together with the nitrogen atom joining them form a heterocyclic ring which is selected from the group consisting of

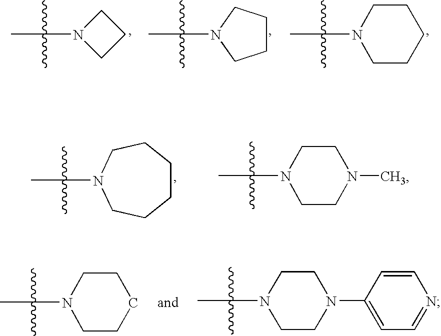

R⁶ represents phenyl or pyridinyl or a phenyl or pyridinyl bonded via —(CH₂)—, —(CH₂)₂— or —(CH₂)₃—, wherein the phenyl or pyridinyl is in each case unsubstituted or substituted one or more times by identical or different substituents selected independently of one another from the group consisting of methyl, ethyl, methoxy, ethoxy, F, Cl, Br, I, CN, CF₃, OCF₃ and OH;
R⁷ represents H, methyl or cyclopropyl,
   optionally in the form of an individual enantiomer or of an individual diastereomer, of the racemate, of the enantiomers, of the diastereomers, mixtures of the enantiomers and/or diastereomers, and in each case in the form of their bases and/or physiologically acceptable salts.

Compounds of the following general formulas Ib, Ic, Id, Ie, If, Ig, Ih and Io are also particularly preferred:

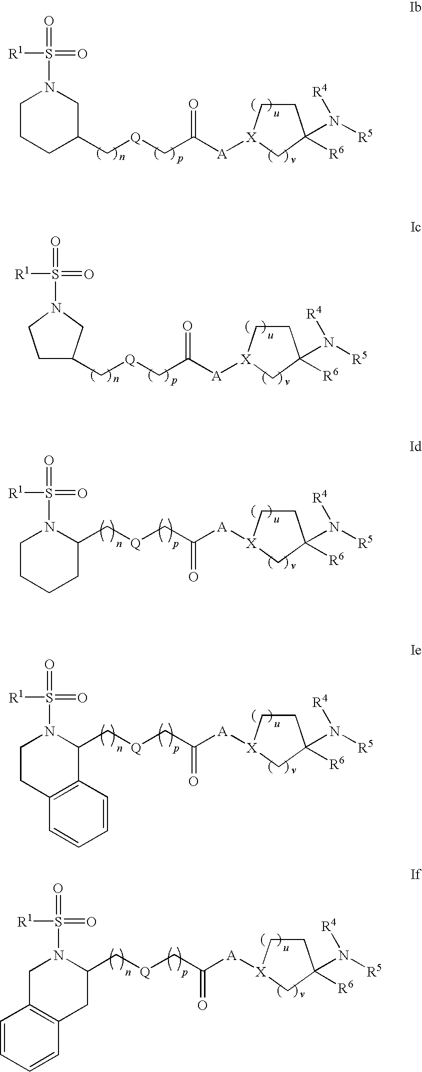

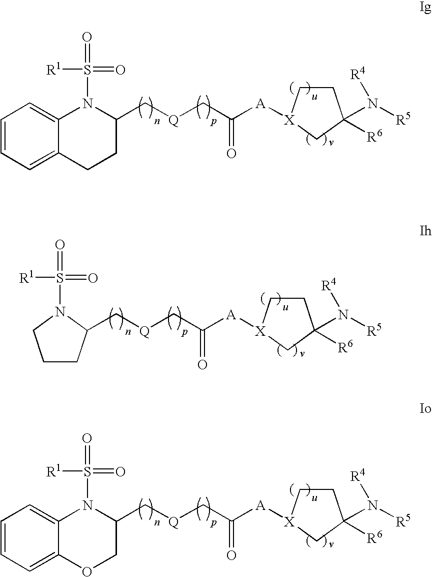

wherein R¹, n, Q, p, A, X, u, v, R⁴, R⁵ and R⁶ each have one of the meanings described herein.

Compounds corresponding to the following formulas Ii and Ij are also particularly preferred:

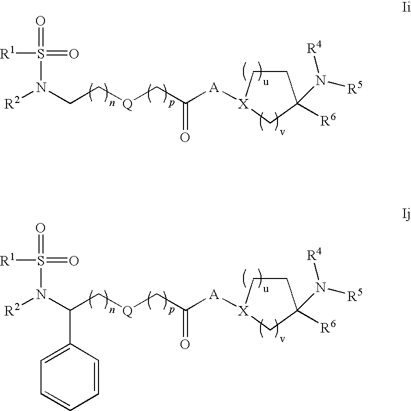

wherein R¹, R², n, Q, p, A, X, u, v, R⁴, R⁵ and R⁶ each have one of the meanings described herein.

Compounds corresponding to the following formulas Ik and Il are also particularly preferred:

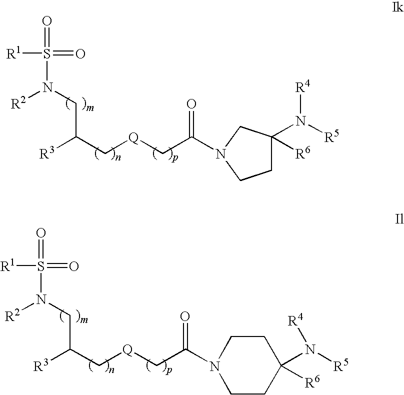

wherein R¹, R², R³, m, n, Q, p, R⁴, R⁵ and R⁶ each have one of the meanings described herein.

Compounds of the following general formula Im are also particularly preferred

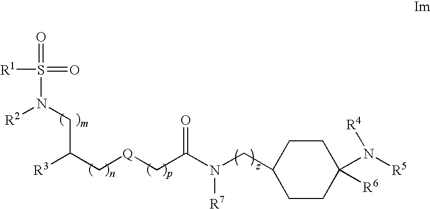

wherein z represents 0, 1, 2 or 3 and
R¹, R², R³, m, n, Q, p, R⁴, R⁵, R⁶ and R⁷ each have one of the meanings described herein.

Substituted sulfonamide compounds according to the invention which are particularly preferred are those corresponding to the following formula Ia:

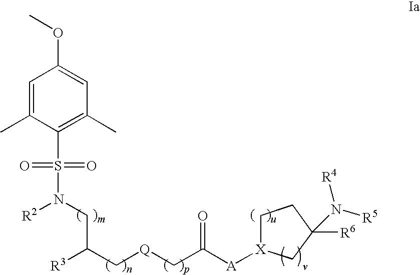

wherein R², R³, m, n, Q, p, A, X, u, v, R⁴, R⁵ and R⁶ each have one of the meanings described herein.

Substituted sulfonamide compounds corresponding to formula Ia according to the invention which are also particularly preferred are those wherein
m represents 0 or 1;
n and p each independently represent 0, 1 or 2;
u and v each independently represent 0, 1, 2, 3 or 4, with the proviso that u+v=1, 2, 3 or 4;
Q represents a single bond, —CH₂— or —O—;
R² represents H, methyl, ethyl, n-propyl, iso-propyl, n-butyl, iso-butyl, sec-butyl, tert-butyl, cyclopropyl, phenyl or benzyl, preferably R² represents H, methyl, ethyl, n-propyl, iso-propyl, n-butyl, iso-butyl, sec-butyl, tert-butyl or cyclopropyl,
R³ represents H or phenyl, or
R² and R³ together with the —N—(CH₂)ₘ—CH— group joining them form a 4-, 5-, 6- or 7-membered heterocyclic ring, which can be fused with one or two 6-membered aromatic ring(s) (benzo group); wherein the heterocyclic ring is saturated or at least monounsaturated, but not aromatic, and can contain, in addition to the N hetero atom to which R² is bonded, at least one oxygen atom, preferably R² and R³ together with the —N—(CH₂)ₘ—CH— group joining them form a 4-, 5-, 6- or 7-membered heterocyclic ring, which can be fused with one or two 6-membered aromatic ring(s) (benzo group);
A represents a single bond and X represents N, or
A represents —N(R⁷)—(CH₂)_{0, 1, 2 or 3}— and X represents CH;
R⁴ and R⁵ each independently represent a group selected from the group consisting of H, methyl, ethyl, n-propyl, iso-propyl, n-butyl, iso-butyl, sec-butyl and tert-butyl—alkyl, or
the group —NR⁴R⁵ represents a heterocylic ring corresponding to the formula IIa:

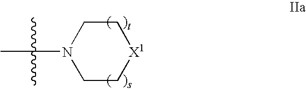

wherein
X¹ represents O, S, NR¹², CH₂ or C(halogen)₂, wherein halogen preferably denotes F, Cl or Br, R¹² represents H; C₁₋₆-alkyl, phenyl, naphthyl or pyridinyl;
s and t each independently represent 0, 1 or 2, with the proviso that s+t=0, 1, 2 or 3,
wherein if X¹ denotes O, S or NR¹², s and t preferably each represent 1;
R⁶ represents phenyl, naphthyl, furyl, thienyl or pyridinyl or a phenyl, naphthyl, furyl, thienyl or pyridinyl bonded via a C₁₋₃-alkylene group, wherein the phenyl, naphthyl, furyl, thienyl and pyridinyl are in each case unsubstituted or substituted one or more times by identical or different substituents selected independently of one another from the group consisting of C₁₋₄-alkyl, O—C₁₋₄-alkyl, F, Cl, Br, I, CF₃, OCF₃, OH, —NO₂ and —CN;
R⁷ represents H, methyl, ethyl, n-propyl, iso-propyl, n-butyl, iso-butyl, sec-butyl, tert-butyl or cyclopropyl;
   optionally in the form of an individual enantiomer or of an individual diastereomer, of the racemate, of the enantiomers, of the diastereomers, mixtures of the enantiomers and/or diastereomers, and in each case in the form of their bases and/or physiologically acceptable salts.

Sulfonamide compounds according to the invention which are very particularly preferred are selected from the group consisting of:
(1) 2-(2-(3,4-dichlorophenylsulfonyl)-1,2,3,4-tetrahydroisoquinolin-1-yl)-N-(4-(dimethylamino)-4-phenethylcyclohexyl)acetamide
(2) N-(4-(dimethylamino)-4-phenethylcyclohexyl)-2-((1-(4-methoxyphenylsulfonyl)piperidin-2-yl)methoxy)acetamide
(3) N-(4-(dimethylamino)-4-phenethylcyclohexyl)-2-(2-(4-methoxyphenylsulfonyl)-1,2,3,4-tetrahydroisoquinolin-1-yl)acetamide
(4) N-(4-(dimethylamino)-4-(2-methylbenzyl)cyclohexyl)-2-((1-(4-methoxyphenylsulfonyl)piperidin-2-yl)methoxy)acetamide
(5) N-(4-(dimethylamino)-4-phenethylcyclohexyl)-2-(1-(4-methoxy-2,6-dimethylphenylsulfonyl)pyrrolidin-3-yloxy)acetamide
(6) N-(4-(dimethylamino)-4-(3-fluorophenyl)cyclohexyl)-2-(2-(4-methoxy-N,2,6-trimethylphenylsulfonamido)ethoxy)acetamide
(7) N-(4-(dimethylamino)-4-phenethylcyclohexyl)-2-(2-(4-methoxy-N,2,6-trimethylphenylsulfonamido)ethoxy)acetamide
(8) N-(4-(dimethylamino)-4-phenethylcyclohexyl)-2-(2-(N-ethyl-4-methoxy-2,3,6-trimethylphenylsulfonamido)ethoxy)acetamide
(9) N-(4-(dimethylamino)-4-(4-fluorobenzyl)cyclohexyl)-2-(2-(4-methoxy-N,2,6-trimethylphenylsulfonamido)ethoxy)acetamide
(10) N-(4-(dimethylamino)-4-(2-methylbenzyl)cyclohexyl)-2-(2-(4-methoxy-N,2,6-trimethylphenylsulfonamido)ethoxy)acetamide
(11) 2-(2-(4-methoxy-N,2,6-trimethylphenylsulfonamido)ethoxy)-N-(4-phenyl-4-(piperidin-1-yl)cyclohexyl)acetamide
(12) 2-(2-(3,4-dichlorophenylsulfonyl)-1,2,3,4-tetrahydroisoquinolin-1-yl)-N-(4-(dimethylamino)-4-(2-methylbenzyl)cyclohexyl)acetamide
(13) 2-(2-(2,6-dichloro-N-methylphenylsulfonamido)ethoxy)-N-(4-(dimethylamino)-4-phenethylcyclohexyl)acetamide
(14) N-(4-(dimethylamino)-4-(2-methylbenzyl)cyclohexyl)-2-(1-(4-methoxy-2,6-dimethylphenylsulfonyl)pyrrolidin-3-yloxy)acetamide
(15) N-(4-benzyl-4-(piperidin-1-yl)cyclohexyl)-2-(2-(3,4-dichlorophenylsulfonyl)-1,2,3,4-tetrahydroisoquinolin-1-yl)acetamide
(16) N-(4-(azepan-1-yl)-4-benzylcyclohexyl)-2-(2-(3,4-dichlorophenylsulfonyl)-1,2,3,4-tetrahydroisoquinolin-1-yl)acetamide
(17) N-(4-benzyl-4-(piperidin-1-yl)cyclohexyl)-2-(2-(4-methoxy-N,2,6-trimethylphenylsulfonamido)ethoxy)acetamide
(18) N-(4-benzyl-4-(piperidin-1-yl)cyclohexyl)-2-(1-(4-methoxy-2,6-dimethylphenylsulfonyl)pyrrolidin-3-yloxy)acetamide
(19) N-(4-(dimethylamino)-4-phenylcyclohexyl)-2-(2-(1-(4-methoxyphenylsulfonyl)piperidin-2-yl)ethoxy)acetamide
(20) N-(4-(azepan-1-yl)-4-benzylcyclohexyl)-2-(2-(4-methoxy-N,2,6-trimethylphenylsulfonamido)ethoxy)acetamide
(21) 2-(2-(2,4-dichloro-N-methylphenylsulfonamido)ethoxy)-N-(4-(dimethylamino)-4-(3-fluorophenyl)cyclohexyl)acetamide
(22) 2-(2-(2,4-dichloro-N-methylphenylsulfonamido)ethoxy)-N-(4-(dimethylamino)-4-phenethylcyclohexyl)acetamide
(23) N-(4-(dimethylamino)-4-phenethylcyclohexyl)-2-(2-(2,4,6-trichloro-N-methylphenylsulfonamido)ethoxy)acetamide
(24) N-(4-(dimethylamino)-4-phenethylcyclohexyl)-2-(2-(4-methoxy-N,2,3,6-tetramethylphenylsulfonamido)ethoxy)acetamide
(25) N-(4-(dimethylamino)-4-phenethylcyclohexyl)-2-(2-(N,2,4,6-tetramethylphenylsulfonamido)ethoxy)acetamide
(26) 2-(2-(3,4-dichlorophenylsulfonyl)-1,2,3,4-tetrahydroisoquinolin-1-yl)-N-(4-(dimethylamino)-4-phenylcyclohexyl)acetamide
(27) 2-(1-(4-methoxy-2,6-dimethylphenylsulfonyl)pyrrolidin-3-yloxy)-N-(4-phenyl-4-(piperidin-1-yl)cyclohexyl)acetamide
(28) 2-(2-(4-methoxy-N,2,3,6-tetramethylphenylsulfonamido)ethoxy)-N-(4-phenyl-4-(piperidin-1-yl)cyclohexyl)acetamide
(29) N-(4-(dimethylamino)-4-phenethylcyclohexyl)-2-(1-(mesitylsulfonyl)pyrrolidin-3-yloxy)acetamide
(30) 2-(2-(2,4-dichloro-N-methylphenylsulfonamido)ethoxy)-N-(4-(dimethylamino)-4-(2-methylbenzyl)cyclohexyl)acetamide
(31) N-(4-(dimethylamino)-4-phenethylcyclohexyl)-2-(2-(N-methyl-3-(trifluoromethyl)phenylsulfonamido)ethoxy)acetamide
(32) 2-((1-(4-methoxy-2,6-dimethylphenylsulfonyl)piperidin-2-yl)methoxy)-N-methyl-N-(3-(4-phenyl-4-(pyrrolidin-1-yl)cyclohexyl)propyl)acetamide
(33) N-methyl-N-((4-phenyl-4-(pyrrolidin-1-yl)cyclohexyl)methyl)-2-(1-(3-(trifluoromethyl)phenylsulfonyl)piperidin-2-yl)acetamide
(34) 2-(2-(4-methoxy-N,2,6-trimethylphenylsulfonamido)ethoxy)-N-methyl-N-(2-(4-phenyl-4-(pyrrolidin-1-yl)cyclohexyl)ethyl)acetamide
(35) 2-(2-(4-methoxy-N,2,6-trimethylphenylsulfonamido)ethoxy)-N-methyl-N-((4-phenyl-4-(pyrrolidin-1-yl)cyclohexyl)methyl)acetamide
(36) 1-(4-benzyl-4-(dimethylamino)piperidin-1-yl)-2-((1-(3,4-dichlorophenylsulfonyl)-1,2,3,4-tetrahydroquinolin-2-yl)methoxy)ethanone
(37) N-methyl-N-(3-(4-phenyl-4-(pyrrolidin-1-yl)cyclohexyl)propyl)-2-(1-(3-(trifluoromethyl)phenylsulfonyl)piperidin-2-yl)acetamide
(38) N-methyl-N-((4-phenethyl-4-(pyrrolidin-1-yl)cyclohexyl)methyl)-2-(1-(3-(trifluoromethyl)phenylsulfonyl)piperidin-2-yl)acetamide
(39) N-(2-(4-benzyl-4-(pyrrolidin-1-yl)cyclohexyl)ethyl)-2-(2-(4-methoxy-N,2,6-trimethylphenylsulfonamido)ethoxy)-N-methylacetamide
(40) N-methyl-3-(naphthalene-2-sulfonamido)-3-phenyl-N-(2-(4-phenyl-4-(pyrrolidin-1-yl)cyclohexyl)ethyl)propanamide
(41) 4-methoxy-N,2,6-trimethyl-N-(2-(2-(4-(4-methylpiperazin-1-yl)-4-phenylpiperidin-1-yl)-2-oxoethoxy)ethyl)benzenesulfonamide
(42) N-(2-(2-(4-(4-fluorophenyl)-4-(4-methylpiperazin-1-yl)piperidin-1-yl)-2-oxoethoxy)ethyl)-4-methoxy-N,2,6-trimethylbenzenesulfonamide
(43) N-methyl-N-(2-(4-phenethyl-4-(pyrrolidin-1-yl)cyclohexyl)ethyl)-2-(1-(3-(trifluoromethyl)phenylsulfonyl)piperidin-2-yl)acetamide
(44) N-(2-(2-(4-(3-fluorophenyl)-4-(4-methylpiperazin-1-yl)piperidin-1-yl)-2-oxoethoxy)ethyl)-4-methoxy-N,2,6-trimethylbenzenesulfonamide
(45) N-((4-benzyl-4-(pyrrolidin-1-yl)cyclohexyl)methyl)-2-(2-(4-methoxy-N,2,6-trimethylphenylsulfonamido)ethoxy)-N-methylacetamide
(46) 2-((1-(4-methoxy-2,6-dimethylphenylsulfonyl)piperidin-2-yl)methoxy)-N-methyl-N-(2-(4-phenethyl-4-(pyrrolidin-1-yl)cyclohexyl)ethyl)acetamide
(47) 2-(2-(4-methoxy-N,2,6-trimethylphenylsulfonamido)ethoxy)-N-methyl-N-((4-phenethyl-4-(pyrrolidin-1-yl)cyclohexyl)methyl)acetamide
(48) N-(2-(2-(4-(dimethylamino)-4-phenethylpiperidin-1-yl)-2-oxoethoxy)ethyl)-4-methoxy-N,2,6-trimethylbenzenesulfonamide
(49) 2-((1-(4-methoxy-2,6-dimethylphenylsulfonyl)piperidin-2-yl)methoxy)-1-(4-(4-methyl piperazin-1-yl)-4-phenylpiperidin-1-yl)ethanone
(50) 1-(4-(dimethylamino)-4-phenethylpiperidin-1-yl)-2-((1-(4-methoxy-2,6-dimethylphenylsulfonyl)piperidin-2-yl)methoxy)ethanone
(51) 2-((1-(4-methoxy-2,6-dimethylphenylsulfonyl)piperidin-2-yl)methoxy)-N-methyl-N-((4-phenethyl-4-(pyrrolidin-1-yl)cyclohexyl)methyl)acetamide
(52) N-(2-(4-(dimethylamino)-4-phenethylcyclohexyl)ethyl)-2-(2-(4-methoxy-N,2,6-trimethylphenylsulfonamido)ethoxy)-N-methylacetamide
(53) N-(2-(4-benzyl-4-(dimethylamino)cyclohexyl)ethyl)-2-(2-(4-methoxy-N,2,6-trimethylphenylsulfonamido)ethoxy)-N-methylacetamide
(54) N-(2-(2-(4-(dimethylamino)-4-phenylpiperidin-1-yl)-2-oxoethoxy)ethyl)-4-methoxy-N,2,6-trimethylbenzenesulfonamide
(55) N-(3-(4-(4-methylpiperazin-1-yl)-4-phenethylpiperidin-1-yl)-3-oxo-1-phenylpropyl)naphthalene-2-sulfonamide
(56) N-(2-(4-benzyl-4-(pyrrolidin-1-yl)cyclohexyl)ethyl)-N-methyl-3-(naphthalene-2-sulfonamido)-3-phenylpropanamide
(57) 1-(4-benzyl-4-(4-methylpiperazin-1-yl)piperidin-1-yl)-2-((1-(4-methoxy-2,6-dimethylphenylsulfonyl)piperidin-2-yl)methoxy)ethanone
(58) 2-(2-(4-methoxy-N,2,6-trimethylphenylsulfonamido)ethoxy)-N-methyl-N-(2-(4-phenethyl-4-(pyrrolidin-1-yl)cyclohexyl)ethyl)acetamide
(59) 2-((1-(4-methoxy-2,6-dimethylphenylsulfonyl)piperidin-2-yl)methoxy)-N-methyl-N-(3-(4-phenethyl-4-(pyrrolidin-1-yl)cyclohexyl)propyl)acetamide
(60) 2-((1-(4-methoxy-2,6-dimethylphenylsulfonyl)piperidin-2-yl)methoxy)-N-methyl-N-(2-(4-phenyl-4-(pyrrolidin-1-yl)cyclohexyl)ethyl)acetamide
(61) N-(2-(4-benzyl-4-(dimethylamino)cyclohexyl)ethyl)-2-((1-(3,4-dichlorophenylsulfonyl)-1,2,3,4-tetrahydroquinolin-2-yl)methoxy)-N-methylacetamide
(62) 2-((1-(4-methoxy-2,6-dimethylphenylsulfonyl)piperidin-2-yl)methoxy)-1-(4-phenyl-4-(4-(pyridin-4-yl)piperazin-1-yl)piperidin-1-yl)ethanone
(63) N-(2-(2-(4-benzyl-4-(dimethylamino)piperidin-1-yl)-2-oxoethoxy)ethyl)-4-methoxy-N,2,6-trimethylbenzenesulfonamide
(64) 1-(4-(4-methylpiperazin-1-yl)-4-phenylpiperidin-1-yl)-2-(1-(3-(trifluoromethyl)phenylsulfonyl)piperidin-2-yl)ethanone
(65) 1-(4-benzyl-4-(dimethylamino)piperidin-1-yl)-2-((1-(4-methoxy-2,6-dimethylphenylsulfonyl)piperidin-2-yl)methoxy)ethanone
(66) 2-((1-(4-methoxy-2,6-dimethylphenylsulfonyl)piperidin-2-yl)methoxy)-N-methyl-N-((4-phenyl-4-(pyrrolidin-1-yl)cyclohexyl)methyl)acetamide
(67) 1-(4-(3-fluorophenyl)-4-(4-methylpiperazin-1-yl)piperidin-1-yl)-2-((1-(4-methoxy-2,6-dimethylphenylsulfonyl)piperidin-2-yl)methoxy)ethanone
(68) N-(2-(4-benzyl-4-(pyrrolidin-1-yl)cyclohexyl)ethyl)-2-((1-(4-methoxy-2,6-dimethylphenylsulfonyl)piperidin-2-yl)methoxy)-N-methylacetamide
(69) 4-methoxy-N,2,6-trimethyl-N-(2-(2-oxo-2-(4-phenyl-4-(4-(pyridin-4-yl)piperazin-1-yl)piperidin-1-yl)ethoxy)ethyl)benzenesulfonamide
(70) 1-(4-(4-fluorophenyl)-4-(4-methylpiperazin-1-yl)piperidin-1-yl)-2-((1-(4-methoxy-2,6-dimethylphenylsulfonyl)piperidin-2-yl)methoxy)ethanone
(71) 1-(4-(dimethylamino)-4-phenethylpiperidin-1-yl)-2-(1-(3-(trifluoromethyl)phenylsulfonyl)piperidin-2-yl)ethanone
(72) N-(2-(4-(dimethylamino)-4-phenylcyclohexyl)ethyl)-2-(2-(4-methoxy-N,2,6-trimethylphenylsulfonamido)ethoxy)-N-methylacetamide
(73) N-(2-(4-(dimethylamino)-4-phenethylcyclohexyl)ethyl)-2-((1-(4-methoxy-2,6-dimethylphenylsulfonyl)piperidin-2-yl)methoxy)-N-methylacetamide
(74) N-(3-(4-benzyl-4-(pyrrolidin-1-yl)cyclohexyl)propyl)-N-methyl-3-(naphthalene-2-sulfonamido)-3-phenylpropanamide
(75) 1-(4-(dimethylamino)-4-phenylpiperidin-1-yl)-2-((1-(4-methoxy-2,6-dimethylphenylsulfonyl)piperidin-2-yl)methoxy)ethanone
(76) N-(2-(4-(dimethylamino)-4-phenethylcyclohexyl)ethyl)-N-methyl-2-(1-(3-(trifluoromethyl)phenylsulfonyl)piperidin-2-yl)acetamide
(77) 2-((1-(4-methoxy-2,6-dimethylphenylsulfonyl)piperidin-2-yl)methoxy)-1-(4-(4-methylpiperazin-1-yl)-4-phenethylpiperidin-1-yl)ethanone
(78) N-((4-benzyl-4-(pyrrolidin-1-yl)cyclohexyl)methyl)-2-((1-(4-methoxy-2,6-dimethylphenylsulfonyl)piperidin-2-yl)methoxy)-N-methylacetamide
(79) N-methyl-3-(naphthalene-2-sulfonamido)-N-(3-(4-phenethyl-4-(pyrrolidin-1-yl)cyclohexyl)propyl)-3-phenylpropanamide
(80) 1-(4-benzyl-4-(dimethylamino)piperidin-1-yl)-3-(1-(4-chloro-2,5-dimethylphenylsulfonyl)piperidin-2-yl)propan-1-one
(81) N-(2-(4-(dimethylamino)-4-phenylcyclohexyl)ethyl)-2-((1-(4-methoxy-2,6-dimethylphenylsulfonyl)piperidin-2-yl)methoxy)-N-methylacetamide
(82) N-(3-(4-(4-methylpiperazin-1-yl)-4-phenylpiperidin-1-yl)-3-oxo-1-phenylpropyl)naphthalene-2-sulfonamide
(83) 2-(2-(4-methoxy-N,2,6-trimethylphenylsulfonamido)ethoxy)-N-methyl-N-(3-(4-phenyl-4-(pyrrolidin-1-yl)cyclohexyl)propyl)acetamide
(84) N-(2-(4-benzyl-4-(dimethylamino)cyclohexyl)ethyl)-2-((1-(4-methoxy-2,6-dimethylphenylsulfonyl)piperidin-2-yl)methoxy)-N-methylacetamide
(85) N-(3-(4-benzyl-4-(pyrrolidin-1-yl)cyclohexyl)propyl)-2-(2-(4-methoxy-N,2,6-trimethylphenylsulfonamido)ethoxy)-N-methylacetamide
(86) N-(2-(4-benzyl-4-(dimethylamino)cyclohexyl)ethyl)-N-methyl-3-(naphthalene-2-sulfonamido)-3-phenylpropanamide
(87) 4-methoxy-N,2,6-trimethyl-N-(2-(2-(4-(4-methylpiperazin-1-yl)-4-phenethylpiperidin-1-yl)-2-oxoethoxy)ethyl)benzenesulfonamide
(88) N-(3-(4-benzyl-4-(pyrrolidin-1-yl)cyclohexyl)propyl)-2-((1-(4-methoxy-2,6-dimethylphenylsulfonyl)piperidin-2-yl)methoxy)-N-methylacetamide
(89) N-methyl-3-(naphthalene-2-sulfonamido)-N-((4-phenethyl-4-(pyrrolidin-1-yl)cyclohexyl)methyl)-3-phenylpropanamide
(90) N-(2-(4-(dimethylamino)-4-phenylcyclohexyl)ethyl)-N-methyl-3-(naphthalene-2-sulfonamido)-3-phenylpropanamide
(91) 2-(2-(4-methoxy-N,2,6-trimethylphenylsulfonamido)ethoxy)-N-methyl-N-(3-(4-phenethyl-4-(pyrrolidin-1-yl)cyclohexyl)propyl)acetamide
(92) N-methyl-3-(naphthalene-2-sulfonamido)-3-phenyl-N-((4-phenyl-4-(pyrrolidin-1-yl)cyclohexyl)methyl)propanamide
(93) N-(3-(4-benzyl-4-(pyrrolidin-1-yl)cyclohexyl)propyl)-N-methyl-2-(1-(3-(trifluoromethyl)phenylsulfonyl)piperidin-2-yl)acetamide
(94) N-((4-benzyl-4-(pyrrolidin-1-yl)cyclohexyl)methyl)-N-methyl-2-(1-(3-(trifluoromethyl)phenylsulfonyl)piperidin-2-yl)acetamide
(95) N-(2-(4-benzyl-4-(dimethylamino)cyclohexyl)ethyl)-N-methyl-2-(1-(3-(trifluoromethyl)phenylsulfonyl)piperidin-2-yl)acetamide
(96) N-(3-(4-(4-fluorophenyl)-4-(4-methylpiperazin-1-yl)piperidin-1-yl)-3-oxo-1-phenylpropyl)naphthalene-2-sulfonamide
(97) 1-(4-benzyl-4-(dimethylamino)piperidin-1-yl)-2-(1-(3-(trifluoromethyl)phenylsulfonyl)piperidin-2-yl)ethanone
(98) N-(3-oxo-1-phenyl-3-(4-phenyl-4-(4-(pyridin-4-yl)piperazin-1-yl)piperidin-1-yl)propyl)naphthalene-2-sulfonamide
(99) 3-(1-(4-chloro-2,5-dimethylphenylsulfonyl)piperidin-2-yl)-1-(4-phenyl-4-(4-(pyridin-4-yl)piperazin-1-yl)piperidin-1-yl)propan-1-one
(100) 2-((1-(3,4-dichlorophenylsulfonyl)-1,2,3,4-tetrahydroquinolin-2-yl)methoxy)-1-(4-phenyl-4-(4-(pyridin-4-yl)piperazin-1-yl)piperidin-1-yl)ethanone
(101) 2-((1-(3,4-dichlorophenylsulfonyl)-1,2,3,4-tetrahydroquinolin-2-yl)methoxy)-N-(2-(4-(dimethylamino)-4-phenethylcyclohexyl)ethyl)-N-methylacetamide
(102) N-(2-(4-(dimethylamino)-4-phenethylcyclohexyl)ethyl)-N-methyl-3-(naphthalene-2-sulfonamido)-3-phenylpropanamide
(103) 2-((1-(3,4-dichlorophenylsulfonyl)-1,2,3,4-tetrahydroquinolin-2-yl)methoxy)-1-(4-(3-fluorophenyl)-4-(4-methylpiperazin-1-yl)piperidin-1-yl)ethanone
(104) N-(2-(4-benzyl-4-(pyrrolidin-1-yl)cyclohexyl)ethyl)-2-((1-(3,4-dichlorophenylsulfonyl)-1,2,3,4-tetrahydroquinolin-2-yl)methoxy)-N-methylacetamide
(105) N-(3-(4-(dimethylamino)-4-phenethylpiperidin-1-yl)-3-oxo-1-phenylpropyl)naphthalene-2-sulfonamide
(106) N-methyl-N-(2-(4-phenyl-4-(pyrrolidin-1-yl)cyclohexyl)ethyl)-2-(1-(3-(trifluoromethyl)phenylsulfonyl)piperidin-2-yl)acetamide
(107) 3-(1-(4-chloro-2,5-dimethylphenylsulfonyl)piperidin-2-yl)-N-(2-(4-(dimethylamino)-4-phenylcyclohexyl)ethyl)-N-methylpropanamide
(108) 1-(4-benzyl-4-(4-methylpiperazin-1-yl)piperidin-1-yl)-2-((1-(3,4-dichlorophenylsulfonyl)-1,2,3,4-tetrahydroquinolin-2-yl)methoxy)ethanone
(109) 2-((1-(3,4-dichlorophenylsulfonyl)-1,2,3,4-tetrahydroquinolin-2-yl)methoxy)-1-(4-(dimethylamino)-4-phenylpiperidin-1-yl)ethanone
(110) 3-(1-(4-chloro-2,5-dimethylphenylsulfonyl)piperidin-2-yl)-1-(4-(4-methylpiperazin-1-yl)-4-phenylpiperidin-1-yl)propan-1-one
(111) 2-((1-(3,4-dichlorophenylsulfonyl)-1,2,3,4-tetrahydroquinolin-2-yl)methoxy)-N-(2-(4-(dimethylamino)-4-phenylcyclohexyl)ethyl)-N-methylacetamide
(112) N-((4-benzyl-4-(pyrrolidin-1-yl)cyclohexyl)methyl)-N-methyl-3-(naphthalene-2-sulfonamido)-3-phenylpropanamide
(113) 1-(4-phenyl-4-(4-(pyridin-4-yl)piperazin-1-yl)piperidin-1-yl)-2-(1-(3-(trifluoromethyl)phenylsulfonyl)piperidin-2-yl)ethanone
(114) N-methyl-3-(naphthalene-2-sulfonamido)-N-(2-(4-phenethyl-4-(pyrrolidin-1-yl)cyclohexyl)ethyl)-3-phenylpropanamide
(115) 2-((1-(3,4-dichlorophenylsulfonyl)-1,2,3,4-tetrahydroquinolin-2-yl)methoxy)-1-(4-(4-methylpiperazin-1-yl)-4-phenylpiperidin-1-yl)ethanone
(116) 2-((1-(3,4-dichlorophenylsulfonyl)-1,2,3,4-tetrahydroquinolin-2-yl)methoxy)-1-(4-(4-methylpiperazin-1-yl)-4-phenethylpiperidin-1-yl)ethanone
(117) N-(2-(4-benzyl-4-(pyrrolidin-1-yl)cyclohexyl)ethyl)-3-(1-(4-chloro-2,5-dimethylphenylsulfonyl)piperidin-2-yl)-N-methylpropanamide
(118) N-(3-(4-benzyl-4-(4-methylpiperazin-1-yl)piperidin-1-yl)-3-oxo-1-phenylpropyl)naphthalene-2-sulfonamide
(119) 1-(4-(4-methylpiperazin-1-yl)-4-phenethylpiperidin-1-yl)-2-(1-(3-(trifluoromethyl)phenylsulfonyl)piperidin-2-yl)ethanone
(120) N-(3-(4-(3-fluorophenyl)-4-(4-methylpiperazin-1-yl)piperidin-1-yl)-3-oxo-1-phenylpropyl)naphthalene-2-sulfonamide
(121) N-(2-(4-benzyl-4-(pyrrolidin-1-yl)cyclohexyl)ethyl)-N-methyl-2-(1-(3-(trifluoromethyl)phenylsulfonyl)piperidin-2-yl)acetamide
(122) 3-(1-(4-chloro-2,5-dimethylphenylsulfonyl)piperidin-2-yl)-1-(4-(4-methylpiperazin-1-yl)-4-phenethylpiperidin-1-yl)propan-1-one
(123) 3-(1-(4-chloro-2,5-dimethylphenylsulfonyl)piperidin-2-yl)-1-(4-(dimethylamino)-4-phenylpiperidin-1-yl)propan-1-one
(124) N-(3-(4-benzyl-4-(pyrrolidin-1-yl)cyclohexyl)propyl)-3-(1-(4-chloro-2,5-dimethylphenylsulfonyl)piperidin-2-yl)-N-methylpropanamide
(125) N-(2-(4-(dimethylamino)-4-phenylcyclohexyl)ethyl)-N-methyl-2-(1-(3-(trifluoromethyl)phenylsulfonyl)piperidin-2-yl)acetamide
(126) 2-((1-(3,4-dichlorophenylsulfonyl)-1,2,3,4-tetrahydroquinolin-2-yl)methoxy)-1-(4-(4-fluorophenyl)-4-(4-methylpiperazin-1-yl)piperidin-1-yl)ethanone
(127) N-((4-benzyl-4-(pyrrolidin-1-yl)cyclohexyl)methyl)-2-((1-(3,4-dichlorophenylsulfonyl)-1,2,3,4-tetrahydroquinolin-2-yl)methoxy)-N-methylacetamide
(128) N-methyl-3-(naphthalene-2-sulfonamido)-3-phenyl-N-(3-(4-phenyl-4-(pyrrolidin-1-yl)cyclohexyl)propyl)propanamide
(129) N-((4-benzyl-4-(pyrrolidin-1-yl)cyclohexyl)methyl)-3-(1-(4-chloro-2,5-dimethylphenylsulfonyl)piperidin-2-yl)-N-methylpropanamide
(130) 1-(4-benzyl-4-(4-methylpiperazin-1-yl)piperidin-1-yl)-3-(1-(4-chloro-2,5-dimethylphenylsulfonyl)piperidin-2-yl)propan-1-one
(131) 3-(1-(4-chloro-2,5-dimethylphenylsulfonyl)piperidin-2-yl)-N-methyl-N-(2-(4-phenethyl-4-(pyrrolidin-1-yl)cyclohexyl)ethyl)propanamide
(132) 3-(1-(4-chloro-2,5-dimethylphenylsulfonyl)piperidin-2-yl)-N-(2-(4-(dimethylamino)-4-phenethylcyclohexyl)ethyl)-N-methylpropanamide
(133) 2-((1-(3,4-dichlorophenylsulfonyl)-1,2,3,4-tetrahydroquinolin-2-yl)methoxy)-N-methyl-N-(3-(4-phenyl-4-(pyrrolidin-1-yl)cyclohexyl)propyl)acetamide
(134) N-methyl-N-(3-(4-phenethyl-4-(pyrrolidin-1-yl)cyclohexyl)propyl)-2-(1-(3-(trifluoromethyl)phenylsulfonyl)piperidin-2-yl)acetamide
(135) 3-(1-(4-chloro-2,5-dimethylphenylsulfonyl)piperidin-2-yl)-1-(4-(3-fluorophenyl)-4-(4-methylpiperazin-1-yl)piperidin-1-yl)propan-1-one
(136) 3-(1-(4-chloro-2,5-dimethylphenylsulfonyl)piperidin-2-yl)-N-methyl-N-(3-(4-phenyl-4-(pyrrolidin-1-yl)cyclohexyl)propyl)propanamide
(137) 3-(1-(4-chloro-2,5-dimethylphenylsulfonyl)piperidin-2-yl)-1-(4-(dimethylamino)-4-phenethylpiperidin-1-yl)propan-1-one
(138) 3-(1-(4-chloro-2,5-dimethylphenylsulfonyl)piperidin-2-yl)-1-(4-(4-fluorophenyl)-4-(4-methylpiperazin-1-yl)piperidin-1-yl)propan-1-one
(139) 3-(1-(4-chloro-2,5-dimethylphenylsulfonyl)piperidin-2-yl)-N-methyl-N-(2-(4-phenyl-4-(pyrrolidin-1-yl)cyclohexyl)ethyl)propanamide
(140) 3-(1-(4-chloro-2,5-dimethylphenylsulfonyl)piperidin-2-yl)-N-methyl-N-((4-phenyl-4-(pyrrolidin-1-yl)cyclohexyl)methyl)propanamide
(141) 3-(1-(4-chloro-2,5-dimethylphenylsulfonyl)piperidin-2-yl)-N-methyl-N-((4-phenethyl-4-(pyrrolidin-1-yl)cyclohexyl)methyl)propanamide
(142) N-(4-phenyl-4-(pyrrolidin-1-yl)cyclohexyl)-2-((1-(2,4,6-trichlorophenylsulfonyl)piperidin-2-yl)methoxy)acetamide
(143) N-((4-benzyl-4-(4-methylpiperazin-1-yl)cyclohexyl)methyl)-2-((1-(4-methoxy-2,6-dimethylphenylsulfonyl)piperidin-2-yl)methoxy)-N-methylacetamide
(144) 2-((1-(4-methoxy-2,6-dimethylphenylsulfonyl)piperidin-2-yl)methoxy)-N-methyl-N-((4-(4-methylpiperazin-1-yl)-4-phenethylcyclohexyl)methyl)acetamide
(145) 2-((1-(4-methoxy-2,6-dimethylphenylsulfonyl)pyrrolidin-2-yl)methoxy)-N-(4-phenyl-4-(pyrrolidin-1-yl)cyclohexyl)acetamide
(146) N-(4-phenyl-4-(pyrrolidin-1-yl)cyclohexyl)-2-((1-(2,4,6-trichlorophenylsulfonyl)pyrrolidin-2-yl)methoxy)acetamide
(147) 2-((1-(4-methoxy-2,6-dimethylphenylsulfonyl)piperidin-2-yl)methoxy)-N-(4-phenyl-4-(pyrrolidin-1-yl)cyclohexyl)acetamide
(148) 2-((1-(4-methoxy-2,6-dimethylphenylsulfonyl)pyrrolidin-2-yl)methoxy)-N-methyl-N-((4-(4-methylpiperazin-1-yl)-4-phenethylcyclohexyl)methyl)acetamide
(149) N-(4-benzyl-4-(pyrrolidin-1-yl)cyclohexyl)-2-((1-(4-methoxy-2,6-dimethylphenylsulfonyl)piperidin-2-yl)methoxy)acetamide
(150) 2-(2-(4-methoxy-N,2,6-trimethylphenylsulfonamido)ethoxy)-N-(4-phenyl-4-(pyrrolidin-1-yl)cyclohexyl)acetamide
(151) N-(4-benzyl-4-(pyrrolidin-1-yl)cyclohexyl)-2-((1-(4-methoxy-2,6-dimethylphenylsulfonyl)pyrrolidin-2-yl)methoxy)acetamide
(152) 2-((1-(4-methoxy-2,6-dimethylphenylsulfonyl)pyrrolidin-2-yl)methoxy)-N-((4-morpholino-4-phenylcyclohexyl)methyl)acetamide
(153) 2-(1-(4-methoxy-2,6-dimethylphenylsulfonyl)pyrrolidin-3-yloxy)-N-(4-phenyl-4-(pyrrolidin-1-yl)cyclohexyl)acetamide
(154) N-((4-benzyl-4-(4-methylpiperazin-1-yl)cyclohexyl)methyl)-2-((1-(4-methoxy-2,6-dimethylphenylsulfonyl)pyrrolidin-2-yl)methoxy)-N-methylacetamide
(155) N-(4-benzyl-4-morpholinocyclohexyl)-2-((1-(4-methoxy-2,6-dimethylphenylsulfonyl)piperidin-2-yl)methoxy)acetamide
(156) N-(4-benzyl-4-(pyrrolidin-1-yl)cyclohexyl)-2-((1-(2,4,6-trichlorophenylsulfonyl)pyrrolidin-2-yl)methoxy)acetamide
(157) 2-((1-(4-methoxy-2,6-dimethylphenylsulfonyl)piperidin-2-yl)methoxy)-N-((4-morpholino-4-phenylcyclohexyl)methyl)acetamide
(158) 2-((1-(4-methoxy-2,6-dimethylphenylsulfonyl)piperidin-2-yl)methoxy)-N-(4-morpholino-4-phenylcyclohexyl)acetamide
(159) N-(4-benzyl-4-morpholinocyclohexyl)-2-((1-(4-methoxy-2,6-dimethylphenylsulfonyl)pyrrolidin-2-yl)methoxy)acetamide
(160) 2-((1-(4-methoxy-2,6-dimethylphenylsulfonyl)pyrrolidin-2-yl)methoxy)-N-(4-morpholino-4-phenylcyclohexyl)acetamide
(161) N-methyl-N-((4-(4-methylpiperazin-1-yl)-4-phenethylcyclohexyl)methyl)-2-((1-(2,4,6-trichlorophenylsulfonyl)piperidin-2-yl)methoxy)acetamide
(162) N-((4-benzyl-4-(4-methylpiperazin-1-yl)cyclohexyl)methyl)-N-methyl-2-((1-(2,4,6-trichlorophenylsulfonyl)piperidin-2-yl)methoxy)acetamide
(163) N-(4-benzyl-4-(pyrrolidin-1-yl)cyclohexyl)-2-((1-(2,4,6-trichlorophenylsulfonyl)piperidin-2-yl)methoxy)acetamide
(164) N-(4-morpholino-4-phenylcyclohexyl)-2-((1-(2,4,6-trichlorophenylsulfonyl)pyrrolidin-2-yl)methoxy)acetamide
(165) 2-(2-(4-methoxy-N,2,6-trimethylphenylsulfonamido)ethoxy)-N-methyl-N-((4-(4-methylpiperazin-1-yl)-4-phenethylcyclohexyl)methyl)acetamide
(166) N-(4-benzyl-4-(pyrrolidin-1-yl)cyclohexyl)-2-(1-(4-methoxy-2,6-dimethylphenylsulfonyl)pyrrolidin-3-yloxy)acetamide
(167) 2-(2-(4-methoxy-N,2,6-trimethylphenylsulfonamido)ethoxy)-N-(4-morpholino-4-phenylcyclohexyl)acetamide
(168) N-((4-benzyl-4-(4-methylpiperazin-1-yl)cyclohexyl)methyl)-N-methyl-2-((1-(2,4,6-trichlorophenylsulfonyl)pyrrolidin-2-yl)methoxy)acetamide
(169) N-((4-benzyl-4-morpholinocyclohexyl)methyl)-2-((1-(4-methoxy-2,6-dimethylphenylsulfonyl)pyrrolidin-2-yl)methoxy)acetamide
(170) N-(4-benzyl-4-(pyrrolidin-1-yl)cyclohexyl)-2-(2-(4-methoxy-N,2,6-trimethylphenylsulfonamido)ethoxy)acetamide
(171) N-(4-benzyl-4-morpholinocyclohexyl)-2-(2-(4-methoxy-N,2,6-trimethylphenylsulfonamido)ethoxy)acetamide
(172) N-methyl-N-((4-(4-methylpiperazin-1-yl)-4-phenethylcyclohexyl)methyl)-2-((1-(2,4,6-trichlorophenylsulfonyl)pyrrolidin-2-yl)methoxy)acetamide
(173) 2-(1-(4-methoxy-2,6-dimethylphenylsulfonyl)pyrrolidin-3-yloxy)-N-((4-morpholino-4-phenylcyclohexyl)methyl)acetamide
(174) N-((4-benzyl-4-(4-methylpiperazin-1-yl)cyclohexyl)methyl)-2-(1-(4-methoxy-2,6-dimethylphenylsulfonyl)pyrrolidin-3-yloxy)-N-methylacetamide
(175) N-((4-benzyl-4-(4-methylpiperazin-1-yl)cyclohexyl)methyl)-N-methyl-2-(1-(2,4,6-trichlorophenylsulfonyl)piperidin-3-yloxy)acetamide
(176) 2-(1-(4-methoxy-2,6-dimethylphenylsulfonyl)piperidin-3-yloxy)-N-(4-phenyl-4-(pyrrolidin-1-yl)cyclohexyl)acetamide
(177) 2-(1-(4-methoxy-2,6-dimethylphenylsulfonyl)pyrrolidin-3-yloxy)-N-methyl-N-((4-(4-methylpiperazin-1-yl)-4-phenethylcyclohexyl)methyl)acetamide
(178) N-(4-phenyl-4-(pyrrolidin-1-yl)cyclohexyl)-2-(1-(2,4,6-trichlorophenylsulfonyl)pyrrolidin-3-yloxy)acetamide
(179) 2-(2-(2,4-dichloro-N-methylphenylsulfonamido)ethoxy)-N-(4-phenyl-4-(pyrrolidin-1-yl)cyclohexyl)acetamide
(180) N-(4-benzyl-4-(pyrrolidin-1-yl)cyclohexyl)-2-((1-(4-methoxy-2,6-dimethylphenylsulfonyl)piperidin-3-yl)methoxy)acetamide
(181) N-(4-benzyl-4-(pyrrolidin-1-yl)cyclohexyl)-2-(1-(4-methoxy-2,6-dimethylphenylsulfonyl)piperidin-3-yloxy)acetamide
(182) 2-((1-(4-methoxy-2,6-dimethylphenylsulfonyl)piperidin-3-yl)methoxy)-N-methyl-N-((4-(4-methylpiperazin-1-yl)-4-phenethylcyclohexyl)methyl)acetamide
(183) N-((4-benzyl-4-(4-methylpiperazin-1-yl)cyclohexyl)methyl)-2-(2-(4-methoxy-N,2,6-trimethylphenylsulfonamido)ethoxy)-N-methylacetamide
(184) N-((4-benzyl-4-(4-methylpiperazin-1-yl)cyclohexyl)methyl)-2-(1-(4-methoxy-2,6-dimethylphenylsulfonyl)piperidin-3-yloxy)-N-methylacetamide
(185) 2-(2-(4-methoxy-N,2,6-trimethylphenylsulfonamido)ethoxy)-N-((4-morpholino-4-phenylcyclohexyl)methyl)acetamide
(186) N-((4-benzyl-4-(4-methylpiperazin-1-yl)cyclohexyl)methyl)-N-methyl-2-(2-(2,4,6-trichloro-N-methylphenylsulfonamido)ethoxy)acetamide
(187) 2-((1-(4-methoxy-2,6-dimethylphenylsulfonyl)piperidin-3-yl)methoxy)-N-(4-phenyl-4-(pyrrolidin-1-yl)cyclohexyl)acetamide
(188) N-((4-benzyl-4-morpholinocyclohexyl)methyl)-2-(2-(4-methoxy-N,2,6-trimethylphenylsulfonamido)ethoxy)acetamide
(189) N-(4-benzyl-4-(pyrrolidin-1-yl)cyclohexyl)-2-(2-(2,4,6-trichloro-N-methylphenylsulfonamido)ethoxy)acetamide
(190) N-(4-benzyl-4-morpholinocyclohexyl)-2-(1-(4-methoxy-2,6-dimethylphenylsulfonyl)piperidin-3-yloxy)acetamide
(191) N-(4-phenyl-4-(pyrrolidin-1-yl)cyclohexyl)-2-(2-(2,4,6-trichloro-N-methylphenylsulfonamido)ethoxy)acetamide
(192) N-(4-phenyl-4-(pyrrolidin-1-yl)cyclohexyl)-2-((1-(2,4,6-trichlorophenylsulfonyl)piperidin-3-yl)methoxy)acetamide
(193) N-(4-benzyl-4-(pyrrolidin-1-yl)cyclohexyl)-2-(2-(2,4-dichloro-N-methylphenylsulfonamido)ethoxy)acetamide
(194) N-(4-phenyl-4-(pyrrolidin-1-yl)cyclohexyl)-2-(1-(2,4,6-trichlorophenylsulfonyl)piperidin-3-yloxy)acetamide
(195) N-methyl-N-((4-(4-methylpiperazin-1-yl)-4-phenethylcyclohexyl)methyl)-2-(2-(2,4,6-trichloro-N-methylphenylsulfonamido)ethoxy)acetamide
(196) N-(2-(2-(4-Amino-4-phenylpiperidin-1-yl)-2-oxoethoxy)ethyl)-4-methoxy-N,2,6-trimethylbenzenesulfonamide
(197) N-(2-(2-(3-benzyl-3-(4-methylpiperazin-1-yl)pyrrolidin-1-yl)-2-oxoethoxy)ethyl)-4-methoxy-N,2,6-trimethylbenzenesulfonamide
(198) N-(4-(dimethylamino)-4-phenylcyclohexyl)-2-(1-(2,4,6-trichlorophenylsulfonyl)pyrrolidin-3-yloxy)acetamide
(199) N-(4-(dimethylamino)-4-phenylcyclohexyl)-2-(2-(2,4,6-trichloro-N-methylphenylsulfonamido)ethoxy)acetamide
(200) (S)-2-((1-(4-methoxy-2,6-dimethylphenylsulfonyl)piperidin-2-yl)methoxy)-N-methyl-N-(2-(4-phenyl-4-(pyrrolidin-1-yl)cyclohexyl)ethyl)acetamide
(201) (S)-N-(2-(4-(azetidin-1-yl)-4-phenylcyclohexyl)ethyl)-2-((1-(4-methoxy-2,6-dimethylphenylsulfonyl)piperidin-2-yl)methoxy)-N-methylacetamide
(202) 1-(4-(dimethylamino)-4-phenylpiperidin-1-yl)-2-((1-(4-methoxy-2,6-dimethylphenylsulfonyl)piperidin-2-yl)methoxy)ethanone
(203) N-(3-(4-(dimethylamino)-4-phenylcyclohexyl)propyl)-2-((1-(4-methoxy-2,6-dimethylphenylsulfonyl)piperidin-2-yl)methoxy)-N-methylacetamide
(204) N-(3-(4-(3-fluorophenyl)-4-(pyrrolidin-1-yl)cyclohexyl)propyl)-2-((1-(4-methoxy-2,6-dimethylphenylsulfonyl)piperidin-2-yl)methoxy)-N-methylacetamide
(205) N-(3-(4-(azetidin-1-yl)-4-phenylcyclohexyl)propyl)-2-((1-(4-methoxy-2,6-dimethylphenylsulfonyl)piperidin-2-yl)methoxy)-N-methylacetamide
(206) N-(2-(2-(4-(dimethylamino)-4-(pyridin-4-yl)piperidin-1-yl)-2-oxoethoxy)ethyl)-4-methoxy-N,2,6-trimethylbenzenesulfonamide
(207) N-(2-(4-(Dimethylamino)-4-(pyridin-3-yl)cyclohexyl)ethyl)-2-(2-(4-methoxy-N,2,6-trimethylphenylsulfonamido)ethoxy)-N-methylacetamide
(208) N-(2-(2-(4-(Dimethylamino)-4-(pyridin-3-yl)piperidin-1-yl)-2-oxoethoxy)ethyl)-4-methoxy-N,2,6-trimethylbenzenesulfonamide
(209) 4-Methoxy-N,2,6-trimethyl-N-(2-(2-(4-(methylamino)-4-(pyridin-4-yl)piperidin-1-yl)-2-oxoethoxy)ethyl)benzenesulfonamide
(210) N-(2-(2-(4-(3-Fluorophenyl)-4-(4-methylpiperazin-1-yl)piperidin-1-yl)-2-oxoethoxy)ethyl)-4-methoxy-N,2,6-trimethylbenzenesulfonamide
(211) 1-(4-(3-Fluorophenyl)-4-(4-methylpiperazin-1-yl)piperidin-1-yl)-2-((1-(4-methoxy-2,6-dimethylphenylsulfonyl)piperidin-2-yl)methoxy)ethanone
(212) 4-(1-(2-Chloro-6-methylphenylsulfonyl)piperidin-2-yl)-1-(4-(3-fluorophenyl)-4-(4-methylpiperazin-1-yl)piperidin-1-yl)butan-1-one
(213) 1-(4-(3-Fluorophenyl)-4-(4-methylpiperazin-1-yl)piperidin-1-yl)-4-(1-(2-(trifluoromethyl)phenylsulfonyl)piperidin-2-yl)butan-1-one
(214) 1-(4-(3-Fluorophenyl)-4-(4-methylpiperazin-1-yl)piperidin-1-yl)-4-(1-(4-methoxy-2,6-dimethylphenylsulfonyl)piperidin-2-yl)butan-1-one
(215) 1-(4-(3-Fluorophenyl)-4-(4-methylpiperazin-1-yl)piperidin-1-yl)-4-(1-(naphthalen-1-ylsulfonyl)piperidin-2-yl)butan-1-one
(216) 1-(4-(3-Fluorophenyl)-4-(4-methylpiperazin-1-yl)piperidin-1-yl)-4-(1-(naphthalen-2-ylsulfonyl)piperidin-2-yl)butan-1-one
(217) 1-(4-(3-Fluorophenyl)-4-(4-methylpiperazin-1-yl)piperidin-1-yl)-2-(1-(4-methoxy-2,6-dimethylphenylsulfonyl)pyrrolidin-3-yloxy)ethanone
(218) N-Benzyl-N-(2-(2-(4-(3-fluorophenyl)-4-(4-methylpiperazin-1-yl)piperidin-1-yl)-2-oxoethoxy)ethyl)-4-methoxy-2,6-dimethylbenzenesulfonamide
(219) N-(2-(2-(4-(3-Fluorophenyl)-4-(4-methylpiperazin-1-yl)piperidin-1-yl)-2-oxoethoxy)ethyl)-4-methoxy-2,6-dimethyl-N-phenylbenzenesulfonamide
(220) 1-(4-(3-Fluorophenyl)-4-(4-methylpiperazin-1-yl)piperidin-1-yl)-2-((1-(4-methoxy-2,6-dimethylphenylsulfonyl)-1,2,3,4-tetrahydroquinolin-2-yl)methoxy)ethanone
(221) 1-(4-(3-Fluorophenyl)-4-(4-methylpiperazin-1-yl)piperidin-1-yl)-2-((4-(4-methoxy-2,6-dimethylphenylsulfonyl)-3,4-dihydro-2H-benzo[b][1,4]oxazin-3-yl)methoxy)ethanone
(222) 2-((4-(2-Chloro-6-methylphenylsulfonyl)-3,4-dihydro-2H-benzo[b][1,4]oxazin-3-yl)methoxy)-1-(4-(3-fluorophenyl)-4-(4-methylpiperazin-1-yl)piperidin-1-yl)ethanone
(223) 1-(4-(3-Fluorophenyl)-4-(4-methylpiperazin-1-yl)piperidin-1-yl)-2-((4-(2-(trifluoromethyl)phenylsulfonyl)-3,4-dihydro-2H-benzo[b][1,4]oxazin-3-yl)methoxy)ethanone
(224) N-(2-(2-(4-(3-Fluorophenyl)-4-(4-methylpiperazin-1-yl)piperidin-1-yl)-2-oxoethoxy)ethyl)-4-methoxy-N,2,3,6-tetramethylbenzenesulfonamide
(225) 1-(4-(3-Fluorophenyl)-4-(4-methylpiperazin-1-yl)piperidin-1-yl)-2-((1-(2-(trifluoromethyl)phenylsulfonyl)piperidin-2-yl)methoxy)ethanone
(226) 1-(4-(3-Fluorophenyl)-4-(4-methylpiperazin-1-yl)piperidin-1-yl)-3-((1-(4-methoxy-2,6-dimethylphenylsulfonyl)piperidin-2-yl)methoxy)propan-1-one
(227) 1-(4-(3-Fluorophenyl)-4-(4-methylpiperazin-1-yl)piperidin-1-yl)-2-(2-(1-(4-methoxy-2,6-dimethylphenylsulfonyl)piperidin-2-yl)ethoxy)ethanone
(228) 1-(4-(3-Fluorophenyl)-4-(4-methylpiperazin-1-yl)piperidin-1-yl)-2-((1-(naphthalen-2-ylsulfonyl)-1,2,3,4-tetrahydroquinolin-2-yl)methoxy)ethanone optionally in the form of an individual enantiomer or of an individual diastereomer, of the racemate, of the enantiomers, of the diastereomers, mixtures of the enantiomers and/or diastereomers, and in each case in the form of their bases and/or physiologically acceptable salts such as hydrochlorides.

The numbering of the individual embodiments of the compounds according to the invention used above is retained in the following explanations of the present invention, in particular in the description of the examples.

The compounds according to the invention have an antagonistic action on the human B1R receptor or the B1R receptor of the rat. In a preferred embodiment of the invention, the compounds according to the invention have an antagonistic action both on the human B1R receptor (hB1R) and on the B1R receptor of the rat (rB1R).

Compounds which show an inhibition of at least 15%, 25%, 50%. 70%, 80% or 90% on the human B1R receptor and/or on the B1R receptor of the rat in the FLIPR assay at a concentration of 10 μm are particularly preferred. Compounds which show an inhibition on the human B1R receptor and on the B1R receptor of the rat of at least 70%, in particular of at least 80% and particularly preferably of at least 90% at a concentration of 10 μm are very particularly preferred.

The agonistic or antagonistic action of substances can be quantified on the bradykinin 1 receptor (B1R) of the human and rat species with ectopically expressing cell lines (CHO K1 cells) and with the aid of a Ca²⁺-sensitive dyestuff (Fluo-4) in a fluorescent imaging plate reader (FLIPR). The figure in % activation is based on the Ca²⁺ signal after addition of Lys-Des-Arg⁹-bradykinin (0.5 nM) or Des-Arg⁹-bradykinin (100 nM). Antagonists lead to a suppression of the Ca²⁺ inflow after addition of the agonist. % inhibition compared with the maximum achievable inhibition is stated.

The substances according to the invention act, for example, on the B1R relevant in connection with various diseases, so that they are suitable as a pharmaceutical active compound in pharmaceutical compositions. The invention therefore also provides pharmaceutical compositions containing at least one substituted sulfonamide compound according to the invention and optionally suitable additives and/or auxiliary substances and/or optionally further active compounds.

Compounds which show an inhibition of at least 5%, 10%, 15%, 25%, 50%, 70%, 80% or 90% on the human p opioid receptor at a concentration of 1 μM are also particularly preferred. The inhibition is determined with the aid of conventional methods known to the person skilled in the art, in particular with the aid of the assays described below.

The pharmaceutical compositions according to the invention optionally contain, in addition to at least one substituted sulfonamide compound according to the invention, suitable additives and/or auxiliary substances, that is to say also carrier materials, fillers, solvents, diluents, dyestuffs and/or binders, and can be administered as liquid pharmaceutical composition forms in the form of injection solutions, drops or juices or as semi-solid pharmaceutical composition forms in the form of granules, tablets, pellets, patches, capsules, plasters/spray-on plasters or aerosols. The choice of auxiliary substances etc. and the amounts thereof to be employed depend on whether the pharmaceutical composition is to be administered orally, perorally, parenterally, intravenously, intraperitoneally, intradermally, intramuscularly, nasally, buccally, rectally or topically, for example to the skin, the mucous membranes or into the eyes. Formulations in the form of tablets, coated tablets, capsules, granules, drops, juices and syrups are suitable for oral administration, and solutions, suspensions, easily reconstitutable dry formulations and sprays are suitable for parenteral, topical and inhalatory administration. Sulfonamide compounds according to the invention in a depot, in dissolved form or in a plaster, optionally with the addition of agents which promote penetration through the skin, are suitable formulations for percutaneous administration. Formulation forms which can be used orally or percutaneously can release the substituted sulfonamide compounds according to the invention in a delayed manner. The substituted sulfonamide compounds according to the invention can also be used in parenteral long-term depot forms, such as e.g. implants or implanted pumps. In principle, other further active compounds known to the person skilled in the art can be added to the pharmaceutical compositions according to the invention.

The amount of active compound to be administered to patients varies as a function of the weight of the patient, the mode of administration, the indication and the severity of the disease. 0.00005 to 50 mg/kg, preferably 0.01 to 5 mg/kg of at least one substituted sulfonamide compound according to the invention are conventionally administered.

In a preferred form of the pharmaceutical composition, a substituted sulfonamide compound according to the invention contained therein is present as the pure diastereomer and/or enantiomer, as a racemate or as a non-equimolar or equimolar mixture of the diastereomers and/or enantiomers.

B1R is involved in particular in the pain event. The substituted sulfonamide compounds according to the invention can accordingly be used for the preparation of a pharmaceutical composition for treatment of pain, in particular acute, visceral, neuropathic or chronic pain. The substituted sulfonamide compounds according to the invention can also be used for the preparation of a pharmaceutical composition for treatment of inflammatory pain.

The invention therefore also provides the use of a substituted sulfonamide compound according to the invention for the preparation of a pharmaceutical composition for treatment of pain, in particular acute, visceral, neuropathic or chronic pain. Furthermore the invention also provides the use of a substituted sulfonamide compound according to the invention for the preparation of a pharmaceutical composition for treatment of inflammatory pain.

The invention also provide the use of a substituted sulfonamide compound according to the invention for the preparation of a pharmaceutical composition for treatment of diabetes, diseases of the respiratory tract, for example bronchial asthma, allergies, COPD/chronic obstructive pulmonary disease or cystic fibrosis; inflammatory intestinal diseases, for example ulcerative colitis or CD/Crohn's disease; neurological diseases, for example multiple sclerosis or neurodegeneration; inflammations of the skin, for example atopic dermatitis, psoriasis or bacterial infections; rheumatic diseases, for example rheumatoid arthritis or osteoarthritis; septic shock; reperfusion syndrome, for example following cardiac infarction or stroke, obesity; and as an angiogenesis inhibitor.

In this context, in one of the above uses it may be preferable for a substituted sulfonamide compound which is used to be present as the pure diastereomer and/or enantiomer, as a racemate or as a non-equimolar or equimolar mixture of the diastereomers and/or enantiomers.

The invention also provides a method for the treatment, in particular in one of the abovementioned indications, of a non-human mammal or a human requiring treatment by administration of a therapeutically active dose of a substituted sulfonamide compound according to the invention, or of a pharmaceutical composition according to the invention.

The invention also provides, in particular, a method for the treatment of pain, in a non-human mammal or a human in need thereof, comprising administering a therapeutically effective dose of a substituted sulfonamide compound according to the invention, or of a pharmaceutical composition according to the invention. The term pain includes particularly includes one or more of inflammatory pain, acute pain, visceral pain, neuropathic pain or chronic pain.

The invention also provides a process for the preparation of the substituted sulfonamide compounds according to the invention as described hereinafter.

In one aspect of the present invention, the substituted sulfonamide compounds according to the invention are prepared by the process described in the following reaction scheme:

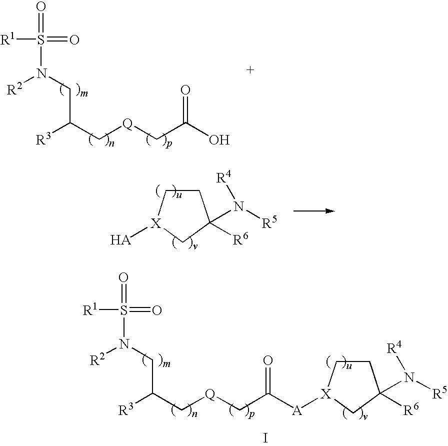

The free amines and the carboxylic acids are reacted in an amide formation in the presence at least of a dehydrating agent and optionally an organic base in an organic solvent (reaction medium) to give the compounds according to the invention.

Dehydrating agents which can be used include, for example, sodium sulfate or magnesium sulfate, phosphorus oxide or reagents such as, for example, CDI, DCC (optionally polymer-bonded), TBTU, EDCl, PyBOP or PFPTFA, also in the presence of HOAt or HOBt. Organic bases which can be used are, for example, triethylamine, DIPEA or pyridine, and organic solvents which can be used are THF, methylene chloride, diethyl ether, dioxane, DMF or acetonitrile. The temperature in the amide formation step can preferably be 0 to 50° C.

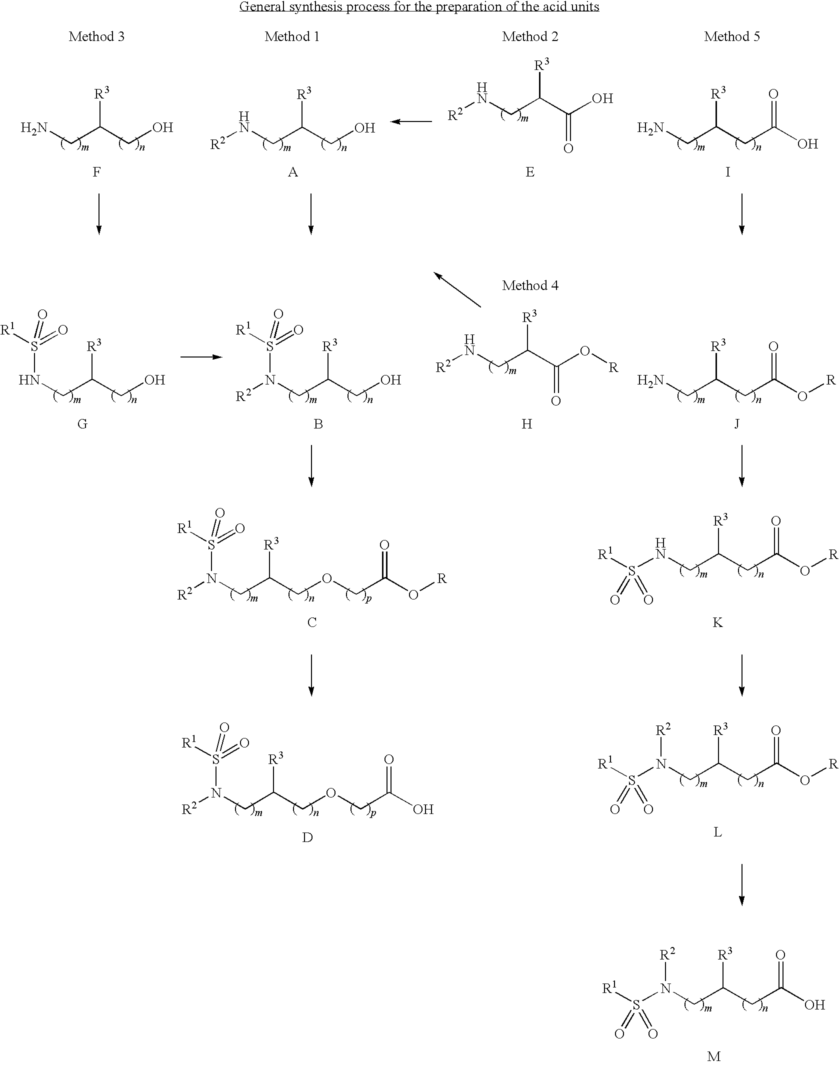

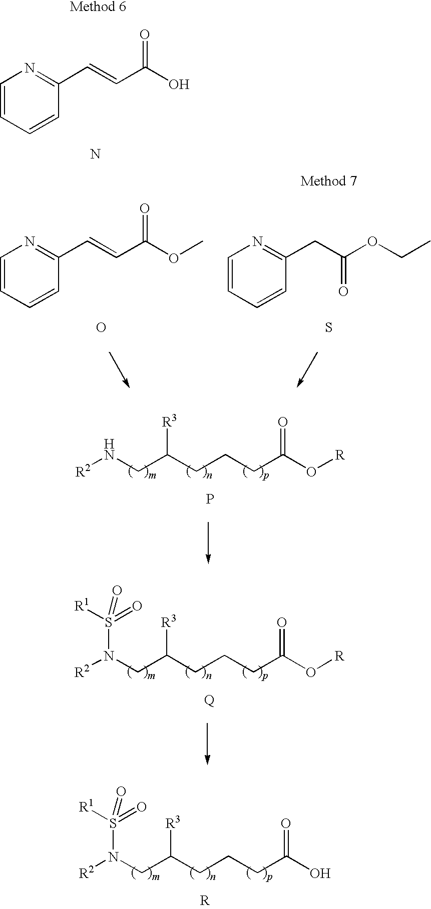

In Method 1, the racemic (R and S configuration) or enantiomerically pure (R or S configuration) amino alcohols A are reacted in a sulfonylation with sulfonyl chlorides, bromides or pentafluorophenolate R¹SO₂X (X=Cl, Br, OPFP), optionally in the presence of an organic or inorganic base, for example potassium carbonate, sodium carbonate, sodium bicarbonate, diisopropylethylamine, triethylamine, pyridine, dimethylaminopyridine, diethylamine or DBU, preferably in an organic solvent, for example acetone, acetonitrile, methylene chloride or tetrahydrofuran, and at a temperature of from 0° C. to the reflux temperature, to give the sulfonylated amino alcohols B.

The sulfonylated amino alcohols B are reacted in an alkylation reaction with halogenated ester derivatives, using tetrabutylammonium chloride or bromide or tetrabutylammonium hydrogen sulfate, in a phase transfer reaction using an organic solvent, such as THF, toluene, benzene or xylene, and an inorganic base, such as potassium hydroxide, sodium hydroxide, sodium carbonate, sodium bicarbonate, potassium carbonate, or in the presence of an organic or inorganic base, conventional inorganic bases are metal alcoholates, such as sodium methanolate, sodium ethanolate, potassium tert-butylate, lithium bases or sodium bases, such as lithium diisopropylamide, butyllithium, tert-butyllithium, sodium methylate, or metal hydrides, such as potassium hydride, lithium hydride, sodium hydride, conventional organic bases are diisopropylethylamine, triethylamine, in an organic solvent, such as methylene chloride, THF or diethyl ether, at 0° C. to the reflux temperature, to give the products of the general structure C.

In Method 2, the racemic (R and S configuration) or enantiomerically pure (R or S configuration) amino acids E are converted by a reduction into an amino alcohol A using metal hydrides as reducing agents, such as, for example, LiAlH₄, BH₃ x DMS or NaBH₄, in an organic solvent, such as THF or diethyl ether, at temperatures of from 0° C. to the reflux temperature. The further procedure corresponds to Method 1.

In Method 3, the racemic (R and S configuration) or enantiomerically pure (R or S configuration) amino alcohols F are reacted in a sulfonylation with sulfonyl chlorides, bromides or pentafluorophenolate R¹SO₂X (X=Cl, Br, OPFP), optionally in the presence of an organic or inorganic base, for example potassium carbonate, sodium bicarbonate, diisopropylethylamine, triethylamine, pyridine, dimethylaminopyridine, diethylamine or DBU, preferably in an organic solvent, for example acetone, acetonitrile, methylene chloride or tetrahydrofuran, and at a temperature of from 0° C. to the reflux temperature, to give the sulfonylated amino alcohols G. The sulfonylated amino alcohols G are then reacted in an alkylation reaction with alkyl halides (RX, X=I, Br, Cl), mesylates or alternative alkylating reagents, optionally in the presence of an organic or inorganic base, for example sodium hydride, potassium carbonate, caesium carbonate, DBU or DIPEA, preferably in an organic solvent, for example dimethylformamide, acetone, THF, acetonitrile, dioxane or these solvents as mixtures, at a temperature of from 0° C. to the reflux temperature, to give the sulfonylated amino alcohols B. The further process corresponds to Method 1.

In Method 4, the racemic (R and S configuration) or enantiomerically pure (R or S configuration) amino acid esters H are converted by a reduction into an amino alcohol A using metal hydrides as reducing agents, such as, for example, LiAlH₄, BH₃ x DMS or NaBH₄, in an organic solvent, such as THF or diethyl ether, at temperatures of from 0° C. to the reflux temperature. The further procedure corresponds to Method 1.

In Method 5, the racemic (R and S configuration) or enantiomerically pure (R or S configuration) acids I are esterified using dehydrating reagents, for example inorganic acids, such as H₂SO₄ or phosphorus oxides, or organic reagents, such as thionyl chloride, in organic solvents, such as THF, diethyl ether, methanol, ethanol or methylene chloride, to give stage J, at temperatures of from room temperature to the reflux temperature. The amino acid esters J are reacted in a sulfonylation with sulfonyl chlorides, bromides or pentafluorophenolate R₃SO₂X (X=Cl, Br, OPFP), optionally in the presence of an organic or inorganic base, for example potassium carbonate, sodium carbonate, sodium bicarbonate, diisopropylethylamine, triethylamine, pyridine, dimethylaminopyridine, diethylamine or DBU, preferably in an organic solvent, for example acetone, acetonitrile, methylene chloride or tetrahydrofuran, and at a temperature of from 0° C. to the reflux temperature, to give the sulfonylated amino esters K. The sulfonylated amino esters K are then reacted in an alkylation reaction with alkyl halides (RX, X=I, Br, Cl), mesylates or alternative alkylating reagents, optionally in the presence of an organic or inorganic base, for example sodium hydride, potassium carbonate, caesium carbonate, DBU or DIPEA, preferably in an organic solvent, for example dimethylformamide, acetone, THF, acetonitrile, dioxane or these solvents as mixtures, at a temperature of from 0° C. to the reflux temperature, to give the sulfonylated amino esters L.

In Method 6, 3-(pyridin-2-yl)acrylic acid N is esterified using dehydrating reagents, for example inorganic acids, such as H₂SO₄ or phosphorus oxides, or organic reagents, such as thionyl chloride, in organic solvents, such as THF, diethyl ether, methanol, ethanol or methylene chloride, to give stage 0, at temperatures of from room temperature to the reflux temperature.

In Methods 6 and 7, the ester stages O and S are hydrogenated in a hydrogenation under conditions known to the person skilled in the art in organic solvents, such as THF, chloroform, and in the presence of catalysts, such as platinum oxides, with hydrogen under normal pressure or increased pressure to give the intermediates P.

In Methods 6 and 7, stage P is reacted further in a sulfonylation with sulfonyl chlorides, bromides or pentafluorophenolate R¹SO₂X (X=Cl, Br, OPFP), optionally in the presence of an organic or inorganic base, for example potassium carbonate, sodium bicarbonate, diisopropylethylamine, triethylamine, pyridine, diethylamine or DBU, preferably in an organic solvent, for example acetonitrile, methylene chloride or tetrahydrofuran, at 0° C. to the reflux temperature, to give the sulfonylated amino esters Q.

In Methods 1-6, the ester derivatives C, L and Q are reacted in an ester cleavage using organic acids, such as trifluoroacetic acid, or aqueous inorganic acids, such as hydrochloric acid, or using aqueous inorganic bases, such as lithium hydroxide, potassium hydroxide, sodium hydroxide, sodium carbonate, sodium bicarbonate, potassium carbonate, in organic solvents, such as methanol, dioxane, methylene chloride, THF, diethyl ether or these solvents as mixtures, at 0° C. to room temperature, to give the acid stages of the general formula D, M and R.

### General Synthesis Process for the Preparation of the Amine Units:

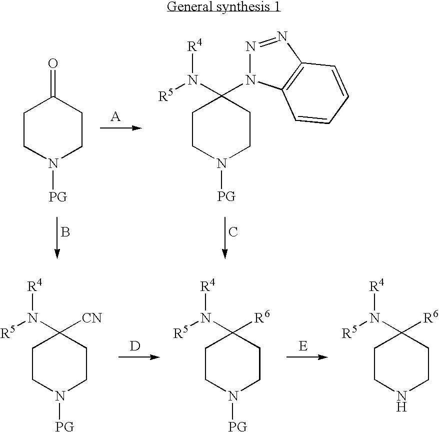

A: The protected piperidin-4-one is reacted in an aminal formation reaction by a reaction known to the person skilled in the art with an amine and 1H-benzotriazole to give the benzotriazole aminal. It is known to the person skilled in the art that the benzotriazole aminal can be present in equilibrium both in the 1H and in the 2H form. Suitable solvents are benzene, toluene, ethanol, diethyl ether or THF. The use of a Dean-Stark water separator, a molecular sieve or other dehydrating agents may be necessary. The reaction time can be between 1 and 20 h at a reaction temperature of from +20° C. to +110° C.

B: The protected piperidin-4-one is converted into the nitrile by addition of an amine and a source of cyanide. The reaction can be carried out in one or two stages, as is known to the person skilled in the art. In the two-stage variant, a nitrile alcohol is first formed and isolated. The nitrile alcohol can be formed by reaction of the protected piperidin-4-one with HCN, KCN or NaCN. Typical solvents are water, methanol, ethanol, THF, piperidine, diethyl ether or a mixture of these solvents. If NaCN and KCN are used, the cyanide required can typically be liberated by addition of, for example, sodium hydrogen sulfite, sulfuric acid, acetic acid or hydrochloric acid. Trimethylsilyl cyanide, for example, is likewise suitable as a source of nitrile. In this case the cyanide can be liberated, for example, by boron trifluoride etherate, InF₃ or HCl. Typical solvents here are water or toluene. (Cyano-C)diethylaluminium, for example, is suitable as a further source of cyanide. THF, toluene or a mixture of the two solvents can be used as the solvent.

The reaction temperature can be between −78° C. and +25° C. for all the variants. Alcohols, such as methanol or ethanol, are particularly suitable as the solvent for the reaction of the nitrile alcohol with the amine. The reaction temperature can be between 0° C. and +25° C. In the one-stage variant, the nitrile alcohol primarily formed is formed in situ and reacted with the amine.

C/D: Both the benzotriazole aminal obtained from the reaction A and the nitrile obtained from the reaction B can be reacted, as is known to the person skilled in the art, with metal organyls, such as magnesium, zinc or lithium organyls, in organic solvents, for example diethyl ether, dioxane or THF, to give 4-substituted 4-aminopiperidines.

E: The method for splitting off of the protective group depends on the nature of the protective group used. Suitable protective groups are, for example, the Boc, Cbz, Fmoc or benzyl protective group.

BOC protective groups can be split off, for example, by reaction with HCl in organic solvents, such as, for example, dioxane, methanol, ethanol, acetonitrile or ethyl acetate, or by reaction with TFA or methanesulfonic acid in methylene chloride or THF at a temperature of from 0° C. to 110° C. over a reaction time of 0.5-20 h. The Cbz protective group can be split off, for example, under acidic conditions. This acidic splitting off can be carried out, for example, by reaction with an HBr/glacial acetic acid mixture, a mixture of TFA in dioxane/water or HCl in methanol or ethanol. However, reagents such as, for example, Me₃SiI, in solvents, such as, for example, MC, chloroform or acetonitrile, BF₃ etherate with the addition of ethanethiol or Me₂S, in solvents, such as, for example, MC, a mixture of aluminium chloride/anisole in a mixture of MC and nitromethane or triethylsilane/PdCl₂ in methanol with the addition of triethylamine, are also suitable. A further method is the hydrogenolytic splitting off of the protective group under increased pressure or normal pressure with the aid of catalysts, such as, for example, Pd on charcoal, Pd(OH)₂, PdCl₂, Raney nickel or PtO₂, in solvents, such as, for example, methanol, ethanol, 2-propanol, THF, acetic acid, ethyl acetate, chloroform, optionally with the addition of HCl, formic acid or TFA. The Fmoc protective group is as a rule split off under basic conditions in solvents, such as, for example, acetonitrile, DMF, THF, diethyl ether, methanol, ethanol, 1-octanethiol, MC or chloroform. Suitable bases are, for example, diethylamine, piperidine, 4-aminomethylpiperidine, pyrrolidine, DBU, NaOH or LiOH. However, reagents such as, for example, Ag₂O/MeI can also be used. A benzylic protective group can be split off, for example, by catalytic hydrogenation. Suitable catalysts are, for example, Pd on charcoal, PtO₂ or Pd(OH)₂. The reaction can be carried out in solvents, such as, for example, ethanol, methanol, 2-propanol, acetic acid, THF or DMF, optionally with the addition of acids, such as, for example, ammonium formate, maleic acid or formic acid, or in mixtures of the solvents.

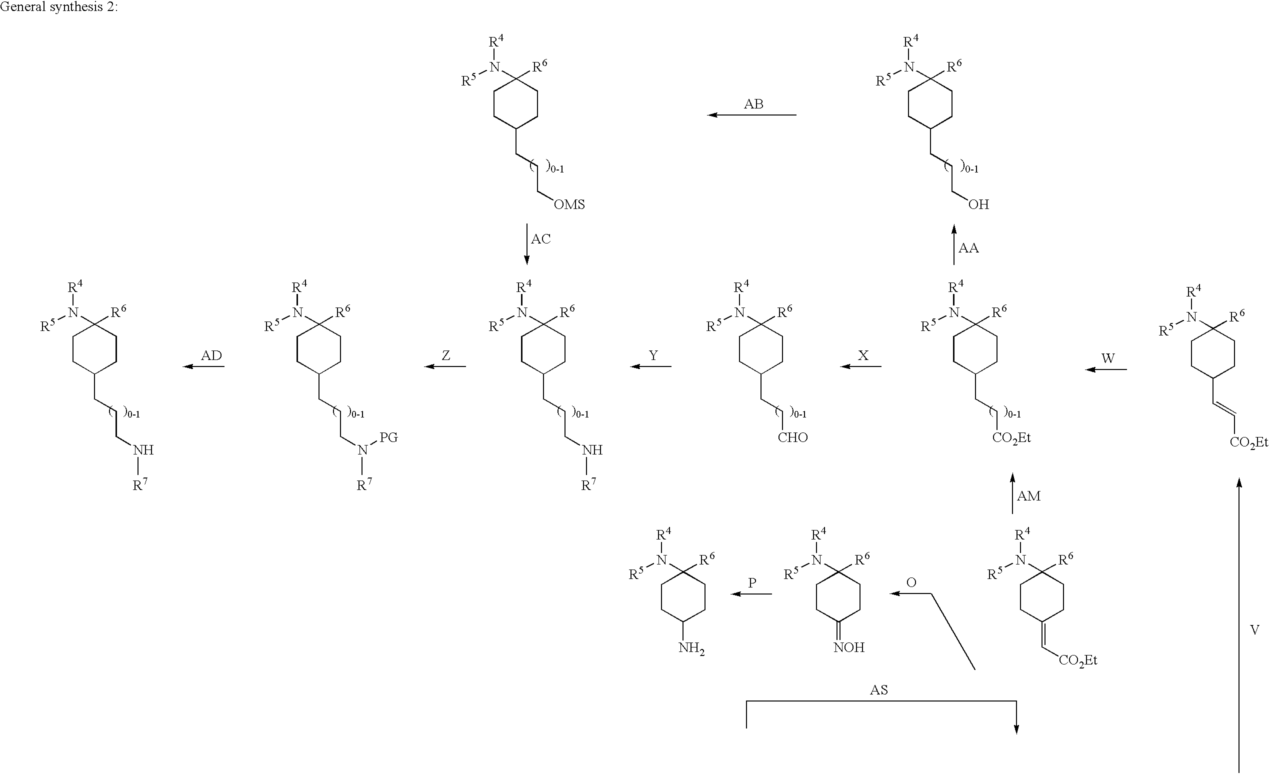

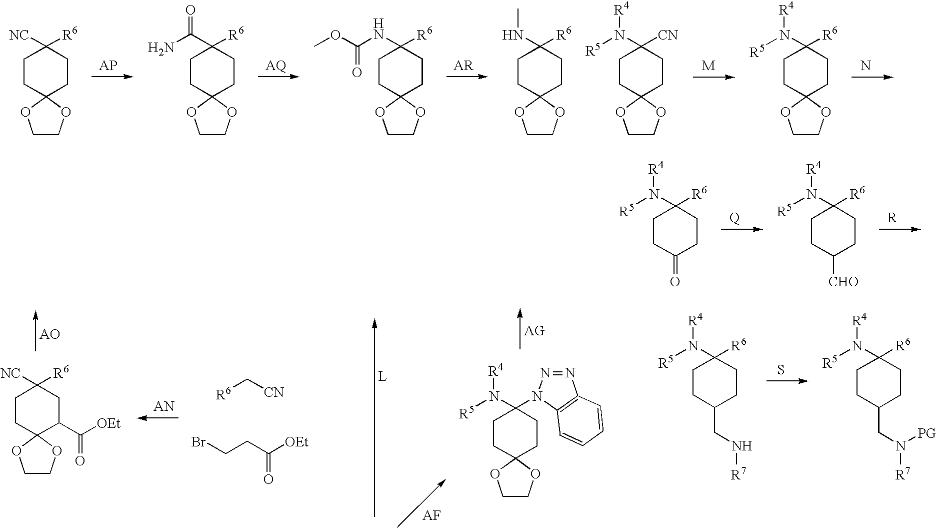

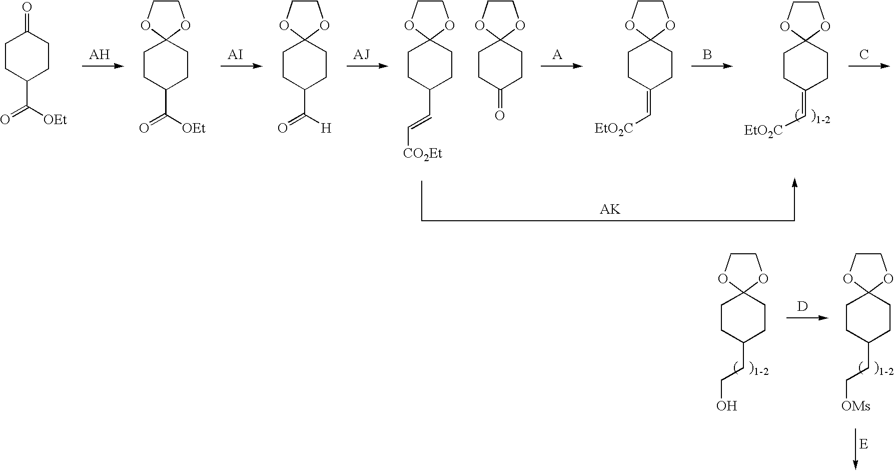

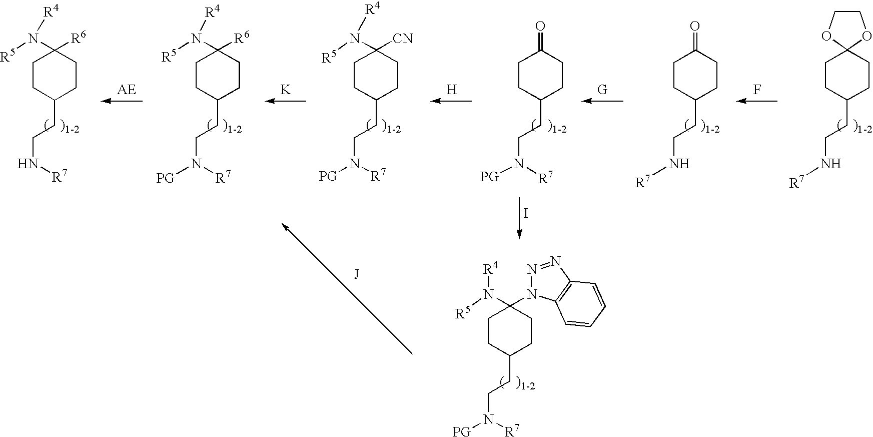

A/V/AJ/AL: The unsaturated ester can be prepared, as is known to the person skilled in the art, in a Wittig-Horner reaction from the keto acetal and ethyl 2-(dimethoxyphosphoryl)acetate or methyl 2-(diethylphosphino)acetate using bases, such as, for example, NaH, K₂CO₃, sodium methanolate, potassium tert-butylate, lithium diisopropylamide or n-butyllithium, in solvents, such as, for example, water, THF, diethyl ether, diisopropyl ether, hexane, benzene, toluene, 1,2-dimethoxyethane, DMF or DMSO. Reagents such as, for example, MgBr₂, triethylamine or HMPT are optionally added.

B/W/AK/AM: The double bond of the unsaturated ester can be reduced, as is known to the person skilled in the art, by hydrogenolysis with homogeneous or heterogeneous catalysts or by reaction with reducing agents. A suitable homogeneous catalyst is, for example, tris(triphenylphosphane)rhodium chloride in solvents, such as, for example, benzene or toluene. Heterogeneous catalysts which can be used are, for example, Pt on charcoal, palladium on charcoal, Raney nickel or Pt₂O in solvents, such as, for example, acetic acid, methanol, ethanol, ethyl acetate, hexane, chloroform or mixtures of these solvents. Acids, such as, for example, sulfuric acid or hydrochloric acid, can optionally be added. A suitable reducing agent is, for example, L-selectride in, for example, THF.

C: The reduction of the ester function to give the alcohol can be carried out with the aid of various reducing agents. Suitable reducing agents are, for example, LiBH₄ or NaBH₄ in solvents, such as, for example, diethyl ether, toluene, THF, water, methanol, ethanol or mixtures of these solvents, optionally with the addition of auxiliary reagents, such as, for example, boric acid esters. However, Zn(BH₄)₂ in, for example, DME can also be used as a further borohydride. The reduction can also be carried out, however, with BH₃-Me₂S complex in solvents, such as, for example, THF or MC. In addition to the boron compounds, the complex aluminium hydrides, such as, for example, DIBAH or LAH, in solvents, such as, for example, diethyl ether, benzene, toluene, THF, MC, DME, hexane or mixtures of these solvents, are also suitable for reduction of the ester function to the alcohol.

D/AB: The mesylation is carried out, as is known to the person skilled in the art, in solvents, such as, for example, chloroform, MC, diethyl ether, THF or toluene, optionally with the addition of bases, such as, for example, triethylamine, pyridine or diisopropylethylamine, and optionally with the addition of auxiliary reagents, such as, for example, DMAP. Alternatively to converting the hydroxyl functional group into a mesylate as a suitable leaving group it may also be converted to any other leaving group (e.g. halogen) known to a person skilled in the art.

E/AC: The subsequent substitution reaction with an amine can be carried out, as is known to the person skilled in the art, in solvents, such as, for example, acetonitrile, benzene, toluene, water, methanol, ethanol, 1-butanol, THF, dioxane, DME, DMF, DMSO or mixtures of the solvents, optionally with the addition of bases, such as, for example, Na₂CO₃, K₂CO₃, triethylamine or diisopropylethylamine, and optionally with the addition of auxiliary reagents, such as, for example, KI.

F/N: The ketone is obtained under conditions known to the person skilled in the art in an acetal cleavage reaction under acidic conditions. Suitable acids are both inorganic Broenstedt or Lewis acids, such as hydrochloric acid, sulfuric acid, ammonium chloride or hydrogen sulfate or AlCl₃, and organic acids, such as e.g. p-toluenesulfonic acid, acetic acid, oxalic acid, trifluoromethanesulfonic acid, formic acid, trifluoroacetic acid or citric acid. The reaction can be carried out in various solvents, such as, for example, toluene, THF, chloroform, MC, xylene, acetonitrile, water, dioxane, acetone, diethyl ether or ethyl acetate, at temperatures of from −10° C. to room temperature.

G/S/Z: The amine function is protected with the aid of a protective group. As is known to the person skilled in the art, carbamates, such as, for example, the Boc, Fmoc or Cbz (Z) protective group, or a benzylic protective group are suitable as protective groups.

The introduction of the BOC protective group by means of di-tert-butyl dicarbonate can be carried out in solvents, such as, for example, dioxane, MC, THF, DMF, water, benzene, toluene, methanol, acetonitrile or mixtures of these solvents, optionally with the addition of sodium hydroxide, triethylamine, diisopropylethylamine, sodium bicarbonate, sodium carbonate or DMAP, at temperatures of between 0° C. and 100° C.

The Fmoc protective group is introduced by reaction of 9H-fluoren-9-ylmethyl chloroformate in solvents, such as, for example, MC, DCE, diethyl ether, THF, dioxane, acetone, acetonitrile, DMF or water, optionally with the addition of a base, such as, for example, diisopropylethylamine, triethylamine, pyridine, N-methylmorpholine, sodium carbonate or sodium bicarbonate, and optionally under irradiation with microwaves.

The Cbz protective group can be introduced by reaction of chloroformic acid benzyl ester in solvents, such as, for example, diethyl ether, THF, DMF, benzene, toluene, dioxane, water, acetone, ethyl acetate, MC or chloroform, optionally with the addition of a base, such as, for example, sodium carbonate, sodium bicarbonate, potassium carbonate, sodium hydroxide or triethylamine, optionally with the addition of a coupling reagent, such as, for example, HOBt.

The benzylic protective group can be introduced by alkylation by means of chloro- or bromobenzyl compounds or by reductive amination with benzaldehydes. The alkylation can be carried out in solvents, such as, for example, ethanol, methanol, water, acetonitrile, MC, THF, DMSO or mixtures of these solvents. If appropriate, a base, such as, for example, diethylamine, sodium bicarbonate, sodium carbonate, potassium carbonate or caesium carbonate, and if appropriate an auxiliary reagent, such as, for example, potassium iodide or sodium iodide, must be added. The reductive amination is carried out in solvents, such as, for example, methanol, ethanol, DCE or MC. Suitable reducing agents are, for example, sodium cyanoborohydride or sodium triacetoxyborohydride, optionally with the addition of acetic acid.

H/L: The ketone is converted into the aminonitrile by addition of an amine and a source of cyanide. The reaction can be carried out in one or two stages, as is known to the person skilled in the art. In the two-stage variant, a nitrile alcohol is first formed and isolated. The nitrile alcohol can be formed by reaction of the protected diketone with HCN, KCN or NaCN. Typical solvents are water, methanol, ethanol, THF, piperidine, diethyl ether or a mixture of these solvents. If NaCN and KCN are used, the cyanide required can typically be liberated by addition of, for example, sodium hydrogen sulfite, sulfuric acid, acetic acid or hydrochloric acid. Trimethylsilyl cyanide, for example, is likewise suitable as a source of nitrile. In this case the cyanide can be liberated, for example, by boron trifluoride etherate, InF₃ or HCl. Typical solvents here are water or toluene.

(Cyano-C)diethylaluminium, for example, is suitable as a further source of cyanide. THF, toluene or a mixture of the two solvents can be used as the solvent.

The reaction temperature can be between −78° C. and +25° C. for all the variants. Alcohols, such as methanol or ethanol, are particularly suitable as the solvent for the reaction of the nitrile alcohol with the amine. The reaction temperature can be between 0° C. and +25° C. In the one-stage variant, the nitrile alcohol primarily formed is formed in situ and reacted with the amine.

I/AF: The ketone can be reacted in an aminal formation reaction by the reaction known to the person skilled in the art with an amine and 1H-benzotriazole to give the benzotriazole aminal. It is known to the person skilled in the art that the benzotriazole aminal can be present in equilibrium both in the 1H and in the 2H form. Suitable solvents are benzene, toluene, ethanol, diethyl ether or THF. The use of a Dean-Stark water separator, a molecular sieve or other dehydrating agents may be necessary. The reaction time can be between 1 and 20 h at a reaction temperature of from +20° C. to +110° C.

J/K/M/AG: Both the benzotriazole aminal obtained from reactions I and AF and the nitriles obtained from reactions H and L can be reacted, as is known to the person skilled in the art, with metal organyls, such as magnesium, zinc or lithium organyls, in organic solvents, for example diethyl ether, dioxane or THF, to give the corresponding protected amine.

O/T: The ketone or the aldehyde is reacted in an oxime formation reaction under the conditions known to the person skilled in the art with hydroxylamine hydrochloride, sulfate or acetate in an organic solvent, for example ethanol, methanol, 2-propanol, 2-methyl-propan-2-ol or acetonitrile, with the addition of an organic base, such as, for example, pyridine, sodium acetate, triethylamine, DMAP or potassium t-butylate, or an aqueous solution of an inorganic base, such as sodium bicarbonate, sodium carbonate, potassium carbonate, sodium hydroxide or potassium hydroxide, or the basic ion exchanger Amberlyst, to give the oximes.

P/U: The amines can be obtained by a reduction reaction, known to the person skilled in the art, of the oximes with a reducing agent, such as, for example, LAH, sodium, zinc, borane dimethylsulfide, sodium borohydride/nickel(II) chloride hexahydrate, in ethanol, methanol, glacial acetic acid, THF, diethyl ether or dioxane, or by catalytic hydrogenation with palladium or platinum oxide as a heterogeneous catalyst, with the addition of HCl in an alcohol, such as methanol or ethanol.

Q: The aldehyde is obtained under the conditions, known to the person skilled in the art, of a Wittig reaction using a corresponding phosphonium compound, for example (methoxymethyl)triphenyl-phosphonium chloride, and a strong base, for example potassium tert-butylate, n-butyllithium, s-butyllithium, phenyllithium, lithium diisopropylamide or lithium hexamethyldisilazide, in organic solvents, such as THF, diethyl ether, cyclohexane, toluene or a mixture of the solvents, at a temperature of from −78° C. to +30° C., after acidic working up of the reaction mixture.

R/Y: The subsequent reductive amination can be carried out, as is known to the person skilled in the art, by reaction with amines and subsequent reduction with reducing agents, such as, for example, NaBH(OAc)₃, NaBH₄, LiBH₃CN, NaBH₃CN, borane-pyridine complex or α-picoline-borane complex, in solvents, such as, for example, ethanol, methanol, MC, DCE, THF, DMF, benzene, toluene or mixtures of these solvents, optionally with the addition of acids, such as, for example, HCl or acetic acid. Alternatively, the aldehyde can be reacted with a corresponding amine to give the imine, optionally with the addition of dehydrating agents, and then converted into the amine by catalytic hydrogenation. Suitable catalysts are, for example, Pt₂O, Pd on charcoal or Raney nickel, in solvents, such as, for example, ethanol or methanol.

X: The reduction of the ester function can be carried out hydrogenolytically with Pd on charcoal as a heterogeneous catalyst in solvents, such as, for example, DME, ethanol or a solvent mixture. It is moreover known to the person skilled in the art that the reduction of the ester to the aldehyde can be carried out with the aid of reducing agents, such as, for example, DIBAH in, for example, toluene or sodium tris(diethylamino)aluminium hydride in, for example, THF.

AA: The reduction to give the alcohol can be carried out employing various reducing agents. The reduction can be carried out, with BH₃-Me₂S complex in solvents, such as, for example, THF or MC. In addition complex aluminium hydrides, such as, for example, DIBAH or LAH, in solvents, such as, for example, diethyl ether, benzene, toluene, THF, MC, DME, hexane or mixtures of these solvents, are also suitable for reduction to the alcohol.

AD/AE: The method for removing protective groups (PG) depends on the nature of the protective group used. For example, carbamates, such as, for example, the Boc, Fmoc or Cbz(Z) protective group, or also benzylic protective groups are suitable.

The BOC protective group can be split off, for example, by reaction with HCl in organic solvents, such as dioxane, methanol, ethanol, acetonitrile or ethyl acetate, or by reaction with TFA or methanesulfonic acid in methylene chloride or THF at a temperature of from 0° C. to 110° C. over a reaction time of 0.5-20 h.

The Cbz protective group can be split off, for example, under acidic conditions. This acidic splitting off can be carried out, for example, by reaction with an HBr/glacial acetic acid mixture, a mixture of TFA in dioxane/water or HCl in methanol or ethanol. However, reagents such as, for example, Me₃SiI, in solvents, such as, for example, MC, chloroform or acetonitrile, BF₃ etherate with the addition of ethanethiol or Me₂S, in solvents, such as, for example, MC, a mixture of aluminium chloride/anisole in a mixture of MC and nitromethane, or triethylsilane/PdCl₂ in methanol, with the addition of triethylamine, are also suitable. A further method is the hydrogenolytic splitting off of the protective group under increased pressure or normal pressure with the aid of catalysts, such as, for example, Pd on charcoal, Pd(OH)₂, PdCl₂, Raney nickel or PtO₂, in solvents, such as, for example, methanol, ethanol, 2-propanol, THF, acetic acid, ethyl acetate, chloroform, optionally with the addition of HCl, formic acid or TFA. The Fmoc protective group is as a rule split off under basic conditions in solvents, such as, for example, acetonitrile, DMF, THF, diethyl ether, methanol, ethanol, 1-octanethiol, MC or chloroform. Suitable bases are, for example, diethylamine, piperidine, 4-aminomethylpiperidine, pyrrolidine, DBU, NaOH or LiOH. However, reagents such as, for example, Ag₂O/MeI can also be used.

A benzylic protective group can be removed, for example, by catalytic hydrogenation. Suitable catalysts are, for example, Pd on charcoal, PtO₂ or Pd(OH)₂. The reaction can be carried out in solvents, such as, for example, ethanol, methanol, 2-propanol, acetic acid, THF or DMF, with the addition of acids, such as, for example, ammonium formate, maleic acid or formic acid, or in mixtures of the solvents. Protecting groups can be selected from a large variety of possibilities and can be cleaved according to the literature, e.g as described in:
Philip J. Kocienski, Protecting Groups, 3rd Edition, Georg Thieme Verlag, 2005 (ISBN 3-13-135603-0), in particular pages 505-524, 528-534, 570-585, 606-618 and 625, and
Peter G. M. Wuts, Theodora W. Greene, Greene's Protective Groups in Organic Synthesis, 4th Edition, Wiley-Interscience, 2007 (ISBN-13: 978-0-471-69754-1), in particular pages 696-932.

AH: The protecting group can be introduced according to standard literature procedures:

The ketone is reacted with ethane-1,2-diol in presence of a protic acid catalyst (for example p-toluenesulfonic acid or an acid exchange resin) in for example benzene or toluene under Dean Stark conditions or in the presence of molecular sieves, a chemical dehydrating agent, such as magnesium sulfate or calcium sulfate.

Any alternative suitable ketone protecting group may be employed instead, see
Philip J. Kocienski, Protecting Groups, 3rd Edition, Georg Thieme Verlag, 2005 (ISBN 3-13-135603-0), in particular pages 50-110
Peter G. M. Wuts, Theodora W. Greene, Greene's Protective Groups in Organic Synthesis, 4th Edition, Wiley-Interscience, 2007 (ISBN-13: 978-0-471-69754-1), in particular pages 431-432.

AI: The reduction of the ester (although an ethyl ester is employed any other suitable estar may also be employed instead (e.g. methyl ester)) is carried out employing complex aluminium hydrides, such as, for example, DIBAH, in solvents, such as, for example, diethyl ether, benzene, toluene, THF, MC, DME, hexane or mixtures of these solvents, are also suitable.

AN: The nitrile was reacted with the ethyl ester (any other suitable ester, such as a methyl ester, may also be chosen) under basic conditions employing potassium tert-butoxide or sodium amide in a suitable solvent or solvent mixture, such as DMF, toluene, diethyl ether or THF.

AO: This transformation is carried out under acidic conditions in the presence of hydrochloric acid and acetic acid in aqueous solution.

AP: The hydrolysis of the nitrile to the corresponding amide can be carried out under acidic or basic conditions, employing for example hydrogen chloride, sulfuric acid, polyphosphoric acid, hydrogen bromide, lithium hydroxide, sodium hydroxide, potassium hydroxide or potassium trimethylsilanolate, sometimes in the presence of metal salts, such as for example copper salts, in a suitable solvent or solvent mixture, selected from, methanol, ethanol, dichloromethane, DMSO, water and THF.

AQ: This transformation is carried out in the presence of KF/Al₂O₃ and sodium hypochlorite solution in solvents such as methanol, ethanol, water or mixtures thereof.

AR: The transformation is carried out employing complex aluminium hydrides, such as, for example, LAH, in solvents, such as, for example, diethyl ether, benzene, toluene, THF, MC, DME, hexane or mixtures of these solvents.

AS: The reductive amination is carried out by reaction of aldehydes with amines and subsequent reduction with reducing agents, such as, for example, NaBH(OAc)₃, NaBH₄, LiBH₃CN, NaBH₃CN, borane-pyridine complex or α-picoline-borane complex, in solvents, such as, for example, ethanol, methanol, MC, DCE, THF, DMF, benzene, toluene or mixtures of these solvents, optionally with the addition of acids, such as, for example, HCl or acetic acid. Alternatively, the aldehyde can be reacted with a corresponding amine to give the imine, optionally with the addition of dehydrating agents, and then converted into the amine by catalytic hydrogenation. Suitable catalysts are, for example, Pt₂O, Pd on charcoal or Raney nickel, in solvents, such as, for example, ethanol or methanol.

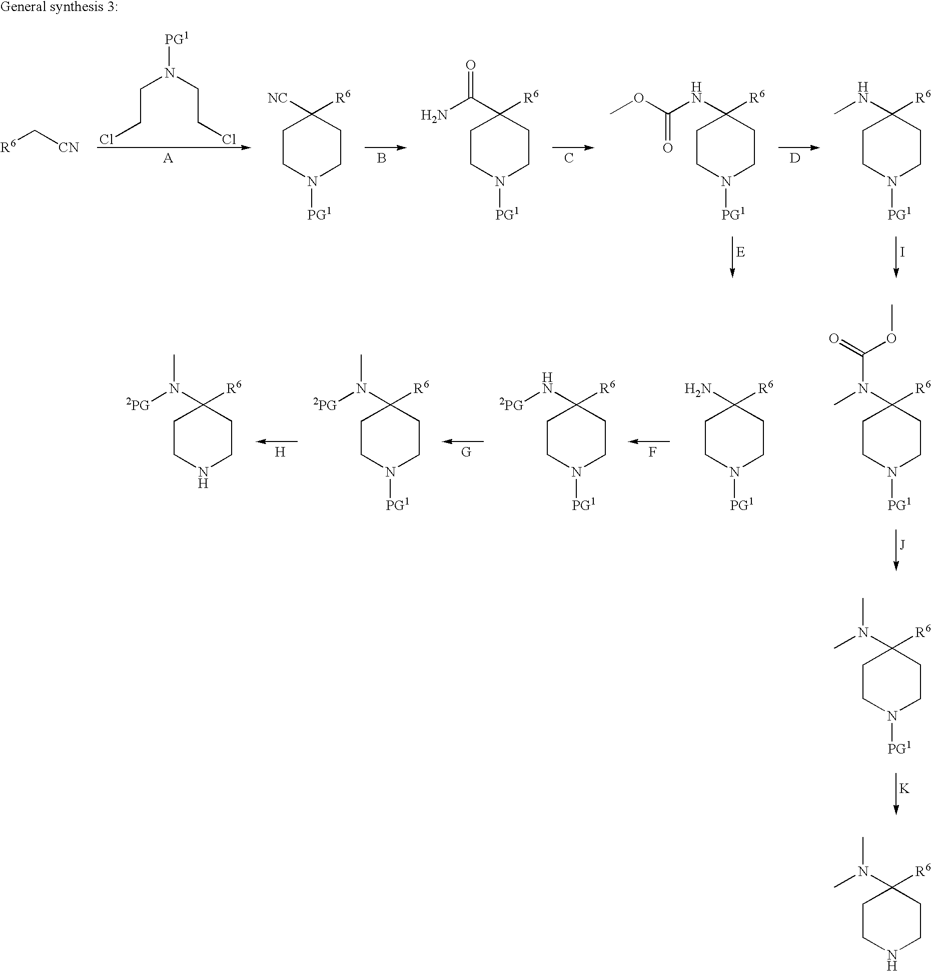

A: The cyanide is reacted with the halide (instead of the chloride other suitable leaving groups, such as bromide or mesylate may also be employed) in the presence of a suitable base such as potassium hydroxide, sodium hydroxide, sodium hydride, potassium carbonate or potassium tert-butoxide, sometimes in the presence of 18-crown-6, tetra-butylammonium chloride or triethylbenzylammonium chloride, in solvents such as benzene, toluene, water, acetonitrile, 1,2-dimethoxyethane, DMF or mixtures thereof.

B: The hydrolysis of the nitrile to the corresponding amide can be carried out under acidic or basic conditions, employing for example hydrogen chloride, sulfuric acid, polyphosphoric acid, hydrogen bromide, lithium hydroxide, sodium hydroxide, potassium hydroxide or potassium trimethylsilanolate, sometimes in the presence of metal salts, such as for example copper salts, in a suitable solvent or solvent mixture, selected from, methanol, ethanol, dichloromethane, DMSO, water and THF.

C: This transformation is carried out in the presence of KF/Al₂O₃ and sodium hypochlorite solution in solvents such as methanol, ethanol, water or mixtures thereof.

D/J: The transformation is carried out employing complex aluminium hydrides, such as, for example, LAH, in solvents, such as, for example, diethyl ether, benzene, toluene, THF, MC, DME, hexane or mixtures of these solvents.

E: The methyl carbonate group is removed in the presence of bases, such as potassium hydroxide, sodium hydroxide or lithium hydroxide in solvents such as methanol, ethanol, water or mixtures thereof.

F: The method for removing protective groups (PG) depends on the nature of the protective group used. For example, carbamates, such as, for example, the Boc, Fmoc or Cbz(Z) protective group, or also benzylic protective groups are suitable.

The preferred BOC protective group can be split off, for example, by reaction with HCl in organic solvents, such as dioxane, methanol, ethanol, acetonitrile or ethyl acetate, or by reaction with TFA or methanesulfonic acid in methylene chloride or THF at a temperature of from 0° C. to 110° C. over a reaction time of 0.5-20 h.

G: The alkylation is carried out in the presence of a suitable base, such as sodium hydride or potassium carbonate, as well as a suitable methylating agent, such as methyl iodide, methyl bromide or dimethyl sulfate, in solvents such as DMF, THF or mixtures thereof.

I: The transformation with methyl chloroformate is carried out in the presence of a suitable base, such as for example sodium hydride, triethylamine, Hünig base, sodium hydroxide or potassium carbonate, in a suitable solvent, such as for example THF, dichloromethane, acetone, diethylether, chloroform or mixtures thereof.

H/K: The method for removing protective groups (PG) depends on the nature of the protective group used. For example, carbamates, such as, for example, the Boc, Fmoc or Cbz(Z) protective group, or also benzylic protective groups are suitable.

The BOC protective group can be split off, for example, by reaction with HCl in organic solvents, such as dioxane, methanol, ethanol, acetonitrile or ethyl acetate, or by reaction with TFA or methanesulfonic acid in methylene chloride or THF at a temperature of from 0° C. to 110° C. over a reaction time of 0.5-20 h.

The Cbz protective group can be removed, for example, under acidic conditions. This acidic splitting off can be carried out, for example, by reaction with an HBr/glacial acetic acid mixture, a mixture of TFA in dioxane/water or HCl in methanol or ethanol. However, reagents such as, for example, Me₃SiI, in solvents, such as, for example, MC, chloroform or acetonitrile, BF₃ etherate with the addition of ethanethiol or Me₂S, in solvents, such as, for example, MC, a mixture of aluminium chloride/anisole in a mixture of MC and nitromethane, or triethylsilane/PdCl₂ in methanol, with the addition of triethylamine, are also suitable. A further method is the hydrogenolytic splitting off of the protective group under increased pressure or normal pressure with the aid of catalysts, such as, for example, Pd on charcoal, Pd(OH)₂, PdCl₂, Raney nickel or PtO₂, in solvents, such as, for example, methanol, ethanol, 2-propanol, THF, acetic acid, ethyl acetate, chloroform, optionally with the addition of HCl, formic acid or TFA. The Fmoc protective group is as a rule split off under basic conditions in solvents, such as, for example, acetonitrile, DMF, THF, diethyl ether, methanol, ethanol, 1-octanethiol, MC or chloroform. Suitable bases are, for example, diethylamine, piperidine, 4-aminomethylpiperidine, pyrrolidine, DBU, NaOH or LiOH. However, reagents such as, for example, Ag₂O/MeI can also be used.

A benzylic protective group can be removed, for example, by catalytic hydrogenation. Suitable catalysts are, for example, Pd on charcoal, PtO₂ or Pd(OH)₂. The reaction can be carried out in solvents, such as, for example, ethanol, methanol, 2-propanol, acetic acid, THF or DMF, with the addition of acids, such as, for example, ammonium formate, maleic acid or formic acid, or in mixtures of the solvents.

### Pharmacological Studies

### 1. Functional Investigation on the Bradykinin 1 Receptor (B1R)

The agonistic or antagonistic action of substances can be determined on the bradykinin 1 receptor (B1R) of the human and rat species with the following assay. In accordance with this assay, the Ca²⁺ inflow through the channel is quantified with the aid of a Ca²⁺-sensitive dyestuff (type Fluo-4, Molecular Probes Europe BV, Leiden, Holland) in a fluorescent imaging plate reader (FLIPR, Molecular Devices, Sunnyvale, USA).

### Method:

Chinese hamster ovary cells (CHO K1 cells) transfected stably with the human B1R gene (hB1R cells, Euroscreen s.a., Gosselies, Belgium) or the B1R gene of the rat (rB1R cells, Axxam, Milan, Italy) are used. For functional studies, these cells are plated out on black 96-well plates with a clear base (BD Biosciences, Heidelberg, Germany) in a density of 20,000-25,000 cells/well. The cells are incubated overnight at 37° C. and 5% CO₂ in culture medium (hB1R cells: Nutrient Mixture Ham's F12, Gibco Invitrogen GmbH, Karlsruhe, Germany; rB1R cells: D-MEM/F12, Gibco Invitrogen GmbH, Karlsruhe, Germany) with 10 vol. % of FBS (foetal bovine serum, Gibco Invitrogen GmbH, Karlsruhe, Germany). On the following day, the cells are loaded for 60 min at 37° C. with 2.13 μM Fluo-4 (Molecular Probes Europe BV, Leiden, Holland) in HBSS buffer (Hank's buffered saline solution, Gibco Invitrogen GmbH, Karlsruhe, Germany) with 2.5 mM probenecid (Sigma-Aldrich, Taufkirchen, Germany) and 10 mM HEPES (Sigma-Aldrich, Taufkirchen, Germany).

The plates are then washed 2× with HBSS buffer, and HBSS buffer which additionally contains 0.1% of BSA (bovine serum albumin; Sigma-Aldrich, Taufkirchen, Germany), 5.6 mM glucose and 0.05% of gelatine (Merck KGaA, Darmstadt, Germany) is added. After a further incubation of 20 minutes at room temperature, the plates are inserted into the FLIPR for the Ca²⁺ measurement. The Ca²⁺-dependent fluorescence is measured here before and after addition of substances (λₑₓ=488 nm, λₑₘ=540 nm). Quantification is by measurement of the highest fluorescence intensity (FC, fluorescence counts) over time.

### 2. FLIPR Assay:

The FLIPR protocol consists of 2 additions of substance. Test substances (10 μM) are first pipetted on to the cells and the Ca²⁺ inflow is compared with the control (hB1R: Lys-Des-Arg⁹-bradykinin 0.5 nM; rB1R: Des-Arg⁹-bradykinin 100 nM). This gives the figure in % activation based on the Ca²⁺ signal after addition of Lys-Des-Arg⁹-bradykinin (0.5 nM) or Des-Arg⁹-bradykinin (100 nM). After incubation for 10 minutes, 0.5 nM Lys-Des-Arg⁹-bradykinin (hB1R) or 100 nM Des-Arg⁹-bradykinin (rB1R) is applied and the inflow of Ca²⁺ is likewise determined. Antagonists lead to a suppression of the Ca²⁺ inflow. % inhibition compared with the maximum achievable inhibition is calculated. The compounds show a good activity on the human and on the rat receptor.

### 3. Method for Determination of the Affinity for the Human μ Opiate Receptor

The receptor affinity for the human μ opiate receptor is determined in a homogeneous set-up in microtitre plates. For this, dilution series of the substances to be tested are incubated with a receptor membrane preparation (15-40 μg of protein/250 μl of incubation batch) of CHO-K1 cells which express the human p opiate receptor (RB-HOM receptor membrane preparation from PerkinElmer Life Sciences, Zaventem, Belgium) in the presence of 1 nmol/l of the radioactive ligand [³H]-naloxone (NET719, PerkinElmer Life Sciences, Zaventem, Belgium) and 1 mg of WGA-SPA-Beads (wheat germ agglutinin SPA beads from Amersham/Pharmacia, Freiburg, Germany) in a total volume of 250 μl for 90 minutes at room temperature. 50 mmol/l of Tris-HCl supplemented with 0.06% of bovine serum albumin are used as the incubation buffer. 100 μmol/l of naloxone are additionally added for determination of the non-specific binding. After the end of the ninety-minute incubation time, the microtitre plates are centrifuged for 20 minutes at 1,000 g and the radioactivity is measured in a β-counter (Microbeta-Trilux, Perkin Elmer Wallac, Freiburg, Germany). The percentage displacement of the radioactive ligand from its binding to the human p opiate receptor is determined at a concentration of the test substances of 1 μmol/l and stated as the percentage inhibition of the specific binding. Starting from the percentage displacement by various concentrations of the test substances, IC₅₀ inhibitory concentrations which cause a 50 percent displacement of the radioactive ligand are calculated. By conversion by means of the Cheng-Prusoff relationship, Kᵢ values for the test substances are obtained.

### EXAMPLES

The invention will be explained in further detail hereinafter with reference to illustrative examples. These explanations are given merely by way of example and do not limit the overall scope of the invention.

### LIST OF ABBREVIATIONS

DIBAH diisobutylaluminium hydride
DIPEA diisopropylethylamine
EDCl N-ethyl-N′-(3-dimethylaminopropyl)-carbodiimide hydrochloride
wt. % percent by weight
h hour(s)
HOBt 1-hydroxy-1H-benzotriazole
conc. concentrated
LAH lithium aluminium hydride
Mes mesyl
min minute(s)
N normal
RT room temperature.
THF tetrahydrofuran
TFA trifluoroacetic acid
abs. absolute
eq. equivalent(s)
Boc tert-butyl carbamate
MC methylene chloride
HOAt 1-hydroxy-7-azabenzotriazole
M molar
DME dimethoxyethane
EtOAc ethyl acetate
Et₃N triethylamine
n-Bu₄NCl tetra-n-butylammonium chloride
Fmoc 9-fluorenyl methylcarbamate
Cbz benzyl carbamate
DMF dimethylformamide
DMAP 4-dimethylaminopyridine
DCE 1,2-dichloroethane
DMSO dimethylsulfoxide
HMPT hexamethylphosphorotriamide
OPFP O-pentafluorophenyl
DBU 1,8-diazabicyclo[5.4.0]undec-7-ene
LAH lithium aluminium hydride

The chemicals and solvents employed were obtained commercially from the conventional suppliers (e.g. Acros, Avocado, Aldrich, Bachem, Fluka, Lancaster, Maybridge, Merck, Sigma, TCI etc.) or synthesized by methods known to persons skilled in the art.

Commercially obtainable materials, for example Al₂O₃ or silica gel [for example from E. Merck, Darmstadt, Germany] were employed as the stationary phase for the column chromatography. The thin layer chromatography investigations were carried out with commercially obtainable HPTLC precoated plates (for example silica gel 60 F 254 from E. Merck, Darmstadt). The mixing ratios of solvents, mobile phases or for chromatography investigations are, unless indicated otherwise, always stated in volume/volume.

Analytical methods for individual compounds (i.e. compounds not prepared via parallel synthesis methods):
NMR experiments were carried out on a Bruker 440 MHz or 600 MHz machine or on a Varian 400 MHz machine.
The analytical studies were also carried out by mass spectroscopy. Equipment and Methods for HPLC-MS Analytics:
HPLC: Waters Alliance 2795 with PDA Waters 2998; MS: Micromass Quattro Micro™ API; Column: Waters Atlantis® T3, 3 μm, 100 Å, 2.1×30 mm; temp.: 40° C., Eluent A: water+0.1% formic acid; Eluent B: acetonitrile+0.1% formic acid; Gradient: 0% B to 100% B in 8.8 min, 100% B for 0.4 min, 100% B to 0% B in 0.01 min, 0% B for 0.8 min; Flow: 1.0 mL/min; Ionisation: ES+, 25 V; Make up: 100 μL/min 70% Methanol+0.2% formic acid; UV: 200-400 nm.

### Acid Units

The following acid units were synthesized and employed for synthesis of the compounds according to the invention:

| Acid unit | Structure | Name |
|---|---|---|
| AC1 | 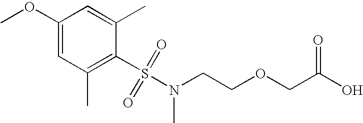 | 2-(2-(4-Methoxy-N,2,6- trimethylphenylsulfonamide)ethoxy)- acetic acid |
| AC2 | 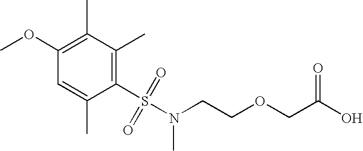 | 2-(2-(4-Methoxy-N,2,3,6- tetramethylphenylsulfonamide)- ethoxy)-acetic acid |
| AC3 | 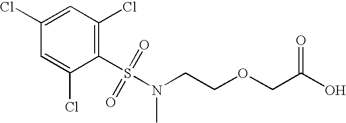 | 2-(2-(2,4,6-Trichloro-N- methylphenylsulfonamide)ethoxy)- acetic acid |
| AC4 | 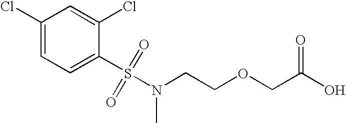 | 2-(2-(2,4-Dichloro-N- methylphenylsulfonamide)ethoxy)- acetic acid |
| AC5 | 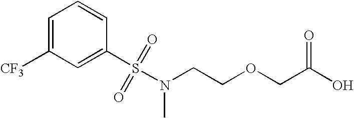 | 2-(2-(N-Methyl-3- (trifluoromethyl)phenylsulfonamide)eth- oxy)-acetic acid |
| AC6 | 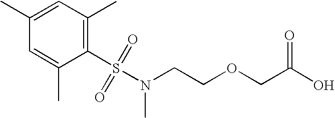 | 2-(2-(N,2,4,6- Tetramethylphenylsulfonamide)ethoxy)- acetic acid |
| AC7 | 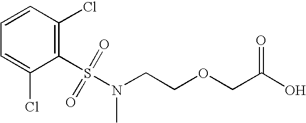 | 2-(2-(2,6-Dichloro-N- methylphenylsulfonamide)ethoxy)- acetic acid |
| AC8 | 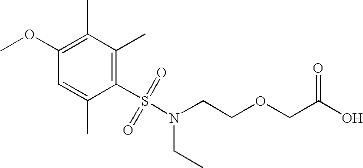 | 2-(2-(N-Ethyl-4-methoxy-2,3,6- trimethylphenylsulfonamide)ethoxy)- acetic acid |
| AC9 | 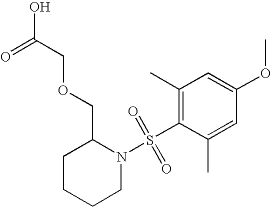 | 2-((1-(4-Methoxy-2,6- dimethylphenylsulfonyl)piperidin-2- yl)methoxy)acetic acid |
| AC10 | 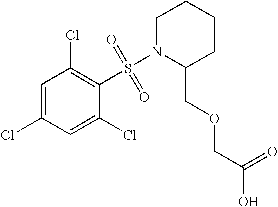 | 2-((1-(2,4,6- Trichlorophenylsulfonyl)piperidin-2- yl)methoxy)acetic acid |
| AC11 | 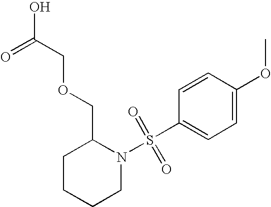 | 2-((1-(4- Methoxyphenylsulfonyl)piperidin-2- yl)methoxy)acetic acid |
| AC12 | 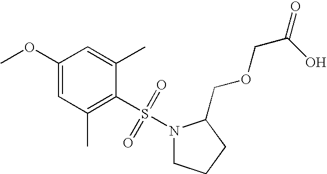 | 2-((1-(4-Methoxy-2,6- dimethylphenylsulfonyl)pyrrolidin-2- yl)methoxy)acetic acid |
| AC13 | 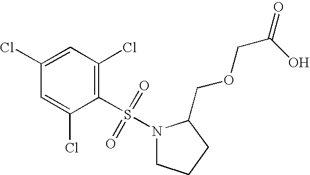 | 2-((1-(2,4,6- Trchlorophenylsulfonyl)pyrrolidin-2- yl)methoxy)acetic acid |
| AC14 | 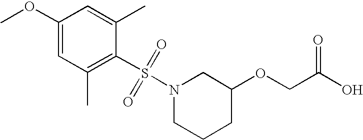 | 2-(1-(4-Methoxy-2,6- dimethylphenylsulfonyl)piperidin-3- yloxy)acetic acid |
| AC15 | 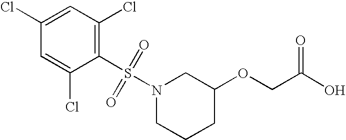 | 2-(1-(24,6- Trichlorophenylsulfonyl)piperidin-3- yloxy)acetic acid |
| AC16 | 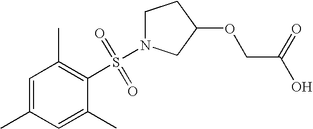 | 2-(1-(Mesitylsulfonyl)pyrrolidin-3- yloxy)acetic acid |
| AC17 | 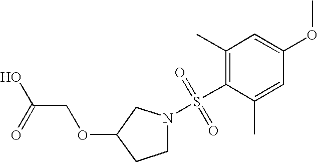 | 2-(1-(4-Methoxy-2,6- dimethylphenylsulfonyl)pyrrolidin-3- yloxy)acetic acid |
| AC18 | 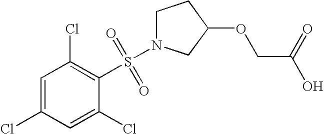 | 2-(1-(2,4,6- Trichlorophenylsulfonyl)pyrrolidin-3- yloxy)acetic acid |
| AC19 | 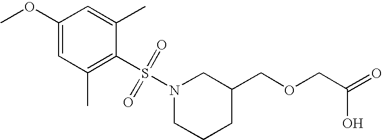 | 2-((1-(4-Methoxy-2,6- dimethylphenylsulfonyl)piperidin-3- yl)methoxy)acetic acid |
| AC20 | 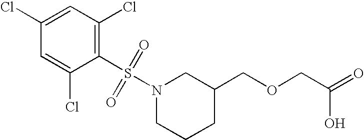 | 2-((1-(2,4,6- Trichlorophenylsulfonyl)piperidin-3- yl)methoxy)acetic acid |
| AC21 | 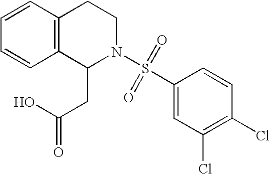 | 2-(2-(3,4-Dichlorophenylsulfonyl)- 1,2,3,4-tetrahydroisochinolin-1- yl)acetic acid |
| AC22 | 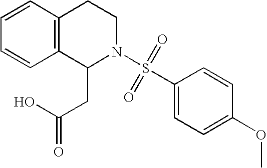 | 2-(2-(4-Methoxyphenylsulfonyl)- 1,2,3,4-tetrahydroisoquinolin-1- yl)acetic acid |
| AC23 | 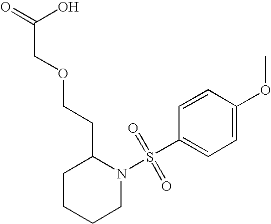 | 2-(2-(1-(4- Methoxyphenylsulfonyl)piperidin-2- yl)ethoxy)acetic acid |
| AC24 | 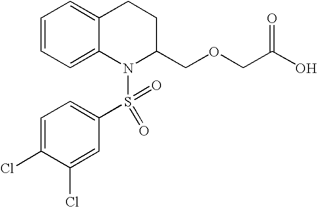 | 2-((1-(3,4-Dichlorophenylsulfonyl)- 1,2,3,4-tetrahydroquinolin-2- yl)methoxy)acetic acid |
| AC25 | 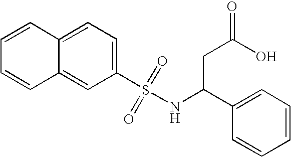 | 3-(Naphthalene-2-sulfonamide)-3- phenylpropionic acid |
| AC26 | 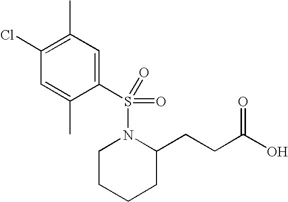 | 3-(1-(4-Chloro-2,5- dimethylphenylsulfonyl)piperidin-2- yl)propionic acid |
| AC27 | 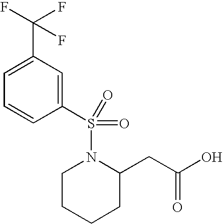 | 2-(1-(3- (Trifluoromethyl)phenylsulfonyl)piperi- din-2-yl)acetic acid |
| AC28 | 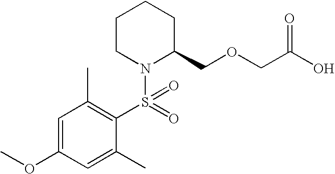 | (S)-2-((1-(4-Methoxy-2,6- dimethylphenylsulfonyl)piperidin-2- ylmethoxy)acetic acid |
| AC29 | 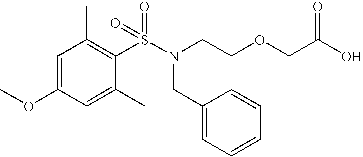 | 2-(2-(N-Benzyl-4-methoxy-2,6- dimethylphenylsulfonamido)ethoxy) acetic acid |
| AC30 | 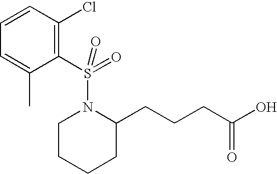 | 4-(1-(2-Chloro-6- methylphenylsulfonyl)piperidin-2- yl)butanoic acid |
| AC31 | 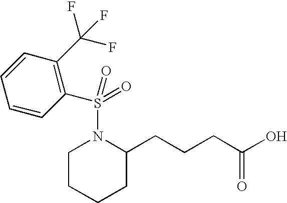 | 4-(1-(2- (trifluoromethyl)phenylsulfonyl)piperi din-2-yl)butanoic acid |
| AC32 | 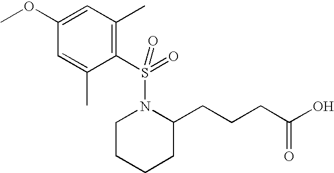 | 4-(1-(4-Methoxy-2,6- dimethylphenylsulfonyl)piperidin-2- yl)butanoic acid |
| AC33 | 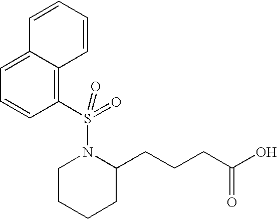 | 4-(1-(Naphthalen-2- ylsulfonyl)piperidin-2-yl)butanoic acid |
| AC34 | 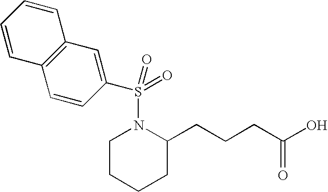 | 4-(1-(Naphthalen-2- ylsulfonyl)piperidin-2-yl)buanoic acid |
| AC35 | 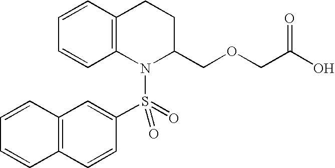 | 2-((1-(Naphthalen-2-ylsulfonyl)- 1,2,3,4-tetrahydroquinolin-2- yl)methoxy)acetic acid |
| AC36 | 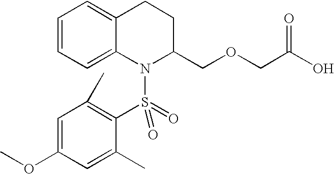 | 2-((1-(4-Methoxy-2,6- dimethylphenylsulfonyl)-1,2,3,4- tetrahydroquinolin-2- yl)methoxy)acetic acid |
| AC37 | 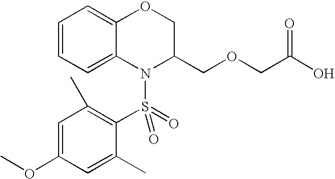 | 2-((4-(4-Methoxy-2,6- dimethylphenylsulfonyl)-3,4-dihydro- 2H-benzo[b][1,4]oxazin-3- yl)methoxy)acetic acid |
| AC38 | 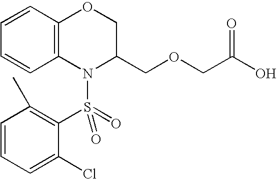 | 2-((4-(2-Chloro-6- methylphenylsulfonyl)-3,4-dihydro- 2H-benzo[b][1,4]oxazin-3- yl)methoxy)acetic acid |
| AC39 | 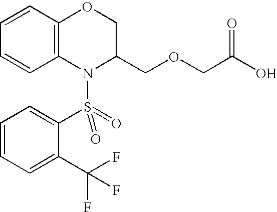 | 2-((4-(2- (Trifluoromethyl)phenylsulfonyl)-3,4- dihydro-2H-benzo[b][1,4]oxazin-3- yl)methoxy)acetic acid |
| AC40 | 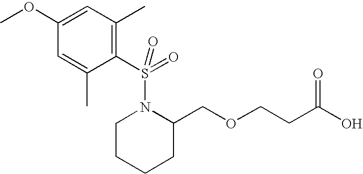 | 3-((1-(4-Methoxy-2,6- dimethylphenylsulfonyl)piperidin-2- yl)methoxy)propanoic acid |
| AC41 | 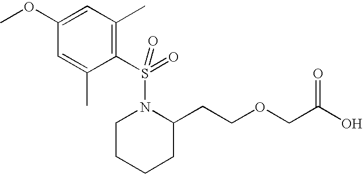 | 2-(2-(1-(4-Methoxy-2,6- dimethylphenylsulfonyl)piperidin-2- yl)ethoxy)acetic acid |
| AC43 | 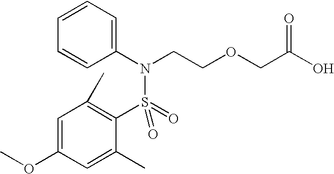 | 2-(2-(4-Methoxy-2,6-dimethyl-N- phenylphenylsulfonamido)ethoxy)ace- tic acid |
| AC44 | 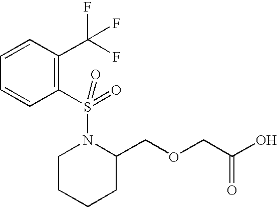 | 2-((1-(2- (Trifluoromethyl)phenylsulfonyl)piperi- din-2-yl)methoxy)acetic acid |

### Synthesis of 4-methoxy-2,6-dimethylbenzene-1-sulfonyl chloride

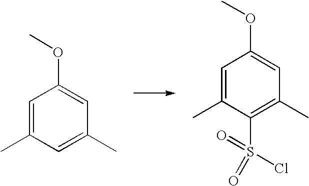

A solution of 3,5-dimethylanisole (102.5 g, 753 mmol) in MC (1,000 ml) was cooled to 0° C. A solution of chlorosulfonic acid (251 ml, 3.763 mmol) in MC (250 ml) was added dropwise to this solution. After a reaction time of 10 min, the reaction solution was introduced into an ice bath (1 l), the phases were separated and extraction was carried out once more with MC (250 ml). The combined organic phases were washed with water (1 l) and saturate sodium chloride solution (1 ml), dried over Na₂SO₄ and concentrated. The product was purified by column chromatography over silica gel (heptane/MC 5:1). Yield: 63.5 g, 36%.

### b) Preparation of the Acid Units

| | | Ester | |
|---|---|---|---|
| | Synthesis | cleavage | |
| Designation | method | variant | Name |
| AC1 | 1 | A | 2-(2-(4-Methoxy-N,2,6- |
| | | | trimethylphenylsulfonamide)ethoxy)acetic acid |
| AC2 | 4 | — | 2-(2-(4-Methoxy-N,2,3,6-tetramethylphenyl- |
| | | | sulfonamide)ethoxy)acetic acid |
| AC3 | 1 | C | 2-(2-(2,4,6-Trichloro-N-methylphenyl- |
| | | | sulfonamide)ethoxy)acetic acid |
| AC4 | 1 | C | 2-(2-(2,4-Dichloro-N-methylphenyl- |
| | | | sulfonamide)ethoxy)acetic acid |
| AC5 | 1 | A | 2-(2-(N-Methyl-3-(trifluoromethyl)phenyl- |
| | | | sulfonamide)ethoxy)-acetic acid |
| AC6 | 1 | A | 2-(2-(N,2,4,6-Tetramethylphenylsulfonamide)- |
| | | | ethoxy)-acetic acid |
| AC7 | 1 | C | 2-(2-(2,6-Dichloro-N-methylphenyl- |
| | | | sulfonamide)ethoxy)acetic acid |
| AC8 | 3 | C | 2-(2-(N-Ethyl-4-methoxy-2,3,6- |
| | | | trimethylphenylsulfonamide)ethoxy)acetic acid |
| AC9 | 2 | B | 2-((1-(4-Methoxy-2,6-dimethylphenylsulfonyl)- |
| | | | piperidin-2-yl)methoxy)acetic acid |
| AC10 | 2 | C | 2-((1-(2,4,6-Trichlorophenylsulfonyl)piperidin-2- |
| | | | yl)methoxy)acetic acid |
| AC11 | 2 | B | 2-((1-(4-Methoxyphenylsulfonyl)piperidin-2- |
| | | | yl)methoxy)acetic acid |
| AC12 | 2 | B | 2-((1-(4-Methoxy-2,6-dimethylphenylsulfonyl)- |
| | | | pyrrolidin-2-yl)methoxy)acetic acid |
| AC13 | 2 | B | 2-((1-(2,4,6-Trichlorophenylsulfonyl)pyrrolidin- |
| | | | 2-yl)methoxy)acetic acid |
| AC14 | 1 | C | 2-(1-(4-Methoxy-2,6-dimethylphenylsulfonyl)- |
| | | | piperidin-3-yloxy)acetic acid |
| AC15 | 1 | C | 2-(1-(2,4,6-Trichlorophenylsulfonyl)piperidin-3- |
| | | | yloxy)acetic acid |
| AC16 | 1 | B | 2-(1-(Mesitylsulfonyl)pyrrolidin-3-yloxy)acetic |
| | | | acid |
| AC17 | 1 | B | 2-(1-(4-Methoxy-2,6-dimethylphenylsulfonyl)- |
| | | | pyrrolidin-3-yloxy)acetic acid |
| AC18 | 1 | C | 2-(1-(2,4,6-Trichlorophenylsulfonyl)pyrrolidin-3- |
| | | | yloxy)acetic acid |
| AC19 | 1 | C | 2-((1-(4-Methoxy-2,6-dimethylphenylsulfonyl)- |
| | | | piperidin-3-yl)methoxy)acetic acid |
| AC20 | 1 | C | 2-((1-(2,4,6-Trichlorophenylsulfonyl)piperidin-3- |
| | | | yl)methoxy)acetic acid |
| AC21 | 5 | — | 2-(2-(3,4-Dichlorophenylsulfonyl)-1,2,3,4- |
| | | | tetrahydroisoquinolin-1-yl)acetic acid |
| AC22 | 5 | — | 2-(2-(4-Methoxyphenylsulfonyl)-1,2,3,4- |
| | | | tetrahydroisoquinolin-1-yl)acetic acid |
| AC23 | 1 | C | 2-(2-(1-(4-Methoxyphenylsulfonyl)piperidin-2- |
| | | | yl)ethoxy)acetic acid |

General Preparation of Sulfonylated Acid Units Starting from Amino Alcohols (Method 1)

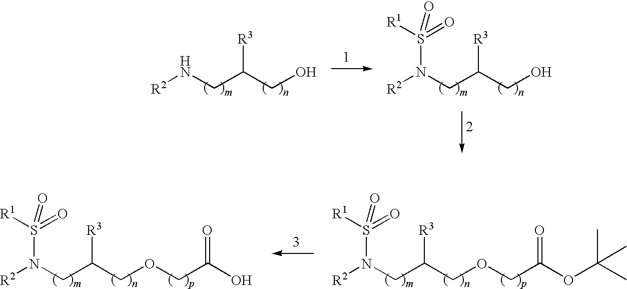

Stage 1. Et₃N (80 mmol) was added to a solution of the amino alcohol (35 mmol) in CH₂Cl₂ (200 ml) and the mixture was cooled to 0° C. using an ice bath. The sulfonyl chloride (32 mmol) was then added and the mixture was stirred at RT for 3 h. After addition of 0.5 M HCl (100 ml), the organic phase was separated off, washed with water, dried over Na₂SO₄ and filtered and the solvent was removed in vacuo. The crude product was used in the next stage without further purification.

Stage 2. n-Bu₄NCl (10 mmol) was added to a solution of the product from stage 1 (30 mmol) in toluene (125 ml), the mixture was cooled to 0° C. and first aqueous 35% strength NaOH (150 ml) and then bromoacetic acid tert-butyl ester (45 mmol) in toluene (25 ml) were added dropwise. The reaction mixture was stirred for 3 h and then washed neutral with water and dried with Na₂SO₄ and the organic solvent was removed in vacuo. The crude product was used in the next stage without further purification or was purified by column chromatography.

General Preparation of Sulfonylated Acid Units Starting from Amino Acids (Method 2)

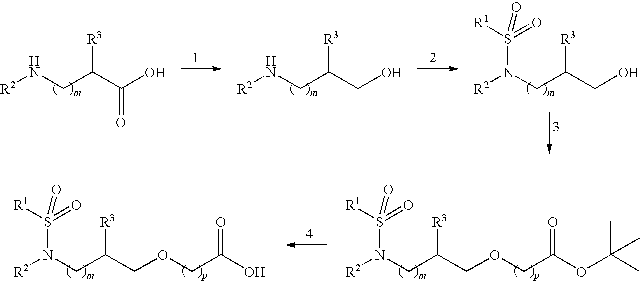

Stage 1. LiAlH₄ (100 ml, 1.0 M in diethyl ether) was added successively to a suspension of the amino acid (100 mmol) in THF (150 ml) under an argon atmosphere, while stirring and at a temperature of between −10° C. and RT. The reaction mixture was stirred for 16 h, during which it warmed up to RT. It was then cooled again to 0° C. and ethyl acetate (20 ml), water (8 ml), 15% strength aqueous NaOH (8 ml) and water (20 ml) were added, while stirring. After filtration, the residue was washed with diethyl ether. The solvent of the combined organic phases was removed in vacuo and the product was employed in the next stage without further purification.

Stage 2. Et₃N (125 mmol) was added to a solution of the amino alcohol (100 mmol) in CH₂Cl₂ (200 ml) and the mixture was cooled to 0° C. using an ice bath. The particular sulfonyl chloride (50 mmol) was then added undiluted or as a solution in CH₂Cl₂ (100 ml) and the mixture was stirred at RT for 3 h. After addition of 0.5 M hydrochloric acid (100 ml), the organic phase was separated off, washed with water, dried over Na₂SO₄ and filtered and the solvent was removed in vacuo. The crude product was used in the next stage without further purification or was purified by column chromatography.

Stage 3. n-Bu₄NCl (10 mmol) was added to a solution of the product from stage 2 (31 mmol) in toluene (200 ml), the mixture was cooled to 0° C. and first aqueous 35% strength NaOH (200 ml) and then bromoacetic acid tert-butyl ester (46 mmol) were added dropwise. The reaction mixture was stirred for 3 h and then washed neutral with water and dried with Na₂SO₄ and the organic solvent was removed in vacuo. The crude product was used in the next stage without further purification or was purified by column chromatography.

General Preparation of Sulfonylated Acid Units Starting from Amino Alcohols (Method 3)

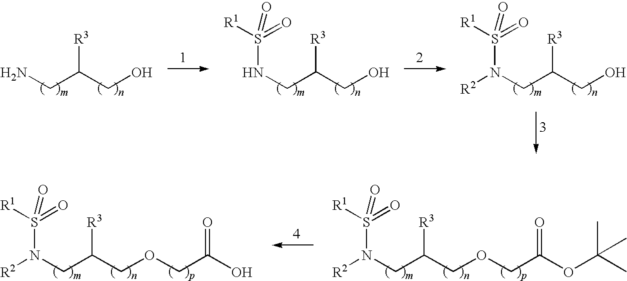

Stage 1. Et₃N (80 mmol) was added to a solution of the amino alcohol (35 mmol) in CH₂Cl₂ (200 ml) and the mixture was cooled to 0° C. using an ice bath. The sulfonyl chloride (32 mmol) was then added and the mixture was stirred at RT for 3 h. After addition of 0.5 M HCl (100 ml), the organic phase was separated off, washed with water, dried over Na₂SO₄ and filtered and the solvent was removed in vacuo. The crude product was used without further purification.

Stage 2. Solid K₂CO₃ (50 mmol) was added to a solution of the product from stage 1 (26 mmol) and alkyl halide (50 mmol) in acetone (200 ml) and the reaction mixture was stirred at 40° C. overnight. After filtration and removal of the solvent, the product was obtained and was either used without further purification or purified via chromatography.

Stage 3. n-Bu₄NCl (10 mmol) was added to a solution of the product from stage 2 (30 mmol) in toluene (125 ml), the mixture was cooled to 0° C. and first aqueous 35% strength NaOH (150 ml) and then bromoacetic acid tert-butyl ester (45 mmol) in toluene (25 ml) were added dropwise. The reaction mixture was stirred for 3 h and then washed neutral with water and dried with Na₂SO₄ and the organic solvent was removed in vacuo. The crude product was used in the next stage without further purification or was purified by column chromatography.

### Methods for Ester Cleavage

### Variant A

The educt (20 mmol) was dissolved in 4 N hydrochloric acid in dioxane (80 mmol) and the solution was stirred at RT overnight. The solvent was largely distilled off and the crude product was purified by recrystallization or chromatography.

### Variant B

The educt (30 mmol) was dissolved in CH₂Cl₂ (200 ml), TFA (30 ml) was added and the mixture was stirred at RT for 2 h. The solvent was largely distilled off and the crude product was purified by recrystallization or chromatography.

### Variant C

The educt (30 mmol) was dissolved in THF (100 ml) and MeOH (100 ml), 6 N NaOH (150 ml) was added and the reaction mixture was stirred at RT for 1 h. The solvent was largely distilled off and 6 N HCl (155 ml) was added at 0° C. After extraction with CH₂Cl₂, drying over Na₂SO₄, filtering off of the drying agent and distilling off of the solvent, the crude product was obtained and purified via column chromatography.

### Method 4

### Synthesis Instructions for the Preparation of 2-(2-(4-methoxy-N,2,3,6-tetramethylphenylsulfonamide)ethoxy)acetic acid AC2

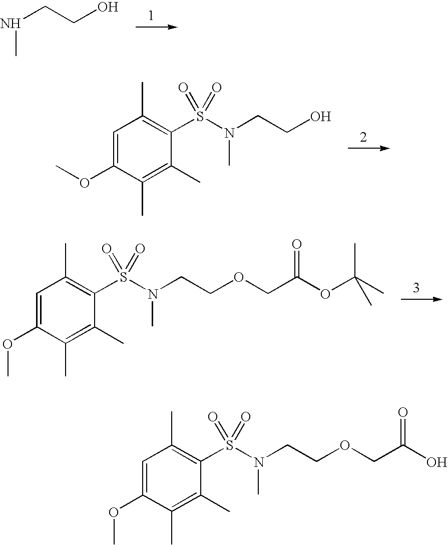

Stage 1. A solution of 4-methoxy-2,3,6-trimethylbenzenesulfonyl chloride (2.29 g, 9.19 mmol) in THF (30 ml) was added dropwise to a solution of 2-methylaminoethanol (0.89 g, 0.95 ml, 11.8 mmol) and Et₃N (5 ml) in THF (15 ml) at 0° C. The mixture was subsequently stirred at RT for 5 h and then concentrated in vacuo, the residue was taken up in NaHCO₃ solution. and the mixture was extracted with EtOAc (3×30 ml). The combined organic phases were dried with Na₂SO₄ and concentrated in vacuo. Yield: 2.38 g (90%)

Stage 2.35% aq. sodium hydroxide solution (40 ml) was added to a solution of N-(2-hydroxyethyl)-4-methoxy-2,3,6,N-tetramethylbenzenesulfonamide (2.34 g, 8.2 mmol) and tetra-n-butylammonium hydrogen sulfate (611 mg, 1.8 mmol) in toluene (40 ml) at 0° C. A solution of bromoacetic acid tert-butyl ester (2.40 g, 1.82 ml, 12.3 mmol) in toluene (35 ml) was then added dropwise to the intensively stirred two-phase system. The mixture was subsequently stirred at RT for 2 h, the aqueous phase was then separated off and the organic phase was washed neutral with water (3×40 ml). The organic phase was dried with Na₂SO₄ and concentrated in vacuo and the residue was purified by flash chromatography with EtOAc/cyclohexane (1:3). Yield: 2.50 g (76%)

Stage 3. First triethylsilane (1.12 g, 1.54 ml, 9.6 mmol) and then trifluoroacetic acid (5 ml) were added to a solution of {2-[(4-methoxy-2,3,6-trimethylbenzenesulfonyl)-methylamino]-ethoxy}-acetic acid tert-butyl ester (2.48 g, 6.18 mmol) in MC (50 ml) and the mixture was stirred at RT for 5 h. The mixture was then concentrated in vacuo, the residue was taken up repeatedly in toluene and the mixture was in each case concentrated again. The crude product was dissolved in EtOAc and the solution was extracted with 5% NaHCO₃ solution (3×50 ml). The combined aqueous phases were adjusted to pH 1 with conc. hydrochloric acid and extracted again with EtOAc (3×50 ml). The combined EtOAc phases were dried with Na₂SO₄ and concentrated in vacuo. Yield: 2.41 g (>99%)

### Method 5

### Synthesis Instructions for the Preparation of 2-(2-(4-methoxyphenylsulfonyl)-1,2,3,4-tetrahydroisoquinolin-1-yl)acetic acid AC2

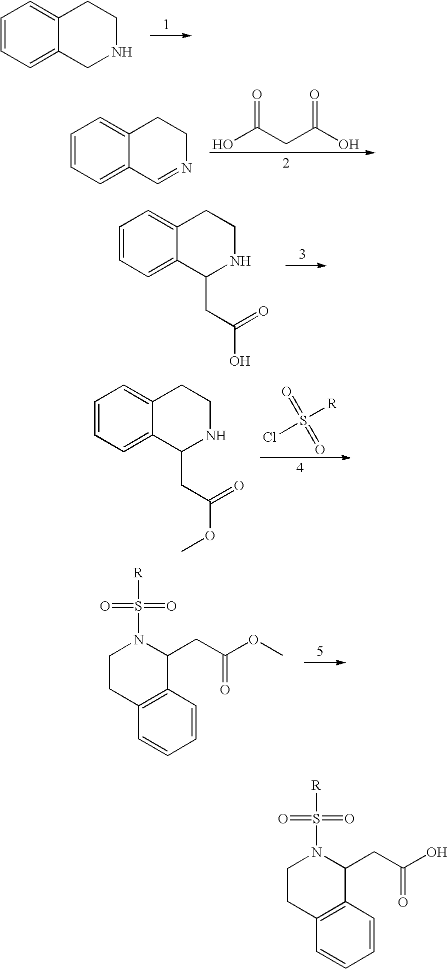

Stage 1. N-Bromosuccinimide (19.0 g, 107 mmol) was added in portions to a solution of 1,2,3,4-tetrahydroisoquinoline (12.94 g, 97 mmol) in CH₂Cl₂ (200 ml) over a period of 15 min. The reaction mixture was stirred until educt was no longer present according to TLC control (CH₂Cl₂/CH₃OH 9/1). NaOH (50 ml, 30% aqueous solution) was added and the mixture was stirred for 1 h. The organic phase was separated off and washed with water (100 ml). The product was extracted with 10% HCl (2×100 ml). The combined acid extracts were extracted with CH₂Cl₂ (100 ml) and the extract was rendered basic (pH 9) with concentrated ammonia and extracted with CH₂Cl₂ (2×100 ml). After drying over Na₂SO₄ and filtration, the solvent was removed in vacuo. Yield: 12.0 g, 94%.

Stage 2. 3,4-Dihydroisoquinoline (12.0 g, 92 mmol) and malonic acid (9.6 g, 92 mmol) were brought together and the mixture was stirred at RT and then at 120° C. for 30 min. After cooling to RT, the solid residue was crystallized out from aqueous 2-propanol (2 weeks at 4° C.), filtered off and washed with small portions of 2-propanol. Yield: 11.54 g, 66%.

Stage 3. H₂SO₄ (6.4 ml, 120 mmol) was added to a solution of the acid (11.50 g, 60.1 mmol) in MeOH (250 ml) under an N₂ atmosphere and the mixture was stirred under reflux for 5 h. The reaction mixture was cooled to RT overnight. The solvent was removed in vacuo and the residue was dissolved in ethyl acetate (250 ml). The organic phase was washed with aqueous saturated NaHCO₃ solution (250 ml). The organic phase was separated off, dried over Na₂SO₄ and filtered off and the solvent was removed in vacuo. Yield: 10.53 g, 85%.

Stage 4. Et₃N (14.9 ml, 106 mmol) was added to a solution of the ester (9.55 g, 46.5 mmol) in CH₂Cl₂ (150 ml). The reaction mixture was cooled to 0° C. and a solution of the sulfonyl halide (42 mmol) in CH₂Cl₂ (100 ml) was added dropwise. After stirring at RT overnight, 0.5 M HCl (100 ml) was added and the organic phase was separated off and washed with water. After drying over Na₂SO₄ and filtration, the solvent was removed in vacuo. The product was purified via column chromatography (silica, CH₂Cl₂). Yield: 15.22 g 96%.

Stage 5. 6 M NaOH (120 ml) was added to a mixture of the ester (15.22 g, 40.54 mmol) in THF (200 ml) and water (120 ml) and the mixture was stirred at RT overnight. The reaction mixture was concentrated in vacuo and 6 M HCl (125 ml) and CH₂Cl₂ (400 ml) were added. After the organic phase had been separated off, it was washed with saturated aqueous NaCl solution and dried over Na₂SO₄ and, after filtration, the solvent was removed in vacuo. Yield: 14.65 g, 100%.

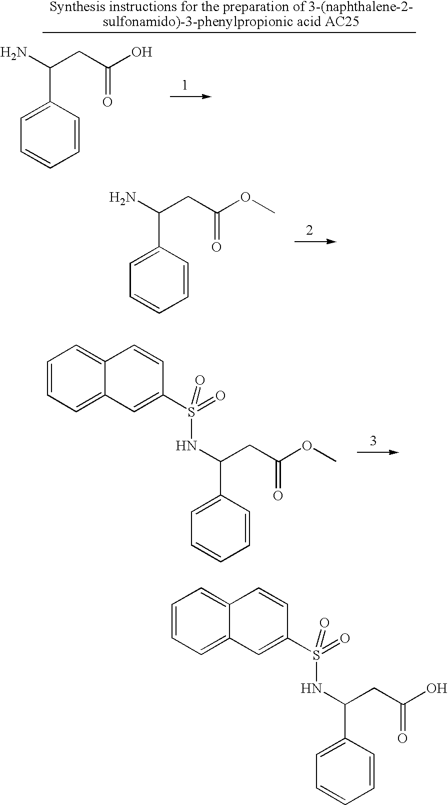

Stage 1. Thionyl chloride (19.1 g, 162 mmol) was added dropwise to a solution, cooled to 0° C., of 3-amino-3-phenylpropionic acid (8.9 g, 54 mmol) in methanol (3 ml/mmol). The reaction mixture was then heated under reflux for 12 h (TLC control). The solvent was removed completely and the residue was dried in vacuo. The crude product was employed in the next stage without further purification.

Stage 2. Triethylamine (9.7 g, 96 mmol) was added to a solution, cooled to 0° C., of methyl 3-amino-phenylpropionate (5.73 g, 32 mmol) in MC. Naphthalene-2-sulfonyl chloride (8.7 g, 38.4 mmol), dissolved in MC (50 ml), was added to this reaction solution. The reaction mixture was stirred at RT for 3 h (TLC control). When the reaction had ended, the reaction mixture was diluted with MC, washed with water and saturated NaCl solution and dried over Na₂SO₄. The solvent was stripped off and the crude product was purified by column chromatography (silica gel, ethyl acetate/hexane, 3:7).

Stage 3. LiOH x H₂O (0.25 g, 18 mmol) was added to a solution of the methyl 3-(naphthalene-2-sulfonamido)-3-phenylpropionate (3.3 g, 9 mmol) in a methanol/water mixture (3:1, 90 ml) at a reaction temperature of 0° C. The reaction mixture was stirred at RT for 16 h. The solvent was stripped off under reduced pressure, the residue was taken up in water and the mixture was washed with MC. The aqueous phase was then cautiously acidified with HCl (1 N) and extracted with ethyl acetate. The organic phase was washed with water and saturated NaCl solution and dried over Na₂SO₄. After removal of the solvent, the product was obtained in an adequate purity.

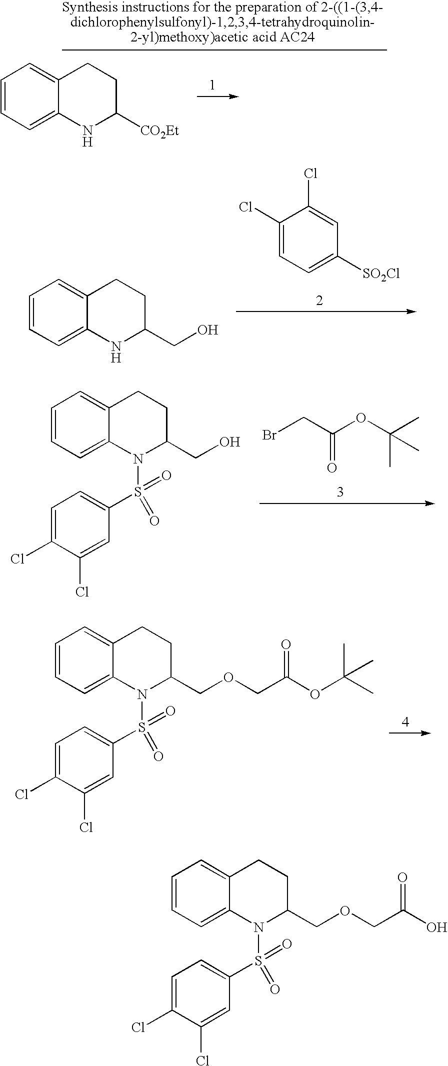

Stage 1. 1,2,3,4-Tetrahydroquinoline-2-carboxylic acid ethyl ester (25 mmol) in THF (5 ml/mol) was added dropwise to a suspension of LAH (2 eq.) in THF (50 ml) at 0° C. The reaction mixture was stirred at RT for 1 h and then heated under reflux for 4 h. After addition of aqueous saturated sodium sulfate solution, the mixture was filtered and the organic solvent was removed in vacuo. The product was purified via column chromatography (3:7 ethyl acetate/hexane). Yield: 50%.

Stage 2. Pyridine (5 eq.), DMAP (0.5 eq.) and 3,4-dichlorobenzenesulfonyl chloride (1.2 eq.), dissolved in MC (50 ml), were added to a suspension, cooled to 0° C., of the alcohol (16 mmol) in MC (5 ml/mmol). After stirring at 0° C. for 5 h, MC was added and the mixture was washed with aqueous copper sulfate solution, water and saturated sodium chloride solution. After drying over sodium sulfate and filtration, the solvent was removed in vacuo. The product was purified via column chromatography (5:95 ethyl acetate/MC). Yield: 80%.

Stage 3. A solution of the sulfonamide (16 mmol) dissolved in THF (100 ml) was added dropwise to a suspension, cooled to 0° C., of NaH (2 eq.) in THF (300 ml), while stirring. After stirring at this temperature for 45 min, a solution of bromoacetic acid tert-butyl ester (1.5 eq.) in THF (50 ml) was added. The reaction mixture was heated at 50° C. for 20 h. It was then cooled to 0° C., ice was added and the mixture was extracted with ethyl acetate. The organic phase was washed with aqueous saturated sodium chloride solution and dried over sodium sulfate. After filtration, the solvent was removed in vacuo. The product was purified via column chromatography (1:9 ethyl acetate/hexane). Yield: 50%.

Stage 4. TFA (13 eq.) was added to a solution of the tert-butyl ester (1 eq.) in MC (10 ml/mmol) at a temperature of 0° C., while stirring. After stirring at 0° C. for 3 h, the solvent was removed in vacuo. The crude product was used without further working up.

### Synthesis Instructions for the Preparation of 3-(1-(4-chloro-2,5-dimethylphenylsulfonyl)piperidin-2-yl)propionic acid AC26

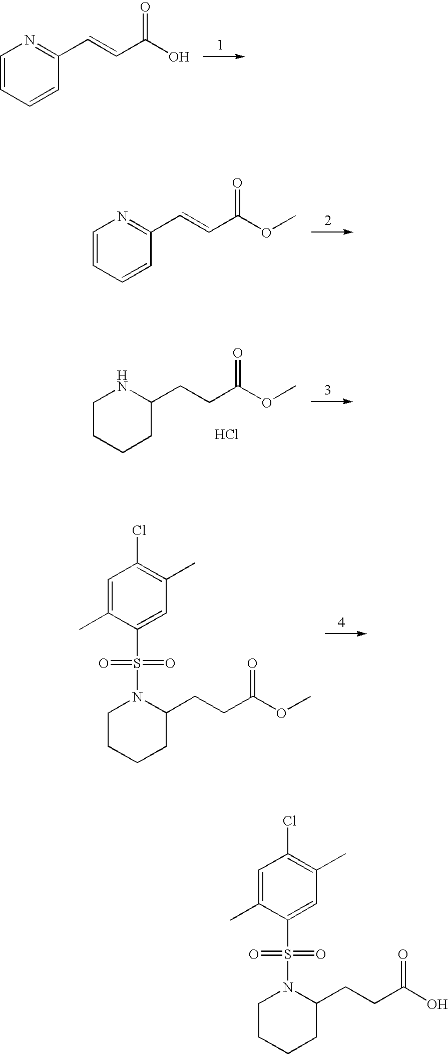

Stage 1. H₂SO₄ (12.8 ml, 240 mmol) was added to a solution of 3-(2-pyridyl)-acrylic acid (23.88 g, 160 mmol) in methanol (750 ml). The reaction mixture was heated under reflux overnight and, after cooling to RT, was poured into saturated aqueous NaHCO₃ solution (1 ml). The methanol was stripped off on a rotary evaporator and the aqueous phase was extracted twice with ethyl acetate (400 ml). The organic phase was washed with saturated sodium chloride solution (500 ml), dried over sodium sulfate and concentrated. The crude product was employed in the next stage without further purification. Yield: 22.19 g, 85%.

Stage 2. Methyl 3-(pyridin-2-yl)acrylate (22.15 g, 136 mmol) was dissolved in THF (300 ml) and chloroform (10.9 ml), and PtO₂ (3.08 g, 13.6 mmol, 0.1 eq.) was added under a nitrogen atmosphere. The solution was first flushed with nitrogen for 10 min and then stirred under an H₂ atmosphere (8 bar) overnight. After cooling, the mixture was first flushed again with nitrogen, the catalyst was removed by filtering over filtering earth and rinsed with MC and the filtrate was concentrated to dryness in vacuo. The methyl 3-(piperidin-2-yl)propionate hydrochloride was employed in the next stage without further purification. Yield: 27.95 g, 99%.

Stage 3. A solution of triethylamine (14.7 ml, 104.5 mmol) dissolved in MC (150 ml) was added to a solution of methyl 3-(piperidin-2-yl)propionate hydrochloride (8.69 g, 41.8 mmol) and 4-chloro-2,5-dimethylbenzenesulfonyl chloride (10 g, 41.8 mmol) in MC (150 ml). The reaction mixture was stirred at RT overnight and then washed with HCl (1 M, 300 ml). The organic phase was dried over sodium sulfate and concentrated. The crude product was purified by column chromatography over silica gel (heptane/ethyl acetate 6:1 to 3:1). Yield: 12.82 g, 82%.

Stage 4. Aqueous NaOH solution (6 M, 100 ml) was added to a solution of methyl 3-(1-(4-chloro-2,5-dimethylphenylsulfonyl)piperidin-2-yl)propionate (12.82 g, 34.3 mmol) in THF (100 ml). After a reaction time of 1 h, the solvent was removed on a rotary evaporator and the residue was cooled to 0° C. HCl (6 M, 100 ml) was added and the mixture was extracted with ethyl acetate. The organic phase was dried over sodium sulfate and concentrated. Yield: 12.36 g, 100%.

Synthesis instructions for the preparation of 2-(1-(3-trifluoromethyl)phenylsulfonyl)piperidin-2-yl)acetic acid AC27

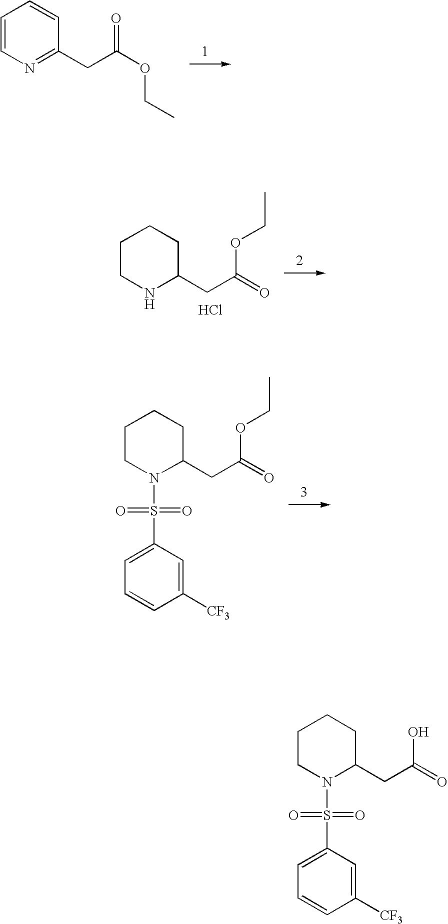

Stage 1. Ethyl 2-(pyridin-2-yl)acetate (24.51 g, 148.4 mmol) was dissolved in ethanol (130 ml), and PtO₂ (3.37 g, 14.84 mmol, 0.1 eq.) and chloroform (20 ml) were added. The suspension was stirred under an H₂ atmosphere (8 bar) at 40° C. overnight. According to TLC control (silica gel, MC/methanol 95:5), the reaction was not complete, so that further chloroform (15 ml) was added and the mixture was stirred under an H₂ atmosphere (8 bar) at 40° C. for a further 2 d (TLC control). After cooling, the catalyst was first removed by filtering over filtering earth and the filtrate was concentrated to dryness in vacuo. The ethyl 2-(piperidin-2-yl)acetate hydrochloride was employed in the next stage without further purification. Yield: 31.51 g>100%.

Stage 2. The ethyl 2-(piperidin-2-yl)acetate hydrochloride (7.5 g, max. 36.1 mmol) was dissolved in MC (225 ml) and triethylamine (11 ml, 78.3 mmol) was added. 3-(Trifluoromethyl)benzene-1-sulfonyl chloride (9.72 g, 39.7 mmol) was then added dropwise and the mixture was stirred at RT overnight. When the reaction had ended (TLC control, MC/methanol 98:2), the reaction mixture was diluted with MC (275 ml) and washed successively with KHSO₄ solution (0.5 M, 500 ml) and saturated sodium chloride solution (500 ml). The organic phase was dried over sodium sulfate and concentrated. The crude product was purified by column chromatography over silica gel (MC). Yield: 10.45 g, 76% over 2 stages.

Stage 3. The ethyl 2-(1-(3-(trifluoromethyl)phenylsulfonyl)piperidin-2-yl)acetate (10.45 g, 27.5 mmol) was dissolved in a mixture of methanol (150 ml), dioxane (40 ml) and aqueous NaOH solution (4 M, 41.3 ml, 165.2 mmol, 6 eq.) and the solution was stirred overnight. When the reaction had ended (TLC control, MC/methanol 95:5), the solution was concentrated. The crude product was taken up in ethyl acetate (600 ml) and the mixture was with KHSO₄ solution (0.5 M, 600 ml). The aqueous phase was extracted once more with ethyl acetate (100 ml) and the combined organic phases were washed with saturated sodium chloride solution (500 ml), dried over sodium sulfate and concentrated. Yield: 9.4 g, 97%.

### Synthesis Instructions for the Preparation of (S)-2-((1-(4-methoxy-2,6-dimethylphenylsulfonyl)piperidin-2-yl)methoxy)acetic acid AC28

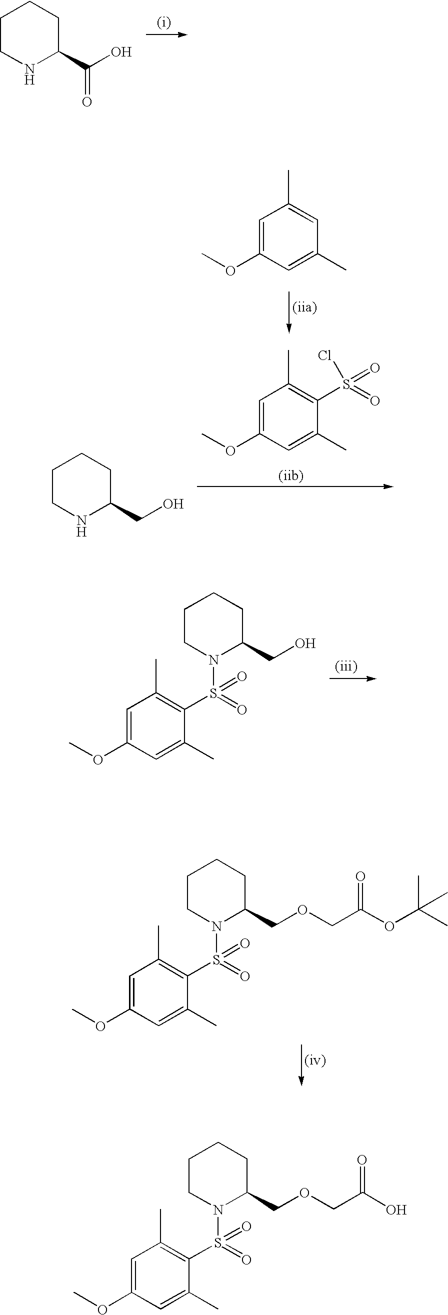

Stage (i): (S)-Piperidine-2-carboxylic acid (2 g, 15.5 mmol) was initially introduced into tetrahydrofuran (20 ml) and boron trifluoride etherate (2.1 ml, 117.1 mmol) was added, followed by boron dimethylsulfide in tetrahydrofuran (dropwise, 3 ml, 30.9 mmol). The reaction mixture was then refluxed for 16 h. The mixture was quenched with ice-cooled methanol (10 ml), hydrogen chloride solution (conc. aq., 3 ml) was added dropwise and the mixture was refluxed for 30 min. After cooling, the mixture was rendered alkaline with dilute sodium hydroxide solution (4%) and extracted with methylene chloride (3×50 ml). The combined organic phases were dried over sodium sulfate and concentrated in vacuo. The crude product was employed in the next stage without further purification.

Yield: 44%

Stage (iia): Chlorosulfonic acid (2.3 eq.) in methylene chloride (0.5 ml/mmol) was slowly added dropwise to a solution, cooled to 0° C., of 3.5-dimethylanisole (1 eq.) in methylene chloride (1 ml/mmol) over a period of 10 min. The reaction mixture was stirred for a further 10 min and then slowly added dropwise to ice-water (5 eq. with respect to the chlorosulfonic acid). The phases were separated and the aqueous phase was extracted with methylene chloride (several times, UV control). The combined organic phases were dried (Na₂SO₄) and concentrated in vacuo.

Yield: 82%

Stage (iib): (S)-Piperidin-2-ylmethanol (1.1 eq.) was dissolved in methylene chloride (4 ml/mmol) and triethylamine (2.5 eq.) was added. A solution of 4-methoxy-2,6-dimethylbenzenesulfonyl chloride (1 eq.) in methylene chloride (2 ml/mmol) was added dropwise at 0° C. and the mixture was then stirred at room temperature for 90 min. Hydrogen chloride solution (aq., 0.5 mol/l, 2 ml/mmol) was added, the mixture was stirred for 15 min and the phases were separated. The organic phase was washed with water, dried over sodium sulfate and concentrated in vacuo. The crude product was employed in the next stage without further purification.

Yield: 20%

Stage (iii): tetra-n-Butylammonium chloride (0.33 eq.) and sodium hydroxide solution (5 ml/mmol, 35%) were added to a cooled solution of (S)-(1-(4-methoxy-2,6-dimethylphenylsulfonyl)piperidin-2-yl)methanol (1 eq.) in toluene (5 ml/mmol) at 0° C.). tert-Butyl bromoacetate (1.5 eq.) was then slowly added dropwise at 0° C. After stirring at room temperature for 90 min, the phases were separated and the organic phase was washed with water to pH neutrality, dried over sodium sulfate and concentrated in vacuo. The crude product was employed in the next stage without further purification.

Yield: 64%

Stage (iv): (S)-tert-Butyl 2-((1-(4-methoxy-2,6-dimethylphenylsulfonyl)piperidin-2-yl)methoxy)acetate (1 eq.) was dissolved in methylene chloride (10 ml/mmol), the solution was cooled and trifluoroacetic acid (13 eq.) was slowly added at 0° C. After stirring at room temperature for 2 h, the reaction mixture was concentrated in vacuo and the residue was dried. The crude product was employed in the next stage without further purification. Yield: quantitative

### Synthesis of Acid Building Block AC-29: 2-(2-(N-Benzyl-4-methoxy-2,6-dimethylphenylsulfonamido)ethoxy)acetic acid (AC-29)

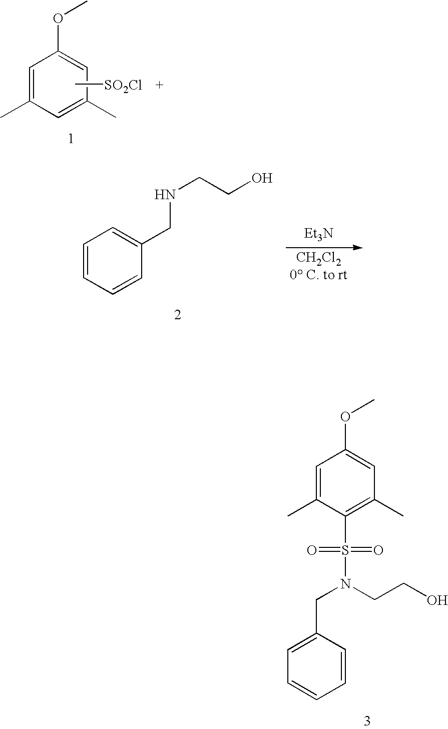

3. To a solution of N-benzylaminoethanol (2, 10.0 mL, 70.3 mmol) in CH₂Cl₂ (200 mL) was added Et₃N (22.5 mL, 160 mmol). The mixture was cooled to 0° C. after which a solution of compound I (15.0 g, 63.9 mmol) in CH₂Cl₂ (100 mL) was added dropwise. The mixture was stirred for 3 h at room temperature. Aqueous 1 M HCl (150 mL) was added. After phase separation the organic layer was washed with water (100 mL), dried (Na₂SO₄) and evaporated under reduced pressure. Purification by column chromatography (silica, heptane/EtOAc, 2:1) afforded sulfonamide 3 (14.93 g, 67%).

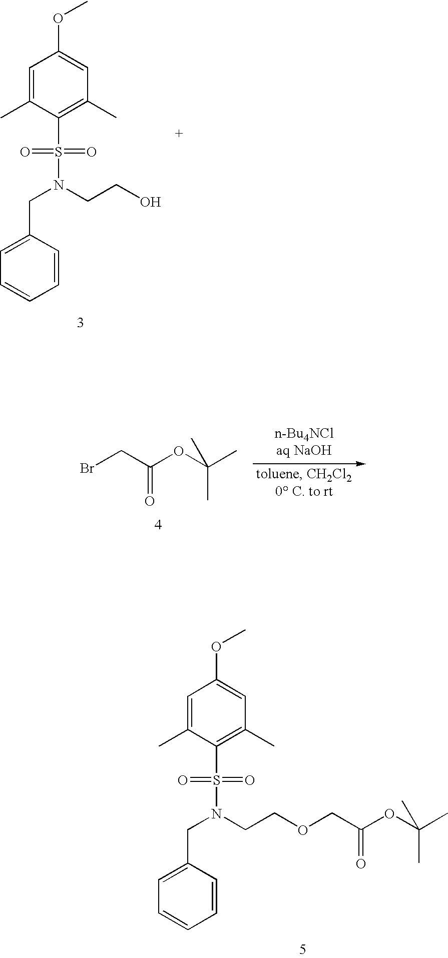

5. To a solution of compound 3 (14.9 g, 42.6 mmol) in toluene (100 mL) and CH₂Cl₂ (100 mL) was added n-Bu₄NCl (3.95 g, 14.2 mmol). After cooling to 0° C., an aqueous 35% NaOH solution (175 mL) was added, followed by a dropwise addition of tert-butyl bromoacetate (4, 9.32 mL, 64 mmol). The reaction mixture was stirred at room temperature for 3 h. The organic layer was separated and washed with H₂O (3×300 mL), dried (Na₂SO₄) and evaporated to dryness. Purification by column chromatography (silica, heptane/EtOAc, 3:1) afforded compound 5 (19.40 g, 98%).

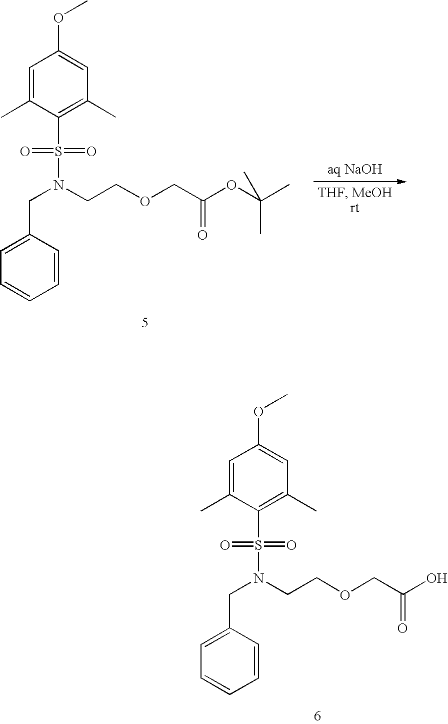

6. To a solution of compound 5 (19.4 g, 41.8 mmol) in THF (165 mL) and MeOH (150 mL) was added aqueous 6 M NaOH (150 mL, 900 mmol). The reaction mixture was stirred at room temperature. After 1 h the organic solvents were evaporated and aqueous 6 M HCl (155 mL) was added at 0° C. The aqueous layer was extracted with EtOAc (2×150 mL). The organic layers were combined, dried (Na₂SO₄) and evaporated to dryness. The product was co-evaporated with Et₂O and i-Pr₂O (2×) to yield compound 6 (17.05 g, 100%).

Synthesis of acid building blocks AC-30, AC-31, AC-32, AC-34: 4-(1-(2-Chloro-6-methylphenylsulfonyl)piperidin-2- yl)butanoic acid (AC-30), 4-(1-(2-(trifluoromethyl)phenylsulfonyl)piperidin-2-yl)butanoic acid (AC-31), 4-(1-(4-methoxy-2,6- dimethylphenylsulfonyl)piperidin-2-yl)butanoic acid (AC-32) and 4-(1-(naphthalen-2-ylsulfonyl)piperidin-2-yl)butanoic acid (AC-34)

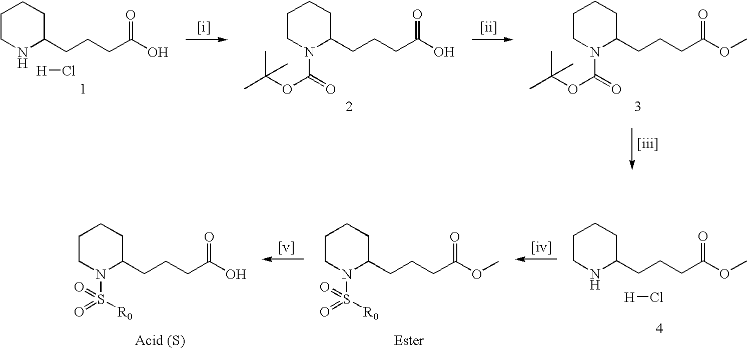

### Step (i): 4-(1-tert-Butoxycarbonyl)piperidin-2-yl)butanoic acid (2)

4-Piperidin-2-ylbutanoic acid Hydrochloride (10.0 g, 48.3 mmol), and K₂CO₃ (26.6 g, 193.1 mmol) was dissolved in dest. water (70 mL) and Dioxane (124 mL). The reaction mixture was cooled to 0° C. and at this temperature Di-tert-butyldicarbonate (11.4 g, 53.1 mmol) was added slowly. The reaction mixture was stirred for 24 h at room temperature. After completion of the reaction water and Ethylacetate were added, the two phases were separated. The aqueous Phase was extracted once with Ethylacetate. Afterwards the aqueous Phase was triturated with 2 M HCL (aqueous) to reach pH=2. At this pH the aqueous phase was extracted 4× with Dichloromethane. The combined organic layers were dried over Magnesium sulfate, filtered off and evaporated to complete dryness to give (2) (13.13 g, 100%).

### Step (ii): tert-Butyl-2-(4-methoxy-4-oxobutyl)piperidine-1-carboxylate (3)

To a solution of 4-(1-tert-Butoxycarbonyl)piperidin-2-yl)butanoic acid (2) (26 g, 95.8 mmol) in Dichloromethane 1,1′-carbonyldiimidazole (23.3 g, 143.7 mmol) was added. The reaction mixture was stirred for 1 h at room temperature. Subsequently Methanol (19.4 mL, 479 mmol) was added and the reaction mixture was stirred over night. The completion of the reaction was controlled via Thin-layer chromatography. After completion the reaction mixture was washed 3× with saturated solution NH₄CL (aqueous) and 2× with brine. The organic layer was dried over Magnesium sulfate, filtered off and evaporated in vacuum to afford tert-Butyl-2-(4-methoxy-4-oxobutyl)piperidine-1-carboxylate (3) (25.67 g, 94%).

### Step (iii): Methyl 4-(piperidin-2-yl)butanoate hydrochloride (4)

Acetyl chloride was added dropwise to a solution of tert-butyl-2-(4-methoxy-4-oxobutyl)piperidine-1-carboxylate (3) (25.67 g, 89.9 mmol) in methano. The reaction mixture was stirred for 5 h at room temperature. The completion of the reaction was monitored by thin-layer chromatography. After completion the reaction mixture was evaporated in vacuum to give Methyl 4-(piperidin-2-yl)butanoate hydrochloride (4) (20.14 g, 100%)

### General Procedure GP I—Sulfonylation (Ester 30-34)

### Step (iv):

To a solution of methyl 4-(piperidin-2-yl)butanoate hydrochloride (4) (1 Equiv.) in Dichloromethane the sulfonyl chloride (3 Equiv.) was added. Subsequently N-Ethyl-diisopropylamine (3 Equiv.) was added dropwise. The reaction mixture was stirred overnight at room temperature. The completion of the reaction was controlled via Thin-layer chromatography. After completion the reaction mixture was made acidic with 1 M HCl (aqueous) and the aqueous phase was saturated with brine and then extracted 3× with Dichloromethane. The combined organics layers were dried over Magnesium sulfate, filtered off and evaporated in vacuum. Purification by column chromatography (Aluminiumoxide; Hexan/Ethylacetate) gave us the desired product.

TABLE 1
Synthesis of the sulfonylated amino acid ester

| Ester No. | Structure | Name | Aminoacid ester (4) | Sulfonylchloride |
|---|---|---|---|---|
| Ester 34 | 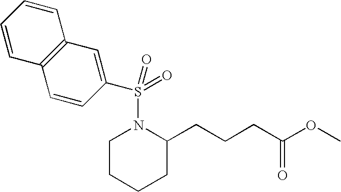 | Methyl 4-(1- (naphthalene-2- ylsulfonyl)pipe- ridin-2- yl)butanoate ((iv-01) | Methyl 4-(piperidin-2- yl)butanoate hydrochloride (4) | Naphthalene-2-sulfonylchloride |
| Ester 32 | 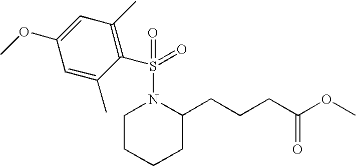 | Methyl 4-(1-(4-meth- oxy-2,6- dimethylphenylsulfo- nyl)pipe- ridin-2-yl)butanoate (iv-02) | Methyl 4-(pipe- ridin-2- yl)butanoate hydrochloride (4) | 4-methoxy-2,6-dimethylphenyl- 1-sulfonyl chlorid |
| Ester 30 | 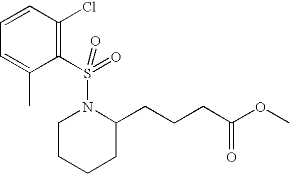 | Methyl 4-(1-(2- chloro-6- methylphenylsulf- onyl)pipe- ridin-2-yl)butanoate (iv- 03) | Methyl 4-(pipe- ridin-2- yl)butanoate hydrochloride (4) | 2-Chloro-6-methylbenzene-1- sulfonylchloride |
| Ester 31 | 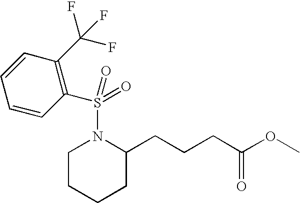 | Methyl 4-(1-(2- (trifluoro- methyl)phenylsulfo- nyl)piperidin-2- yl)butanoate (iv-04) | Methyl 4-(pipe- ridin-2- yl)butanoate hydrochloride (4) | 2-(trifluoromethyl-(benzene-1- sulfonylchloride |

| | Snthesis | | |
|---|---|---|---|
| Ester No. | according to | Yield | Comment |
| Ester 34 | GP I | 80% | Columnchromatography: |
| | | (18.1 mmol) | Aluminiumoxid; |
| | | | Hexane/Ethylacetate |
| | | | 5:1 → 4:1 |
| Ester 32 | GP I | 63% | Columnchromatography: |
| | | (14.3 mmol) | Aluminiumoxid; |
| | | | Hexane/Ethylacetate |
| | | | 96:2 → 8:2 |
| Ester 30 | GP I | 93% | Columnchromatography: |
| | | (10.4 mmol) | Aluminiumoxid; |
| | | | Hexane/Ethylacetate |
| | | | 98:2 → 8:2 |
| Ester 31 | GP I | 61% | Columnchromatography: |
| | | (11.5 mmol) | Aluminiumoxid; |
| | | | Hexane/Ethylacetatee |
| | | | 5 → 8:2 |

### General Procedure GP III—Saponification (AC30-34):

### Step (v):

To a solution of (Ester 30-34) (1 Equiv.) in Methanol/Water Lithium hydroxide was added and the reaction mixture was stirred over night at room temperature. The completion of the reaction was controlled via Thin-layer chromatography. After completion the Methanol was evaporated in vacuum, and the residue was triturated with Ethylacetate. The mixture was made acidic with diluted HCl. The aqueous layer was extracted 2× with Ethylacetate, the combined organic layers were dried over sodium sulfate and were evaporated in vacuum to give the desired Product (AC30-AC34)

TABLE 2
Synthesis of Sulfonamide acids

| Acid No. | Structure | Name | Sulfonamidester(ester) |
|---|---|---|---|
| AC-34 | 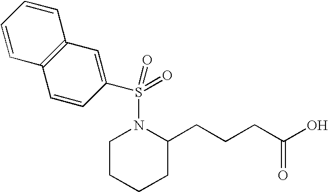 | 4-(1-naphthalen-2- ylsulfonyl)piperidin-2- yl)butanoic acid (v-01) | Methyl 4-(1- (naphthalene-2- ylsulfonyl)piperidin-2- yl)butanoate (Ester 34) |
| AC-32 | 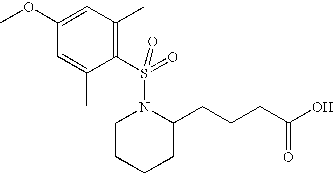 | 4-(1-(4-methoxy-2,6- dimethylphenylsulfonyl)pipe- ridin-2-yl)butanoic acid (v-02) | Methyl 4-(1-(4-methoxy- 2,6- dimethylphenylsulfonyl)pipe- ridin-2-yl)butanoate (Ester 32) |
| AC-30 | 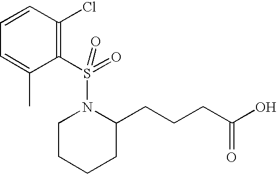 | 4-(1-(2-chloro-6- methylphenylsulfonyl)pipe- ridin-2-yl)butanoic acid (v-03) | Methyl 4-(1-(2-chloro-6- methylphenylsulfonyl)pipe- ridin-2-yl)butanoate (Ester 30) |
| AC-31 | 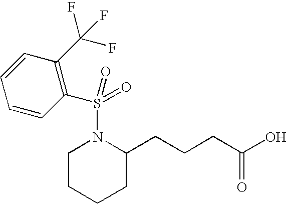 | 4-(1-(2- (trifluoromethyl)phenylsulfo- nyl)piperidin-2- yl)butanoic acid | Methyl 4-(1-(2- (trifluoromethyl)phenylsulf- nyl)piperidin-2- yl)butanoate (Ester 31) |

| | Synthesis | | |
|---|---|---|---|
| Acid No. | according to | Yield | Comments |
| AC-34 | GP II | 102% | |
| | | (23.2 mmol) | |
| AC-32 | GP II | 90% | |
| | | (14.6 mmol) | |
| AC-30 | GP II | 112% | |
| | | (8.22 mmol) | |
| AC-31 | GP II | 125 | |
| | | (11.1 mmol) | |

### Synthesis of Acid Building Block AC-33

### 4-(1-(Naphthalen-1-ylsulfonyl)piperidin-2-yl)butanoic acid (AC-33)

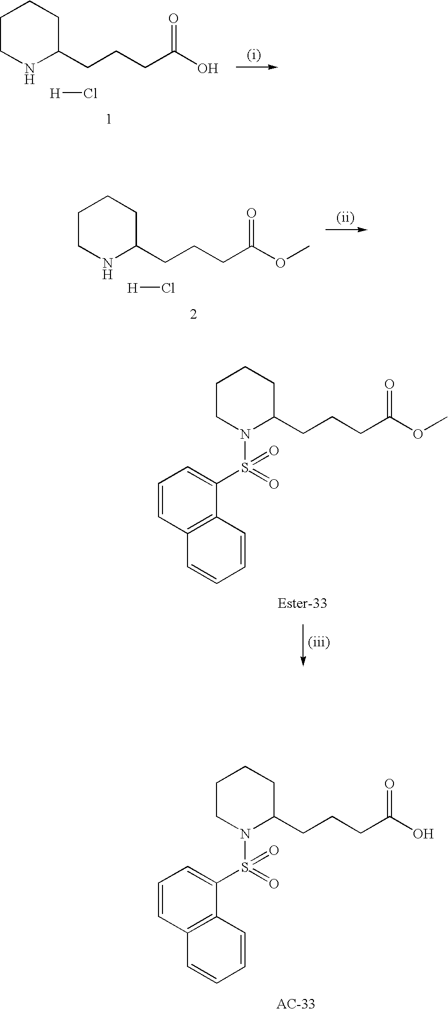

### Step (i): Methyl 4-(piperidin-2-yl)butanoate hydrochloride (2)

A solution of 4-(2-piperidinyl)butanoic acid hydrochloride (5.95 g, 34.8 mmol) in Methanol (104 mL) is cooled to 0° C. At this temperature thionylchloride (7.54 mL, 104.3 mmol) is added slowly. The reaction mixture is heated to reflux for 12 h. The solvent is evaporated in vacuum. The residue is suspended in Ethylacetate and is heated to reflux. The suspension is filtered off while it is still hot. In the filtrate a white solid dropped out, which was filtered off and dried in vacuum to give Methyl 4-(piperidin-2-yl)butanoate hydrochloride (2) (3.49 g, 45%)

### Step (ii): Methyl 4-(1-(naphthalene-1-ylsulfonyl)piperidin-2-yl)butanoate (Ester-33)

To a solution of Methyl 4-(piperidin-2-yl)butanoate hydrochloride (2) (3.74 g, 20.2 mmol) in Dichloromethane (143 mL) Naphthalene-1-sulfonylchloride (13.7 g, 60.55 mmol) was added. Subsequently N-Ethyl-diisopropylamine (10.2 mL, 60 55 mmol.) was added dropwise. The reaction mixture was stirred overnight at room temperature. The completion of the reaction was controlled via Thin-layer chromatography. After completion the reaction mixture was made acidic with 1 M HCl (aqueous) and the aqueous phase was saturated with brine and then extracted 4× with Dichloromethane. The combined organics layers were dried over Magnesium sulfate, filtered off and evaporated in vacuum. Purification by column chromatography (Aluminiumoxid; Hexan/Ethylacetate 97:3-9:1) gave us the desired Product Methyl 4-(1-(naphthalene-1-ylsulfonyl)piperidin-2-yl)butanoate (Ester 33) (4.95 g, 65%)

### Step (iii): 4-(1-(Naphthalen-1-ylsulfonyl)piperidin-2-yl)butanoic acid (AC-33)

To a solution of Methyl 4-(1-(naphthalene-1-ylsulfonyl)piperidin-2-yl)butanoate (Ester-33) (4.95 g, 13.18 mmol.) in Methanol/Water (54 mL/36 mL) Lithium hydroxide (1.58 g, 65.9 mmol) was added and the reaction mixture was stirred over night at room temperature. The completion of the reaction was controlled via Thin-layer chromatography. After completion the Methanol was evaporated in vacuum, and the residue was triturated with Ethylacetate. The mixture was made acidic with diluted HCl. The aqueous layer was extracted 2× with Ethylacetate, the combined organic layers were dried over Sodium sulfate and were evaporated in vacuum to give the desired Product 4-(1-(Naphthalen-1-ylsulfonyl)piperidin-2-yl)butanoic acid (AC-33) (4.38 g, 91%).

### Synthesis of acid building block AC-35: 2-((1-(Naphthalen-2-ylsulfonyl)-1,2,3,4-tetrahydroquinolin-2-yl)methoxy)acetic acid (AC-35)

Synthesis of acid building block AC-35 was performed in analogy to the synthesis of building block AC-36 with naphthalene-2-sulfonyl chloride instead of 4-methoxy-2,6-dimethylbenzene-1-sulfonyl chloride.

### Synthesis of acid building block AC-36: 2-((1-(4-Methoxy-2,6-dimethylphenylsulfonyl)-1,2,3,4-tetrahydroquinolin-2-yl)methoxy)acetic acid (AC-36)

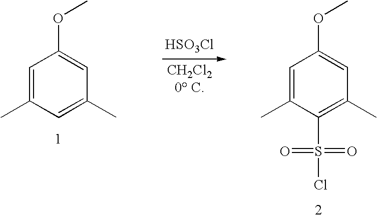

2. A solution of chlorosulfonic acid (247 mL, 3687 mmol) in CH₂Cl₂ (250 mL) was added dropwise to a solution of 3,5-dimethylanisole (1, 100.44 g, 737 mmol) in CH₂Cl₂ (1 L) at 0° C. After 15 min, the reaction mixture was poured into ice-water (1.5 L) and extracted with CH₂Cl₂ (250 mL). The organic layer was quickly washed with ice-cold H₂O (1 L), ice-cold aqueous saturated NaHCO₃ (1 L), dried (Na₂SO₄) and concentrated under reduced pressure. Purification by column chromatography (silica, heptane/CH₂Cl₂, 5:1) afforded sulfonyl chloride 2 (79.64 g, 46%) as a yellow oil which crystallised at −20° C. in the freezer overnight. The product was stored under argon in a freezer due to instability issues.

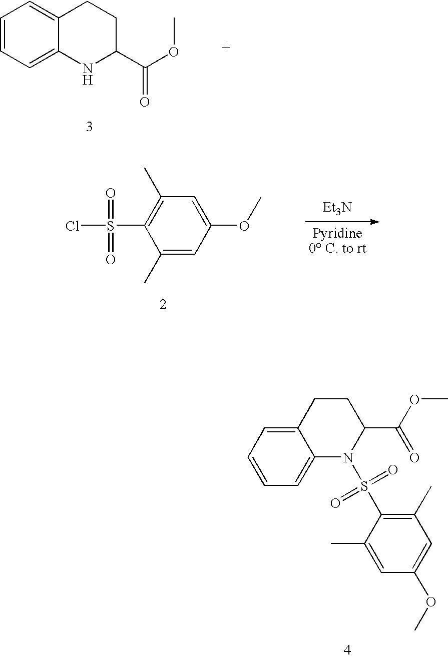

4. To a mixture of ester 3 (8.24 g, 43.1 mmol) in dry pyridine (10.5 mL, 129 mmol) was added sulfonyl chloride 2 (20.23 g, 86 mmol) and the mixture was stirred overnight at 40° C. CH₂Cl₂ (100 mL) was added and the reaction mixture was washed with aqueous 1 M HCl (100 mL), dried (Na₂SO₄) and evaporated to dryness under reduced pressure. Purification by column chromatography (silica, toluene/EtOAc, 24:1) afforded sulfonamide 4 (14.39 g, 86%).

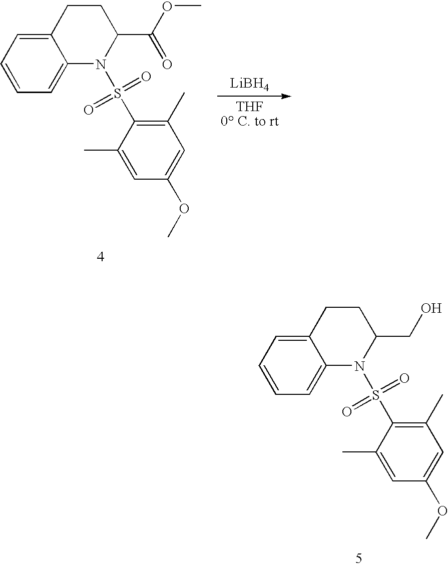

5. Sulfonamide 4 (14.29 g, 36.7 mmol) was dissolved in dry THF (100 mL). After cooling to 0° C. a solution of 2 M LiBH₄ in THF (33.0 mL, 66.0 mmol) was added dropwise slowly and the reaction mixture was stirred at room temperature overnight. The reaction was not complete according to TLC (silica, heptane/EtOAc, 1:1), additional 2 M LiBH₄ in THF (18.35 mL, 36.7 mmol) was added and the reaction mixture was stirred at room temperature overnight. The reaction was complete according to TLC. The reaction mixture was quenched by adding Na₂SO₄.10H₂O and H₂O, additional Na₂SO₄ was added to remove any residual H₂O, filtered, dried (Na₂SO₄) and evaporated to dryness under reduced pressure. The residue was dissolved in CH₂Cl₂ (100 mL), washed with H₂O (100 mL) and evaporated to dryness under reduced pressure to afford alcohol 5 (14.01 g, ‘106’%).

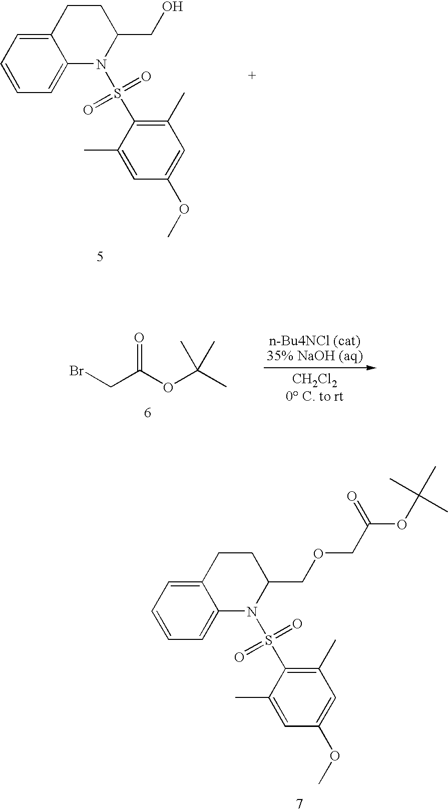

7. To a solution of alcohol 5 (13.23 g, max 34.7 mmol) in CH₂Cl₂ (80 mL) was added n-Bu₄NCl (3.36 g, 12.1 mmol). The reaction mixture was cooled to 0° C. after which aqueous 35% NaOH (84 mL) was added, followed by the addition of tert-butyl 2-bromoacetate (6, 6.40 mL, 43.9 mmol). After stirring for 4 h at room temperature no more starting material was observed on TLC (silica, heptane/EtOAc, 1:1). The organic layer was separated, washed with H₂O (3×150 mL) and brine (150 mL) until neutral, dried (Na₂SO₄) and concentrated under reduced pressure. Purification was carried out by subjecting the crude compound twice to column chromatography (silica, heptane/EtOAc, 4:1) and afforded ester 7 (14.90 g, 90% over 2 steps).

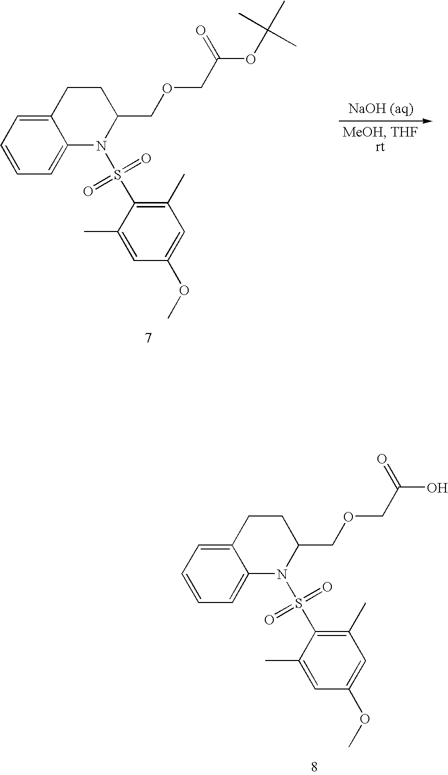

8. A mixture of ester 7 (14.82 g, 31.2 mmol), MeOH (110 mL), THF (110 mL) and aqueous 4 M NaOH (117 mL, 467 mmol) was stirred at room temperature for 2 h. The reaction was complete according to TLC (silica, heptane/EtOAc 2:1). The solution was then concentrated under reduced pressure to remove the organic solvents. The resulting suspension was acidified with aqueous 6 M HCl (120 mL) while cooling at 0° C. CH₂Cl₂ (250 mL) was added and after separation of the layers, the organic layer was dried (Na₂SO₄) and evaporated to dryness under reduced pressure affording carboxylic acid 8 (12.64 g, 97%).

### Synthesis of acid building block AC-37: 2-((4-(4-Methoxy-2,6-dimethylphenylsulfonyl)-3,4-dihydro-2H-benzo[b][1,4]oxazin-3-yl)methoxy)acetic acid (AC-37)

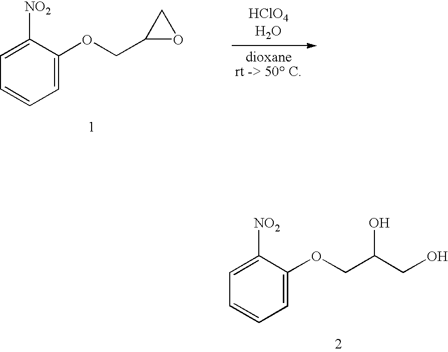

2. Perchloric acid (3.30 mL, 38.2 mmol) was added to a solution of 1 (37.3 g, 191 mmol) in dioxane (746 mL) and H₂O (568 mL) and the reaction mixture was stirred at 50° C. overnight. The reaction mixture was concentrated to half its volume and aqueous saturated NaHCO₃ was added. The H₂O layer was extracted with CH₂Cl₂ (2×) and the combined organic layer was washed with brine, dried (Na₂SO₄) and concentrated. Purification by column chromatography (silica, heptane/EtOAc, 2:3) yielded 2 (30.6 g, 75%).

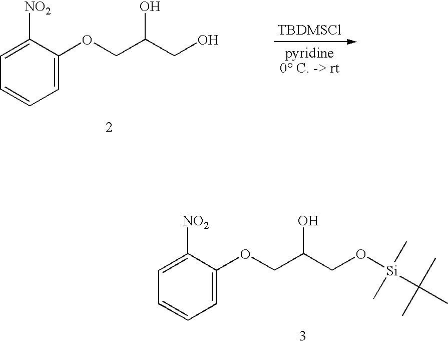

3. To a solution of 2 (30.6 g, 143 mmol) in pyridine (75 mL) was added tert-butyldimethylsilyl chloride (23.8 g, 158 mmol) while cooling with an icebath. The reaction mixture was stirred at room temperature for 2 h and afterwards concentrated and co-evaporated with toluene. The residue was dissolved in EtOAc, washed with H₂O, brine, dried (Na₂SO₄) and concentrated to give 3 (46.7 g, 99%).

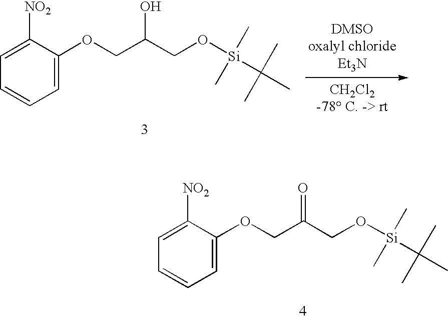

4. A solution of DMSO (21.24 mL, 299 mmol) in CH₂Cl₂ (600 mL) was dropwise added to a solution of oxalyl chloride (15.0 mL, 171 mmol) in CH₂Cl₂ (300 mL) in 30 min while maintaining the internal temperature below −65° C. A solution of 3 (46.7 g, 142 mmol) in CH₂Cl₂ (300 mL) was added dropwise in 15 min. while maintaining the temperature below −65° C. The reaction mixture was stirred an additional 45 minutes at −78° C., after which Et₃N (99.0 mL, 712 mmol) was added. After the reaction mixture was stirred at −78° C. for 45 min, the reaction mixture was allowed to warm to room temperature and stirring was continued for an additional hour. The reaction mixture was washed with H₂O and brine, dried (Na₂SO₄) and concentrated. The residue was dissolved in Et₂O, filtrated and the filtrate was concentrated and crystallized (Et₂O/heptane) to result in 4 (30.9 g, 67%). The mother liquor was concentrated and crystallized (Et₂O/heptane) and gave extra 4 (2.27 g, 5%).

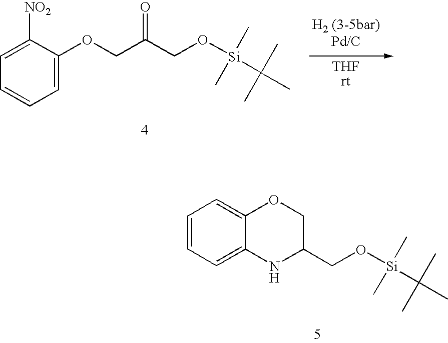

5. A mixture of 4 (18 g, 55.3 mmol) and 10% Pd/C (1.8 g, 1.7 mmol) in dry THF (150 mL) was stirred under an hydrogen atmosphere of ˜3 bar for 2 days and then under an hydrogen atmosphere of 5 bar for 1 d. The reaction mixture was filtrated over Celite and eluted with THF. The filtrate was concentrated and 10% Pd/C (1.8 g, 1.7 mmol) was added to the residue in dry THF (150 mL) and the resulting reaction mixture was stirred under an hydrogen atmosphere of ˜5 bar for 1 d. The reaction mixture was filtrated over Celite and eluted with THF. The filtrate was concentrated and purified by column chromatography (silica, heptane/Et₂O, 9:1) to yield 5 (7.11 g, 46%).

Another batch of 4 (15.06 g, 46.3 mmol) and Pd/C 10% Pd/C (1.5 g, 1.4 mmol) in dry THF (150 mL) was stirred under an hydrogen atmosphere (˜5 bar) for 2 days. The reaction mixture was filtrated over Celite and eluted with THF. The filtrate was concentrated and purified by column chromatography (silica, heptane/Et₂O, 9:1) to yield extra 5 (3.20 g, 25%).

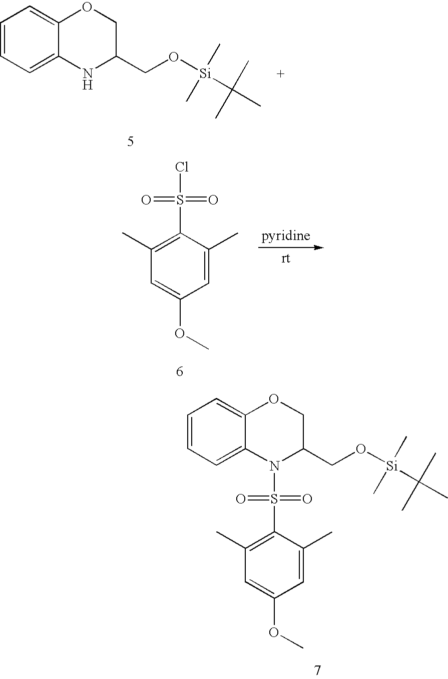

7. Sulfonyl chloride 6 (8.96 g, 38.2 mmol) was added to a solution of 5 (9.70 g, 34.7 mmol) in pyridine (8.42 mL) and the reaction mixture was stirred at room temperature for 2 d. The reaction mixture was concentrated, dissolved in CH₂Cl₂ and washed with H₂O, brine, dried (Na₂SO₄) and concentrated to give crude 7, which was directly used in the next step.

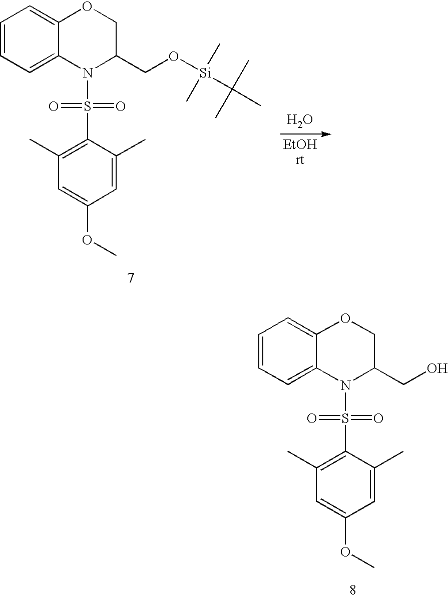

8. Crude 7 was dissolved in EtOH (˜100 mL) and H₂O (˜100 mL) with heating and was left standing overnight. The reaction mixture was concentrated, dissolved in CH₂Cl₂, washed with aqueous saturated NaHCO₃, brine, dried (Na₂SO₄) and concentrated. The residue was solidified with EtOAc/heptane (2:1) and some CH₂Cl₂. The resulting precipitate was washed with EtOAc/heptane (2:1) and dried on filter to yield 8 (9.68 g, 77% over 2 steps).

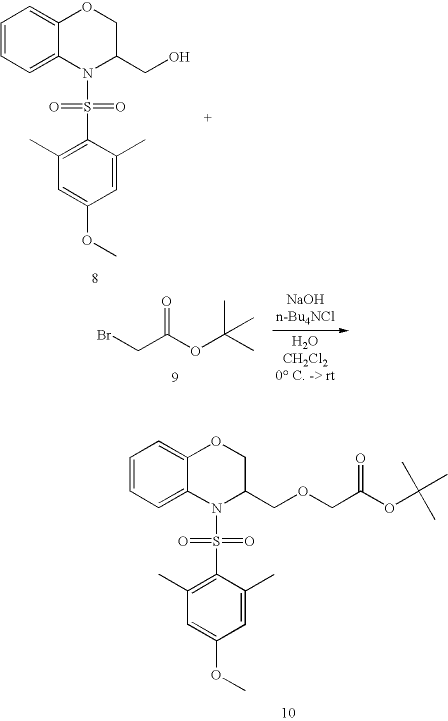

10. To an ice-cooled solution of 8 (9.68 g, 26.6 mmol) and n-Bu₄NCl (2.44 g, 8.79 mmol) in CH₂Cl₂ (130 mL) was sequentially added aqueous 35% NaOH solution (130 mL) and tert-butyl bromoacetate (9, 11.6 mL, 80.0 mmol). The reaction mixture was stirred at room temperature for 4.5 h, after which H₂O was added. The organic layer was separated, washed with H₂O (2×), dried (Na₂SO₄) and concentrated. The residue was purified by column chromatography (silica, heptane/EtOAc, 4:1->3:1) to provide 10 (11.9 g, 94%).

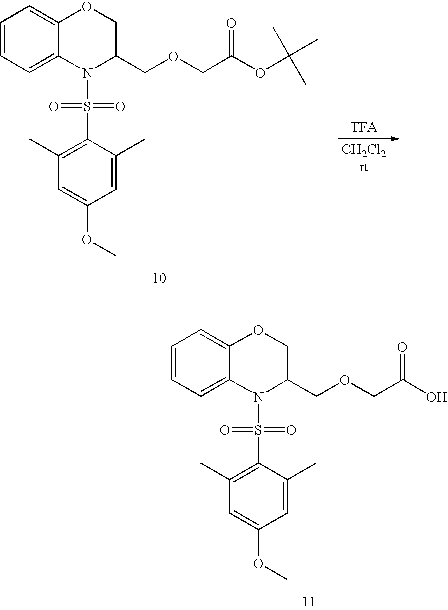

11. A solution of 10 (11.80 g, 24.7 mmol) and TFA (25 mL, 324 mmol) in CH₂Cl₂ (125 mL) was stirred at room temperature for 2.5 h. The reaction mixture was concentrated, co-evaporated with toluene (2×) and CH₂Cl₂ (2×). The residue was dried under vacuum for 1 day to furnish 11 (10.26 g, 99%).

### Synthesis of Acid Building Block AC-38: 2-((4-(2-Chloro-6-methylphenylsulfonyl)-3,4-dihydro-2H-benzo[b][1,4]oxazin-3-yl)methoxy)acetic acid (AC-38

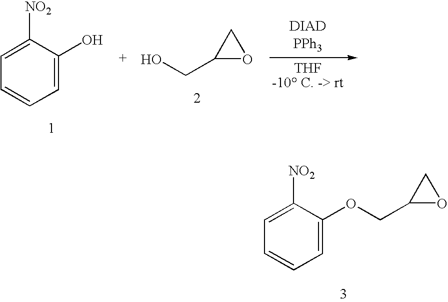

3. A solution of DIAD (149 mL, 719 mmol) in dry THF (200 mL) was added in 30 min to a solution of 2-nitrophenol (1,100 g, 719 mmol), glycidol (2, 50.0 mL, 719 mmol) and PPh₃ (189 g, 719 mmol) in dry THF (800 mL) while keeping the temperature between −10° C. and −5° C. The reaction mixture was stirred for 1 h at this temperature range, after which stirring was continued at room temperature overnight. The reaction mixture was concentrated and the residue was stirred up in toluene, filtrated and concentrated. Purification by column chromatography (silica, toluene/acetone, 95:5) afforded 3 (114.25 g, 81%).

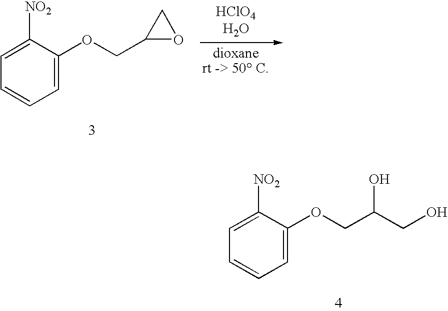

4. Perchloric acid (4.96 mL, 57.4 mmol) was added to a solution of 3 (56.02 g, 287 mmol) in dioxane (1124 mL) and H₂O (856 mL) and the reaction mixture was stirred at 50° C. overnight. The reaction mixture was concentrated to half its volume and aqueous saturated NaHCO₃ was added. The H₂O layer was extracted with CH₂Cl₂ (2×) and the combined organic layer was washed with brine, dried (Na₂SO₄) and concentrated. Purification by column chromatography (silica, heptane/EtOAc, 2:3->1:2) yielded 4 (47.45 g, 78%).

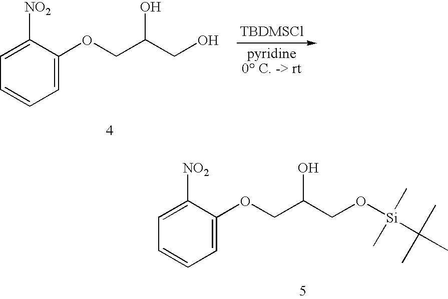

5. To a solution of 4 (47.45 g, 223 mmol) in pyridine (117 mL) was added tert-butyldimethylsilyl chloride (36.9 g, 245 mmol) while cooling with an icebath. The reaction mixture was stirred at room temperature for 2 h and afterwards concentrated and co-evaporated with toluene. The residue was dissolved in EtOAc, washed with H₂O, brine, dried (Na₂SO₄) and concentrated to give 5 (77.94 g, 100%).

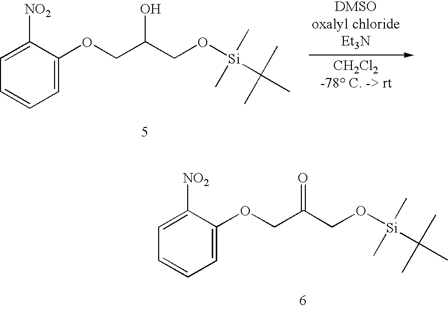

6. A solution of DMSO (35.0 mL, 500 mmol) in CH₂Cl₂ (1 L) was dropwise added to a solution of oxalyl chloride (25.0 mL, 286 mmol) in CH₂Cl₂ (500 mL) in 1 h while maintaining the internal temperature below −65° C. A solution of 5 (77.94 g, 221 mmol) in CH₂Cl₂ (500 mL) was added dropwise in 30 min. while maintaining the temperature below −65° C. The reaction mixture was stirred an additional 45 minutes at −78° C., after which Et₃N (166 mL, 1.190 mol) was added. After the reaction mixture was stirred at −78° C. for 45 min, the reaction mixture was allowed to warm to room temperature and stirring was continued for an additional hour. The reaction mixture was washed with H₂O and brine, dried (Na₂SO₄) and concentrated. The residue was dissolved in Et₂O, filtrated and the filtrate was concentrated. The residue was filtered over a small layer of silica (heptane/EtOAc, 4:1) and crystallized (i-Pr₂O/heptane) to result in 6 (23.15 g, 32.1%). The mother liquor was concentrated and crystallized (heptane) to give extra 6 (3.20 g, 4%). The mother liquor was concentrated and purified by column chromatography (silica, heptane/EtOAc, 4:1->3:1), followed by crystallization (Et₂O/heptane) to yield extra 6 (4.16 g, 6%). All crystals were combined to give 6 (30.51 g, 42%).

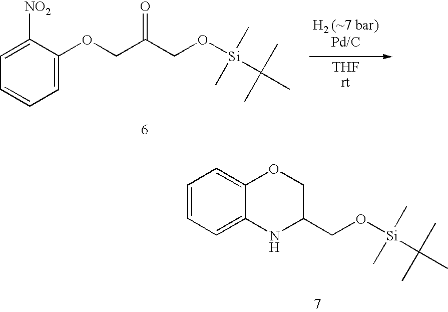

7. A mixture of 6 (24.36 g, 74.9 mmol) and 10% Pd/C (2.4 g, 23 mmol) in EtOH (350 mL) in a 1 L autoclave was stirred at 60° C. under a nitrogen atmosphere. After pressurizing the reaction vessel with hydrogen to ˜7 bar, the pressure dropped rapidly while stirring vigorously. The pressurizing the reaction vessel with hydrogen to 7 bar was repeated until the pressure remained almost constant for 10 min. The reaction mixture was then stirred at 60° C. and 4 bar overnight. The reaction mixture was filtrated over Celite and eluted with EtOH. The filtrate was concentrated, co-evaporated with heptane and purified by column chromatography (silica, heptane/i-Pr₂O, 9:1->4:1) to yield 7 (14.75 g, 71%).

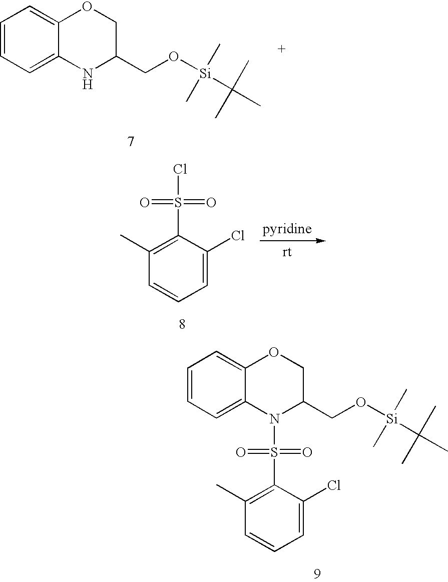

7. 2-chloro-6-methylbenzenesulfonyl chloride (8, 7.82 g, 34.8 mmol) was added to a solution of 7 (8.83 g, 31.6 mmol) in pyridine (7.67 mL, 95.0 mmol) and the reaction mixture was stirred at room temperature overnight. CH₂Cl₂ and H₂O were added to the reaction mixture and the organic layer was separated, washed with H₂O, brine, dried (Na₂SO₄) and concentrated to give crude 9, which was directly used as such in the next step.

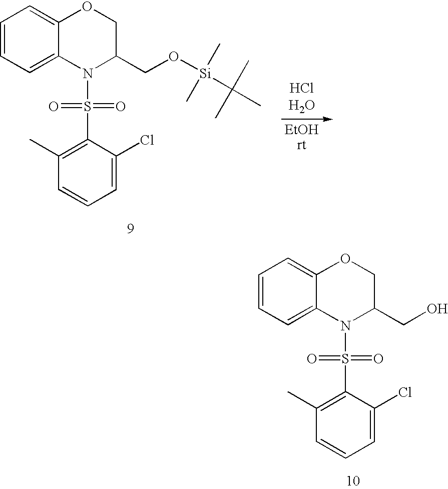

10. Aqueous 1 M HCl (50 mL, 50 mmol) was added to crude 9 in EtOH (200 mL) and the reaction mixture was stirred at room temperature overnight. The reaction mixture was concentrated, dissolved in CH₂Cl₂, washed with aqueous saturated NaHCO₃, dried (Na₂SO₄) and concentrated. The residue was purified by column chromatography (silica, heptane/EtOAc: 2:1) to yield 10 (7.75 g, 69%, 2 steps).

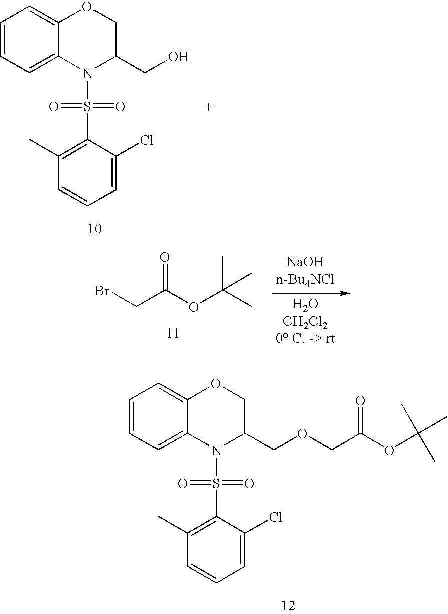

12. To an ice-cooled solution of 10 (7.75 g, 21.9 mmol) and n-Bu₄NCl (2.00 g, 7.23 mmol) in CH₂Cl₂ (110 mL) was sequentially added aqueous 35% NaOH solution (110 mL) and tert-butyl bromoacetate (11, 9.57 mL, 65.7 mmol). The reaction mixture was stirred at room temperature for 4 h, after which H₂O was added. The organic layer was separated, washed with H₂O and brine, dried (Na₂SO₄) and concentrated. The residue was purified by column chromatography (silica, heptane/EtOAc, 4:1) to provide 12 (9.98 g, 92%).

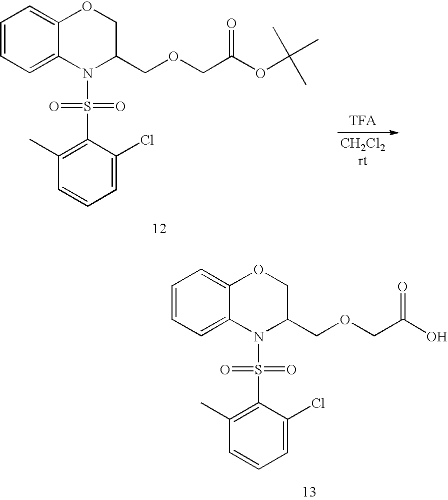

13. A solution of 12 (9.88 g, 20.1 mmol) and TFA (20 mL, 260 mmol) in CH₂Cl₂ (100 mL) was stirred at room temperature for 2 h. The reaction mixture was concentrated, co-evaporated with toluene (2×) and CH₂Cl₂ (2×). The residue was transferred to a jar with CH₂Cl₂, concentrated and dried under vacuum overnight to furnish 13 (8.50 g, ‘103’%).

### Synthesis of acid building block AC-39: 2-((4-(2-(Trifluoromethyl)phenylsulfonyl)-3,4-dihydro-2H-benzo[b][1,4]oxazin-3-yl)methoxy)acetic acid (AC-39)

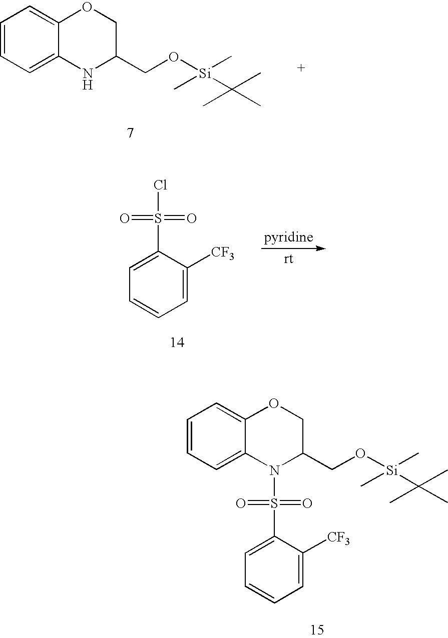

15. 2-(trifluoromethyl)benzenesulfonyl chloride (14, 8.50 g, 34.8 mmol) was added to a solution of 7 (8.83 g, 31.6 mmol) in pyridine (7.67 mL, 95.0 mmol) and the reaction mixture was stirred at room temperature overnight. CH₂Cl₂ and H₂O were added to the reaction mixture and the organic layer was separated, washed with brine and concentrated to give crude 15, which was directly used as such in the next step.

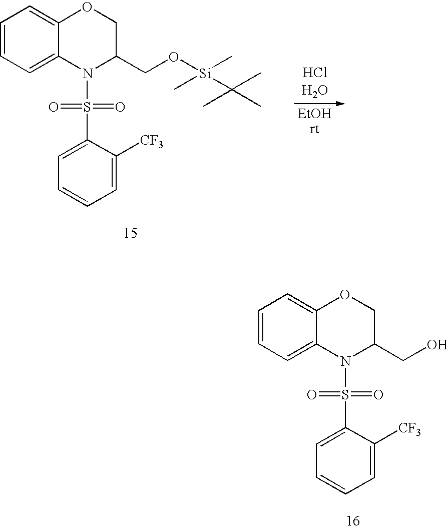

16. Aqueous 1 M HCl (50 mL, 50 mmol) was added to crude 15 in EtOH (200 mL) and the reaction mixture was stirred at room temperature overnight. The reaction mixture was concentrated, dissolved in CH₂Cl₂, washed with aqueous saturated NaHCO₃, dried (Na₂SO₄) and concentrated. The residue was purified by column chromatography (silica, heptane/EtOAc: 2:1) to yield 16 (10.29 g, 78%, 2 steps).

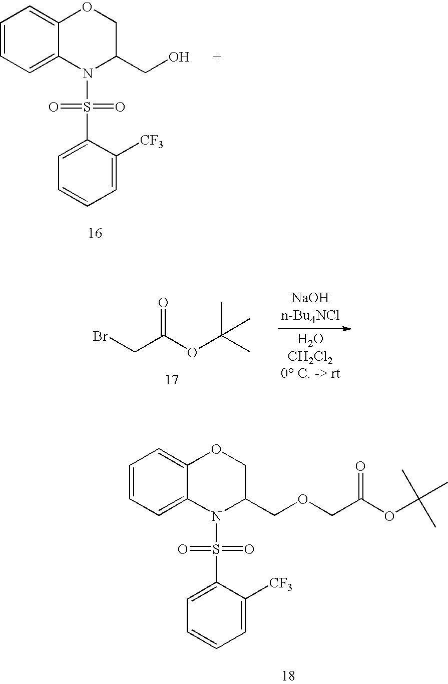

18. To an ice-cooled solution of 16 (10.29 g, 24.81 mmol) and n-Bu₄NCl (2.28 g, 8.19 mmol) in CH₂Cl₂ (125 mL) was sequentially added aqueous 35% NaOH solution (125 mL) and tert-butyl bromoacetate (17, 10.83 mL, 74.4 mmol). The reaction mixture was stirred at room temperature for 4 h, after which H₂O was added. The organic layer was separated, washed with H₂O and brine, dried (Na₂SO₄) and concentrated. The residue was purified by column chromatography (silica, heptane/EtOAc, 4:1) to provide 18 (11.65 g, 93%).

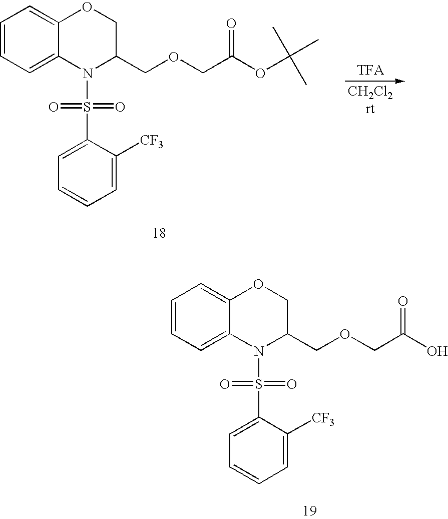

19. A solution of 18 (11.55 g, 22.98 mmol) and TFA (20 mL, 260 mmol) in CH₂Cl₂ (100 mL) was stirred at room temperature for 2 h. The reaction mixture was concentrated, co-evaporated with toluene (2×) and CH₂Cl₂ (2×). The residue was transferred to a jar with CH₂Cl₂, concentrated and dried under vacuum overnight to furnish 19 (10.18 g, ‘103’%).

### Synthesis of acid building block AC-40: 3-((1-(4-Methoxy-2,6-dimethylphenylsulfonyl)piperidin-2-yl)methoxy)propanoic acid (AC-40)

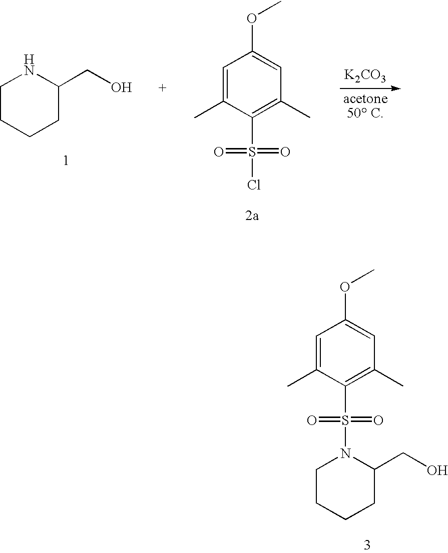

3. 2-Piperidinemethanol (1, 8.1 g, 70.11 mmol) was suspended in acetone (350 mL). K₂CO₃ (19.4 g, 140.22 mmol) was added followed by sulfonyl chloride 2a (18.1 g, 77.12 mmol). The mixture was stirred overnight at 50° C. After cooling to room temperature, the reaction mixture was filtered and the filtrate was evaporated to dryness. Purification by column chromatography (silica, heptane/EtOAc 2:1) gave 3 (12.9 g, 59%) as a white solid.

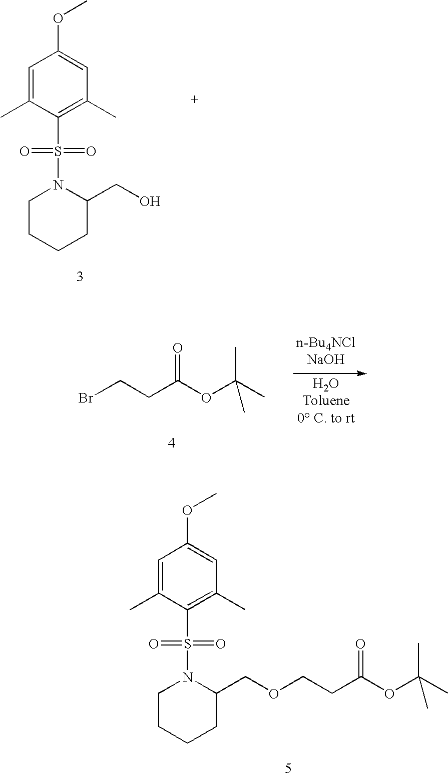

5. To a solution of alcohol 3 (12.8 g, 40.84 mmol) in toluene (200 mL) was added Bu₄NCl (3.7 g, 13.48 mmol). The reaction mixture was cooled to 0° C. after which aqueous 35% NaOH (250 mL) was added followed by a dropwise addition of tert-butyl 3-bromopropionate (4, 8.2 mL, 49.01 mmol) in toluene (50 mL). The mixture was stirred overnight at room temperature. The organic layer was separated and washed with H₂O until neutral, dried (Na₂SO₄), concentrated and co-evaporated with CH₂Cl₂ (3×). Purification by column chromatography (silica, heptane/EtOAc 4:1) gave 5 (11.2 g, 62%) as a yellow oil.

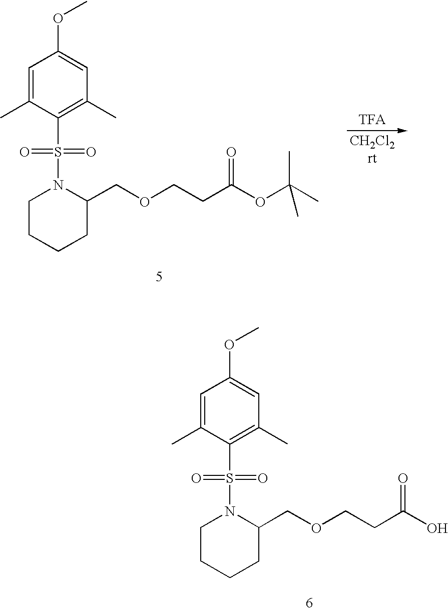

6. tert-Butyl ester 5 (10.9 g, 24.68 mmol) was dissolved in CH₂Cl₂ (150 mL). TFA (75 mL) was added and the mixture was stirred overnight at room temperature. The reaction mixture was concentrated in vacuo and co-evaporated with toluene (3×) and CH₂Cl₂ (3×).

The crude product was purified by column chromatography (silica, heptane/EtOAc 2:1+2% HOAc). Co-evaporation with toluene (2×) and CH₂Cl₂ (3×) gave 6 (9.2 g, 97%) as a yellow oil.

### Synthesis of acid building block AC-41: 2-(2-(1-(4-Methoxy-2,6-dimethylphenylsulfonyl)piperidin-2-yl)ethoxy)acetic acid (AC-41)

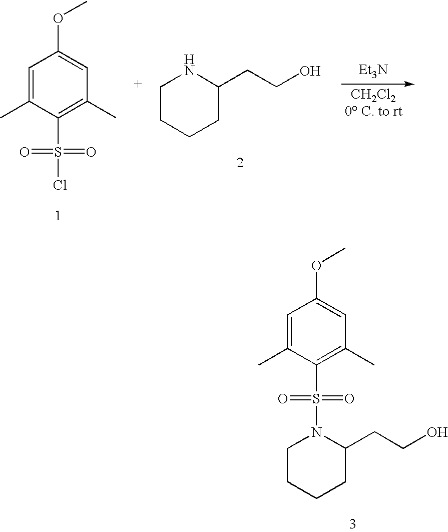

3. To a solution of 2-piperidineethanol (2, 5.63 g, 43.6 mmol) in CH₂Cl₂ (200 mL) was added Et₃N (14.1 mL, 109 mmol). At 0° C. was added 4-methoxy-2,6-dimethylbenzenesulfonyl chloride (1, 10.23 g, 43.6 mmol). The reaction mixture was stirred for 1 h at 0° C. and overnight at room temperature. Aqueous 1 M HCl (150 mL) was added and after separation of the layers the organic layer was washed with brine (150 mL), dried (Na₂SO₄) and evaporated to dryness to afford compound 3 (14.85 g, ‘104%’).

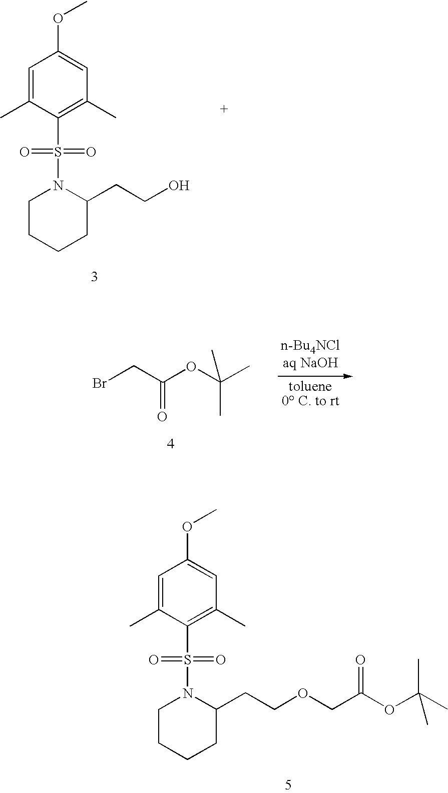

5. To a solution of alcohol 3 (14.8 g, max. 43.6 mmol) in toluene (200 mL) was added n-Bu₄NCl (4.04 g, 14.5 mmol). After cooling to 0° C., an aqueous 35% NaOH solution (200 mL) was added, followed by a dropwise addition of tert-butyl bromoacetate (4, 9.53 mL, 65.4 mmol). The reaction mixture was stirred at room temperature for 3 h. The organic layer was separated and washed with H₂O (3×200 mL), dried (Na₂SO₄) and evaporated to dryness. Purification by column chromatography (silica, heptane/EtOAc, 4:1) yielded compound 5 (12.90 g, 67%, 2 steps).

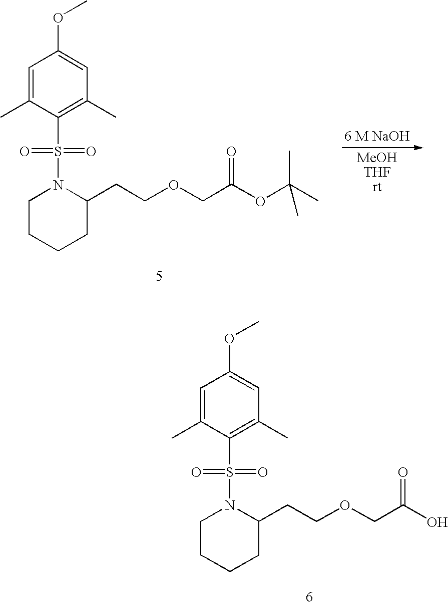

6. To a solution of ester 5 (12.90 g, 29.2 mmol) in THF (95 mL) and MeOH (95 mL) was added aqueous 6 M NaOH (95 mL). After 1 h organic solvents were evaporated and aqueous 6 M HCl (95 mL) was added at 0° C. The mixture was extracted with EtOAc (500 mL), dried (Na₂SO₄) and co-evaporated with Et₂O (2×) to afford compound 6 (11.07 g, 98%).

### Synthesis of Acid Building Block AC-43: 2-(2-(4-Methoxy-2,6-dimethyl-N-phenylphenylsulfonamido)ethoxy)acetic acid (AC-43)

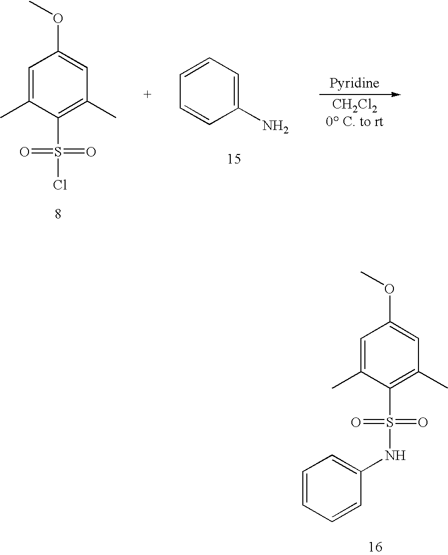

16. A solution of sulfonyl chloride 8 (10.1 g, 43.0 mmol) in CH₂Cl₂ (100 mL) was added dropwise to a stirred and cooled (0° C.) solution of aniline (15, 3.92 mL, 43.0 mmol) and pyridine (10.4 mL, 129 mmol) in CH₂Cl₂ (250 mL) and the reaction mixture was stirred at room temperature for 3 h. The mixture was washed with aqueous 0.5 M KHSO₄ (100 mL) and saturated aqueous NaHCO₃ (100 mL), dried (Na₂SO₄) and evaporated to dryness to afford crude sulfonamide 16 (14.87 g, ‘119%’).

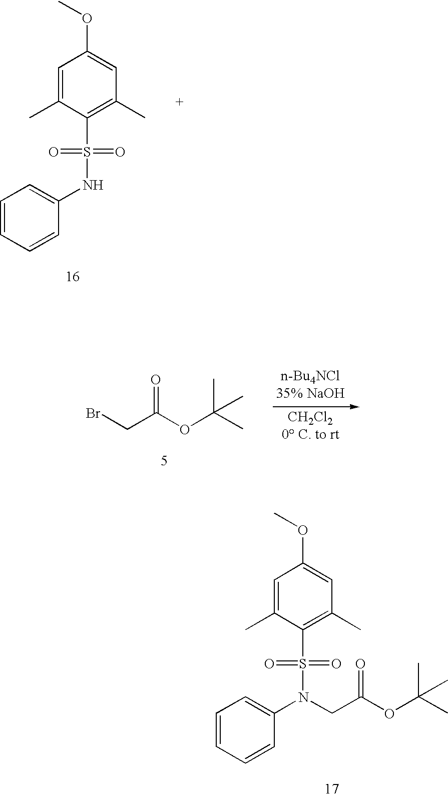

17. A solution of sulfonamide 16 (14.72 g, max. 43.0 mmol) and n-Bu₄NCl (1.50 g, 5.40 mmol) in CH₂Cl₂ (150 mL) was cooled to 0° C. and aqueous 35% NaOH (150 mL) was added. After 10 min tert-butyl bromoacetate (5, 11.2 mL, 76.0 mmol) was added and the mixture was stirred at room temperature for 3 h. The layers were separated and the organic layer was washed with H₂O (3×200 mL). The organic layer was dried (Na₂SO₄) and evaporated to dryness to afford crude ester 17 (22.6 g, ‘130%’).

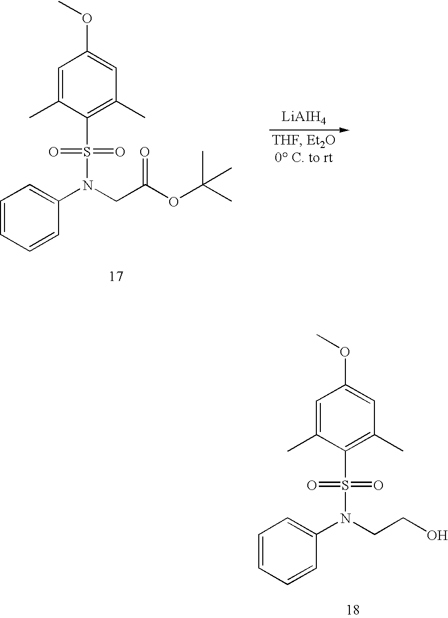

18. A solution of 4 M LiAlH₄ in Et₂O (20.9 mL, 84.0 mmol) was added dropwise to a stirred and cooled (0° C.) solution of ester 17 (22.6 g, max. 43.0 mmol) in THF (225 mL). The reaction mixture was stirred for 15 min at 0° C. after complete addition and Na₂SO₄.10H₂O was added until gas evolution stopped and was stirred at room temperature overnight. The mixture was filtered over a small pad of Na₂SO₄ and the filtrate was evaporated to dryness. The crude product was purified by column chromatography (silica, heptane/EtOAc, 2:1) to afford alcohol 18 (11.25 g, 78% over 3 steps).

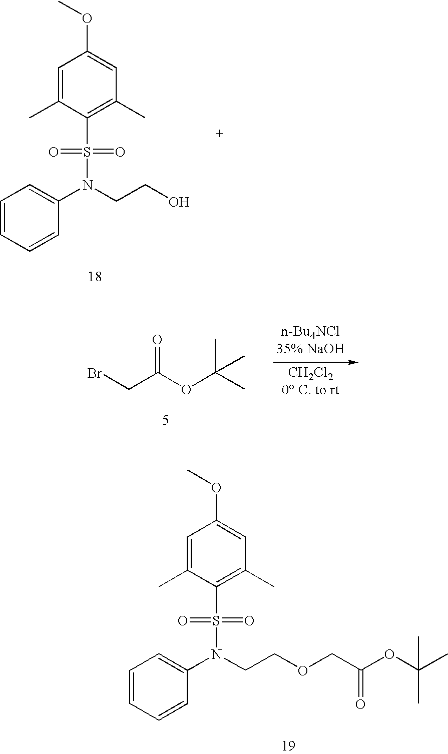

19. To a solution of alcohol 18 (11.24 g, 33.5 mmol) and n-Bu₄NCl (992 mg, 3.57 mmol) in CH₂Cl₂ (120 mL) was added aqueous 35% NaOH (120 mL) at 0° C. followed by tert-butyl bromoacetate (5, 7.43 mL, 50.3 mmol) and the reaction mixture was then stirred at room temperature. After 3 h the layers were separated and the organic phase was washed with H₂O (3×250 mL). The organic layer was dried (Na₂SO₄) and evaporated to dryness. Purification by column chromatography (silica, heptane/EtOAc, 3:1) afforded ester 19 (12.00 g, 80%) as a yellow oil.

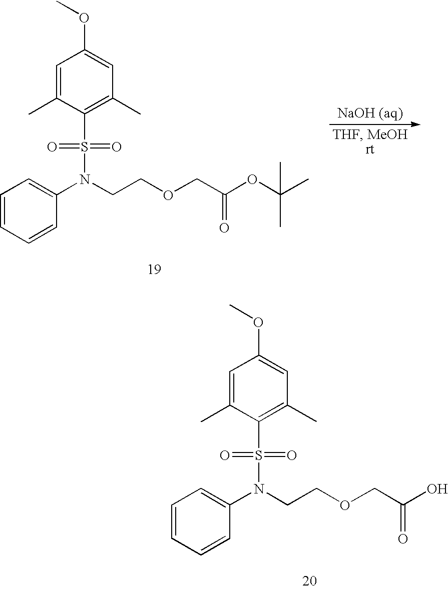

20. To a solution of ester 19 (12.00 g, 26.70 mmol) in MeOH (200 mL) and THF (200 mL) was added aqueous 4 M NaOH (200 mL, 800 mmol) and the reaction mixture was stirred at room temperature. After 3 h the organic solvents were evaporated and the aqueous layer was acidified with aqueous 6 M HCl (250 mL). The aqueous layer was extracted with CH₂Cl₂ (200 mL) and the combined organic layers were dried (Na₂SO₄) and evaporated to dryness to afford building block 20 (11.27 g, ‘107%’).

### Synthesis of Acid Building Block AC-44: 2-((1-(2-(Trifluoromethyl)phenylsulfonyl)piperidin-2-yl)methoxy)acetic acid (AC-44) (AC1000454/ME20060001-1-41)

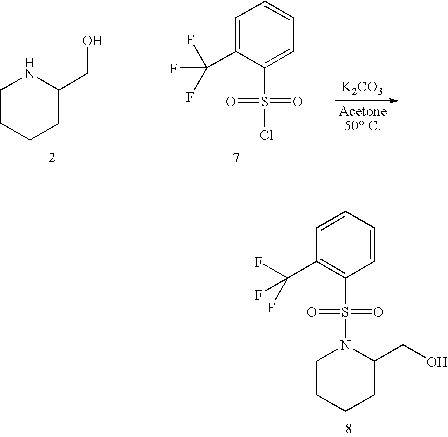

8. Alcohol 2 (4.3 g, 37.2 mmol) was suspended in acetone (150 mL). K₂CO₃ (10.27 g, 74.3 mmol) and 2-(trifluoromethyl)benzenesulfonyl chloride (7.10 g, 40.9 mmol) were subsequently added. The mixture was stirred overnight at 50° C. The reaction mixture was filtrated after cooling to room temperature and the filtrate was evaporated to dryness under reduced pressure. The crude product was purified by column chromatography (silica, heptane/EtOAc 2:1) to afford 8.95 g (75%) of alcohol 8.

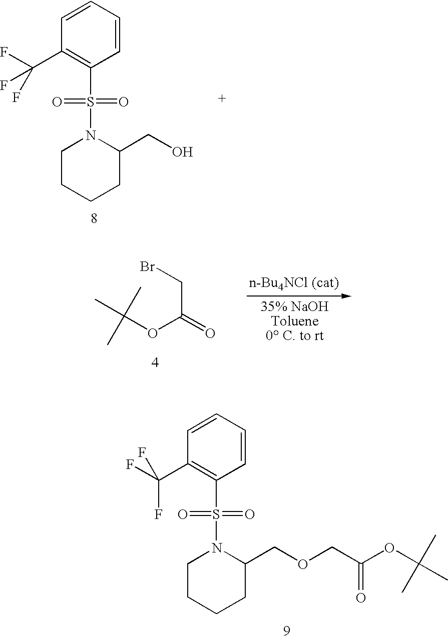

9. To a solution of alcohol 8 (8.95 g, 27.7 mmol) in toluene (100 mL) was added n-Bu₄NCl (2.54 g, 9.1 mmol). The reaction mixture was cooled to 0° C. after which aqueous 35% NaOH (100 mL) was added, followed by the addition of tert-butyl bromoacetate (4, 6.05 mL, 41.5 mmol). After stirring for 3 h at room temperature no more starting material was seen on TLC (silica, heptane/EtOAc, 2:1). The organic layer was separated and washed with H₂O (4×200 mL) and brine (200 mL) until neutral, dried (Na₂SO₄) and concentrated under reduced pressure. Purification by column chromatography (silica, heptane/EtOAc 4:1) afforded 11.57 g (96%) of ester 9.

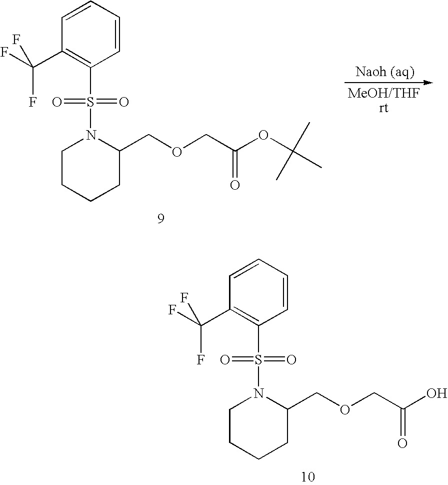

10. A mixture of ester 9 (11.57 g, 26.4 mmol), aqueous 6 M NaOH (88 mL, 528 mmol), MeOH (85 mL) and THF (85 mL) was stirred at room temperature for 30 min. The reaction was complete according to TLC (silica, heptane/EtOAc 2:1). The solution was then concentrated under reduced pressure to remove MeOH. The resulting suspension was acidified with aqueous 6 M HCl (120 mL) at 0° C. CH₂Cl₂ (300 mL) was added and after separation of the layers, the aqueous layer was extracted with CH₂Cl₂ (100 mL). The combined organic layers were dried (Na₂SO₄) and evaporated to dryness under reduced pressure affording 9.89 g (98%) of carboxylic acid 10.

### Amine Units

The following amine units were prepared and employed for synthesis of the compounds according to the invention:

| Amine | | |
|---|---|---|
| unit | Structure | Name |
| AM1 | 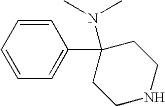 | N,N-Dimethyl-4-phenylpiperidin-4- amine |
| AM2 | 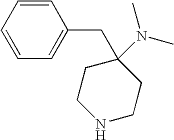 | 4-Benzyl-N,N-dimethylpiperidin-4- amine |
| AM3 | 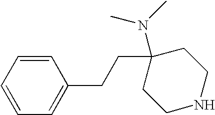 | N,N-Dimethyl-4-phenethylpiperidin- 4-amine |
| AM4 | 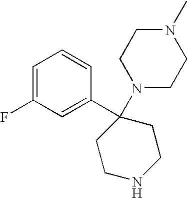 | 1-(4-(3-Fluorophenyl)piperidin-4-yl)- 4-methylpiperazine |
| AM5 | 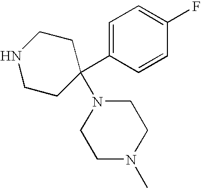 | 1-(4-(4-Fluorophenyl)piperidin-4-yl)- 4-methylpiperazine |
| AM6 | 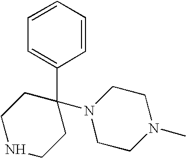 | 1-Methyl-4-(4-phenylpiperidin-4- yl)piperazine |
| AM7 | 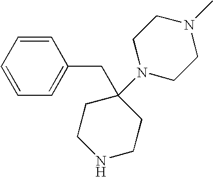 | 1-(4-Benzylpiperidin-4-yl)-4- methylpiperazine |
| AM8 | 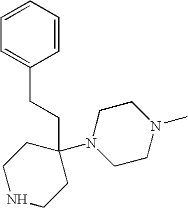 | 1-Methyl-4-(4-phenethylpiperidin-4- yl)piperazine |
| AM9 | 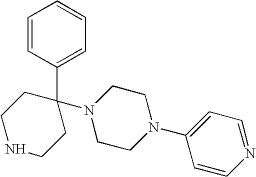 | 1-(4-Phenylpiperidin-4-yl)-4-(pyridin- 4-yl)piperazine |
| AM10 | 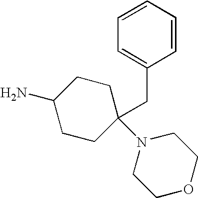 | 4-Benzyl-4- morpholinocyclohexanamine |
| AM11 | 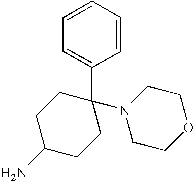 | 4-Morpholino-4- phenylcyclohexanamine |
| AM12 | 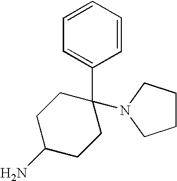 | 4-Phenyl-4-(pyrrolidin-1- yl)cyclohexanamine |
| AM13 | 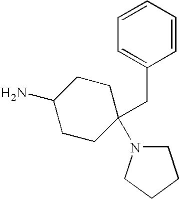 | 4-Benzyl-4-(pyrrolidin-1- yl)cyclohexanamine |
| AM14 | 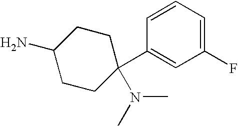 | 1-(3-Fluorophenyl)-N1,N1- dimethylcyclohexane-1,4-diamine |
| AM15 | 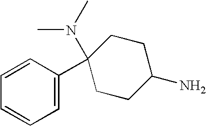 | N1,N1-Dimethyl-1- phenylcyclohexane-1,4-diamine |
| AM16 | 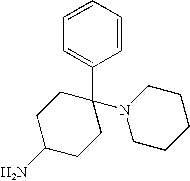 | 4-Phenyl-4-(piperidin-1- yl)cyclohexanamine |
| AM17 | 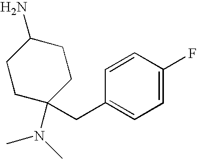 | 1-(4-Fluorobenzyl)-N1,N1- dimethylcyclohexane-1,4-diamine |
| AM18 | 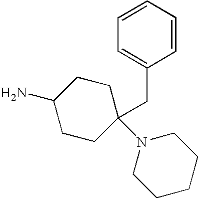 | 4-Benzyl-4-(piperidin-1- yl)cyclohexanamine |
| AM19 | 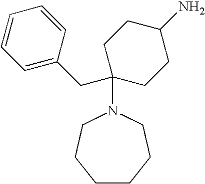 | 4-(Azepan-1-yl)-4- benzylcyclohexanamine |
| AM20 | 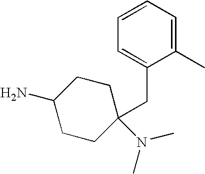 | N1,N1-Dimethyl-1-(2- methylbenzyl)cyclohexane-1,4- diamine |
| AM21 | 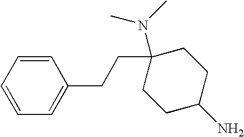 | N1,N1-Dimethyl-1- phenethylcyclohexane-1,4-diamine |
| AM22 | 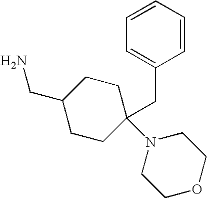 | (4-Benzyl-4- morpholinocyclohexyl)methanamine |
| AM23 | 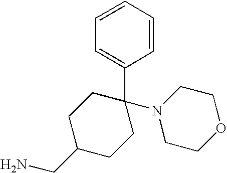 | (4-Morpholino-4- phenylcyclohexyl)methanamine |
| AM24 | 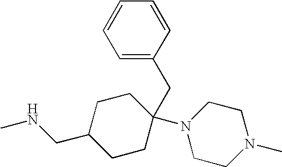 | 1-(4-Benzyl-4-(4-methylpiperazin-1- yl)cyclohexyl)-N- methylmethanamine |
| AM25 | 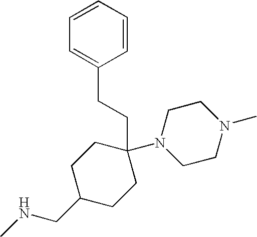 | N-Methyl-1-(4-(4-methylpiperazin-1- yl)-4- phenethylcyclohexyl)methanamine |
| AM26 | 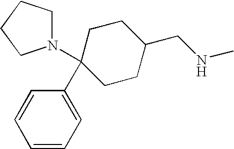 | N-Methyl-1-(4-phenyl-4-(pyrrolidin-1- yl)cyclohexyl)methanamine |
| AM27 | 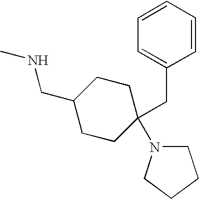 | 1-(4-Benzyl-4-(pyrrolidin-1- yl)cyclohexyl)-N- methylmethanamine |
| AM28 | 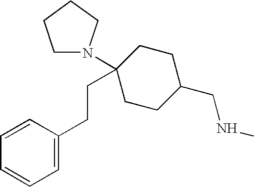 | N-Methyl-1-(4-phenethyl-4- (pyrrolidin-1- yl)cyclohexyl)methanamine |
| AM29 | 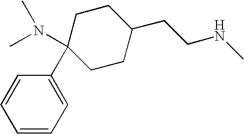 | N,N-Dimethyl-4-(2- (methylamino)ethyl)-1- phenylcyclohexanamine |
| AM30 | 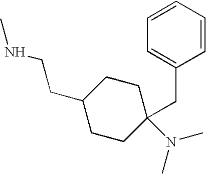 | 1-Benzyl-N,N-dimethyl-4-(2- (methylamino)ethyl)cyclohexanamine |
| AM31 | 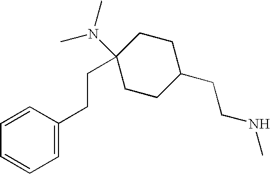 | N,N-Dimethyl-4-(2- (methylamino)ethyl)-1- phenethylcyclohexanamine |
| AM32 | 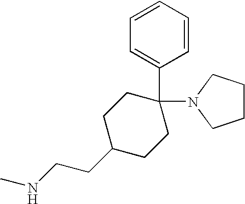 | N-Methyl-2-(4-phenyl-4-(pyrrolidin-1- yl)cyclohexyl)ethanamine |
| AM33 | 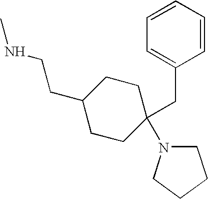 | 2-(4-Benzyl-4-(pyrrolidin-1- yl)cyclohexyl)-N-methylethanamine |
| AM34 | 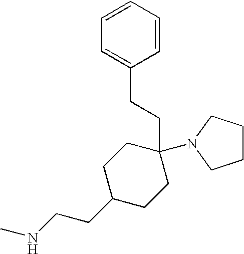 | N-Methyl-2-(4-phenethyl-4- (pyrrolidin-1- yl)cyclohexyl)ethanamine |
| AM35 | 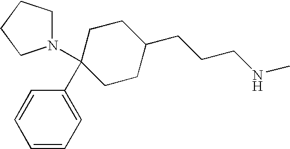 | N-Methyl-3-(4-phenyl-4-(pyrrolidin-1- yl)cyclohexyl)propan-1-amine |
| AM36 | 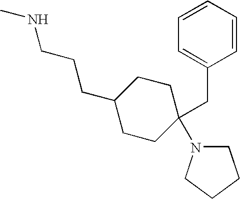 | 3-(4-Benzyl-4-(pyrrolidin-1- yl)cyclohexyl)-N-methylpropan-1- amine |
| AM37 | 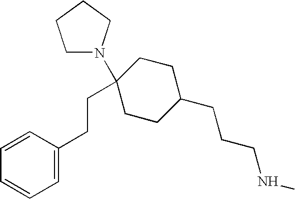 | N-Methyl-3-(4-phenethyl-4- (pyrrolidin-1-yl)cyclohexyl)propan-1- amine |
| AM38 | 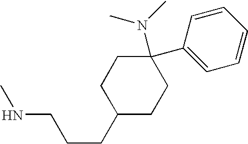 | N,N-Dimethyl-4-(3- (methylamino)propyl)-1- phenylcyclohexanamine |
| AM39 | 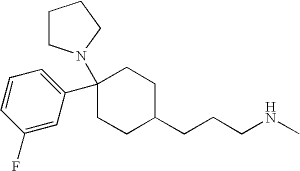 | 3-(4-(3-Fluorophenyl)-4-(pyrrolidin-1- yl)cyclohexyl)-N-methylpropan-1- amine |
| AM40 | 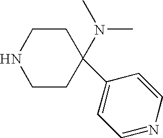 | N,N-Dimethyl-4-(pyridin-4-yl)- piperidin-4-amine |
| AM41 | 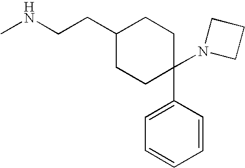 | 2-(4-(Azetidin-1-yl)-4- phenylcyclohexyl)-N- methylethanamine |
| AM42 | 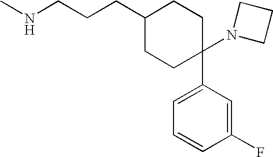 | 3-(4-(Azetidin-1-yl)-4-(3- fluorophenyl)cyclohexyl)-N- methylpropan-1-amine |
| AM43 | 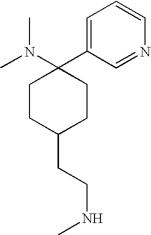 | N,N-Dimethyl-4-(2- (methylamino)ethyl)-1-(pyridin-3- yl)cyclohexanamine |
| AM44 | 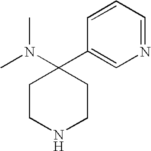 | N,N-Dimethyl-4-(pyridin-3- yl)piperidin-4-amine |
| AM45 | 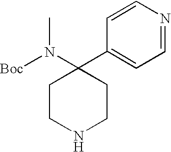 | tert-Butyl methyl(4-(pyridin-4- yl)piperidin-4-yl)carbamate |
| AM46 | 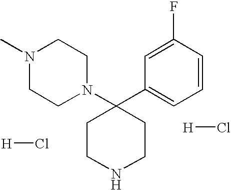 | 1-(4-(3-Fluorophenyl)piperidin-4-yl)- 4-methylpiperazine dihydrochloride |

### Synthesis of the Amines AM1-AM9

### Method A

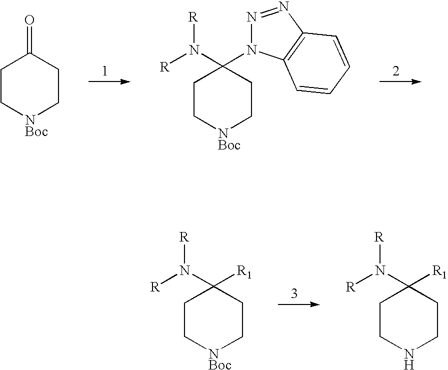

Stage 1. N-Boc-piperidone (15 mmol), the corresponding amine (15 mmol) and benzotriazole (15 mmol) were heated under reflux in benzene (60 ml) using a Dean-Stark water separator. The solvent was then stripped off under reduced pressure. The crude product obtained was used further without further purification.

Stage 2. The corresponding benzotriazole adduct (12 mmol) in THF was added dropwise to a solution of the corresponding Grignard reagent in THF (60 mmol) at 0° C. The reaction mixture was warmed to 25° C. and stirred at this temperature for 16 h (TLC control). It was then cooled to 0° C., saturated ammonium chloride solution was added and the mixture was extracted with ethyl acetate. The organic phase was washed successively with water and saturated NaCl solution and dried over Na₂SO₄. The solvent was removed and the crude product obtained was purified by column chromatography (silica gel, MC/methanol, 98:2-95:5)

Stage 3. TFA (20% in MC, 5 ml/mmol) was added to the Boc-protected compound at 0° C. and the mixture was then stirred at RT for 3 h (TLC control). The solvent was removed completely and the crude product (TFA salt) was used further without further purification.

### Method B

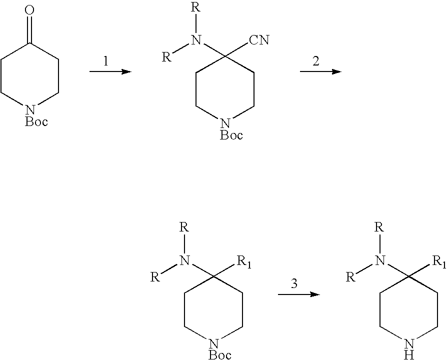

Stage 1. KCN (24 mmol) and dimethylamine (22 mmol) were added to a solution of Boc-piperidone (20 mmol) in a mixture of ethanol (20 ml) and water (10 ml). The reaction mixture was stirred at 25° C. for 72 h (TLC control). The reaction mixture was then diluted with ethyl acetate. The organic phase was washed successively with water, aqueous FeSO₄ solution and saturated NaCl solution and then dried over Na₂SO₄. The solvent was stripped off under reduced pressure. The crude product was used further without further purification.

Stage 2. The aminonitrile (15 mmol) was dissolved in THF (100 ml) and the corresponding Grignard reagent (60 mmol) in THF (30 ml) was added dropwise under an argon atmosphere, while cooling with ice. The reaction mixture was warmed to 25° C. and stirred at this temperature for 36 h (TLC control). When the reaction had ended, ammonium chloride solution (100 ml) was added and the mixture was then extracted with ethyl acetate. The organic phase was washed with water and saturated NaCl solution, dried over Na₂SO₄ and concentrated. The crude product was purified by column chromatography (silica gel, MC/methanol, 98:2-95:5)

Stage 3. TFA (20% in MC, 5 ml/mmol) was added to the Boc-protected compound at 0° C. and the mixture was then stirred at RT for 3 h (TLC control). The solvent was removed completely and the crude product (TFA salt) was used further without further purification.

Amine units AM1-AM9

| No. | NR₂ | R¹ | Method | Name |
|---|---|---|---|---|
| AM1 | NMe₂ | 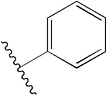 | B | N,N-Dimethyl-4- phenylpiperidin-4-amine |
| AM2 | NMe₂ | 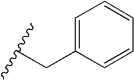 | B | 4-Benzyl-N,N-dimethyl- piperidin-4-amine |
| AM3 | NMe₂ | 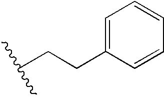 | B | N,N-Dimethyl-4- phenethylpiperidin-4- amine |
| AM4 | 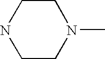 | 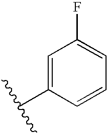 | A | 1-(4-(3- Fluorophenyl)piperidin- 4-yl)-4-methylpiperazine |
| AM5 | 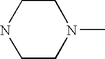 | 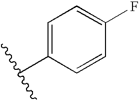 | A | 1-(4-(4- Fluorophenyl)piperidin- 4-yl)-4-methylpiperazine |
| AM6 | 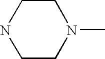 | 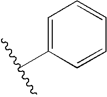 | A | 1-Methyl-4-(4- phenylpiperidin-4- yl)piperazine |
| AM7 | 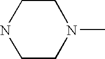 | 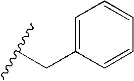 | A | 1-(4-Benzylpiperidin-4- yl)-4-methylpiperazine |
| AM8 | 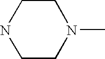 | 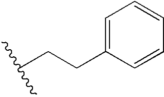 | A | 1-Methyl-4-(4- phenethylpiperidin-4- yl)piperazine |
| AM9 | 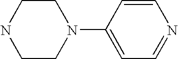 | 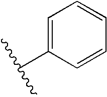 | A | 1-(4-Phenylpiperidin-4- yl)-4-(pyridin-4- yl)piperazine |

### Synthesis of the Amines AM10-AM21

### Method A

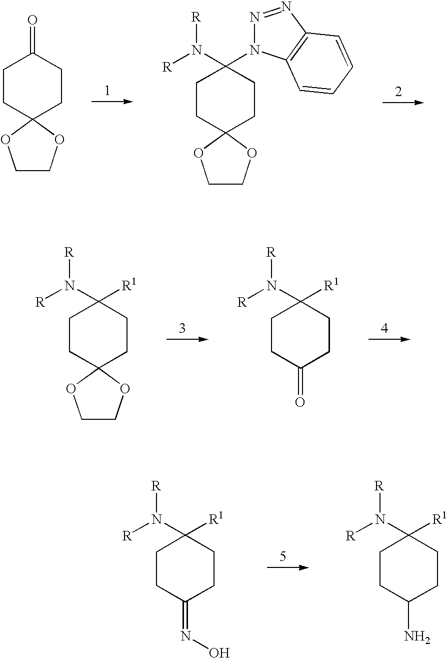

Stage 1. Cyclohexane-1,4-dione monoethylene ketal (15 mmol), the corresponding amine (15 mmol) and benzotriazole (15 mmol) were heated under reflux in benzene (60 ml) using a Dean-Stark water separator. The solvent was then stripped off under reduced pressure. The crude product obtained was used further without further purification.

Stage 2. The corresponding benzotriazole adduct (12 mmol) in THF was added dropwise to a solution of the corresponding Grignard reagent in THF (60 mmol) at 0° C. The reaction mixture was warmed to 25° C. and stirred at this temperature for 16 h (TLC control). It was then cooled to 0° C., saturated ammonium chloride solution was added and the mixture was extracted with ethyl acetate. The organic phase was washed successively with water and saturated NaCl solution and dried over Na₂SO₄. The solvent was removed and the crude product obtained was purified by column chromatography (silica gel, MC/methanol, 98:2-95:5)

Stage 3. The Grignard product (105 mmol) was slowly added to a solution of conc. HCl and water (1:1, 88 ml) at 0° C. and the mixture was then stirred at 25° C. for 20 h. The mixture was then extracted twice with ethyl acetate (100 ml each time). The extract was then rendered basic with aqueous 5 N NaOH and extracted three times with MC (100 ml each time). The organic phase was dried over Na₂SO₄ and concentrated. The product was used without further purification.

Stage 4. The ion exchange resin Amberlyst A21 (40 g) was added to a solution of the ketone (40 mmol) in abs. ethanol (200 ml) at 25° C. The reaction mixture was stirred at 25° C. for 20 h. The ion exchange resin was filtered off and rinsed twice with 200 ml of ethanol each time. The combined organic phases were concentrated. The crude product obtained was used further without further purification.

Stage 5. LAH (77 mmol) was added to dry THF (400 ml) under an argon atmosphere. The reaction mixture was increased to 60° C. and a solution of the oxime (38.5 mmol) in THF (90 ml) was added dropwise. The reaction mixture was stirred at 60° C. for 4 h and then cooled. Water (100 ml) was added dropwise, while cooling with an ice bath. The solution was then filtered over silica gel. The aqueous solution was extracted with ethyl acetate. The combined organic phases were concentrated and the crude product obtained was purified by column chromatography (silica gel, MC/methanol, 95:5→90:10).

### Method B

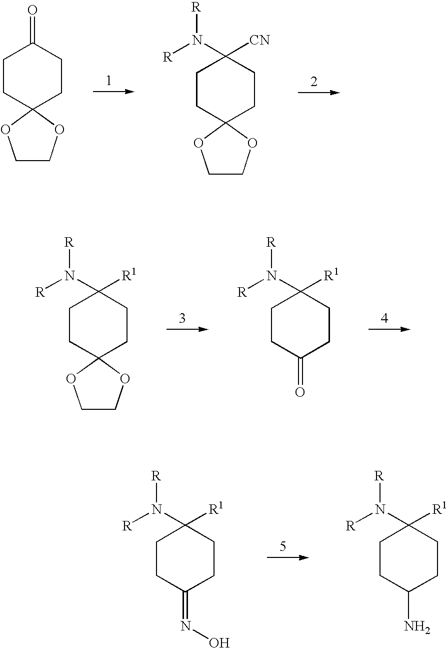

Stage 1. KCN (24 mmol) and the corresponding amine (22 mmol) were added to a solution of cyclohexane-1,4-dione monoethylene ketal (20 mmol) in a mixture of ethanol (20 ml) and water (10 ml). The reaction mixture was stirred at 25° C. for 72 h (TLC control). The reaction mixture was then diluted with ethyl acetate. The organic phase was washed successively with water, aqueous FeSO₄ solution and saturated NaCl solution and then dried over Na₂SO₄. The solvent was stripped off under reduced pressure. The crude product was used further without further purification.

Stage 2. The aminonitrile (15 mmol) was dissolved in THF (100 ml) and the corresponding Grignard reagent (60 mmol) in THF (30 ml) was added dropwise under an argon atmosphere, while cooling with ice. The reaction mixture was warmed to 25° C. and stirred at this temperature for 36 h (TLC control). When the reaction had ended, ammonium chloride solution (100 ml) was added and the mixture was then extracted with ethyl acetate. The organic phase was washed with water and saturated NaCl solution, dried over Na₂SO₄ and concentrated. The crude product was purified by column chromatography (silica gel, MC/methanol, 98:2→95:5)

Stage 3. Aqueous HCl (6 N, 20 ml) was added to the acetal (10 mmol) at 0° C. and the mixture was then warmed to RT and stirred at this temperature for 16 h (TLC control). The aqueous phase was washed with ethyl acetate and adjusted to about pH 14 with aqueous NaOH (6 N). The aqueous phase was extracted with MC and the organic phase was then washed successively with water and saturated NaCl solution. The mixture was dried over Na₂SO₄ and the solvent was stripped off under reduced pressure. The crude product was employed further without further purification.

Stage 4. The ion exchange resin Amberlyst A21 (40 g) was added to a solution of the ketone (40 mmol) in abs. ethanol (200 ml) at 25° C. The reaction mixture was stirred at 25° C. for 20 h. The ion exchange resin was filtered off and rinsed twice with 200 ml of ethanol each time. The combined organic phases were concentrated. The crude product obtained was used further without further purification.

Stage 5. LAH (77 mmol) was added to dry THF (400 ml) under an argon atmosphere. The reaction mixture was increased to 60° C. and a solution of the oxime (38.5 mmol) in THF (90 ml) was added dropwise. The reaction mixture was stirred at 60° C. for 4 h and then cooled. Water (100 ml) was added dropwise, while cooling with an ice bath. The solution was then filtered over silica gel. The aqueous solution was extracted with ethyl acetate. The combined organic phases were concentrated and the crude product obtained was purified by column chromatography (silica gel, MC/methanol, 95:5→90:10).

### Method C

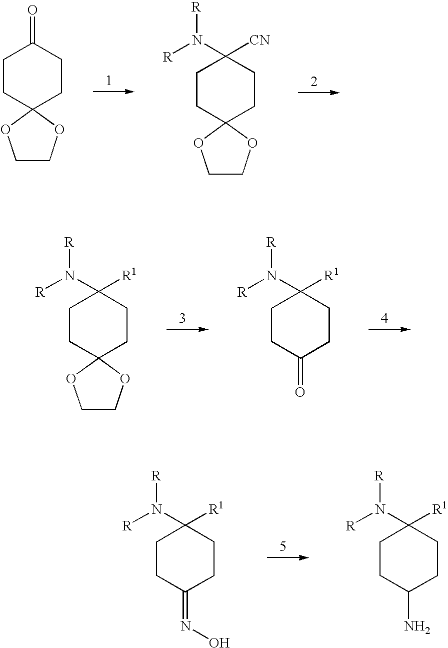

Stage 1. 40 percent aqueous dimethylamine solution (116 ml, 0.92 mol) or the corresponding amine (0.92 mmol), cyclohexane-1,4-dione monoethylene ketal (30.0 g, 0.192 mol) and potassium cyanide (30.0 g, 0.46 mol) were added to a mixture of 4 N hydrochloric acid (50 ml) and methanol (30 ml) while cooling with ice (if the 40 percent dimethylamine solution was not used, water (0.1 ml/mmol of amine) also had to be additionally added.). The mixture was stirred at room temperature for 72 h and then, after addition of water (80 ml), extracted with diethyl ether (4×100 ml). After concentration of the solution, the residue was taken up in MC (200 ml) and dried with MgSO₄ overnight. The organic phase was concentrated and the ketal was obtained as a white solid.

Stage 2. The aminonitrile (0.1 mol), dissolved in THF (210 ml), was added to a solution of the corresponding Grignard reagent (0.198 mol) in the course of 15 min, under argon and while cooling with ice, and the mixture was then stirred at room temperature for 16 h. For working up of the reaction mixture, saturated ammonium chloride solution (150 ml) was added, while cooling with ice, and the mixture was extracted with diethyl ether (3×100 ml). The organic phase was extracted by shaking with water (100 ml) and saturated NaCl solution and concentrated. The crude product was dissolved in ethyl methyl ketone (280 ml) and chlorotrimethylsilane (18.8 ml, 0.15 mol) was added, while cooling with ice. After a reaction time of 6 h, it was possible to isolate the hydrochloride as a white solid.

Stage 3. The hydrochloride (35.2 mmol) was dissolved in 7.5 N hydrochloric acid (36 ml) and the solution was stirred at room temperature for 96 h. When the hydrolysis had ended, the reaction mixture was extracted with diethyl ether (2×50 ml). The aqueous phase was rendered alkaline with 5 N NaOH, while cooling with ice, extracted with MC (3×50 ml) and concentrated. The crude product was used further without further purification.

Stage 4. The ketone (46 mmol) and hydroxylamine hydrochloride (4.8 g, 69 mmol) were dissolved in absolute ethanol (120 ml). The basic ion exchanger Amberlyst A 21 (30.67 g, 127.28 meq.) was then added to the solution and the mixture was stirred at RT. The course of the reaction was monitored by TLC. The ion exchanger was filtered off and washed on the frit with ethanol (3×50 ml). The ethanol was distilled off and the residue was adjusted to pH 11 with 5 N NaOH. The alkaline phase was diluted with water and extracted with ethyl acetate (4×30 ml). The organic phase was dried with Na₂SO₄ and concentrated.

Stage 5. Dry THF (200 ml) was initially introduced into the reaction vessel with exclusion of oxygen, and LAH (1,644 g, 43 mmol) was added. The mixture was heated to 60° C. and the oxime (21.5 mmol) was added in portions. The mixture was stirred at an internal temperature of 60° C. for 8 h. The course of the reaction was monitored by TLC. For working up, H₂O (100 ml) was cautiously added to the mixture and the mixture was then filtered over Celite. The residue on the filter was washed with THF. THF was distilled off on a rotary evaporator. The residue was adjusted to pH 11 with 5 N NaOH and extracted with ethyl acetate (5×20 ml). The organic phase was dried with Na₂SO₄ and evaporated.

Amine units AM10-AM21

| No. | NR₂ | R¹ | Method | Name |
|---|---|---|---|---|
| AM10 | 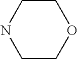 | 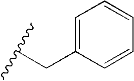 | A | 4-Benzyl-4- morpholino- cyclohexanamine |
| Am11 | 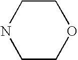 | 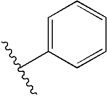 | B | 4-Morpholino-4- phenylcyclo- hexanamine |
| AM12 | 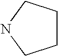 | 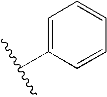 | B | 4-Phenyl-4- (pyrrolidin-1- yl)cyclohexanamine |
| AM13 | 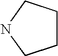 | 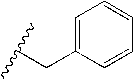 | A | 4-Benzyl-4- (pyrrolidin-1- yl)cyclohexanamine |
| AM14 | NMe₂ | 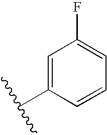 | C | 1-(3-Fluorophenyl)- N1,N1-dimethyl- cyclohexane-1,4- diamine |
| AM15 | NMe₂ | 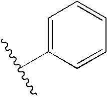 | C | N1,N1-Dimethyl-1- phenylcyclohexane- 1,4-dimaine |
| AM16 | 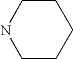 | 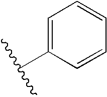 | C | 4-Phenyl-4- (piperidin-1- yl)cyclohexanamine |
| AM17 | NMe₂ | 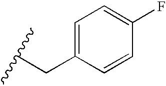 | C | 1-(4-Fluorobenzyl)- N1,N1-dimethylcyclo- hexane-1,4-dimaine |
| AM18 | 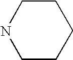 | 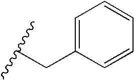 | C | 4-Benzyl-4-(piperi- din-1-yl)cyclo- hexanamine |
| AM19 | 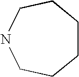 | 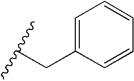 | C | 4-(Azepan-1-yl)-4- benzylcyclo- hexanamine |
| AM20 | NMe₂ | 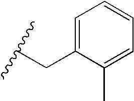 | C | N1,N1-Di- methyl-1-(2- methylbenzyl)cyclo- hexane-1,4-dimaine |
| AM21 | NMe₂ | 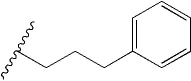 | C | N1,N1-Dimethyl-1- phenethylcyclo- hexane-1,4-diamine |

### Synthesis of the Amines AM22 and AM23

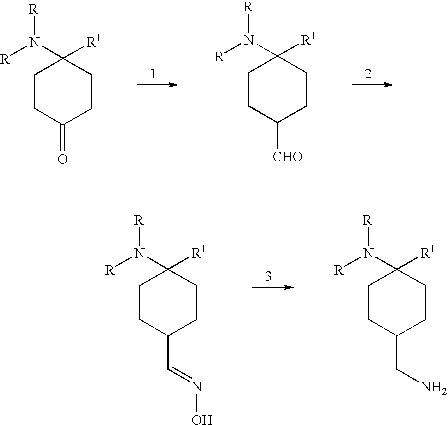

Stage 1. A suspension of (methoxymethyl)triphenyl-phosphonium chloride (10 mmol) in dry THF was added dropwise to a solution of potassium tert-butylate (10 mmol) in dry THF (10 ml) at 0° C. under an argon atmosphere and the mixture was stirred at this temperature for 15 min. A solution of the ketone (6 mmol) in dry THF was added dropwise at 25° C. and the mixture was stirred at this temperature for 16 h. The mixture was cooled to 0° C. and acidified with HCl solution (6 N). After stirring at RT for 1 h, the mixture was extracted with ethyl acetate and the aqueous phase was rendered basic (˜pH 11) with aqueous NaOH solution (5 N) and extracted with MC. After drying over Na₂SO₄, the solvent was stripped off in vacuo and the crude product was employed further without further purification.

Stage 2. The ion exchange resin Amberlyst A21 (40 g) was added to a solution of the aldehyde (40 mmol) in abs. ethanol (200 ml) at 25° C. The reaction mixture was stirred at 25° C. for 20 h. The ion exchange resin was filtered off and rinsed twice with 200 ml of ethanol each time. The combined organic phases were concentrated. The crude product obtained was used further without further purification.

Stage 3. LAH (77 mmol) was added to dry THF (400 ml) under an argon atmosphere. The reaction mixture was increased to 60° C. and a solution of the oxime (38.5 mmol) in THF (90 ml) was added dropwise. The reaction mixture was stirred at 60° C. for 4 h and then cooled. Water (100 ml) was added dropwise, while cooling with an ice bath. The solution was then filtered over silica gel. The aqueous solution was extracted with ethyl acetate. The combined organic phases were concentrated and the crude product obtained was purified by column chromatography (silica gel, MC/methanol, 95:5→90:10).

| No. | NR₂ | R¹ | Name |
|---|---|---|---|
| AM22 | 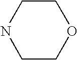 | 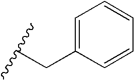 | (4-Benzyl-4-morpholino- cyclohexyl)methanamine |
| AM23 | 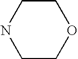 | 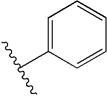 | (4-Morpholino-4-phenyl- cyclohexyl)methanamine |

### Synthesis of the Amines AM24 and AM25

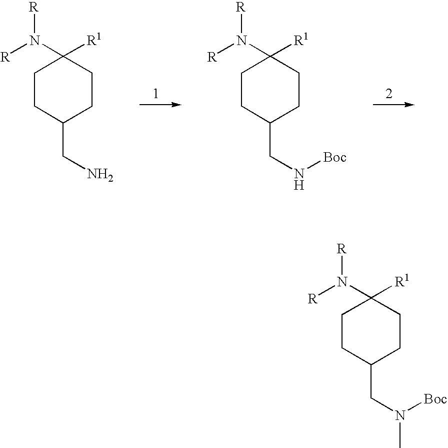

Stage 1. DIPEA (1.5 eq.) and di-tert-butyl dicarbonate (1.5 eq.) were added to a solution of the cyclohexylmethanamine (1 eq.) in MC (3 ml/mmol) at 0° C. The reaction solution was warmed to 25° C. and stirred at this temperature for 6 h (TLC control). When the reaction was complete, the mixture was diluted with MC and the organic phase was washed successively with water and saturated NaCl solution. The mixture was dried over Na₂SO₄ and the solvent was stripped off under reduced pressure. The crude product was purified by column chromatography (silica gel, MC/ethyl acetate, 1:1).

Stage 2. NaH (1.5 eq.) and methyl iodide (10 eq.) were added to a cooled solution of the Boc-protected amine (1 eq.) in THF and the mixture was then stirred at RT for 3 h. When the reaction was complete (TLC control), the mixture was hydrolysed with water and the THF was removed under reduced pressure. The residue was taken up in ethyl acetate and the mixture was washed successively with water and NaCl solution. The organic phase was dried over Na₂SO₄ and then concentrated. The crude product was purified by column chromatography (silica gel, MC/ethyl acetate 8:2).

| No. | NR₂ | R¹ | Name |
|---|---|---|---|
| AM24 | 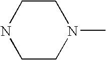 | 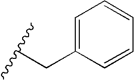 | 1-(4-Benzyl-4-(4- methylpiperazin-1-yl)cyclohexyl)- N-methylmethanamine* |
| AM25 | 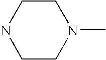 | 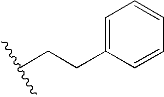 | N-Methyl-1-(4-(4- methylpiperazin-1-yl)-4- phenethylcyclohexyl)- methanamine** |

*The 1-(4-benzyl-4-(4-methylpiperazin-1-yl)cyclohexyl)methanamine required for the synthesis was prepared analogously to the synthesis of the amines 22 and AM23.
** The 1-(4-benzyl-4-(4-methylpiperazin-1-yl)cyclohexyl)methanamine required for the synthesis was prepared analogously to the synthesis of the amines 22 and AM23.

### Synthesis of the Amines AM26-AM28

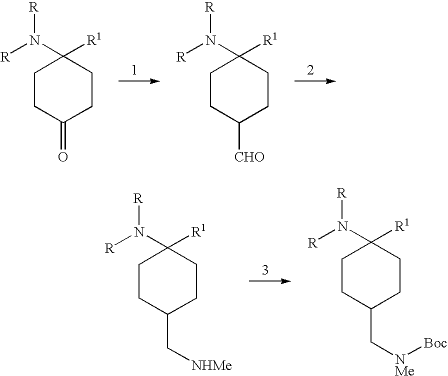

Stage 1. A suspension of (methoxymethyl)triphenyl-phosphonium chloride (10 mmol) in dry THF was added dropwise to a solution of potassium tert-butylate (10 mmol) in dry THF (10 ml) at 0° C. under an argon atmosphere and the mixture was stirred at this temperature for 15 min. A solution of the ketone (6 mmol) in dry THF was added dropwise at 25° C. and the mixture was stirred at this temperature for 16 h. The mixture was cooled to 0° C. and acidified with HCl solution (6 N). After stirring at RT for 1 h, the mixture was extracted with ethyl acetate and the aqueous phase was rendered basic (˜pH 11) with aqueous NaOH solution (5 N) and extracted with MC. After drying over Na₂SO₄, the solvent was stripped off in vacuo and the crude product was employed further without further purification.

Stage 2. A solution of methylamine (2 M in THF, 7.5 ml) and molecular sieve (4 Å, 500 wt. %, based on the aldehyde) was added to a solution of the aldehyde (10 mmol) in MC (50 ml) under an argon atmosphere and the mixture was stirred at 25° C. for 6 h. The reaction solution was filtered, the solvent was stripped off completely, the residue was taken up in dry methanol (50 ml) and the mixture was cooled to 0° C. Sodium borohydride (7.5 mmol) was added in portions to this solution and the mixture was stirred at 25° C. for 16 h. Hydrolysis was carried out with ice, the solvent was stripped off on a rotary evaporator and the residue was taken up in ethyl acetate. The organic phase was washed successively with water and saturated NaCl solution and dried over Na₂SO₄. The solvent was removed and the crude product obtained was employed further without further purification.

Stage 3. DIPEA (25 mmol) and di-tert-butyl dicarbonate (15 mmol) were added to a solution of the amine derivative (10 mmol) in MC (30 ml) at 0° C. The reaction solution was warmed to RT and stirred at this temperature for 16 h (TLC control). When the reaction was complete, the mixture was diluted with MC and the organic phase was washed successively with water and saturated NaCl solution. The mixture was dried over Na₂SO₄ and the solvent was stripped off under reduced pressure. The crude product was purified by column chromatography (silica gel, MC/methanol, 95:5→9:1)

Amine units AM26-AM28

| No. | NR₂ | R¹ | Name |
|---|---|---|---|
| AM26 | 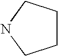 | 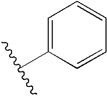 | N-Methyl-1-(4-phenyl- 4-(pyrrolidin- 1-yl)cyclo- hexyl)methanamine |
| AM27 | 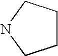 | 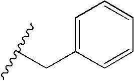 | 1-(4-Benzyl-4-(pyrroli- din-1- yl)cyclohexyl)-N- methylmethanamine |
| AM28 | 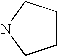 | 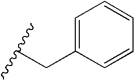 | N-Methyl-1-(4-phenethyl-4- (pyrrolidin-1- yl)cyclo- hexyl)methanamine* |

*The 4-phenethyl-4-(pyrrolidin-1-yl)cyclohexanone required for the synthesis was prepared analogously to the synthesis of the amines AM10-AM21 (Method B).

### Synthesis of the Amines AM29-AM34

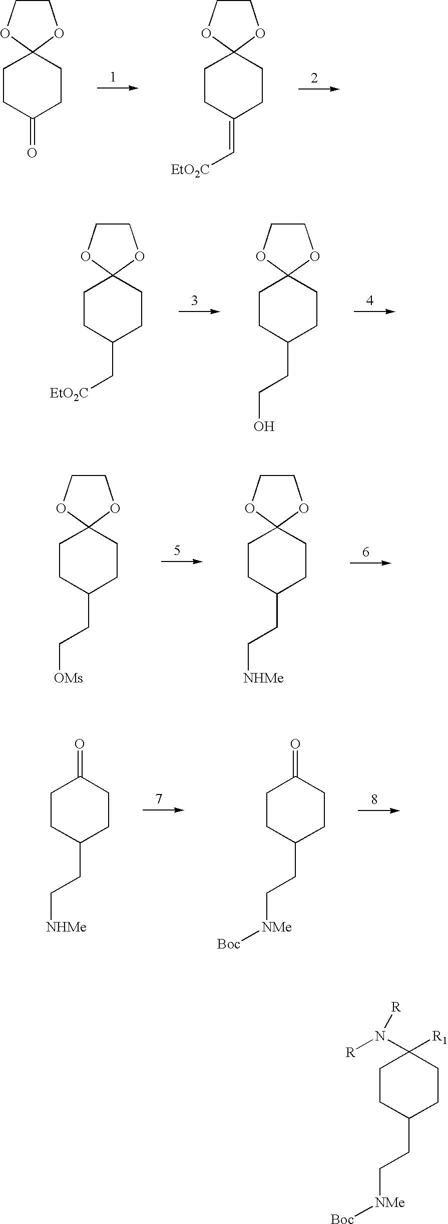

### Method A

Stage 1. A solution of triethylphosphonium acetate (11 mmol) in THF (50 ml) was slowly added to a solution, cooled to 0° C., of NaH (60%, 10 mmol) in dry THF (50 ml) and the mixture was warmed to RT. The reaction mixture was stirred at this temperature for 30 min. It was then cooled to 0° C. and 1,4-dioxa-spiro[4.5]decan-8-one (10 mmol) in dry THF (50 ml) was added dropwise at this temperature. The reaction mixture was warmed to RT and stirred at this temperature for 16 h until the conversion was complete (TLC control). Hydrolysis was then carried out with ice and saturated NaCl solution and the aqueous phase was extracted with ethyl acetate. The organic phase was dried over Na₂SO₄ and concentrated and the crude product was purified by chromatography. (silica gel, hexane/ethyl acetate 8:2)

Stage 2. A solution of the ester (10 mmol) in methanol (30 ml) was first deoxygenated with argon for 15 min and Pd/C (10%, 50 wt. %) was then added. The reaction mixture was then hydrogenated under atmospheric pressure for 16 h (TLC control). The mixture was filtered over kieselguhr, which was rinsed with methanol. The combined organic phases were concentrated. The crude product was employed without further purification.

Stage 3. A solution of the (1,4-dioxa-spiro[4.5]dec-8-yl)-acetic acid ethyl ester (10 mmol) in THF (50 ml) was added to a suspension, cooled to 0° C., of LAH (10 mmol) in dry THF (30 ml) in the course of 30 min. The reaction mixture was warmed to RT and stirred at this temperature for 1 h until the conversion was complete (TLC control). It was then cooled to 0° C. and hydrolysis was carried out with saturated Na₂SO₄ solution. The mixture was filtered over kieselguhr, the solvent was removed and the product was employed further without further purification.

Stage 4. Methanesulfonic acid chloride (11 mmol) was added dropwise to a solution of the alcohol (10 mmol) in MC (50 ml) under an N₂ atmosphere at 0° C. When the addition was complete, the mixture was warmed to RT and stirred at this temperature for 2 h (TLC control). When the reaction had ended, the mixture was diluted with MC. The organic phase was washed successively with water and saturated NaCl solution and dried over Na₂SO₄. The product formed was immediately employed further.

Stage 5. A solution of methylamine in THF (2 M, 10 ml) was added to a solution of the mesylated alcohol (5 mmol) in THF (5 ml). The reaction mixture was heated to 100° C. in a closed reaction vessel for 16 h. The solvent was then removed completely under reduced pressure. The crude product was employed further without further purification.

Stage 6. Aqueous HCl (6 N, 20 ml) was added to the [2-(1,4-dioxa-spiro[4.5]dec-8-yl)-ethyl]-methyl-amine (10 mmol) at 0° C. and the mixture was then warmed to RT and stirred at this temperature for 16 h (TLC control). The aqueous phase was washed with ethyl acetate and adjusted to about pH 14 with aqueous NaOH (6 N). The aqueous phase was extracted with MC and the organic phase was then washed successively with water and saturated NaCl solution. The mixture was dried over Na₂SO₄ and the solvent was stripped off under reduced pressure. The crude product was employed further without further purification.

Stage 7. DIPEA (37.5 mmol) and di-tert-butyl dicarbonate (22.5 mmol) were added to a solution of 4-(2-methylamino-ethyl)-cyclohexanone (15 mmol) in MC (45 ml) at 0° C. The reaction solution was warmed to RT and stirred at this temperature for 16 h (TLC control). When the reaction was complete, the mixture was diluted with MC and the organic phase was washed successively with water and saturated NaCl solution. The mixture was dried over Na₂SO₄ and the solvent was stripped off under reduced pressure. The crude product was purified by column chromatography (silica gel, MC/methanol, 95:5)

Stage 8. KCN (14.4 mmol) and dimethylamine (13.2 mmol) were added to a solution of methyl-[2-(4-oxo-cyclohexyl)-ethyl]-carbonic acid tert-butyl ester (12 mmol) in a mixture of ethanol (12 ml) and water (6 ml). The reaction mixture was stirred at 25° C. for 72 h (TLC control). The reaction mixture was then diluted with ethyl acetate. The organic phase was washed successively with water, aqueous FeSO₄ solution and saturated NaCl solution and then dried over Na₂SO₄. The solvent was stripped off under reduced pressure. The crude product was taken up in THF (50 ml) and the corresponding Grignard reagent (60 mmol) was added, while cooling with ice. The reaction mixture was warmed to 25° C. and stirred at this temperature for 36 h (TLC control). When the reaction had ended, ammonium chloride solution (100 ml) was added and the mixture was then extracted with ethyl acetate. The organic phase was washed with water and saturated NaCl solution, dried over Na₂SO₄ and concentrated. The crude product was purified by column chromatography (silica gel, MC/methanol, 95:5→9:1)

### Method B

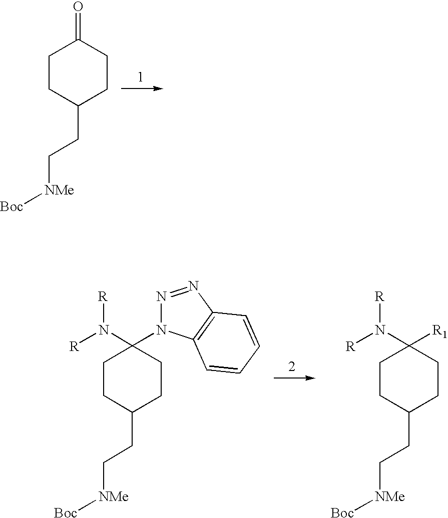

Stage 1. A solution of methyl-[2-(4-oxo-cyclohexyl)-ethyl]-carbonic acid tert-butyl ester (10 mmol, see Method A), the corresponding amine (10 mmol) and benzotriazole (10 mmol) in benzene (100 ml) was heated under reflux using a Dean-Stark water separator. The solvent was then stripped off under reduced pressure. The crude product obtained was used further without further purification.

Stage 2. The corresponding benzotriazole adduct (15 mmol) in dry THF was added dropwise to a solution of the corresponding Grignard reagent in THF (60 mmol) at 0° C. The reaction mixture was warmed to 25° C. and stirred at this temperature for 16 h (TLC control). It was then cooled to 0° C., saturated ammonium chloride solution was added and the mixture was extracted with ethyl acetate. The organic phase was washed successively with water and saturated NaCl solution and dried over Na₂SO₄. The solvent was removed and the crude product obtained was purified by column chromatography (silica gel, MC/methanol, 95:5→9:1)

Amine units AM29-AM34

| No. | NR₂ | R¹ | Method | Name |
|---|---|---|---|---|
| AM29 | NMe₂ | 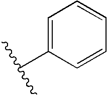 | A | N,N-Dimethyl-4-(2- (methylamino)ethyl)-1- phenylcyclohexanamine |
| AM30 | NMe₂ | 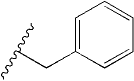 | A | 1-Benzyl-N,N-dimethyl-4-(2- (methylamino)ethyl)cyclohexan amine |
| AM31 | NMe₂ | 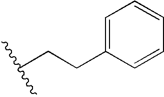 | A | N,N-Dimethyl-4-(2- (methylamino)ethyl)-1- phenethylcyclohexanamine |
| AM32 | 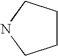 | 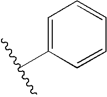 | B | N-Methyl-2-(4-phenyl-4- (pyrrolidin-1- yl)cyclohexyl)ethanamine |
| AM33 | 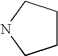 | 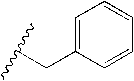 | B | 2-(4-Benzyl-4-(pyrrolidin-1- yl)cyclohexyl)-N- methylethanamine |
| AM34 | 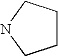 | 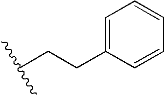 | B | N-Methyl-2-(4-phenethyl-4- (pyrrolidin-1- yl)cyclohexyl)ethanamine |

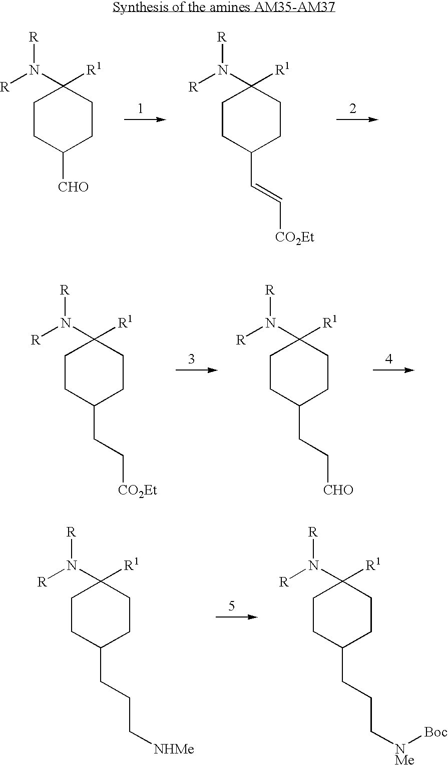

Stage 1. A solution of triethylphosphonium acetate (11 mmol) in THF (50 ml) was slowly added to a solution, cooled to 0° C., of NaH (60% 10 mmol) in dry THF (50 ml) and the mixture was then warmed to RT. The reaction mixture was stirred at this temperature for 30 min. It was then cooled to 0° C. and the aldehyde (10 mmol) in dry THF (50 ml) was added dropwise at this temperature. The reaction mixture was warmed to RT and stirred at this temperature for 16 h until the conversion was complete (TLC control). Hydrolysis was then carried out with ice and saturated NaCl solution and the aqueous phase was extracted with ethyl acetate. The organic phase was dried over Na₂SO₄ and concentrated and the crude product was purified by chromatography. (silica gel, hexane/ethyl acetate 8:2)

Stage 2. A solution of the ester (10 mmol) in methanol (30 ml) was first deoxygenated with argon for 15 min and Pd/C (10%, 50 wt. %) was then added. The reaction mixture was then hydrogenated under atmospheric pressure for 16 h (TLC control). The mixture was filtered over kieselguhr, which was rinsed with methanol. The combined organic phases were concentrated. The crude product was employed further without further purification.

Stage 3. DIBAH (16.5 mmol, 1.5 M solution in toluene) was added dropwise to a solution of the ester (15 mmol) in dry toluene (20 ml) under an argon atmosphere at −70° C. and the mixture was stirred at this temperature for 2 h (TLC control). When the reaction was complete, methanol (10 ml) was added at −70° C. and the mixture was warmed to RT. Saturated NaCl solution (30 ml) was added to this solution and the mixture was filtered over silica gel. The aqueous phase was separated off and extracted with ethyl acetate. The organic phase was washed with saturated NaCl solution, dried over Na₂SO₄ and concentrated and the crude product was employed further without further purification.

Stage 4. A solution of methylamine (2 M in THF, 7.5 ml) and molecular sieve (4 Å, 500 wt. %, based on the aldehyde) was added to a solution of the aldehyde (10 mmol) in MC (50 ml) under an argon atmosphere and the mixture was stirred at 25° C. for 6 h. The reaction solution was filtered, the solvent was stripped off completely, the residue was taken up in dry methanol (50 ml) and the mixture was cooled to 0° C. Sodium borohydride (7.5 mmol) was added in portions to this solution and the mixture was stirred at 25° C. for 16 h. Hydrolysis was carried out with ice, the solvent was stripped off on a rotary evaporator and the residue was taken up in ethyl acetate. The organic phase was washed successively with water and saturated NaCl solution and dried over Na₂SO₄. The solvent was removed and the crude product obtained was employed further without further purification.

Stage 5. DIPEA (25 mmol) and di-tert-butyl dicarbonate (15 mmol) were added to a solution of the amine derivative (10 mmol) in MC (30 ml) at 0° C. The reaction solution was warmed to RT and stirred at this temperature for 16 h (TLC control). When the reaction was complete, the mixture was diluted with MC and the organic phase was washed successively with water and saturated NaCl solution. It was dried over Na₂SO₄ and the solvent was stripped off under reduced pressure. The crude product was purified by column chromatography (silica gel, MC/methanol, 95:5→9:1)

Amine units AM35-AM37

| No. | NR₂ | R¹ | Name |
|---|---|---|---|
| AM35 | 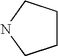 | 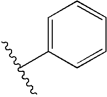 | N-Methyl-3-(4-phenyl-4-(pyrrolidin-1- yl)cyclohexyl)propan-1-amine |
| AM36 | 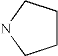 | 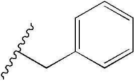 | N-Methyl-3-(4-benzyl-4-(pyrrolidin-1- yl)cyclohexyl)propan-1-amine |
| AM37 | 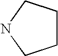 | 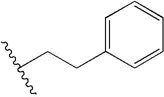 | N-Methyl-3-(4-phenethyl-4-(pyrrolidin-1- yl)cyclohexyl)propan-1-amine |

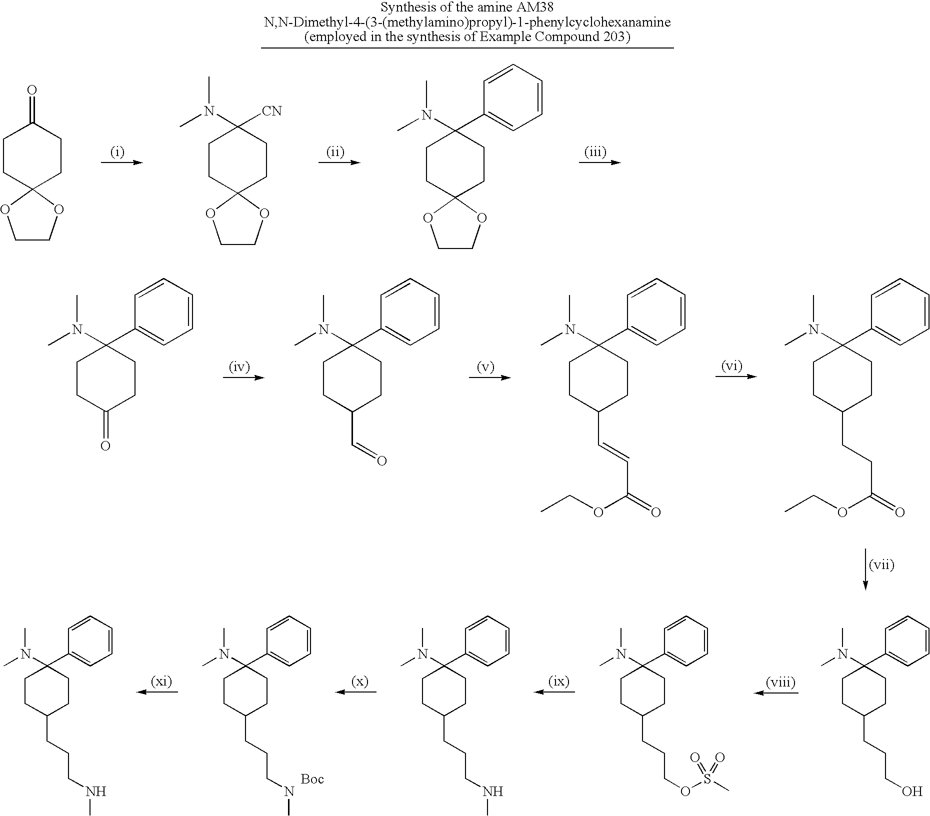

Stage (i): Acetic acid (3 ml) and dimethylamine (40% aq., 20 ml) were added to a solution of 1.4-dioxaspiro[4.5]decan-8-one (2.2 g, 12.8 mmol) in methanol (5 ml). The reaction mixture was cooled and potassium cyanide (2 g, 15.36 mmol) was added at 0° C. under an inert gas. The mixture was stirred for 24 h, during which it was allowed to warm to room temperature. Ammonium hydroxide solution (saturated, 50% diluted, 100 ml) was then added and the mixture was stirred for 30 min and diluted with ethyl acetate (500 ml). It was washed with saturated sodium chloride solution (4 times), with water (4 times), with saturated iron sulfate solution (until this did not lose its colour) and again with saturated sodium chloride solution (once). The organic phase was dried over sodium sulfate, concentrated in vacuo and employed further without purification.

Yield: 50%

Stage (ii): 8-(Dimethylamino)-1,4-dioxaspiro[4.5]decane-8-carbonitrile (1.4 g, 6.66 mmol) was dissolved in tetrahydrofuran (20 ml, dry), the solution was cooled and phenylmagnesium bromide solution (1 mol/l in tetrahydrofuran, 60 ml) was added slowly under an inert gas. After stirring at room temperature for 18 hours, the mixture was cooled again, hydrolysis was carried out with saturated ammonium chloride solution and the mixture was extracted with ethyl acetate (3×100 ml). The combined organic phases were dried over sodium sulfate and concentrated in vacuo. The crude product was purified by column chromatography (silica gel) with 3% methanol in methylene chloride.

Yield: 46%

Stage (iii): N,N-Dimethyl-8-phenyl-1,4-dioxaspiro[4.5]decan-8-amine (1 eq.) was cooled and hydrogen chloride solution (20 eq., 6 mol/l) was slowly added dropwise. The cooling bath was removed and the reaction mixture was stirred for 16 h. It was washed with ethyl acetate (3×50 ml) and the aqueous phase was rendered alkaline with sodium hydroxide solution (6 mol/l) and extracted with methylene chloride (4×100 ml). The combined organic phases were washed with saturated sodium chloride solution, dried over sodium sulfate and concentrated in vacuo. The crude product was employed in the next stage without further purification.

Yield: 67%

Stage (iv): (Methoxymethyl)triphenylphosphine (2 eq.) was initially introduced into tetrahydrofuran (2 ml/mmol, dry) and the mixture was cooled. Potassium tert-butylate (3 eq.), dissolved in tetrahydrofuran (2 ml/mmol), was added dropwise at 0° C. under an inert gas. The mixture was stirred at room temperature for 30 min and then cooled again and 4-(dimethylamine)-4-phenylcyclohexanone (1 eq.), dissolved in tetrahydrofuran (2 ml/mmol), was added dropwise at 0° C. The mixture was stirred at room temperature for 16 h and then cooled and hydrolysis was carried out slowly with hydrogen chloride solution (aq., 6 mol/l, 6 ml/mmol). The aqueous phase was washed with diethyl ether (once), rendered alkaline with sodium hydroxide solution (aq., 5 mol/l) and extracted with methylene chloride (4 times). These organic phases were washed with water and saturated sodium chloride solution, dried over sodium sulfate and concentrated in vacuo. The crude product was employed in the next stage without further purification.

Yield: quantitative

Stage (v): Triethyl phosphonoacetate (1.1 eq., dissolved in tetrahydrofuran 2 ml/mmol) was added dropwise to a cooled (0° C.) suspension of sodium hydride (60% in mineral oil, 1.1 eq.) in tetrahydrofuran (2 ml/mmol, dry) under an inert gas and the mixture was then stirred at room temperature for 30 min. The mixture was cooled again and 4-(dimethylamin)-4-phenylcyclohexanecarbaldehyde (1 eq.), dissolved in tetrahydrofuran (2 ml/mmol), was slowly added dropwise at 0° C. The mixture was stirred at room temperature for 16 h and then cooled, hydrolysis was carried out with ice and the mixture was extracted with ethyl acetate (twice). The combined organic phases were washed with saturated sodium chloride solution, dried over sodium sulfate and concentrated in vacuo. The crude product was employed in the next stage without further purification. Yield: quantitative

Stage (vi): (E)-Ethyl 3-(4-dimethylamino)-4-phenylcyclohexyl)acrylate (1 eq.) was dissolved in methanol (2 ml/mmol) under an inert gas. Pd/C (10%, 0.1 g/mmol) was added and the mixture was stirred under a hydrogen atmosphere (1 atm) for 4 h. The reaction mixture was filtered over Celite (rinsed with methanol) and the filtrate was concentrated in vacuo. The crude product was employed in the next stage without further purification. Yield: 22%

Stage (vii): Lithium aluminium hydride (1.5 eq.) was initially introduced into tetrahydrofuran (40 ml/mmol, dry) and the mixture was cooled and ethyl 3-(4-dimethylamino)-4-phenylcyclohexyl)propanoate (1 eq.), dissolved in tetrahydrofuran (15 ml/mmol), was added dropwise under an inert gas at 0° C. The mixture was then stirred at 0° C. for 30 min, hydrolysis was then carried out with saturated sodium sulfate solution and the mixture was stirred at room temperature for 30 min. It was filtered over Celite (rinsed with ethyl acetate) and concentrated in vacuo and the crude product was employed in the next stage without further purification.

Yield: quantitative

Stage (viii): 3-(4-Dimethylamino)-4-phenylcyclohexyl)propan-1-ol (1.1 eq.) was dissolved in methylene chloride (4 ml/mmol) and triethylamine (2.5 eq.) and the solution was cooled. Methanesulfonyl chloride (1 eq.), dissolved in methylene chloride (2 ml/mmol), was then added dropwise at 0° C. The mixture was stirred at room temperature for 90 min, hydrogen chloride solution (0.5 mol/l, 3 ml/mmol) was added and the mixture was stirred for 15 min. After separation of the phases, the organic phase was washed with water, dried over sodium sulfate and concentrated in vacuo. The crude product was employed in the next stage without further purification.

Yield: quantitative

Stage (ix): 3-(4-Dimethylamino)-4-phenylcyclohexyl)propyl methanesulfonate (1 eq.) and methylamine solution (3 mol/l, 2 eq. in tetrahydrofuran) were heated at 70° C. in a closed vessel for 16 h. The reaction mixture was concentrated in vacuo and the crude product was employed in the next stage without further purification.

Yield: quantitative

Stage (x): Diisopropylethylamine (2.5 eq.) and Boc anhydride (2.2 eq.) were added to a solution of N,N-dimethyl-4-(3-(methylamino)propyl)-1-phenylcyclohexanamine (1 eq.) in methylene chloride (7 ml/mmol) under an inert gas. The reaction mixture was stirred at room temperature for 16 h, diluted with methylene chloride and washed with water and saturated sodium chloride solution. The organic phase was dried over sodium sulfate and concentrated in vacuo. The crude product was purified by column chromatography (silica gel) with 5% methanol in methylene chloride.

Yield: 26% (after 3 stages)

Stage (xi): Trifluoroacetic acid (13 eq.) was added to a solution of tert-butyl 3-(4-(dimethylamino)-4-phenylcyclohexyl)propyl (methyl)carbamate (1 eq.) in methylene chloride (10 ml/mmol) at 0° C., the cooling bath was removed and the mixture was stirred at room temperature for 2 h. It was concentrated in vacuo and the residue was dried. The deprotected amine was employed in the next stage without further purification. Yield: quantitative

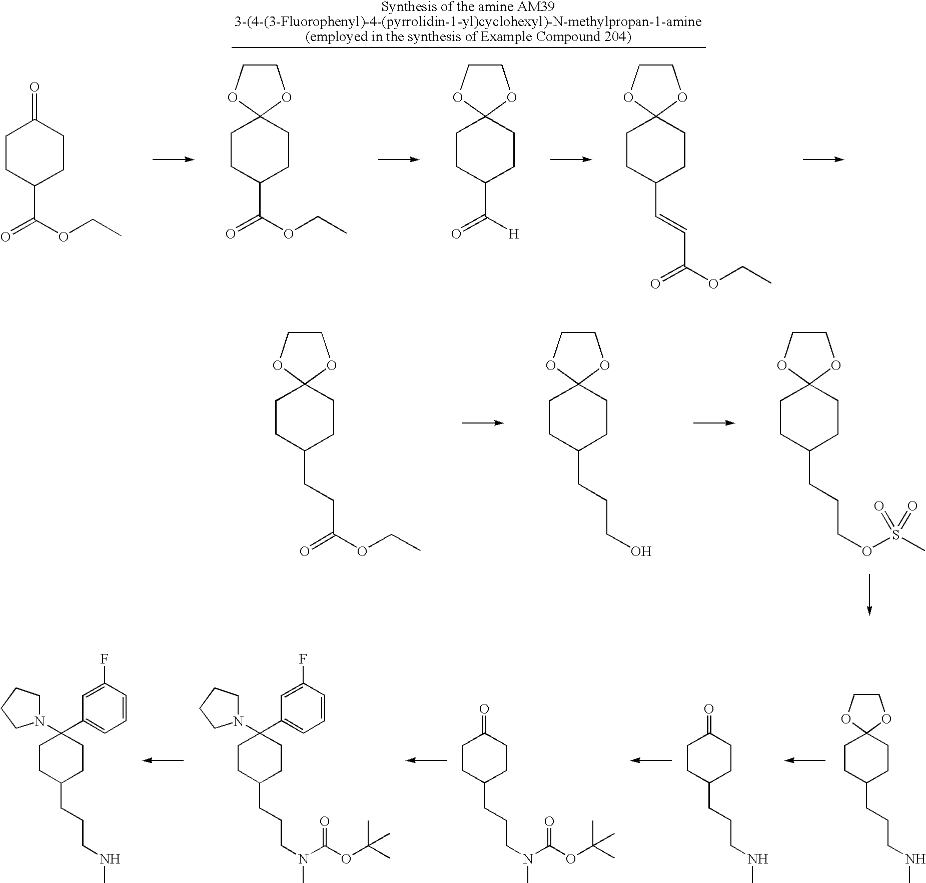

Stage (i): 4-Oxocyclohexanecarboxylic acid (20 g, 117 mmol) was dissolved in toluene (60 ml, dry), and ethylene glycol (23 ml, 411 mmol) and p-toluenesulfonic acid (265 mg) were added at 0° C. The cooling bath was removed, the reaction mixture was stirred at room temperature for 16 h and hydrolysis was then carried out with ice. Extraction was carried out with ethyl acetate (300 ml) and the organic phase was washed with sodium carbonate solution and saturated sodium chloride solution, dried over sodium sulfate and concentrated in vacuo. The crude product was employed in the next stage without further purification. Yield: 90%

Stage (ii): Ethyl 1,4-dioxaspiro[4.5]decane-8-carboxylate (23 g, 107 mmol) was dissolved in toluene (460 ml), the solution was cooled and diisobutylaluminium hydride (118 ml, 1 mol/l in toluene) was added dropwise at −78° C. under an inert gas. The mixture was stirred at the same temperature for 2 h, hydrolysis was then carried out with saturated sodium chloride solution, the cooling bath was removed and the mixture was stirred at room temperature for 1 h. The precipitate was filtered off over Celite (rinsed with ethyl acetate) and the organic phase was washed with saturated sodium chloride solution, dried over sodium sulfate and concentrated in vacuo. The crude product was employed in the next stage without further purification. Yield: 80%

Stage (iii): Triethyl phosphonoacetate (19.6 ml, 99 mmol, dissolved in 250 ml of tetrahydrofuran) was added dropwise to a cooled (0° C.) suspension of sodium hydride (60% in mineral oil, 4.8 g, 99 mmol) in tetrahydrofuran (250 ml, dry) under an inert gas and the mixture was then stirred at room temperature for 30 min. The mixture was cooled again and 1.4-dioxaspiro[4.5]decane-8-carbaldehyde (15.3 g, 90 mmol), dissolved in tetrahydrofuran (250 ml), was slowly added dropwise at 0° C. The mixture was stirred at room temperature for 16 h, hydrolysis was carried out with ice and the mixture was extracted with ethyl acetate (2×300 ml). The combined organic phases were washed with saturated sodium chloride solution, dried over sodium sulfate and concentrated in vacuo. The crude product was purified by column chromatography (silica gel) with 20% ethyl acetate in hexane. Yield: 46%

Stage (iv): (E)-Ethyl 3-(1.4-dioxaspiro[4.5]decan-8-yl)acrylate (10 g) was dissolved in methanol (100 ml) and the solution was flushed with an inert gas. Pd/C (10%, 4.7 g) was added and the mixture was stirred under a hydrogen atmosphere (1 atm) for 4 h. The reaction mixture was filtered over Celite (rinsed with methanol) and the filtrate was concentrated in vacuo. The crude product was employed in the next stage without further purification. Yield: 92%

Stage (v): Lithium aluminium hydride (2.2 g, 5.7 mmol) was initially introduced into tetrahydrofuran (150 ml, dry), the mixture was cooled and ethyl 3-(1.4-dioxaspiro[4.5]decan-8-yl)propanoate (9.3 g, 3.8 mmol), dissolved in tetrahydrofuran (50 ml), was added dropwise under an inert gas. The mixture was stirred at 0° C. for 30 min, hydrolysis was then carried out with saturated sodium sulfate solution and the mixture was stirred at room temperature for 30 min. It was filtered over Celite (rinsed with 250 ml of ethyl acetate) and concentrated in vacuo and the crude product was employed in the next stage without further purification. Yield: quantitative

Stage (vi): 3-(1,4-Dioxaspiro[4.5]decan-8-yl)propan-1-ol (1.1 eq.) was dissolved in methylene chloride (4 ml/mmol) and triethylamine (2.5 eq.), the solution was cooled and methanesulfonyl chloride (1 eq.), dissolved in methylene chloride (2 ml/mmol), was added dropwise at 0° C. The mixture was stirred at room temperature for 90 min, hydrogen chloride solution (aq., 0.5 mol/l, 3 ml/mmol) was added and the mixture was stirred for 15 min. After separation of the phases, the organic phase was washed with water, dried over sodium sulfate and concentrated in vacuo. The crude product was employed in the next stage without further purification. Yield: quantitative

Stage (vii): 3-(1.4-Dioxaspiro[4.5]decan-8-yl)propyl methanesulfonate (5.3 g, 19 mmol) and methylamine solution (100 ml, 3 mol/l in tetrahydrofuran) were heated at 70° C. in a closed vessel for 16 h. The reaction mixture was concentrated in vacuo and the crude product was employed in the next stage without further purification.

Stage (viii): N-Methyl-3-(1.4-dioxaspiro[4.5]decan-8-yl)propan-1-amine (3.8 g, 18 mmol) was cooled and hydrogen chloride solution (70 ml, aq., 6 mol/l) was slowly added dropwise. The cooling bath was removed and the reaction mixture was stirred for 16 h. It was washed with ethyl acetate (3×50 ml) and the aqueous phase was rendered alkaline (pH=14) with sodium hydroxide solution (6 mol/l) and extracted with methylene chloride (4×100 ml). These organic phases were washed with saturated sodium chloride solution, dried over sodium sulfate and concentrated in vacuo. The crude product was employed in the next stage without further purification.

Stage (ix): Diisopropylethylamine (3.7 ml, 22.5 mmol) and Boc anhydride (2.1 g, 19.8 mmol) were added to a cooled solution of 4-(3-(methylamino)propyl)-cyclohexanone (1.5 g, 9 mmol) in methylene chloride (60 ml) under an inert gas. The reaction mixture was stirred at room temperature for 16 h, diluted with methylene chloride (250 ml) and washed with water and saturated sodium chloride solution. The organic phase was dried over sodium sulfate and concentrated in vacuo. The crude product was purified by column chromatography (silica gel) with 5% methanol in methylene chloride. Yield: 58% (after 3 stages)

Stage (x): tert-Butyl methyl-(3-(4-oxocyclohexyl)propyl)carbamate (1.5 g, 5.6 mmol), benzotriazole (0.66 g, 5.6 mmol) and pyrrolidine (0.5 ml, 5.6 mmol) were refluxed in benzene (dry) for 18 h using a water separator. The reaction mixture cooled and was concentrated/dried in vacuo. The crude product was taken up in tetrahydrofuran (dry), the mixture was cooled and 3-fluorophenylmagnesium bromide solution (56 mmol), dissolved in tetrahydrofuran, was added dropwise under an inert gas. The reaction mixture was stirred at room temperature for 18 h and cooled again and hydrolysis was carried out with saturated ammonium chloride solution. Extraction was carried out with ethyl acetate (3×100 ml) and the combined organic phases were washed with saturated sodium chloride solution, dried over sodium sulfate and concentrated in vacuo. The crude product was purified by column chromatography (silica gel) with 5% methanol in methylene chloride. Yield: 15%

Stage (xi): Trifluoroacetic acid (13 eq.) was added to a solution of tert-butyl 3-(4-(3-fluorophenyl)-4-(pyrrolidin-1-yl)cyclohexyl)propyl(methyl)carbamate (1 eq.) in methylene chloride (10 ml/mmol) at 0° C., the cooling bath was removed and the mixture was stirred at room temperature for 2 h. The reaction mixture was concentrated in vacuo, the residue was dried and the deprotected amine was employed in the next stage without further purification. Yield: quantitative

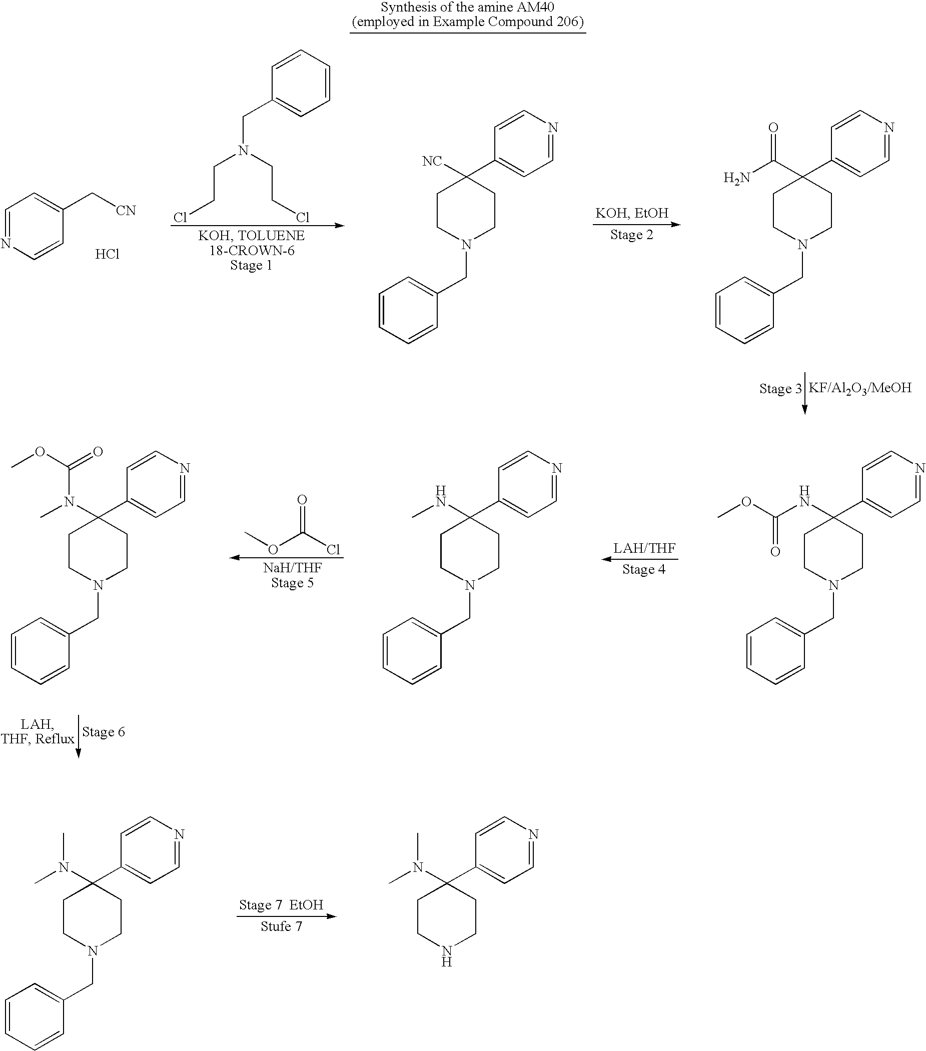

### Stage 1:

2-(Pyridin-4-yl)acetonitrile hydrochloride (2 g, 12 mmol) was added to a suspension of dry, ground potassium hydroxide (3.36 g, 60 mmol) in dry toluene (40 ml) at 25° C. under argon and the reaction mixture was then cooled to 0° C. N-Benzyl-2-chloro-N-(2-chloroethyl)ethanamine (3.6 g, 15 mmol), dissolved in toluene (30 ml), was added dropwise, 18-crown-6 (0.6 g, 2.4 mmol) was then added and the mixture was heated under reflux for 2 h. The reaction mixture was hydrolyzed with crushed ice and extracted with methylene chloride. The organic phase was washed with water (2×) and saturated sodium chloride solution, dried over sodium sulfate, filtered and concentrated in vacuo. The crude product was purified by column chromatography (silica gel, 3% methanol in methylene chloride) and the desired product 1-benzyl-4-(pyridin-4-yl)piperidine-4-carbonitrile was isolated in a pure form. Yield: 50%

### Stage 2:

Potassium hydroxide was added to a solution of 1-benzyl-4-(pyridin-4-yl)piperidine-4-carbonitrile (3 g, 10.8 mmol) in ethanol/water (1/1, 92 ml), while stirring, and the mixture was heated under reflux for 5 h. The solvent was removed in vacuo and the residue was acidified (pH=2) by dropwise addition of dilute acetic acid (45 ml of glacial acetic acid+15 ml of water) at 0° C. Extraction was carried out with chloroform (3×) and the combined organic phases were washed with saturated sodium chloride solution, dried over sodium sulfate, filtered and concentrated in vacuo. The crude product was purified by column chromatography (Alox neutral, 3% methanol in methylene chloride) and the desired product 1-benzyl-4-(pyridin-4-yl)piperidine-4-carboxamide was isolated in a pure form. Yield: 83%

### Stage 3:

KF/Al₂O₃ (10 g) and sodium hypochlorite solution (4% aq., 15 ml) was added to a solution of 1-benzyl-4-pyridin-4-yl)piperidine-4-carboxamide (1.5 g, 5.08 mmol) in methanol (38 ml) and the mixture was stirred at 25° C. for 2 h. The solid was filtered off (and washed with methanol), the methanol was removed in vacuo, the crude product was purified by column chromatography (Alox neutral, 2% methanol in methylene chloride) and the desired product methyl 1-benzyl-4-(pyridin-4-yl)piperidin-ylcarbamate) was isolated in a pure form. Yield: 70% [Preparation of KF/Al₂O₃: Potassium fluoride (15 g) was dissolved in dist. water (200 ml), Alox neutral was added (20 g) and the mixture was stirred at 25° C. for 16 h. Water was removed in vacuo at 50° C. and the residue was dried under a full vacuum for 5 h and then used immediately for the reaction.]

### Stage 4:

A solution of methyl 1-benzyl-4-(pyridin-4-yl)piperidin-4-ylcarbamate (1.2 g, 3.69 mmol) in dry tetrahydrofuran (20 ml) was added dropwise to a suspension of lithium aluminium hydride (0.29 g, 7.38 mmol) in dry tetrahydrofuran (20 ml) at 0° C. When the addition was complete, the mixture was heated under reflux for 1 h. The reaction mixture was then cooled, hydrolysis was carried out with saturated sodium sulfate solution at 0° C., the mixture was filtered over Celite and the residue was washed with ethyl acetate. The solvent was removed in vacuo and the residue was dried. The crude product was purified by column chromatography (Alox neutral, 3% methanol in methylene chloride) and the desired product 1-benzyl-N-methyl-4-(pyridin-4-yl)piperidin-4-amine was isolated in a pure form. Yield: 65%

### Stage 5:

A solution of 1-benzyl-N-methyl-4-(pyridin-4-yl)piperidin-4-amine (0.4 g, 1.4 mmol) in dry tetrahydrofuran (7 ml) was added dropwise to a suspension of sodium hydride (50%, 410 mg, 8.54 mmol) in dry tetrahydrofuran (5 ml) at 0° C. under argon. The reaction mixture was stirred at 25° C. for 1 h and then cooled again to 0° C., methyl chloroformate (0.135 ml, 1.7 mmol) was added dropwise and the mixture was stirred at 25° C. for 16 h. Hydrolysis was carried out with crushed ice at 0° C. and the mixture was diluted with ethyl acetate, washed with saturated sodium chloride solution, dried over sodium sulfate, filtered and concentrated in vacuo. The crude product was purified by column chromatography (Alox neutral, 3% methanol in methylene chloride) and the desired product methyl 1-benzyl-4-(pyridin-4-yl)piperidin-4-yl(methyl)carbamate was isolated in a pure form. Yield: 51%

### Stage 6:

Methyl 1-benzyl-4-(pyridin-4-yl)piperidin-4-yl(methyl)carbamate (0.25 g, 0.73 mmol), dissolved in dry tetrahydrofuran (5 ml), was added dropwise to a suspension of lithium aluminium hydride (57 mg, 1.47 mmol) in dry tetrahydrofuran (5 ml) at 0° C. The solution was heated under reflux for 1 h. Hydrolysis was then carried out with saturated sodium sulfate solution at 0° C., the mixture was filtered over Celite and the residue was washed with ethyl acetate. The filtrate was concentrated in vacuo and the residue was dried. The crude product was purified by column chromatography (Alox neutral, 2% methanol in methylene chloride) and the desired product 1-benzyl-N,N-dimethyl-4-(pyridin-4-yl)piperidin-4-amine was isolated in a pure form. Yield: 68%

### Stage 7:

A solution of 1-benzyl-N,N-dimethyl-4-(pyridin-4-yl)piperidine-4-amine (150 mg, 0.5 mmol) in ethanol (10 ml) was flushed with argon for 10 min and palladium hydroxide (20%, 23 mg) was added in one portion. The reaction mixture was stirred under a hydrogen atmosphere (balloon) for 16 h and filtered over Celite and the residue was washed with ethanol. The filtrate was concentrated in vacuo. The crude product, the desired product N,N-dimethyl-4-(pyridin-4-yl)piperidin-4-amine, was not purified further. Yield: 95%

### Synthesis of Amine AM41

### 2-(4-(Azetidin-1-yl)-4-phenylcyclohexyl)-N-methylethanamine

Synthesis of this amine building block was achieved in analogy to synthesis of amine AM32 (N-methyl-2-(4-phenyl-4-(pyrrolidin-1-yl)cyclohexyl)ethanamine) replacing pyrrolidine with azetidine.

### Synthesis of Amine AM42

### 3-(4-(Azetidin-1-yl)-4-(3-fluorophenyl)cyclohexyl)-N-methylpropan-1-amine

Synthesis of this amine building block was achieved in analogy to synthesis of amine AM39 (3-(4-(3-fluorophenyl)-4-(pyrrolidin-1-yl)cyclohexyl)-N-methylpropan-1-amine) replacing pyrrolidine with azetidine.

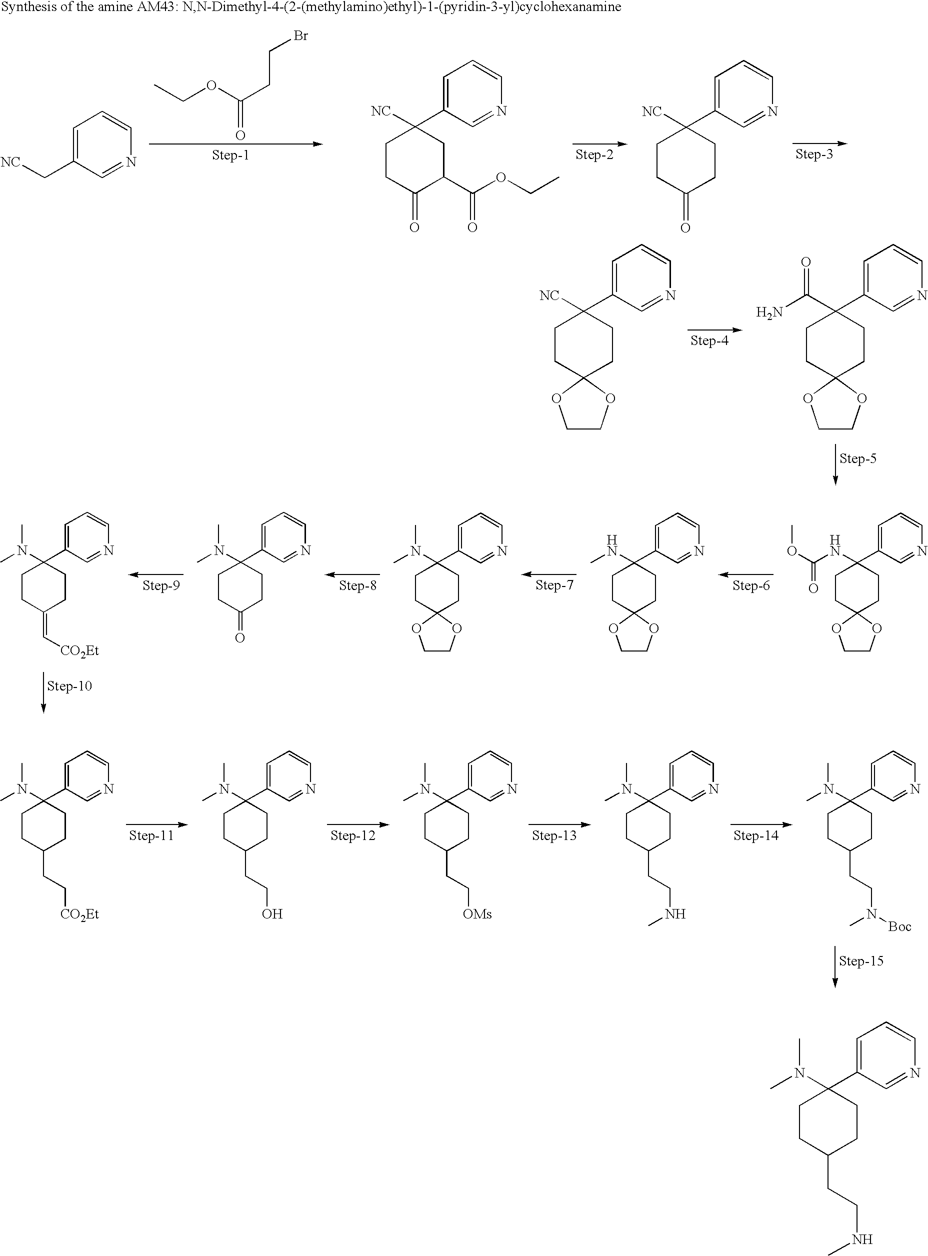

### Step-1: Ethyl 5-cyano-2-oxo-5-(pyridin-3-yl)cyclohexanecarboxylate

To a solution of 2-(pyridin-3-yl)acetonitrile (1.0 g, 8.47 mmol) in DMF (10 ml) at 0° C. was added dropwise ^{t}BuOK (5.47 g, 50.82 mmol) in DMF (30 ml) and the mixture was stirred for 1 h at room temperature. The reaction mixture was cooled to 0° C. and to this was added ethyl 3-bromopropanoate (2.37 ml, 18.63 mmol) in DMF (10 ml) and it was stirred for 16 h at room temperature. It was quenched with ice, the reaction mixture was extracted with ethyl acetate (2×) and the combined organic layers were washed with water and brine. The organic layer was dried over sodium sulfate and the solvent removed in vacuo. The crude product was purified by column chromatography (silica, 20% ethyl acetate in hexane) to give the desired product. Yield: 57%

### Step-2: 4-Oxo-1-(pyridin-3-yl)cyclohexanecarbonitrile

Ethyl 5-cyano-2-oxo-5-(pyridin-3-yl)cyclohexanecarboxylate (614 mg 2.2 mmol) was dissolved in acetic acid (6.8 ml) and conc. HCl (2.9 ml) and stirred at 110° C. for 4 h. The reaction mixture was basified with 5N NaOH in an ice bath. The aqueous layer was extracted with ethyl acetate and the organic layer was dried over anhydrous sodium sulfate and evaporated to give the desired product. Yield: 60%

### Step-3: 8-(Pyridin-3-yl)-1,4-dioxaspiro[4.5]decane-8-carbonitrile

4-Oxo-1-(pyridin-3-yl)cyclohexanecarbonitrile (330 mg, 1.65 mmol) was dissolved in toluene (10 ml) and ethylene glycol (0.182 ml) and a catalytic amount of PTSA was added. The mixture was stirred at 110° C. under Dean-Stark conditions for 2 h. Water (5 ml) was added and the organic layer was washed with sat. NaHCO₃ solution (15 ml), water (15 ml) and brine (15 ml). The organic layer was dried over sodium sulfate and evaporated to give the desired product. Yield: 93%

### Step-4: 8-(Pyridin-3-yl)-1,4-dioxaspiro[4.5]decane-8-carboxamide

8-(Pyridin-3-yl)-1,4-dioxaspiro[4.5]decane-8-carbonitrile (1.00 g, 4.18 mmol) was dissolved in ethanol:water (40 ml, 1:1) and KOH (1.17 g, 20.9 mmol) was added. The reaction mixture was stirred for 48 h at room temperature and 9 h at 80° C. Afterwards the solvent was evaporated. The residue was adjusted to pH 2 with water (5 ml) and acetic acid (15 ml) and the product was extracted with chloroform (4×). The combined organic layers were dried with sodium sulfate and evaporated to give the desired product.

Yield: 90%

### Step-5: Methyl 8-(pyridin-3-yl)-1,4-dioxaspiro[4.5]decan-8-ylcarbamate

8-(Pyridin-3-yl)-1,4-dioxaspiro[4.5]decane-8-carboxamide (2.0 g, 7.63 mmol) was dissolved in methanol (53 ml) and KF/Al₂O₃ (15.2 g) and sodium hypochlorite solution (22 ml) were added. The reaction mixture was stirred for 1 h at room temperature, filtered and washed with methanol. The filtrate was concentrated in vacuo and the crude product was purified by column chromatography on alumina to give the desired product. Yield: 28%

### Step-6: N-Methyl-8-(pyridin-3-yl)-1,4-dioxaspiro[4.5]decan-8-amine

LAH (0.36 g, 8.24 mmol) was dissolved in dry THF (20 ml) under argon. A solution of methyl 8-(pyridin-3-yl)-1,4-dioxaspiro[4.5]decan-8-ylcarbamate (1.00 g, 4.12 mmol) in THF (10 ml) was added slowly and the reaction mixture was stirred for 1 h at reflux. LAH was quenched with aqueous THF. The mixture was filtered through a pad of celite, which was washed with THF. The crude product was purified by column chromatography using silica to give the desired product. Yield: 43%

### Step-7: N,N-Dimethyl-8-(pyridin-3-yl)-1,4-dioxaspiro[4.5]decan-8-amine

37% Aqueous formaldehyde solution (1.37 ml) and Pd/C (138 mg) was added to a solution of N-methyl-8-(pyridin-3-yl)-1,4-dioxaspiro[4.5]decan-8-amine (138 mg 0.55 mmol) in ethanol (20 ml) under argon. The reaction mixture was hydrogenated with a hydrogen balloon (Pd/C, 16 h). The mixture was filtered through celite and the residue was washed with ethanol. Ethanol was evaporated, and the residue diluted with water (10 ml) and extracted with dichloromethane (3×). The organic layer was dried over sodium sulfate, filtered and concentrated in vacuo to give the desired product. Yield: 89%

### Step-8: 4-(Dimethylamino)-4-(pyridin-3-yl)cyclohexanone

6N HCl (2.6 ml/mmol) was added dropwise to N,N-dimethyl-8-(pyridin-3-yl)-1,4-dioxaspiro[4.5]decan-8-amine at 0° C. and the mixture was stirred for 30 min at room temperature. Water was added to the reaction mixture and the aqueous layer was washed with ethyl acetate. The aqueous layer was basified with 5 N NaOH and extracted in dichloromethane (2×). The combined organic layers were dried over sodium sulfate, filtered and concentrated in vacuo to give the desired product. Yield: 66%

### Step-9: Ethyl 2-(4-(dimethylamino)-4-(pyridin-3-yl)cyclohexylidene)acetate

To a cold (0° C.) suspension of 60% NaH (1.1 equiv.) in dry THF (5 ml/mmol) was added slowly a solution of triethyl phosphonoacetate (1.1 equiv.) in THF (5 ml/mmol) and the resulting reaction mixture was allowed to stir at 25° C. for 30 min. It was then cooled to 0° C. and 4-(dimethylamino)-4-(pyridin-3-yl)cyclohexanone (1 equiv.) in dry THF (5 ml/mmol) was added dropwise maintaining the same temperature. The reaction mixture was allowed to stir at 25° C. for another 16 h. It was quenched with ice and brine and was extracted with ethyl acetate. The organic layer was washed successively with water and brine. It was dried over sodium sulfate and evaporated under reduced pressure to obtain the crude product, which was purified by column chromatography (10% methanol in dichloromethane) to give the desired compound. Yield: 70%

### Step-10: Ethyl 2-(4-(dimethylamino)-4-(pyridin-3-yl)cyclohexyl)acetate

A solution of ethyl 2-(4-(dimethylamino)-4-(pyridin-3-yl)cyclohexylidene)acetate (1 equiv.) in methanol (5 ml/mmol) was deoxygenated with argon for 15 min followed by addition of 10% Pd/C (50% by weight). The resulting reaction mixture was hydrogenated under atmospheric pressure for 1 h. It was filtered through a bed of celite, the residue was washed with methanol and the combined organic layers were evaporated completely to yield the crude product, which was purified by column chromatography (10% methanol in dichloromethane) to give the desired compound. Yield: 37%

### Step-11: 2-(4-(Dimethylamino)-4-(pyridin-3-yl)cyclohexyl)ethanol

To a cold (0° C.) suspension of LAH (1.2 equiv.) in THF (3 ml/mmol) under an argon atmosphere was added dropwise a solution of ethyl 2-(4-(dimethylamino)-4-(pyridin-3-yl)cyclohexyl)acetate (1 equiv.) in THF (2 ml/mmol). After addition was complete the reaction mixture was allowed to stir at this temperature for 2 h by which time the starting material was completely consumed (monitored by TLC). The reaction was carefully quenched with a saturated aqueous solution of sodium sulfate and filtered through a bed of celite. The residue was washed with ethyl acetate and the combined organic layers were dried over sodium sulfate and evaporated under reduced pressure to yield the crude alcohol, which was used directly in the next step without any further purification. Yield: 90%

### Step-12: 2-(4-(Dimethylamino)-4-(pyridin-3-yl)cyclohexyl)ethyl methanesulfonate

To a dichloromethane solution (22 ml) of 2-(4-(dimethylamino)-4-(pyridin-3-yl)cyclohexyl)ethanol (5.3 mmol) was added triethylamine (21.2 mmol) and methane sulfonyl chloride (7.95 mmol) at 0° C. and the resulting reaction mixture was allowed to stir at same temperature for 2 h (monitored by TLC). The reaction mixture was diluted with dichloromethane, washed with water and brine, and the organics were dried over sodium sulfate. Evaporation of the organic layer under reduced pressure gave the crude product, which was used directly in the next step without further purification.

### Step-13: N,N-Dimethyl-4-(2-(methylamino)ethyl)-1-(pyridin-3-yl)cyclohexanamine

2-(4-(Dimethylamino)-4-(pyridin-3-yl)cyclohexyl)ethyl methanesulfonate (0.64 mmol) was dissolved in THF (1 ml) and methylamine in THF (10 ml) was added in a sealed tube and the mixture stirred over night. The reaction mixture was concentrated and the crude product obtained was used in the next step without further purification.

### Step-14: tert-Butyl 2-(4-(dimethylamino)-4-(pyridin-3-yl)cyclohexyl)ethyl(methyl)-carbamate

To the stirred solution of N,N-dimethyl-4-(2-(methylamino)ethyl)-1-(pyridin-3-yl)cyclohexanamine (0.6451 mmol) in dichloromethane, cooled to 0° C., was added triethylamine (1.609 mmol). The mixture was stirred for 2 h at room temperature and subsequently diluted with dichloromethane. The organic layer was washed with water and brine, and dried over sodium sulfate. The crude product was purified by column chromatography. Yield: 36%

### Step-15: N,N-Dimethyl-4-(2-(methylamino)ethyl)-1-(pyridin-3-yl)cyclohexanamine (amine A43)

tert-Butyl 2-(4-(dimethylamino)-4-(pyridin-3-yl)cyclohexyl)ethyl (methyl)carbamate (0.235 mmol) was dissolved in dichloromethane, cooled to 0° C., and TFA (2 ml/mmol) was added. The reaction mixture was stirred for another 2 h. The solvent was completely evaporated from the mixture and kept under the high vacuum to give desired product.

Yield: quantitative

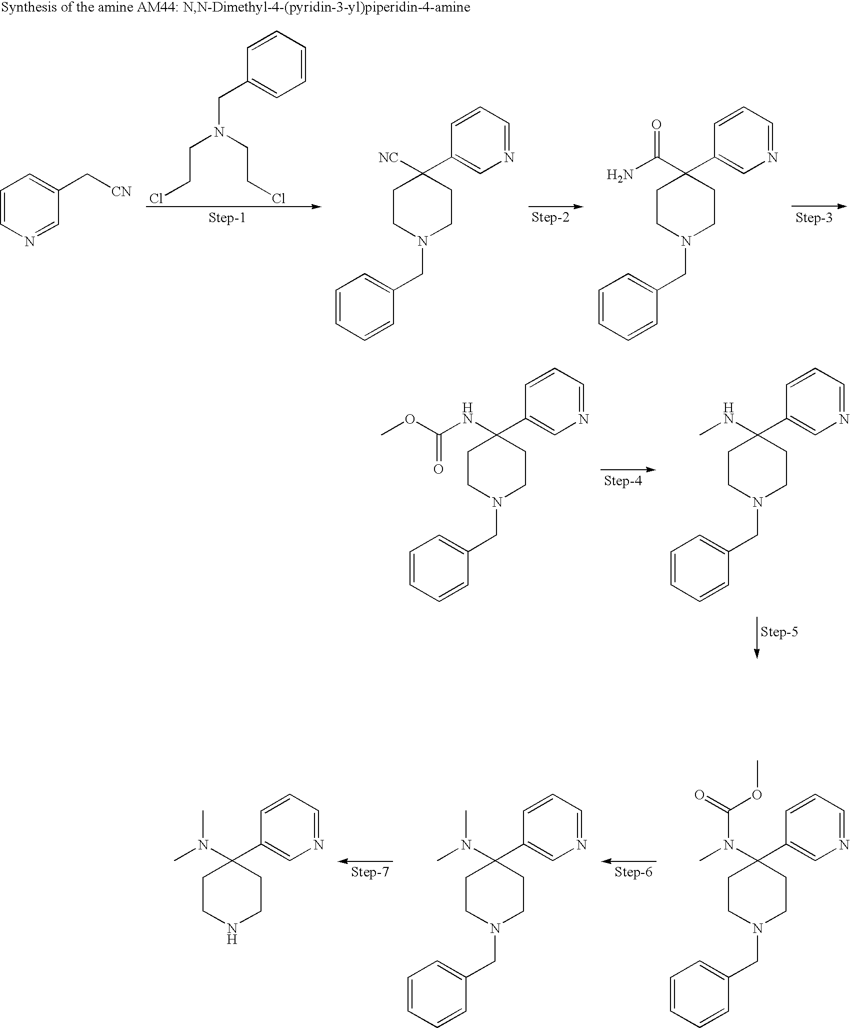

### Step-1: 1-Benzyl-4-(pyridin-3-yl)piperidine-4-carbonitrile

To a suspension of anhydrous powdered potassium hydroxide (4.2 g) in anhydrous toluene (50 ml) was added the hydrochloride salt of 2-(pyridin-3-yl)acetonitrile (1.8 g, 0.0152 mol) at 25° C. under argon atmosphere. The resultant reaction mixture was cooled to 0° C. and N-benzyl-2-chloro-N-(2-chloroethyl)ethanamine (4.2 g, 0.0182 mol) was added followed by the addition of 18-crown-6 (1 g). The mixture was allowed to reflux for 2 h. The reaction was quenched with crushed ice and extracted with dichloromethane. The organic layer was washed with water (2×) and brine, dried over sodium sulfate, filtered and concentrated under reduced pressure to obtain the crude material, which was purified by column chromatography (100-200 mesh silica gel, 3% methanol in dichloromethane) to isolate the desired compound in pure form. Yield: 82%

### Step-2: 1-Benzyl-4-(pyridin-3-yl)piperidine-4-carboxamide

To a stirred solution of 1-Benzyl-4-(pyridin-3-yl)piperidine-4-carbonitrile (3.5 g, 0.0118 mol) in ethanol:water (100 ml, 1:1) was added potassium hydroxide and the mixture was refluxed for 7 h. The solvent was evaporated under reduced pressure and it was azeotroped with toluene. The crude material was purified by column chromatography (neutral alumina, 2% methanol in dichloromethane) to isolate the desired compound in pure form. Yield: 72%

### Step-3: Methyl 1-benzyl-4-(pyridin-3-yl)piperidin-4-ylcarbamate

To a stirred solution of 1-benzyl-4-(pyridin-3-yl)piperidine-4-carboxamide (2.7 g, 0.0091 mol) in methanol (60 ml) was added KF/Al₂O₃ (20 g) and a 4% aqueous solution of sodium hypochlorite (25 ml). The mixture was stirred at 25° C. for 4 h. The solid was filtered off and washed with methanol. The methanol part was evaporated under reduced pressure, to obtain the crude material, which was purified by column chromatography (neutral alumina, 0.5% methanol in dichloromethane) to isolate the desired compound in pure form. Yield: 54%

### Step-4: 1-Benzyl-N-methyl-4-(pyridin-3-yl)piperidin-4-amine

To a suspension of LAH (10.42 mmol) in anhydrous THF (30 ml) was added a solution of methyl 1-benzyl-4-(pyridin-3-yl)piperidin-4-ylcarbamate (1.6 g, 5.21 mmol) in anhydrous THF (30 ml) dropwise at 0° C. The resultant solution was refluxed for 1 h. The reaction mixture was cooled to 0° C., quenched with sat. sodium sulfate solution, filtered through a celite bed and the residue washed with ethyl acetate. The combined organic layers were evaporated to dryness under reduced pressure and the product was purified by column chromatography (neutral alumina, 2% methanol in dichloromethane) to isolate the desired compound in pure form. Yield: 57.9%

### Step-5: Methyl 1-benzyl-4-(pyridin-3-yl)piperidin-4-yl(methyl)carbamate

To a suspension of 50% sodium hydride (819 mg, 17.076 mmol) in anhydrous THF (15 ml) was added a solution of 1-benzyl-N-methyl-4-(pyridin-3-yl)piperidin-4-amine (800 mg, 2.846 mmol) in anhydrous THF (20 ml) dropwise at 0° C. under an argon atmosphere. The reaction mixture was stirred at 25° C. for 1 h and it was then again cooled to 0° C. and methyl chloroformate (0.268 ml, 3.415 mmol) was added dropwise. The resultant solution was allowed to warm to 25° C. and stir for 16 h. The reaction mixture was cooled to 0° C., quenched with crushed ice and diluted with ethyl acetate. The organics were washed with brine, dried over sodium sulfate, filtered and concentrated under reduced pressure to obtain the crude material, which was purified by column chromatography (neutral alumina, 2% methanol in dichloromethane) to isolate the desired compound in pure form.

Yield: 51%

### Step-6: 1-Benzyl-N,N-dimethyl-4-(pyridin-3-yl)piperidin-4-amine

To a suspension of LAH (112 mg, 2.94 mmol) in anhydrous THF (10 ml) was added a solution of methyl 1-benzyl-4-(pyridin-3-yl)piperidin-4-yl(methyl)carbamate (500 mg, 1.470 mmol) in anhydrous THF (10 ml) dropwise at 0° C. The resultant solution was refluxed for 1 h. The reaction mixture was cooled to 0° C. and quenched with sat. sodium sulfate solution. The mixture was filtered over a celite bed and the residue was washed with ethyl acetate. The filtrate was evaporated to dryness under reduced pressure and the crude product purified by column chromatography (neutral alumina, 2% methanol in dichloromethane) to isolate the desired compound in pure form. Yield: 73%

### Step-7: N,N-Dimethyl-4-(pyridin-3-yl)piperidin-4-amine (amine A44)

A solution of 1-benzyl-N,N-dimethyl-4-(pyridin-3-yl)piperidin-4-amine (300 mg, 1.013 mmol) in methanol (100 ml) was purged with argon for 10 min followed by the addition of 20% palladium hydroxide (23 mg) and acetic acid (0.075 ml). The reaction mixture was evacuated and allowed to stir under a hydrogen atmosphere (balloon) for 1 h. The mixture was filtered through a celite bed and washed with ethanol. The filtrate was concentrated under reduced pressure to isolate the desired compound in pure form, which was used for the next step without further purification. Yield: 98%

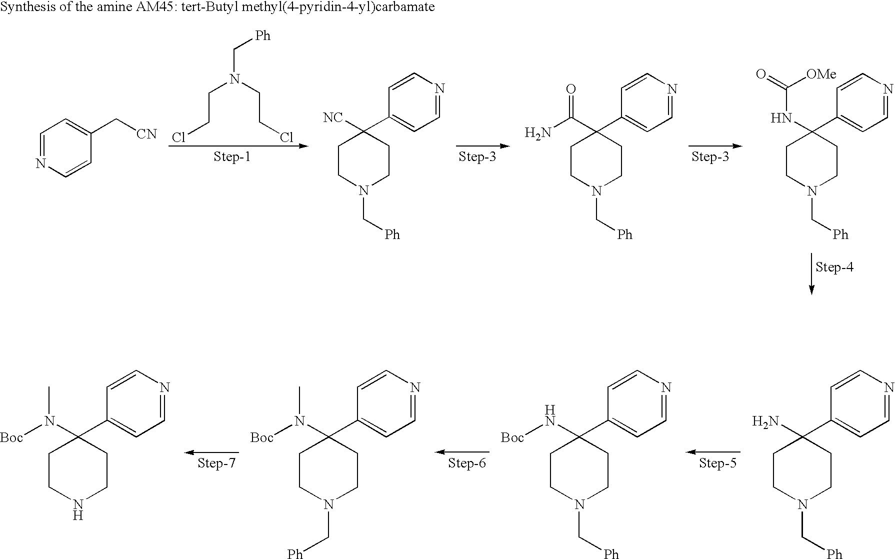

### Step-1: 1-Benzyl-4-(pyridin-4-yl)piperidine-4-carbonitrile

To a suspension of anhydrous powdered potassium hydroxide (4.2 g) in anhydrous toluene (50 ml) was added the hydrochloride salt of 2-(pyridin-3-yl)acetonitrile (1.8 g, 0.0152 mol) at 25° C. under an argon atmosphere. The resultant reaction mixture was cooled to 0° C. and benzyl-bis-(2-chloroethyl)-amine (4.2 g, 0.0182 mol) was added followed by 18-crown-6 (1 g) and the mixture allowed to reflux for 2 h. The reaction was quenched with crushed ice and extracted with dichloromethane. The organic layer was washed with water (2×) and brine, dried over sodium sulfate, filtered and concentrated under reduced pressure to obtain the crude material, which was purified by column chromatography (100-200 mesh silica gel, 3% methanol in dichloromethane) to obtain the desired compound.

Yield: 49%

### Step-2: 1-Benzyl-4-(pyridin-4-yl)piperidine-4-carboxamide

To a stirred solution of 1-benzyl-4-(pyridin-4-yl)piperidine-4-carbonitrile (3.0 g, 10.8 mmol) in ethanol:water (92 ml, 1:1) was added potassium hydroxide (3.02 g) and the mixture was refluxed for 7 h. The solvent was evaporated under reduced pressure and it was azeotroped with toluene. The crude material was purified by column chromatography (neutral alumina, 2% methanol in dichloromethane) to obtain the desired compound in pure form.

Yield: 53%

### Step 3: Methyl 1-benzyl-4-(pyridin-4-yl)piperidin-4-ylcarbamate

To a stirred solution of 1-benzyl-4-(pyridin-4-yl)piperidine-4-carboxamide (1.7 g, 5.743 mmol) in methanol (60 ml) was added KF/Al₂O₃ (12.70 g) and a 4% solution of sodium hypochlorite (18.36 ml). The mixture was stirred at 25° C. for 4 h. The solid was filtered off and washed with methanol. The methanol part was evaporated under reduced pressure to obtain the crude material, which was purified by column chromatography (neutral alumina, 0.5% methanol in dichloromethane) to isolate the desired product in pure form. Yield: 74%

### Step 4: 1-Benzyl-4-(pyridin-4-yl)piperidin-4-amine

To a stirred solution of methyl 1-benzyl-4-(pyridin-4-yl)piperidin-4-ylcarbamate (1.4 g, 4.307 mmol) in methanol was added 60% KOH solution. The resultant reaction mixture was then refluxed for 9 h. The mixture was evaporated to dryness and the crude product purified by column chromatography (neutral alumina, 0.5% methanol in dichloromethane).

Yield: 65%

### Step 5: tert-Butyl 1-benzyl-4-(pyridin-4-yl)piperidin-4-ylcarbamate

To a suspension of 50% sodium hydride (669 mg, 13.95 mmol) in anhydrous THF (15 ml) was added a solution of 1-benzyl-4-(pyridin-4-yl)piperidin-4-amine (750 mg, 2.796 mmol) in anhydrous THF (15 ml) dropwise at 0° C. under an argon atmosphere. The reaction mixture was stirred at 25° C. for 30 min and it was then cooled to 0° C. and (BOC)₂O (0.697 ml, 3.34 mmol) was added dropwise. The resultant solution was allowed to warm to 25° C. and stirred for 48 h. The reaction mixture was cooled to 0° C. and quenched with crushed ice, diluted with ethyl acetate, washed with brine, dried over sodium sulfate, filtered and concentrated under reduced pressure to obtain the crude material, which was purified by column chromatography (neutral alumina, 0.25% methanol in dichloromethane).

Yield: 48.5%

### Step 6: tert-Butyl 1-benzyl-4-(pyridin-4-yl)piperidin-4-yl(methyl)carbamate

To a suspension of 50% sodium hydride (261 mg, 5.448 mmol) in anhydrous THF (10 ml) was added a solution of tert-butyl 1-benzyl-4-(pyridin-4-yl)piperidin-4-ylcarbamate (500 mg, 1.362 mmol) in anhydrous THF (10 ml) dropwise at 0° C. under an argon atmosphere. The reaction mixture was stirred at 25° C. for 30 min, then it was again cooled to 0° C. and methyl iodide (0.254 ml, 4.086 mmol) was added dropwise. The resultant reaction mixture was allowed to stir at room temperature for 48 h. The reaction mixture was cooled to 0° C. and quenched with crushed ice, diluted with ethyl acetate, washed with brine, dried over sodium sulfate, filtered and concentrated under reduced pressure to obtain the crude material, which was purified by column chromatography (neutral alumina, 0.5% methanol in dichloromethane) to isolate the desired compound. Yield: 38%

### Step 7: tert-Butyl methyl(4-(pyridin-4-yl)piperidin-4-yl)carbamate (amine A45)

A solution of tert-butyl 1-benzyl-4-(pyridin-4-yl)piperidin-4-yl(methyl)carbamate (200 mg, 0.524 mmol) in methanol (30 ml) was degassed with argon for 10 min followed by the addition of 20% palladium hydroxide (96 mg) and acetic acid (0.075 ml). The reaction mixture was evacuated and allowed to stir at room temperature under a hydrogen atmosphere for 1 h by using H₂ balloon. The reaction mixture was filtered through a celite bed and washed with methanol. The filtrate was concentrated under reduced pressure to isolate the desired product in pure form, which was used for the next step without further purification. Yield: 86%

### Synthesis of the amine AM-46: 1-(4-(3-Fluorophenyl)piperidin-4-yl)-4-methylpiperazine dihydrochloride

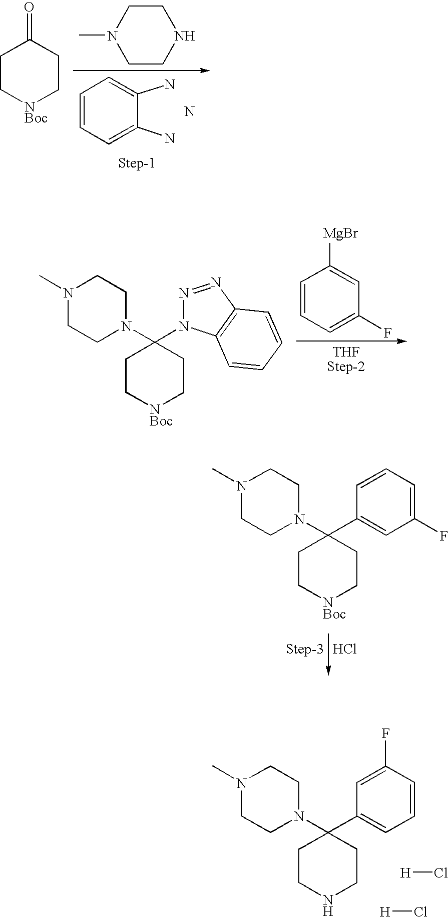

Step-1: N-Boc piperidone (15 mmol), N-methylpiperazine (1 eqv) and 1-H-benzotriazole (1 eqv) in benzene (60 ml) were refluxed for 16 hrs with the azeotropic removal of water using dean-stark apparatus. Solvent was evaporated under reduced pressure and the crude mass so obtained was used directly in the next step.

Yield: 90% (crude)

Step-2: To a THF solution of the Grignard reagent (60 mmol) was added benzotriazole adduct (12 mmol) obtained from step-1 in dry THF dropwise at 0° C. and the resulting reaction mixture was allowed to stir at 25° C. for 16 hrs (monitored by TLC). It was cooled to 0° C., quenched with saturated ammonium chloride solution and extracted with ethyl acetate, organic layer was washed successively with water, brine and finally dried over sodium sulfate. Evaporation of organic layer under reduced pressure gave the crude product which was purified by column chromatography (2% methanol in dichloromethane). Yield: 30%

Step-3: The boc-protected amine from Step 2 (25.8 mmol) was dissolved in Methanol/Tetrahydrofurane (126 mL; 1:1) and cooled to 0° C. At this temperature acetylchloride (129 mmol) was added. The reaction mixture was stirred for 3 h at room temperature (monitored by TLC). After completion the solvent was evaporated under reduced pressure to give the desired product. Yield: 108%

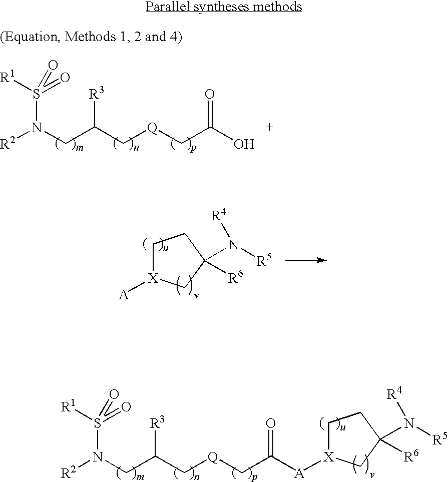

### Method 1

Acid solution (0.05 M in MC, 2 ml) was added to 105 μmol of CDI solution (0.105 M in MC, 1 ml) and the mixture was shaken at RT for 1 h. 100 μmol of the amine solution M in MC) were then added at RT and the mixture was shaken at RT for a further 12 h. 3 ml of water were then added to the reaction mixture, the mixture was shaken for 15 min and the organic phase was separated off. After removal of the solvent by distillation, the crude products were analyzed by LC-MS and purified via HPLC.

### Method 2

The particular amine (50-70 mg, 1.2 eq.), dissolved in MC (3 ml/mmol) and EDCl (1.5 eq.), HOBT (1 eq.), DIPEA (2 eq.) were added to a solution of the acid (50 mg, 1 eq.) in MC (3 ml/mmol), while stirring. When the reaction had ended, the crude products were purified via column chromatography (Biotage parallel purification system).

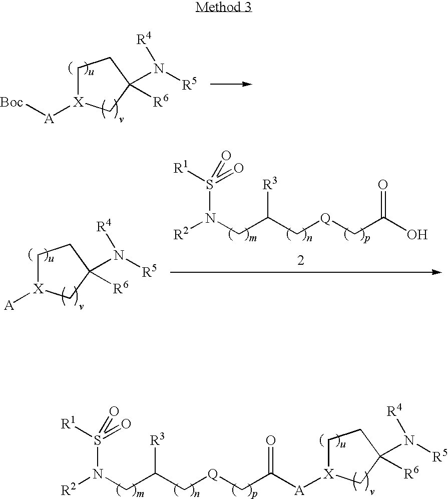

Stage 1. TFA (20% in MC, 5 ml/mmol) was added to the Boc-protected amine (1 eq.) at 0° C. The reaction mixture was warmed to 25° C. and stirred at this temperature for 3 h (TLC control). The solvent was removed completely and dried thoroughly to remove traces of the TFA. The crude product was employed further without further purification.

Stage 2. EDCl (1 eq.), HOBt (0.7 eq.) and DIPEA (2 eq.) were added to a solution of the acid unit (0.7 eq.) in MC (3 ml/mmol) and the mixture was stirred at 25° C. for 15 min. In another reaction vessel, the Boc-deprotected amine (1 eq.) in MC (2 ml/mmol) was cooled to 0° C. and DIPEA (2.5 eq.) was added. The solution obtained in this way was added to the solution of the acid unit. The reaction mixture was stirred at 25° C. for 16 h and then diluted with MC. The organic phase was washed successively with aqueous ammonium chloride solution, aqueous sodium bicarbonate solution and saturated sodium chloride solution. The organic phase was dried over sodium sulfate and concentrated. The crude product was purified via a parallel purification system from Biotage.

### Method 4

EDCl (1 eq.), HOBt (0.7 eq.) and DIPEA (2 eq.) were added to a solution of the acid unit (0.7 eq.) in MC (3 ml/mmol) and the mixture was stirred at 25° C. for 15 min. The amine unit (1 eq.), dissolved in MC (2 ml/mmol), was then The reaction mixture was stirred at 25° C. for 16 h and then diluted with MC. The organic phase was washed successively with aqueous ammonium chloride solution, aqueous sodium bicarbonate solution and saturated sodium chloride solution. The organic phase was dried over sodium sulfate and concentrated. The crude product was purified via a parallel purification system from Biotage.

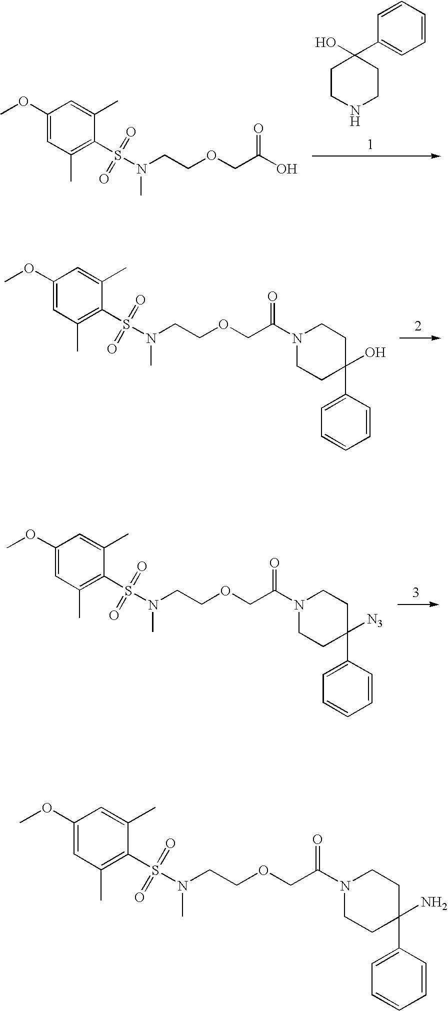

Stage 1. A suspension of the acid AC1 (4.00 g, 12.1 mmol), 4-phenylpiperidin-4-ol (2.14 g, 12.1 mmol), DIPEA (4.0 ml, 24 mmol) and HOAt (165 mg, 1.21 mmol) in MC (250 ml) was cooled to 0° C. EDCl (2.76 g, 14.48 mmol) was added and the mixture was stirred first at 0° C. for 30 min and then at RT overnight. The organic phase was extracted three times with 1 M HCl (100 ml each time) and saturated NaCl solution, dried over Na₂SO₄, filtered and concentrated. The crude product was purified by column chromatography (silica gel, MC/7 M NH₃ in methanol, 98:2).

Stage 2. Trimethylsilyl azide (11.07 ml, 83.4 mmol) was added to a solution of the alcohol (4.09 g, 8.34 mmol) and BF₃Et₂O (2.12 ml, 16.7 mmol) in MC (100 ml). The reaction solution was heated to 40-45° C. and stirred at this temperature overnight. Further trimethylsilyl azide (5.53 ml, 41.7 mmol) was added, the mixture was stirred at 40-45° C. for a further 8 h, trimethylsilyl azide (5.53 ml, 41.7 mmol) was added again and the mixture was stirred at 40-45° C. overnight. After cooling, the organic phase was washed with NH₄Cl solution, dried over sodium sulfate and concentrated. The residue was twice taken up in dry ethanol and the mixture concentrated. The crude product was stored in a freezer compartment and employed in the next stage without further purification.

Stage 3. A solution of the azide (4.71 g, max. 8.34 mmol) in ethanol (100 ml) was first gassed with N₂. Pd (C) (10%, 444 mg, 0.42 mmol) was then added and the reaction mixture was stirred under an H₂ atmosphere for 7 h. The reaction mixture was gassed with N₂ again for 20 min and filtered over Celite. The filter was rinsed with ethanol and the filtrate was concentrated under reduced pressure. The following day, the residue was taken up in ethanol (100 ml), the mixture was gassed with N₂ for 10 min, Pd (C) (10%, 444 mg, 0.42 mmol) was added and the mixture was stirred under an H₂ atmosphere again for 5 h. The reaction mixture was gassed with N₂ again for 10 min and filtered over Celite. The filter was rinsed with ethanol and the filtrate was concentrated under reduced pressure. The crude product obtained was purified by column chromatography (silica gel, ethyl acetate/MC, 1:1→MC→MC/7 M NH₃ in methanol, 95:5).

### Example 197

### Preparation of N-(2-(2-(3-benzyl-3-(4-methylpiperazin-1-yl)pyrrolidin-1-yl)-2-oxoethoxy)ethyl)-4-methoxy-N,2,6-trimethylbenzenesulfonamide

### Preparation of 1-(3-benzylpyrrolidin-3-yl)-4-methylpiperazine

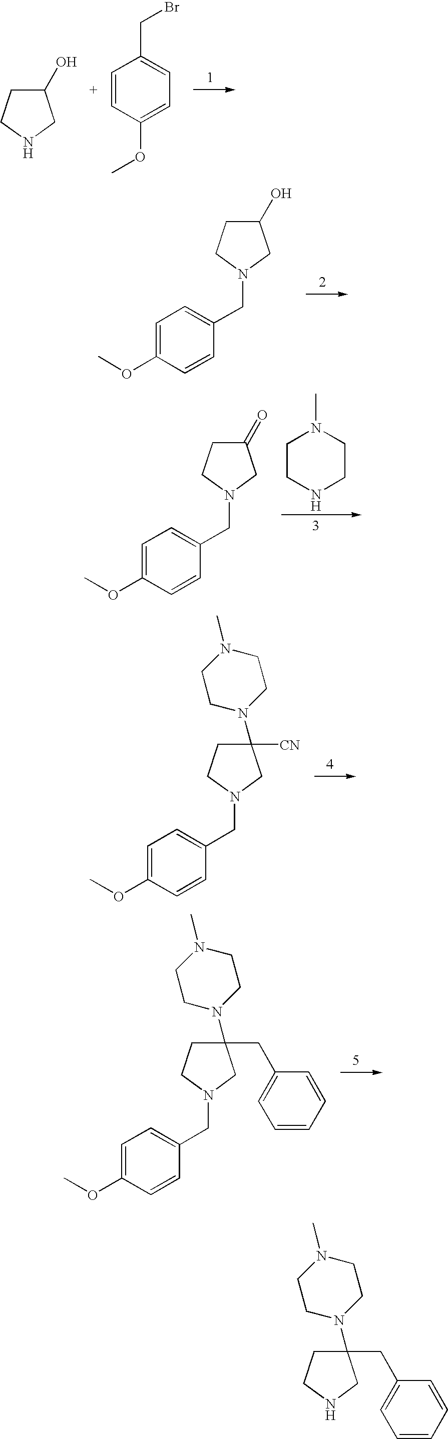

Stage 1. A suspension of DL-3-pyrrolidinol (2.83 g, 32.5 mmol), p-methoxybenzyl bromide (6.53 g, 32.5 mmol) and K₂CO₃ (13.49 g, 97.6 mmol) in acetone (100 ml) was stirred under reflux for 90 min. The reaction mixture was filtered and the filtrate was concentrated under reduced pressure. The crude product was employed in the next stage without further purification.

Stage 2. SO₃-pyridine (15.52 g, 97.5 mmol) was added in portions to a solution of the alcohol (max. 32.5 mmol), triethylamine (27.1 ml, 195 mmol) and DMSO (23 ml, 325 mmol) in MC (100 ml). The reaction mixture was stirred at RT for 3 h. Saturated NH₄Cl solution (100 ml) was then added. After separation of the phases, the aqueous phase was extracted once more with MC. The combined organic phases were dried over Na₂SO₄ and concentrated under reduced pressure. The crude product was purified by column chromatography (silica gel, heptane/ethyl acetate, 2: 1).

Stage 3. A suspension of the ketone (2.62 g, 12.8 mmol) and 1-methylpiperazine (1.28 g, 12.8 mmol) in aqueous HCl (pH 3.5, 5 ml) was stirred at RT for 5 h. KCN (875 mg, 13.44 mmol) was added and the mixture was stirred at RT overnight. Ethyl acetate (50 ml) and saturated NaCl solution (50 ml) were then added and the aqueous phase was extracted with ethyl acetate (50 ml). The combined organic phases were dried over Na₂SO₄ and concentrated under reduced pressure. The residue was twice more taken up in MC and the mixture concentrated. The crude product was employed further without purification.

Stage 4. The reaction was carried out under an N₂ atmosphere. A solution of benzylmagnesium bromide in THF (20 wt. %, 24 g, 31.8 mmol) was cooled to 0° C. and a solution of the nitrile (2.0 g, 6.4 mmol) in THF (15 ml) was added dropwise over a period of approx. 30 min. The reaction solution was then stirred at RT overnight. When the reaction had ended, saturated NH₄Cl solution (50 ml) and water (50 ml) were added. The mixture was extracted three times with ethyl acetate (50 ml each time), dried over Na₂SO₄ and concentrated under reduced pressure. The crude product obtained was purified by column chromatography (silica gel, MC/7 M NH₃ in methanol, 98:2).

Stage 5. 1-Chloroethyl chloroformate (681 μl, 6.31 mmol) was added to a solution of the p-methoxybenzylamine (7.479 mg, 1.26 mmol) in DME (10 ml) under reflux and the mixture was heated under reflux for a further 90 min. The solution obtained was concentrated to dryness, the residue was taken up again in DME and the mixture was concentrated. The residue was taken up in methanol and the mixture was stirred for 60 min and concentrated in vacuo. The crude product was employed further without purification.

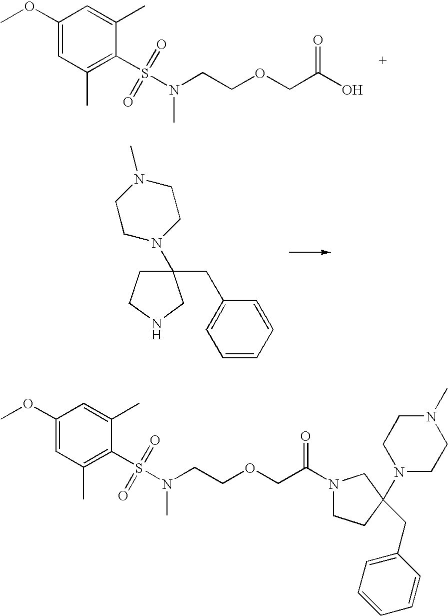

A solution of the amine (max. 1.26 mmol), the carboxylic acid AC1 (418 mg, 1.26 mmol) and DIPEA (625 μl, 3.78 mmol) in MC (10 ml) was cooled to 0° C. HOAt (18 mg, 0.13 mmol) and EDCl (289 mg, 1.51 mmol) were added and the reaction mixture was stirred at 0° C. for 30 min and at RT overnight. The mixture was diluted with MC (50 ml) and the organic phase was washed with saturated NaCl solution. The organic phase was dried over Na₂SO₄ and concentrated under reduced pressure. The crude product obtained was purified first by column chromatography (silica gel, MC/7 M NH₃ in methanol, 98:2) and then via preparative LCMS. The compound obtained was taken up in ethyl acetate (25 ml), the mixture was filtered and the filtrate was concentrated to dryness. The residue was taken up again in ethyl acetate (50 ml) and the mixture was washed twice with saturated NaHCO₃ solution (50 ml each time), dried over Na₂SO₄ and concentrated under reduced pressure.

### Example 198

### Preparation of N-(4-(dimethylamino)-4-phenylcyclohexyl)-2-(1-(2,4,6-trichlorophenylsulfonyl)pyrrolidin-3-yloxy)acetamide

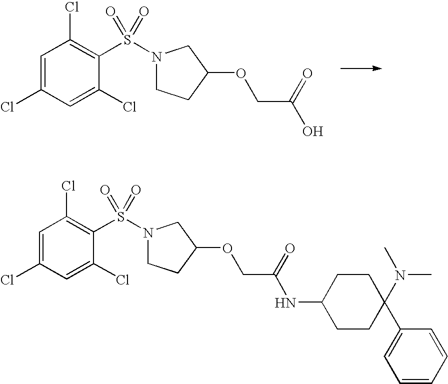

N,N′-Carbonyldiimidazole (164 mg, 1.02 mmol) was added to a solution of carboxylic acid AC18 (331 mg, 0.85 mmol) in tetrahydrofuran (10 ml) and the mixture was stirred at room temperature for 1 h. A solution of N1,N1-dimethyl-1-phenylcyclohexane-1,4-diamine AM15 (205 mg, 0.94 mmol) in tetrahydrofuran (10 ml) was then added to this mixture and the mixture was stirred at room temperature overnight. The mixture was then concentrated in vacuo, the residue was taken up in sodium bicarbonate solution and the mixture was extracted with methylene chloride (3×20 ml). The combined organic phases were dried with sodium sulfate and concentrated. The residue was purified by means of flash chromatography with chloroform/methanol (95:5). Yield: 190 mg, 37%.

¹H-NMR (DMSO-d₆): 1.30-1.55 (m, 6H); 1.55-1.80 (m, 2H); 1.93 (s, 6H); 1.95-2.13 (m, 2H); 2.58 (br d, 2H, J=11.6 Hz); 3.45 (dd, 2H, J=9.6, 4.6 Hz); 3.59 (d, 1H, J=11.0 Hz); 3.83 and 3.84 (2s, 2H); 4.18 (m, 1H); 7.17-7.40 (m, 5H); 7.54 (d, 1H, J=8.4 Hz); 7.90 (2H, s).

### Example 199

### Preparation of N-(4-(dimethylamino)-4-phenylcyclohexyl)-2-(2-(2,4,6-trichloro-N-methylphenylsulfonamido)ethoxy)acetamide

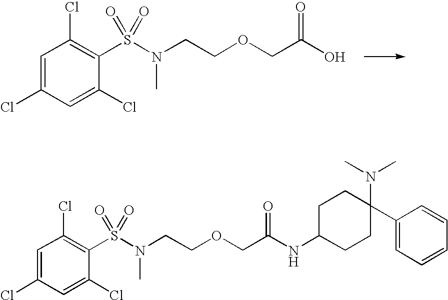

N,N′-Carbonyldiimidazole (154 mg, 0.95 mmol) was added to a solution of carboxylic acid AC3 (298 mg, 0.79 mmol) in tetrahydrofuran (10 ml) and the mixture was stirred at room temperature for 1 h. A solution of N1,N1-dimethyl-1-phenylcyclohexane-1,4-diamine AM15 (190 mg, 0.87 mmol) in tetrahydrofuran (10 ml) was then added to this mixture and the mixture was stirred at RT overnight. The mixture was then concentrated in vacuo, the residue was taken up in sodium bicarbonate solution and the mixture was extracted with methylene chloride (3×20 ml). The combined organic phases were dried with sodium sulfate and concentrated. The residue was purified by flash chromatography with chloroform/methanol (95:5). Yield: 296 mg, 65%.

¹H-NMR (DMSO-d₆): 1.41-1.59 (m, 4H); 1.69 (q, 2H, J=10.9 Hz); 1.77 (d, 1H, J=11.9 Hz); 1.92 (s, 6H); 2.57 (d, 2H, J=14.1 Hz); 2.90 (s, 3H); 3.49 (t, 2H, J=5.1 Hz); 3.61 (t, 3H, J=5.3 Hz); 3.84 (s, 2H); 7.24 (m, 1H); 7.30-7.38 (m, 4H); 7.51 (d, 4H, J=8.0 Hz); 7.90 (s, 2H).

### Preparation of Individual Substances from (S)-2-((1-(4-methoxy-2,6-dimethylphenylsulfonyl)piperidin-2-yl)methoxy)acetic acid

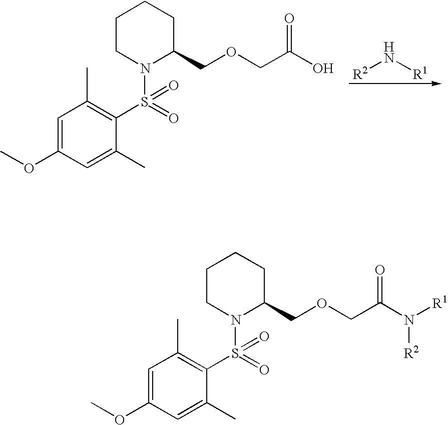

### Example 200

### (S)-2-((1-(4-Methoxy-2,6-dimethylphenylsulfonyl)piperidin-2-yl)methoxy)-N-methyl-N-(2-(4-phenyl-4-(pyrrolidin-1-yl)cyclohexyl)ethyl)acetamide

(S)-2-((1-(4-Methoxy-2,6-dimethylphenylsulfonyl)piperidin-2-yl)methoxy)acetic acid (1 eq.) was dissolved in methylene chloride (5 ml/mmol), the solution was cooled and diisopropylethylamine (2.5 eq.), 1-hydroxybenzotriazole hydrate (1 eq.) and EDCl (1.5 eq.) were added at 0° C. The cooling bath was removed and the reaction mixture was stirred at room temperature for 15 min. The reaction mixture was cooled again and N-methyl-2-(4-phenyl-4-(pyrrolidin-1-yl)cyclohexyl)ethanamine (amine AM32, 1.2 eq.) was added at 0° C. The ice bath was removed and the mixture was stirred at room temperature for 16 h. It was diluted with methylene chloride and washed with saturated ammonium chloride solution, saturated sodium chloride solution, saturated sodium carbonate solution and saturated sodium chloride solution again. The organic phase was dried over sodium sulfate and concentrated in vacuo. The crude product was purified by column chromatography (silica gel) (2% methanol in methylene chloride). Yield: 53%, yellow, finely crystalline MS, Rₜ=4.1 min, m/z=640.3[MH]⁺

The example compounds listed in the following table were prepared from (S)-2-((1-(4-methoxy-2,6-dimethylphenylsulfonyl)piperidin-2-yl)methoxy)acetic acid by reaction with the corresponding amines (R¹R²NH) analogously to the process described for Example 200.

| Example | | | MS, m/z |
|---|---|---|---|
| no. | Amine (R¹R²NH) | Yield (%) | (MH⁺) |
| 201 | 2-(4-(Azetidin-1-yl)-4- | 30 | Rₜ = 3.7 min, |
| | phenylcyclohexyl)-N- | | m/z = 626.2 [MH]⁺ |
| | methylethanamine AM41 | | |
| | (synthesis analogous to | | |
| | amine AM32) | | |

### Preparation of Individual Substances from 2-((1-(4-methoxy-2,6-dimethylphenylsulfonyl)piperidin-2-yl)methoxy)acetic acid (acid AC9)

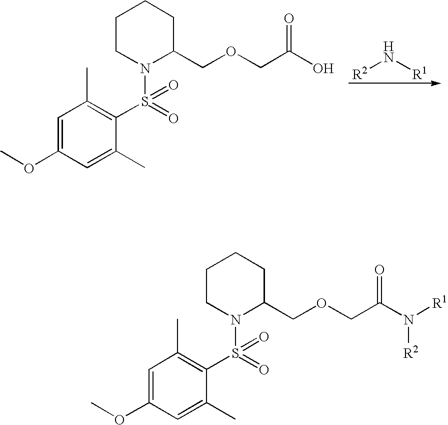

### Example 202

### 1-(4-(Dimethylamino)-4-phenylpiperidin-1-yl)-2-((1-(4-methoxy-2,6-dimethylphenylsulfonyl)piperidin-2-yl)methoxy)ethanone hydrochloride

2-((1-(4-Methoxy-2,6-dimethylphenylsulfonyl)piperidin-2-yl)methoxy)acetic acid (acid AC9) (1 eq.) was dissolved in methylene chloride (5 ml/mmol), the solution was cooled and diisopropylethylamine (2.5 eq.), 1-hydroxybenzotriazole hydrate (1 eq.) and EDCl (1.5 eq.) were added at 0° C. The cooling bath was removed and the reaction mixture was stirred at room temperature for 15 min. The reaction mixture was cooled again and N,N-dimethyl-4-phenylpiperidin-4-amine (amine AM1, 1.2 eq.) was added at 0° C. The ice bath was removed and the mixture was stirred at room temperature for 16 h. It was diluted with methylene chloride and washed with saturated ammonium chloride solution, saturated sodium chloride solution, saturated sodium carbonate solution and saturated sodium chloride solution again. The combined organic phases were dried over sodium sulfate and concentrated in vacuo. The crude product was purified by column chromatography (silica gel) (2% methanol in methylene chloride). The hydrochloride was precipitated from dioxane solution with hydrogen chloride in dioxane (saturated). Yield: 48%, yellow, finely crystalline MS, Rₜ=3.2 min, m/z=558.1 [MH]⁺

The example compounds listed in the following table were prepared from 2-((1-(4-methoxy-2,6-dimethylphenylsulfonyl)piperidin-2-yl)methoxy)acetic acid (acid AC9) by reaction with the corresponding amines (R¹R²NH) analogously to the process described for Example 202.

| Example | | Yield | MS, m/z |
|---|---|---|---|
| no. | Amine (R¹R²NH) | (%) | (MH⁺) |
| 203 | N,N-Dimethyl-4-(3- | 79 | Rₜ = 4.1 min, |
| | (methylamino)propyl)-1- | | m/z = 628.3 [MH]⁺ |
| | phenylcyclohexanamine AM38 | | |
| 204 | 3-(4-(3-Fluorophenyl)-4- | 30 | Rₜ = 4.0 min, |
| | (pyrrolidin-1-yl)cyclohexyl)-N- | | m/z = 672.2 [MH]⁺ |
| | methylpropan-1-amine AM39 | | |
| 205 | 3-(4-(Azetidin-1-yl)-4-(3- | 27 | Rₜ = 3.9 min, |
| | fluorophenyl)cyclohexyl)-N- | | m/z = 640.2 [MH]⁺ |
| | methylpropan-1-amine AM42* | | |

*The synthesis of the amine was carried out analogously to the synthesis of 3-(4-(3-fluorophenyl)-4-(pyrrolidin-1-yl)cyclohexyl)-N-methylpropan-1-amine (employed in the preparation of Example Compound 204).

### Example Compound 206

### N-(2-(2-(4-(Dimethylamino)-4-(pyridin-4-yl)piperidin-1-yl)-2-oxoethoxy)ethyl)-4-methoxy-N,2,6-trimethylbenzenesulfonamide

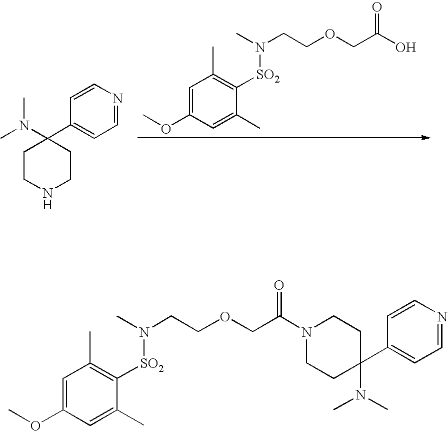

Diisopropylethylamine (2.5 eq.), HOBT (1 eq.) and EDCl (1.5 eq.) were added to a solution of 2-(2-(4-methoxy-N,2,6-trimethylphenylsulfonamido)ethoxy)-acetic acid (AC1) (0.406 mmol) in methylene chloride (10 ml/mmol) at 0° C. The reaction mixture was stirred at 25° C. for 15 min and cooled again to 0° C., N,N-dimethyl-4-(pyridin-4-yl)piperidin-4-amine (1.1 eq.) (AM40) was added and the mixture was stirred at 25° C. for 16 h. It was then diluted with methylene chloride (30 ml) and washed with saturated ammonium chloride solution, saturated sodium chloride solution, saturated sodium carbonate solution and saturated sodium chloride solution again. The organic phase was dried over sodium sulfate and concentrated in vacuo. The crude product was purified by column chromatography (Alox neutral, 2% methanol in methylene chloride). Yield: 30%

MS, Rₜ=2.5 min, m/z=519.2 [MH]⁺

### Example 207

### N-(2-(4-(Dimethylamino)-4-(pyridin-3-yl)cyclohexyl)ethyl)-2-(2-(4-methoxy-N,2,6-trimethylphenylsulfonamido)ethoxy)-N-methylacetamide

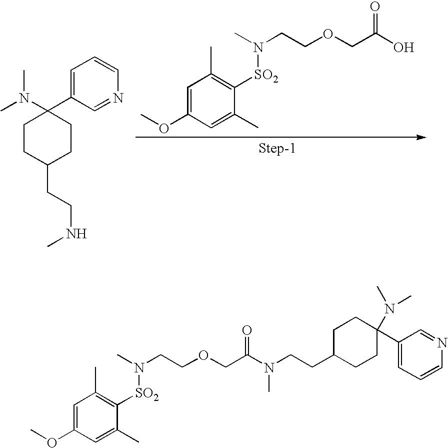

### Step-1: N-(2-(4-(Dimethylamino)-4-(pyridin-3-yl)cyclohexyl)ethyl)-2-(2-(4-methoxy-N,2,6-trimethylphenylsulfonamido)ethoxy)-N-methylacetamide (Example 207)

To a solution of 2-(2-(4-methoxy-N,2,6-trimethylphenylsulfonamido)ethoxy)acetic acid (carboxylic acid S1) (76 mg, 0.1962 mmol) in dichloromethane (10 ml/mmol) was added, diisopropyl ethylamine (0.085 ml, 0.4905 mmol) at 0° C. followed by the addition of HOBT (27 mg, 0.1962 mmol) and EDCl (57 mg, 0.2943 mmol). The resultant solution was allowed to stir at 25° C. for 15 min. It was again cooled to 0° C. and the N,N-dimethyl-4-(2-(methylamino)ethyl)-1-(pyridin-3-yl)cyclohexanamine (85 mg) dissolved in dichloromethane (3 ml) was added. The reaction mixture was allowed to stir for 16 h at 25° C. The mixture was diluted with dichloromethane (30 ml), washed with saturated ammonium chloride solution, brine, saturated sodium bicarbonate and finally again with brine. The organic layer was dried over sodium sulfate and evaporated to dryness under reduced pressure to yield the crude product. The crude material was purified by column chromatography to give the desired product. Yield: 44%

MS, Rₜ=2.5 min, m/z=505.4 [MH]⁺

### Example 208

### N-(2-(2-(4-(Dimethylamino)-4-(pyridin-3-yl)piperidin-1-yl)-2-oxoethoxy)ethyl)-4-methoxy-N,2,6-trimethylbenzenesulfonamide

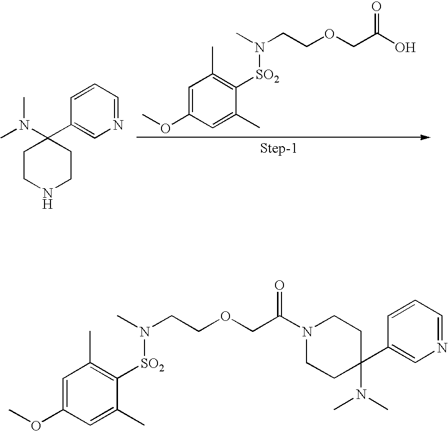

### Step-1: N-(2-(2-(4-(Dimethylamino)-4-(pyridin-3-yl)piperidin-1-yl)-2-oxoethoxy)ethyl)-4-methoxy-N,2,6-trimethylbenzenesulfonamide (Example 208)

To a solution of 2-(2-(4-methoxy-N,2,6-trimethylphenylsulfonamido)ethoxy)acetic acid (carboxylic acid S1) (129 mg, 0.390 mmol) in dichloromethane (10 ml/mmol) was added diisopropyl ethylamine (0.336 ml, 0.487 mmol) at 0° C. followed by the addition of HOBT (65.79 mg, 0.487 mmol) and EDCl (140 mg, 0.730 mmol). The resultant solution was allowed to stir at 25° C. for 15 min. It was again cooled to 0° C. and N,N-dimethyl-4-(pyridin-3-yl)piperidin-4-amine (90 mg) dissolved in dichloromethane and DMF (3 ml and 2 ml) was added. The reaction mixture was allowed to stir for 16 h at 25° C. The mixture was diluted with dichloromethane (30 ml), washed with saturated ammonium chloride solution, brine, saturated sodium bicarbonate and finally again with brine. The organic layer was dried over sodium sulfate and evaporated to dryness under reduced pressure to get the crude product. The crude material was purified by column chromatography (neutral alumina, 0.5% methanol in dichloromethane). Yield: 30%. MS, Rₜ=2.8 min, m/z=575.4 [MH]⁺

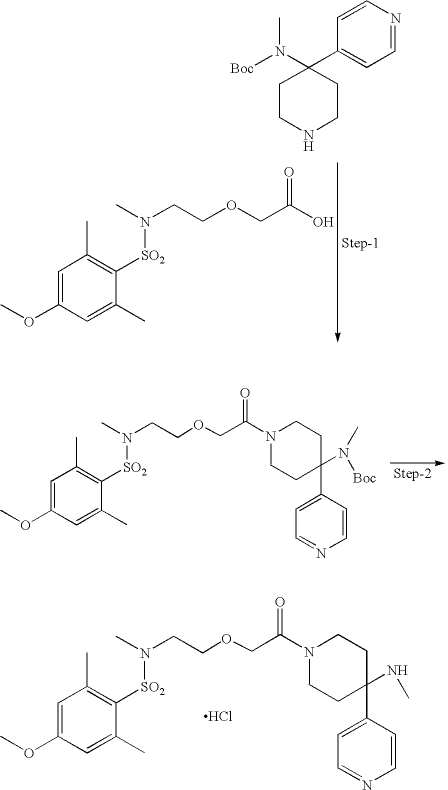

### Step 1: tert-Butyl 1-(2-(2-(4-methoxy-N,2,6-trimethylphenylsulfonamido)ethoxy)-acetyl)-4-(pyridin-4-yl)piperidin-4-yl (methyl)carbamate

To a solution of 2-(2-(4-methoxy-N,2,6-trimethylphenylsulfonamido)ethoxy)acetic acid (carboxylic acid S1) (120 mg, 0.365 mmol) in dichloromethane (10 ml/mmol) was added diisopropyl ethylamine (0.316 ml, 0.457 mmol) at 0° C. followed by the addition of HOBT (61.74 mg, 0.457 mmol) and EDCl (131 mg, 0.685 mmol). The resultant solution was allowed to stir at 25° C. for 15 min. It was again cool to 0° C. and tert-butyl methyl(4-(pyridin-4-yl)piperidin-4-yl)carbamate (130 mg) dissolved in dichloromethane and DMF (3 ml) was added. The reaction mixture was allowed to stir for 16 h at 25° C. The mixture was diluted with dichloromethane (30 ml), washed with saturated ammonium chloride solution, brine, saturated sodium bicarbonate and finally again with brine. The organic layer was dried over sodium sulfate and evaporated to dryness under reduced pressure to get the crude product. The crude material was purified by BIOTAGE column chromatography (2% methanol in dichloromethane) to yield the desired compound. Yield: 14%

### Step 2: 4-Methoxy-N,2,6-trimethyl-N-(2-(2-(4-(methylamino)-4-(pyridin-4-yl)piperidin-1-yl)-2-oxoethoxy)ethyl)benzenesulfonamide hydrochloride (Example 209)

Dioxane-HCl was added dropwise to a solution of tert-butyl 1-(2-(2-(4-methoxy-N,2,6-trimethylphenylsulfonamido)ethoxy)-acetyl)-4-(pyridin-4-yl)piperidin-4-yl(methyl)carbamate in ethyl acetate under cooled conditions and the mixture was stirred at room temperature for 2 h. The solvent was evaporated under reduced pressure and it was azeotroped with toluene (2×) to obtain the desired product. Yield: 90%.

MS, Rₜ=2.5 min, m/z=505.4[MH]⁺

The synthesis methods (parallel syntheses) for the example compounds are shown in the following table.

The Example Compounds (1) to (205) synthesized were analyzed, inter alia, with the aid of their molecular weight. The molecular weights measured by ESI-MS are summarized in the following table.

| | | | Mass |
|---|---|---|---|
| Example | Name | Method | (ESI-MS) |
| 1 | 2-(2-(3,4-Dichlorophenylsulfonyl)-1,2,3,4- | 1 | 627.2 |
| | tetrahydroisoquinolin-1-yl)-N-(4-(dimethylamino)-4- | | |
| | phenethylcyclohexyl)acetamide | | |
| 2 | N-(4-(Dimethylamino)-4-phenethylcyclohexyl)-2-((1- | 1 | 571.3 |
| | (4-methoxyphenylsulfonyl)piperidin-2- | | |
| | yl)methoxy)acetamide | | |
| 3 | N-(4-(Dimethylamino)-4-phenethylcyclohexyl)-2-(2-(4- | 1 | 589.3 |
| | methoxyphenylsulfonyl)-1,2,3,4-tetrahydroisoquinolin- | | |
| | 1-yl)acetamide | | |
| 4 | N-(4-(Dimethylamino)-4-(2-methylbenzyl)cyclohexyl)- | 1 | 571.3 |
| | 2-((1-(4-methoxyphenylsulfonyl)piperidin-2- | | |
| | yl)methoxy)acetamide | | |
| 5 | N-(4-(Dimethylamino)-4-phenethylcyclohexyl)-2-(1-(4- | 1 | 571.3 |
| | methoxy-2,6-dimethylphenylsulfonyl)pyrrolidin-3- | | |
| | yloxy)acetamide | | |
| 6 | N-(4-(Dimethylamino)-4-(3-fluorophenyl)cyclohexyl)- | 1 | 549.3 |
| | 2-(2-(4-methoxy-N,2,6-trimethylphenylsulfonamido)- | | |
| | ethoxy)acetamide | | |
| 7 | N-(4-(Dimethylamino)-4-phenethylcyclohexyl)-2-(2-(4- | 1 | 559.3 |
| | methoxy-N,2,6-trimethylphenylsulfonamido)ethoxy)- | | |
| | acetamide | | |
| 8 | N-(4-(Dimethylamino)-4-phenethylcyclohexyl)-2-(2- | 1 | 587.3 |
| | (N-ethyl-4-methoxy-2,3,6-trimethylphenyl- | | |
| | sulfonamido)ethoxy)acetamide | | |
| 9 | N-(4-(Dimethylamino)-4-(4-fluorobenzyl)cyclohexyl)- | 1 | 563.3 |
| | 2-(2-(4-methoxy-N,2,6-trimethylphenylsulfonamido)- | | |
| | ethoxy)acetamide | | |
| 10 | N-(4-(Dimethylamino)-4-(2-methylbenzyl)cyclohexyl)- | 1 | 559.3 |
| | 2-(2-(4-methoxy-N,2,6-trimethylphenylsulfonamido)- | | |
| | ethoxy)acetamide | | |
| 11 | 2-(2-(4-Methoxy-N,2,6-trimethylphenylsulfonamido)- | 1 | 571.3 |
| | ethoxy)-N-(4-phenyl-4-(piperidin-1-yl)cyclohexyl)- | | |
| | acetamide | | |
| 12 | 2-(2-(3,4-Dichlorophenylsulfonyl)-1,2,3,4-tetrahydro- | 1 | 627.2 |
| | isoquinolin-1-yl)-N-(4-(dimethylamino)-4-(2- | | |
| | methylbenzyl)cyclohexyl)acetamide | | |
| 13 | 2-(2-(2,6-Dichloro-N-methylphenylsulfonamido)- | 1 | 569.2 |
| | ethoxy)-N-(4-(dimethylamino)-4-phenethyl- | | |
| | cyclohexyl)acetamide | | |
| 14 | N-(4-(Dimethylamino)-4-(2-methylbenzyl)cyclohexyl)- | 1 | 571.3 |
| | 2-(1-(4-methoxy-2,6-dimethylphenylsulfonyl)- | | |
| | pyrrolidin-3-yloxy)acetamide | | |
| 15 | N-(4-Benzyl-4-(piperidin-1-yl)cyclohexyl)-2-(2-(3,4- | 1 | 653.2 |
| | dichlorophenylsulfonyl)-1,2,3,4-tetrahydroisoquinolin- | | |
| | 1-yl)acetamide | | |
| 16 | N-(4-(Azepan-1-yl)-4-benzylcyclohexyl)-2-(2-(3,4- | 1 | 667.2 |
| | dichlorophenylsulfonyl)-1,2,3,4-tetrahydroisoquinolin- | | |
| | 1-yl)acetamide | | |
| 17 | N-(4-Benzyl-4-(piperidin-1-yl)cyclohexyl)-2-(2-(4- | 1 | 585.3 |
| | methoxy-N,2,6-trimethylphenylsulfonamido)ethoxy)- | | |
| | acetamide | | |
| 18 | N-(4-Benzyl-4-(piperidin-1-yl)cyclohexyl)-2-(1-(4- | 1 | 597.3 |
| | methoxy-2,6-dimethylphenylsulfonyl)pyrrolidin-3- | | |
| | yloxy)acetamide | | |
| 19 | N-(4-(Dimethylamino)-4-phenylcyclohexyl)-2-(2-(1-(4- | 1 | 557.3 |
| | methoxyphenylsulfonyl)piperidin-2-yl)ethoxy)- | | |
| | acetamide | | |
| 20 | N-(4-(Azepan-1-yl)-4-benzylcyclohexyl)-2-(2-(4- | 1 | 599.3 |
| | methoxy-N,2,6-trimethylphenylsulfonamido)ethoxy)- | | |
| | acetamide | | |
| 21 | 2-(2-(2,4-Dichloro-N-methylphenylsulfonamido)- | 1 | 559.2 |
| | ethoxy)-N-(4-(dimethylamino)-4-(3-fluorophenyl)- | | |
| | cyclohexyl)acetamide | | |
| 22 | 2-(2-(2,4-Dichloro-N-methylphenyl- | 1 | 569.2 |
| | sulfonamido)ethoxy)-N-(4-(dimethylamino)-4- | | |
| | phenethylcyclohexyl)acetamide | | |
| 23 | N-(4-(Dimethylamino)-4-phenethylcyclohexyl)-2-(2- | 1 | 603.2 |
| | (2,4,6-trichloro-N-methylphenylsulfonamido)- | | |
| | ethoxy)acetamide | | |
| 24 | N-(4-(Dimethylamino)-4-phenethylcyclohexyl)-2-(2-(4- | 1 | 573.3 |
| | methoxy-N,2,3,6-tetramethylphenylsulfonamido)- | | |
| | ethoxy)acetamide | | |
| 25 | N-(4-(Dimethylamino)-4-phenethylcyclohexyl)-2-(2- | 1 | 543.3 |
| | (N,2,4,6-tetramethylphenylsulfonamido)ethoxy)- | | |
| | acetamide | | |
| 26 | 2-(2-(3,4-Dichlorophenylsulfonyl)-1,2,3,4- | 1 | 599.2 |
| | tetrahydroisoquinolin-1-yl)-N-(4-(dimethylamino)-4- | | |
| | phenylcyclohexyl)acetamide | | |
| 27 | 2-(1-(4-Methoxy-2,6-dimethylphenylsulfonyl)- | 1 | 583.3 |
| | pyrrolidin-3-yloxy)-N-(4-phenyl-4-(piperidin-1- | | |
| | yl)cyclohexyl)acetamide | | |
| 28 | 2-(2-(4-Methoxy-N,2,3,6-tetramethylphenyl- | 1 | 585.3 |
| | sulfonamido)ethoxy)-N-(4-phenyl-4-(piperidin-1-yl)- | | |
| | cyclohexyl)acetamide | | |
| 29 | N-(4-(Dimethylamino)-4-phenethylcyclohexyl)- | 1 | 555.3 |
| | 2-(1-(mesitylsulfonyl)pyrrolidin-3-yloxy)- | | |
| | acetamide | | |
| 30 | 2-(2-(2,4-Dichloro-N-methylphenylsulfonamido)- | 1 | 569.2 |
| | ethoxy)-N-(4-(dimethylamino)-4-(2-methylbenzyl)- | | |
| | cyclohexyl)acetamide | | |
| 31 | N-(4-(Dimethylamino)-4-phenethylcyclohexyl)-2-(2- | 1 | 569.7 |
| | (N-methyl-3-(trifluoromethyl)phenylsulfonamido)- | | |
| | ethoxy)acetamide | | |
| 32 | 2-((1-(4-Methoxy-2,6-dimethylphenylsulfonyl)- | 3 | 653.4 |
| | piperidin-2-yl)methoxy)-N-methyl-N-(3-(4-phenyl-4- | | |
| | (pyrrolidin-1-yl)cyclohexyl)propyl)acetamide | | |
| 33 | N-Methyl-N-((4-phenyl-4-(pyrrolidin-1-yl)cyclohexyl)- | 3 | 605.3 |
| | methyl)-2-(1-(3-(trifluoromethyl)phenylsulfonyl)- | | |
| | piperidin-2-yl)acetamide | | |
| 34 | 2-(2-(4-Methoxy-N,2,6-trimethylphenylsulfonamido)- | 3 | 599.3 |
| | ethoxy)-N-methyl-N-(2-(4-phenyl-4-(pyrrolidin-1- | | |
| | yl)cyclohexyl)ethyl)acetamide | | |
| 35 | 2-(2-(4-Methoxy-N,2,6-trimethylphenylsulfonamido)- | 3 | 585.3 |
| | ethoxy)-N-methyl-N-((4-phenyl-4-(pyrrolidin-1- | | |
| | yl)cyclohexyl)methyl)acetamide | | |
| 36 | 1-(4-Benzyl-4-(dimethylamino)piperidin-1-yl)-2-((1- | 4 | 629.2 |
| | (3,4-dichlorophenylsulfonyl)-1,2,3,4- | | |
| | tetrahydroquinolin-2-yl)methoxy)ethanone | | |
| 37 | N-Methyl-N-(3-(4-phenyl-4-(pyrrolidin-1-yl)- | 3 | 633.3 |
| | cyclohexyl)propyl)-2-(1-(3-(trifluoromethyl)- | | |
| | phenylsulfonyl)piperidin-2-yl)acetamide | | |
| 38 | N-Methyl-N-((4-phenethyl-4-(pyrrolidin-1-yl)- | 3 | 633.3 |
| | cyclohexyl)methyl)-2-(1-(3-(trifluoromethyl)- | | |
| | phenylsulfonyl)piperidin-2-yl)acetamide | | |
| 39 | N-(2-(4-Benzyl-4-(pyrrolidin-1-yl)cyclohexyl)ethyl)-2- | 3 | 613.4 |
| | (2-(4-methoxy-N,2,6-trimethylphenylsulfonamido)- | | |
| | ethoxy)-N-methylacetamide | | |
| 40 | N-Methyl-3-(naphthalene-2-sulfonamido)-3-phenyl-N- | 3 | 623.3 |
| | (2-(4-phenyl-4-(pyrrolidin-1-yl)cyclohexyl)ethyl)- | | |
| | propanamide | | |
| 41 | 4-Methoxy-N,2,6-trimethyl-N-(2-(2-(4-(4- | 4 | 572.3 |
| | methylpiperazin-1-yl)-4-phenylpiperidin-1-yl)-2- | | |
| | oxoethoxy)ethyl)benzenesulfonamide | | |
| 42 | N-(2-(2-(4-(4-Fluorophenyl)-4-(4-methylpiperazin-1- | 4 | 590.3 |
| | yl)piperidin-1-yl)-2-oxoethoxy)ethyl)-4-methoxy-N,2,6- | | |
| | trimethylbenzenesulfonamide | | |
| 43 | N-Methyl-N-(2-(4-phenethyl-4-(pyrrolidin-1- | 3 | 647.3 |
| | yl)cyclohexyl)ethyl)-2-(1-(3-(trifluoromethyl)- | | |
| | phenylsulfonyl)piperidin-2-yl)acetamide | | |
| 44 | N-(2-(2-(4-(3-Fluorophenyl)-4-(4-methylpiperazin-1- | 4 | 590.3 |
| | yl)piperidin-1-yl)-2-oxoethoxy)ethyl)-4-methoxy-N,2,6- | | |
| | trimethylbenzenesulfonamide | | |
| 45 | N-((4-Benzyl-4-(pyrrolidin-1-yl)cyclohexyl)methyl)-2- | 3 | 599.3 |
| | (2-(4-methoxy-N,2,6-trimethylphenylsulfonamido)- | | |
| | ethoxy)-N-methylacetamide | | |
| 46 | 2-((1-(4-Methoxy-2,6-dimethylphenylsulfonyl)- | 3 | 667.4 |
| | piperidin-2-yl)methoxy)-N-methyl-N-(2-(4-phenethyl- | | |
| | 4-(pyrrolidin-1-yl)cyclohexyl)ethyl)acetamide | | |
| 47 | 2-(2-(4-Methoxy-N,2,6-trimethylphenylsulfonamido)- | 3 | 613.4 |
| | ethoxy)-N-methyl-N-((4-phenethyl-4-(pyrrolidin-1- | | |
| | yl)cyclohexyl)methyl)acetamide | | |
| 48 | N-(2-(2-(4-(Dimethylamino)-4-phenethylpiperidin-1- | 4 | 545.3 |
| | yl)-2-oxoethoxy)ethyl)-4-methoxy-N,2,6- | | |
| | trimethylbenzenesulfonamide | | |
| 49 | 2-((1-(4-Methoxy-2,6-dimethylphenylsulfonyl)- | 4 | 612.3 |
| | piperidin-2-yl)methoxy)-1-(4-(4-methylpiperazin-1-yl)- | | |
| | 4-phenylpiperidin-1-yl)ethanone | | |
| 50 | 1-(4-(Dimethylamino)-4-phenethylpiperidin-1-yl)-2- | 4 | 585.3 |
| | ((1-(4-methoxy-2,6-dimethylphenylsulfonyl)piperidin- | | |
| | 2-yl)methoxy)ethanone | | |
| 51 | 2-((1-(4-Methoxy-2,6-dimethylphenylsulfonyl)- | 3 | 653.4 |
| | piperidin-2-yl)methoxy)-N-methyl-N-((4-phenethyl-4- | | |
| | (pyrrolidin-1-yl)cyclohexyl)methyl)acetamide | | |
| 52 | N-(2-(4-(Dimethylamino)-4-phenethylcyclohexyl)- | 3 | 601.4 |
| | ethyl)-2-(2-(4-methoxy-N,2,6-trimethylphenyl- | | |
| | sulfonamido)ethoxy)-N-methylacetamide | | |
| 53 | N-(2-(4-Benzyl-4-(dimethylamino)cyclohexyl)ethyl)-2- | 3 | 587.3 |
| | (2-(4-methoxy-N,2,6-trimethylphenylsulfonamido)- | | |
| | ethoxy)-N-methylacetamide | | |
| 54 | N-(2-(2-(4-(Dimethylamino)-4-phenylpiperidin-1-yl)-2- | 4 | 517.3 |
| | oxoethoxy)ethyl)-4-methoxy-N,2,6-trimethylbenzene- | | |
| | sulfonamide | | |
| 55 | N-(3-(4-(4-Methylpiperazin-1-yl)-4-phenethylpiperidin- | 4 | 624.3 |
| | 1-yl)-3-oxo-1-phenylpropyl)naphthalene-2- | | |
| | sulfonamide | | |
| 56 | N-(2-(4-Benzyl-4-(pyrrolidin-1-yl)cyclohexyl)ethyl)-N- | 3 | 637.3 |
| | methyl-3-(naphthalene-2-sulfonamido)-3- | | |
| | phenylpropanamide | | |
| 57 | 1-(4-Benzyl-4-(4-methylpiperazin-1-yl)piperidin-1-yl)- | 4 | 626.4 |
| | 2-((1-(4-methoxy-2,6-dimethylphenylsulfonyl)- | | |
| | piperidin-2-yl)methoxy)ethanone | | |
| 58 | 2-(2-(4-Methoxy-N,2,6-trimethylphenylsulfonamido)- | 3 | 627.4 |
| | ethoxy)-N-methyl-N-(2-(4-phenethyl-4-(pyrrolidin-1- | | |
| | yl)cyclohexyl)ethyl)acetamide | | |
| 59 | 2-((1-(4-Methoxy-2,6-dimethylphenylsulfonyl)- | 3 | 681.4 |
| | piperidin-2-yl)methoxy)-N-methyl-N-(3-(4-phenethyl- | | |
| | 4-(pyrrolidin-1-yl)cyclohexyl)propyl)acetamide | | |
| 60 | 2-((1-(4-Methoxy-2,6-dimethylphenylsulfonyl)- | 3 | 639.4 |
| | piperidin-2-yl)methoxy)-N-methyl-N-(2-(4-phenyl-4- | | |
| | (pyrrolidin-1-yl)cyclohexyl)ethyl)acetamide | | |
| 61 | N-(2-(4-Benzyl-4-(dimethylamino)cyclohexyl)ethyl)-2- | 3 | 685.3 |
| | ((1-(3,4-dichlorophenylsulfonyl)-1,2,3,4- | | |
| | tetrahydroquinolin-2-yl)methoxy)-N-methylacetamide | | |
| 62 | 2-((1-(4-Methoxy-2,6-dimethylphenylsulfonyl)- | 4 | 675.4 |
| | piperidin-2-yl)methoxy)-1-(4-phenyl-4-(4-(pyridin-4- | | |
| | yl)piperazin-1-yl)piperidin-1-yl)ethanone | | |
| 63 | N-(2-(2-(4-Benzyl-4-(dimethylamino)piperidin-1-yl)-2- | 4 | 531.3 |
| | oxoethoxy)ethyl)-4-methoxy-N,2,6- | | |
| | trimethylbenzenesulfonamide | | |
| 64 | 1-(4-(4-Methylpiperazin-1-yl)-4-phenylpiperidin-1-yl)- | 4 | 592.3 |
| | 2-(1-(3-(trifluoromethyl)phenylsulfonyl)piperidin-2- | | |
| | yl)ethanone | | |
| 65 | 1-(4-Benzyl-4-(dimethylamino)piperidin-1-yl)-2-((1-(4- | 4 | 571.3 |
| | methoxy-2,6-dimethylphenylsulfonyl)piperidin-2- | | |
| | yl)methoxy)ethanone | | |
| 66 | 2-((1-(4-Methoxy-2,6-dimethylphenylsulfonyl)- | 3 | 625.4 |
| | piperidin-2-yl)methoxy)-N-methyl-N-((4-phenyl-4- | | |
| | (pyrrolidin-1-yl)cyclohexyl)methyl)acetamide | | |
| 67 | 1-(4-(3-Fluorophenyl)-4-(4-methylpiperazin-1-yl)- | 4 | 630.3 |
| | piperidin-1-yl)-2-((1-(4-methoxy-2,6-dimethylphenyl- | | |
| | sulfonyl)piperidin-2-yl)methoxy)ethanone | | |
| 68 | N-(2-(4-Benzyl-4-(pyrrolidin-1-yl)cyclohexyl)ethyl)-2- | 3 | 653.4 |
| | ((1-(4-methoxy-2,6-dimethylphenylsulfonyl)piperidin- | | |
| | 2-yl)methoxy)-N-methylacetamide | | |
| 69 | 4-Methoxy-N,2,6-trimethyl-N-(2-(2-oxo-2-(4- | 4 | 635.3 |
| | phenyl-4-(4-(pyridin-4-yl)piperazin-1-yl)- | | |
| | piperidin-1-yl)ethoxy)ethyl)benzene- | | |
| | sulfonamide | | |
| 70 | 1-(4-(4-Fluorophenyl)-4-(4-methylpiperazin-1-yl)- | 4 | 630.3 |
| | piperidin-1-yl)-2-((1-(4-methoxy-2,6-dimethylphenyl- | | |
| | sulfonyl)piperidin-2-yl)methoxy)ethanone | | |
| 71 | 1-(4-(Dimethylamino)-4-phenethylpiperidin-1-yl)-2-(1- | 4 | 565.3 |
| | (3-(trifluoromethyl)phenylsulfonyl)piperidin-2-yl)- | | |
| | ethanone | | |
| 72 | N-(2-(4-(Dimethylamino)-4-phenylcyclohexyl)ethyl)-2- | 3 | 573.3 |
| | (2-(4-methoxy-N,2,6-trimethylphenylsulfonamido)- | | |
| | ethoxy)-N-methylacetamide | | |
| 73 | N-(2-(4-(Dimethylamino)-4-phenethylcyclohexyl)- | 3 | 641.4 |
| | ethyl)-2-((1-(4-methoxy-2,6-dimethylphenyl- | | |
| | sulfonyl)piperidin-2-yl)methoxy)-N-methylacetamide | | |
| 74 | N-(3-(4-Benzyl-4-(pyrrolidin-1-yl)cyclohexyl)propyl)- | 3 | 651.4 |
| | N-methyl-3-(naphthalene-2-sulfonamido)-3- | | |
| | phenylpropanamide | | |
| 75 | 1-(4-(Dimethylamino)-4-phenylpiperidin-1-yl)-2-((1-(4- | 4 | 557.3 |
| | methoxy-2,6-dimethylphenylsulfonyl)piperidin-2- | | |
| | yl)methoxy)ethanone | | |
| 76 | N-(2-(4-(Dimethylamino)-4-phenethylcyclohexyl)- | 3 | 621.3 |
| | ethyl)-N-methyl-2-(1-(3-(trifluoromethyl)phenyl- | | |
| | sulfonyl)piperidin-2-yl)acetamide | | |
| 77 | 2-((1-(4-Methoxy-2,6-dimethylphenylsulfonyl)- | 4 | 640.4 |
| | piperidin-2-yl)methoxy)-1-(4-(4-methylpiperazin-1-yl)- | | |
| | 4-phenethylpiperidin-1-yl)ethanone | | |
| 78 | N-((4-Benzyl-4-(pyrrolidin-1-yl)cyclohexyl)methyl)-2- | 3 | 639.4 |
| | ((1-(4-methoxy-2,6-dimethylphenylsulfonyl)piperidin- | | |
| | 2-yl)methoxy)-N-methylacetamide | | |
| 79 | N-Methyl-3-(naphthalene-2-sulfonamido)-N-(3-(4- | 3 | 665.4 |
| | phenethyl-4-(pyrrolidin-1-yl)cyclohexyl)propyl)-3- | | |
| | phenylpropanamide | | |
| 80 | 1-(4-Benzyl-4-(dimethylamino)piperidin-1-yl)-3-(1-(4- | 4 | 559.3 |
| | chloro-2,5-dimethylphenylsulfonyl)piperidin-2- | | |
| | yl)propan-1-one | | |
| 81 | N-(2-(4-(Dimethylamino)-4-phenylcyclohexyl)ethyl)-2- | 3 | 613.4 |
| | ((1-(4-methoxy-2,6-dimethylphenylsulfonyl)piperidin- | | |
| | 2-yl)methoxy)-N-methylacetamide | | |
| 82 | N-(3-(4-(4-Methylpiperazin-1-yl)-4-phenylpiperidin-1- | 4 | 596.3 |
| | yl)-3-oxo-1-phenylpropyl)naphthalene-2-sulfonamide | | |
| 83 | 2-(2-(4-Methoxy-N,2,6-trimethylphenylsulfonamido)- | 3 | 613.4 |
| | ethoxy)-N-methyl-N-(3-(4-phenyl-4-(pyrrolidin-1-yl)- | | |
| | cyclohexyl)propyl)acetamide | | |
| 84 | N-(2-(4-Benzyl-4-(dimethylamino)cyclohexyl)ethyl)-2- | 3 | 627.4 |
| | ((1-(4-methoxy-2,6-dimethylphenylsulfonyl)piperidin- | | |
| | 2-yl)methoxy)-N-methylacetamide | | |
| 85 | N-(3-(4-Benzyl-4-(pyrrolidin-1-yl)cyclohexyl)propyl)-2- | 3 | 627.4 |
| | (2-(4-methoxy-N,2,6-trimethylphenylsulfonamido)- | | |
| | ethoxy)-N-methylacetamide | | |
| 86 | N-(2-(4-Benzyl-4-(dimethylamino)cyclohexyl)ethyl)-N- | 3 | 611.3 |
| | methyl-3-(naphthalene-2-sulfonamido)-3-phenyl- | | |
| | propanamide | | |
| 87 | 4-Methoxy-N,2,6-trimethyl-N-(2-(2-(4-(4- | 4 | 600.3 |
| | methylpiperazin-1-yl)-4-phenethylpiperidin-1-yl)-2- | | |
| | oxoethoxy)ethyl)benzenesulfonamide | | |
| 88 | N-(3-(4-Benzyl-4-(pyrrolidin-1-yl)cyclohexyl)propyl)-2- | 3 | 667.4 |
| | ((1-(4-methoxy-2,6-dimethylphenylsulfonyl)piperidin- | | |
| | 2-yl)methoxy)-N-methylacetamide | | |
| 89 | N-Methyl-3-(naphthalene-2-sulfonamido)-N-((4- | 3 | 637.3 |
| | phenethyl-4-(pyrrolidin-1-yl)cyclohexyl)methyl)-3- | | |
| | phenylpropanamide | | |
| 90 | N-(2-(4-(Dimethylamino)-4-phenylcyclohexyl)ethyl)-N- | 3 | 597.3 |
| | methyl-3-(naphthalene-2-sulfonamido)-3- | | |
| | phenylpropanamide | | |
| 91 | 2-(2-(4-Methoxy-N,2,6-trimethylphenylsulfonamido)- | 3 | 641.4 |
| | ethoxy)-N-methyl-N-(3-(4-phenethyl-4-(pyrrolidin-1- | | |
| | yl)cyclohexyl)propyl)acetamide | | |
| 92 | N-Methyl-3-(naphthalene-2-sulfonamido)-3-phenyl-N- | 3 | 609.3 |
| | ((4-phenyl-4-(pyrrolidin-1-yl)cyclohexyl)methyl)- | | |
| | propanamide | | |
| 93 | N-(3-(4-Benzyl-4-(pyrrolidin-1-yl)cyclohexyl)propyl)- | 3 | 647.3 |
| | N-methyl-2-(1-(3-(trifluoromethyl)phenylsulfonyl)- | | |
| | piperidin-2-yl)acetamide | | |
| 94 | N-((4-Benzyl-4-(pyrrolidin-1-yl)cyclohexyl)methyl)-N- | 3 | 619.3 |
| | methyl-2-(1-(3-(trifluoromethyl)phenylsulfonyl)- | | |
| | piperidin-2-yl)acetamide | | |
| 95 | N-(2-(4-Benzyl-4-(dimethylamino)cyclohexyl)ethyl)-N- | 3 | 607.3 |
| | methyl-2-(1-(3-(trifluoromethyl)phenylsulfonyl)- | | |
| | piperidin-2-yl)acetamide | | |
| 96 | N-(3-(4-(4-Fluorophenyl)-4-(4-methylpiperazin-1- | 4 | 614.3 |
| | yl)piperidin-1-yl)-3-oxo-1-phenylpropyl)naphthalene- | | |
| | 2-sulfonamide | | |
| 97 | 1-(4-Benzyl-4-(dimethylamino)piperidin-1-yl)-2-(1-(3- | 4 | 551.2 |
| | (trifluoromethyl)phenylsulfonyl)piperidin-2- | | |
| | yl)ethanone | | |
| 98 | N-(3-Oxo-1-phenyl-3-(4-phenyl-4-(4-(pyridin-4- | 4 | 659.3 |
| | yl)piperazin-1-yl)piperidin-1-yl)propyl)naphthalene-2- | | |
| | sulfonamide | | |
| 99 | 3-(1-(4-Chloro-2,5-dimethylphenylsulfonyl)piperidin-2- | 4 | 663.3 |
| | yl)-1-(4-phenyl-4-(4-(pyridin-4-yl)piperazin-1- | | |
| | yl)piperidin-1-yl)propan-1-one | | |
| 100 | 2-((1-(3,4-Dichlorophenylsulfonyl)-1,2,3,4- | 4 | 733.2 |
| | tetrahydroquinolin-2-yl)methoxy)-1-(4-phenyl-4-(4- | | |
| | (pyridin-4-yl)piperazin-1-yl)piperidin-1-yl)ethanone | | |
| 101 | 2-((1-(3,4-Dichlorophenylsulfonyl)-1,2,3,4-tetrahydro- | 3 | 699.3 |
| | quinolin-2-yl)methoxy)-N-(2-(4-(dimethylamino)-4- | | |
| | phenethylcyclohexyl)ethyl)-N-methylacetamide | | |
| 102 | N-(2-(4-(Dimethylamino)-4-phenethylcyclohexyl)- | 3 | 625.3 |
| | ethyl)-N-methyl-3-(naphthalene-2-sulfonamido)-3- | | |
| | phenylpropanamide | | |
| 103 | 2-((1-(3,4-Dichlorophenylsulfonyl)-1,2,3,4-tetrahydro- | 4 | 688.2 |
| | quinolin-2-yl)methoxy)-1-(4-(3-fluorophenyl)-4-(4- | | |
| | methylpiperazin-1-yl)piperidin-1-yl)ethanone | | |
| 104 | N-(2-(4-Benzyl-4-(pyrrolidin-1-yl)cyclohexyl)ethyl)-2- | 3 | 711.3 |
| | ((1-(3,4-dichlorophenylsulfonyl)-1,2,3,4- | | |
| | tetrahydroquinolin-2-yl)methoxy)-N-methylacetamide | | |
| 105 | N-(3-(4-(Dimethylamino)-4-phenethylpiperidin-1-yl)-3- | 4 | 569.3 |
| | oxo-1-phenylpropyl)naphthalene-2-sulfonamide | | |
| 106 | N-Methyl-N-(2-(4-phenyl-4-(pyrrolidin-1-yl)- | 3 | 619.3 |
| | cyclohexyl)ethyl)-2-(1-(3-(trifluoromethyl)phenyl- | | |
| | sulfonyl)piperidin-2-yl)acetamide | | |
| 107 | 3-(1-(4-Chloro-2,5-dimethylphenylsulfonyl)piperidin-2- | 3 | 601.3 |
| | yl)-N-(2-(4-(dimethylamino)-4-phenylcyclohexyl)- | | |
| | ethyl)-N-methylpropanamide | | |
| 108 | 1-(4-Benzyl-4-(4-methylpiperazin-1-yl)piperidin-1-yl)- | 4 | 684.2 |
| | 2-((1-(3,4-dichlorophenylsulfonyl)-1,2,3,4- | | |
| | tetrahydroquinolin-2-yl)methoxy)ethanone | | |
| 109 | 2-((1-(3,4-Dichlorophenylsulfonyl)-1,2,3,4- | 4 | 615.2 |
| | tetrahydroquinolin-2-yl)methoxy)-1-(4- | | |
| | (Dimethylamino)-4-phenylpiperidin-1-yl)ethanone | | |
| 110 | 3-(1-(4-Chloro-2,5-dimethylphenylsulfonyl)piperidin-2- | 4 | 600.3 |
| | yl)-1-(4-(4-methylpiperazin-1-yl)-4-phenylpiperidin-1- | | |
| | yl)propan-1-one | | |
| 111 | 2-((1-(3,4-Dichlorophenylsulfonyl)-1,2,3,4-tetrahydro- | 3 | 671.2 |
| | quinolin-2-yl)methoxy)-N-(2-(4-(dimethylamino)-4- | | |
| | phenylcyclohexyl)ethyl)-N-methylacetamide | | |
| 112 | N-((4-Benzyl-4-(pyrrolidin-1-yl)cyclohexyl)methyl)-N- | 3 | 623.3 |
| | methyl-3-(naphthalene-2-sulfonamido)-3-phenyl- | | |
| | propanamide | | |
| 113 | 1-(4-Phenyl-4-(4-(pyridin-4-yl)piperazin-1-yl)piperidin- | 4 | 655.3 |
| | 1-yl)-2-(1-(3-(trifluoromethyl)phenylsulfonyl)piperidin- | | |
| | 2-yl)ethanone | | |
| 114 | N-Methyl-3-(naphthalene-2-sulfonamido)-N-(2-(4- | 3 | 651.4 |
| | phenethyl-4-(pyrrolidin-1-yl)cyclohexyl)ethyl)-3- | | |
| | phenylpropanamide | | |
| 115 | 2-((1-(3,4-Dichlorophenylsulfonyl)-1,2,3,4- | 4 | 670.2 |
| | tetrahydroquinolin-2-yl)methoxy)-1-(4-(4- | | |
| | methylpiperazin-1-yl)-4-phenylpiperidin-1-yl)ethanone | | |
| 116 | 2-((1-(3,4-Dichlorophenylsulfonyl)-1,2,3,4-tetrahydro- | 4 | 698.3 |
| | quinolin-2-yl)methoxy)-1-(4-(4-methylpiperazin-1-yl)- | | |
| | 4-phenethylpiperidin-1-yl)ethanone | | |
| 117 | N-(2-(4-Benzyl-4-(pyrrolidin-1-yl)cyclohexyl)ethyl)-3- | 3 | 641.3 |
| | (1-(4-chloro-2,5-dimethylphenylsulfonyl)piperidin-2- | | |
| | yl)-N-methylpropanamide | | |
| 118 | N-(3-(4-Benzyl-4-(4-methylpiperazin-1-yl)piperidin-1- | 4 | 610.3 |
| | yl)-3-oxo-1-phenylpropyl)naphthalene-2-sulfonamide | | |
| 119 | 1-(4-(4-Methylpiperazin-1-yl)-4-phenethylpiperidin-1- | 4 | 620.3 |
| | yl)-2-(1-(3-(trifluoromethyl)phenylsulfonyl)piperidin-2- | | |
| | yl)ethanone | | |
| 120 | N-(3-(4-(3-Fluorophenyl)-4-(4-methylpiperazin-1- | 4 | 614.3 |
| | yl)piperidin-1-yl)-3-oxo-1-phenylpropyl)naphthalene- | | |
| | 2-sulfonamide | | |
| 121 | N-(2-(4-Benzyl-4-(pyrrolidin-1-yl)cyclohexyl)ethyl)-N- | 3 | 633.3 |
| | methyl-2-(1-(3-(trifluoromethyl)phenylsulfonyl)- | | |
| | piperidin-2-yl)acetamide | | |
| 122 | 3-(1-(4-Chloro-2,5-dimethylphenylsulfonyl)piperidin-2- | 4 | 628.3 |
| | yl)-1-(4-(4-methylpiperazin-1-yl)-4-phenethylpiperidin- | | |
| | 1-yl)propan-1-one | | |
| 123 | 3-(1-(4-Chloro-2,5-dimethylphenylsulfonyl)piperidin-2- | 4 | 545.3 |
| | yl)-1-(4-(dimethylamino)-4-phenylpiperidin-1- | | |
| | yl)propan-1-one | | |
| 124 | N-(3-(4-Benzyl-4-(pyrrolidin-1-yl)cyclohexyl)propyl)-3- | 3 | 655.4 |
| | (1-(4-chloro-2,5-dimethylphenylsulfonyl)piperidin-2- | | |
| | yl)-N-methylpropanamide | | |
| 125 | N-(2-(4-(Dimethylamino)-4-phenylcyclohexyl)ethyl)-N- | 3 | 593.3 |
| | methyl-2-(1-(3-(trifluoromethyl)phenylsulfonyl)- | | |
| | piperidin-2-yl)acetamide | | |
| 126 | 2-((1-(3,4-Dichlorophenylsulfonyl)-1,2,3,4-tetrahydro- | 4 | 688.2 |
| | quinolin-2-yl)methoxy)-1-(4-(4-fluorophenyl)-4-(4- | | |
| | methylpiperazin-1-yl)piperidin-1-yl)ethanone | | |
| 127 | N-((4-Benzyl-4-(pyrrolidin-1-yl)cyclohexyl)methyl)-2- | 3 | 697.3 |
| | ((1-(3,4-dichlorophenylsulfonyl)-1,2,3,4- | | |
| | tetrahydroquinolin-2-yl)methoxy)-N-methylacetamide | | |
| 128 | N-Methyl-3-(naphthalene-2-sulfonamido)-3-phenyl-N- | 3 | 637.3 |
| | (3-(4-phenyl-4-(pyrrolidin-1-yl)cyclohexyl)propyl)- | | |
| | propanamide | | |
| 129 | N-((4-Benzyl-4-(pyrrolidin-1-yl)cyclohexyl)methyl)-3- | 3 | 627.3 |
| | (1-(4-chloro-2,5-dimethylphenylsulfonyl)piperidin-2- | | |
| | yl)-N-methylpropanamide | | |
| 130 | 1-(4-Benzyl-4-(4-methylpiperazin-1-yl)piperidin-1-yl)- | 4 | 614.3 |
| | 3-(1-(4-chloro-2,5-dimethylphenylsulfonyl)piperidin-2- | | |
| | yl)propan-1-one | | |
| 131 | 3-(1-(4-Chloro-2,5-dimethylphenylsulfonyl)piperidin-2- | 3 | 655.4 |
| | yl)-N-methyl-N-(2-(4-phenethyl-4-(pyrrolidin-1- | | |
| | yl)cyclohexyl)ethyl)propanamide | | |
| 132 | 3-(1-(4-Chloro-2,5-dimethylphenylsulfonyl)piperidin-2- | 3 | 629.3 |
| | yl)-N-(2-(4-(dimethylamino)-4-phenethylcyclohexyl)- | | |
| | ethyl)-N-methylpropanamide | | |
| 133 | 2-((1-(3,4-Dichlorophenylsulfonyl)-1,2,3,4- | 3 | 711.3 |
| | tetrahydroquinolin-2-yl)methoxy)-N-methyl-N-(3-(4- | | |
| | phenyl-4-(pyrrolidin-1-yl)cyclohexyl)propyl)acetamide | | |
| 134 | N-Methyl-N-(3-(4-phenethyl-4-(pyrrolidin-1- | 3 | 661.4 |
| | yl)cyclohexyl)propyl)-2-(1-(3-(trifluoromethyl)- | | |
| | phenylsulfonyl)piperidin-2-yl)acetamide | | |
| 135 | 3-(1-(4-Chloro-2,5-dimethylphenylsulfonyl)piperidin-2- | 4 | 618.3 |
| | yl)-1-(4-(3-fluorophenyl)-4-(4-methylpiperazin-1- | | |
| | yl)piperidin-1-yl)propan-1-one | | |
| 136 | 3-(1-(4-Chloro-2,5-dimethylphenylsulfonyl)piperidin-2- | 3 | 641.3 |
| | yl)-N-methyl-N-(3-(4-phenyl-4-(pyrrolidin-1- | | |
| | yl)cyclohexyl)propyl)propanamide | | |
| 137 | 3-(1-(4-Chloro-2,5-dimethylphenylsulfonyl)piperidin-2- | 4 | 573.3 |
| | yl)-1-(4-(dimethylamino)-4-phenethylpiperidin-1- | | |
| | yl)propan-1-one | | |
| 138 | 3-(1-(4-Chloro-2,5-dimethylphenylsulfonyl)piperidin-2- | 4 | 618.3 |
| | yl)-1-(4-(4-fluorophenyl)-4-(4-methylpiperazin-1- | | |
| | yl)piperidin-1-yl)propan-1-one | | |
| 139 | 3-(1-(4-Chloro-2,5-dimethylphenylsulfonyl)piperidin-2- | 3 | 627.3 |
| | yl)-N-methyl-N-(2-(4-phenyl-4-(pyrrolidin-1- | | |
| | yl)cyclohexyl)ethyl)propanamide | | |
| 140 | 3-(1-(4-Chloro-2,5-dimethylphenylsulfonyl)piperidin-2- | 3 | 613.3 |
| | yl)-N-methyl-N-((4-phenyl-4-(pyrrolidin-1- | | |
| | yl)cyclohexyl)methyl)propanamide | | |
| 141 | 3-(1-(4-Chloro-2,5-dimethylphenylsulfonyl)piperidin-2- | 3 | 641.3 |
| | yl)-N-methyl-N-((4-phenethyl-4-(pyrrolidin-1- | | |
| | yl)cyclohexyl)methyl)propanamide | | |
| 142 | N-(4-Phenyl-4-(pyrrolidin-1-yl)cyclohexyl)-2-((1- | 2 | 641.2 |
| | (2,4,6-trichlorophenylsulfonyl)piperidin-2- | | |
| | yl)methoxy)acetamide | | |
| 143 | N-((4-Benzyl-4-(4-methylpiperazin-1-yl)cyclohexyl)- | 2 | 668.4 |
| | methyl)-2-((1-(4-methoxy-2,6-dimethylphenyl- | | |
| | sulfonyl)piperidin-2-yl)methoxy)-N-methylacetamide | | |
| 144 | 2-((1-(4-Methoxy-2,6-dimethylphenylsulfonyl)- | 2 | 682.4 |
| | piperidin-2-yl)methoxy)-N-methyl-N-((4-(4-methyl- | | |
| | piperazin-1-yl)-4-phenethylcyclohexyl)methyl)- | | |
| | acetamide | | |
| 145 | 2-((1-(4-Methoxy-2,6- | 2 | 583.3 |
| | dimethylphenylsulfonyl)pyrrolidin-2-yl)methoxy)-N-(4- | | |
| | phenyl-4-(pyrrolidin-1-yl)cyclohexyl)acetamide | | |
| 146 | N-(4-Phenyl-4-(pyrrolidin-1-yl)cyclohexyl)-2-((1- | 2 | 627.2 |
| | (2,4,6-trichlorophenylsulfonyl)pyrrolidin-2- | | |
| | yl)methoxy)acetamide | | |
| 147 | 2-((1-(4-Methoxy-2,6-dimethylphenylsulfonyl)- | 2 | 597.3 |
| | piperidin-2-yl)methoxy)-N-(4-phenyl-4-(pyrrolidin-1- | | |
| | yl)cyclohexyl)acetamide | | |
| 148 | 2-((1-(4-Methoxy-2,6-dimethylphenylsulfonyl)-pyrrolidin- | 2 | 668.4 |
| | 2-yl)methoxy)-N-methyl-N-((4-(4-methyl-piperazin- | | |
| | 1-yl)-4-phenethylcyclohexyl)methyl)-acetamide | | |
| 149 | N-(4-Benzyl-4-(pyrrolidin-1-yl)cyclohexyl)-2-((1-(4- | 2 | 611.3 |
| | methoxy-2,6-dimethylphenylsulfonyl)piperidin-2- | | |
| | yl)methoxy)acetamide | | |
| 150 | 2-(2-(4-Methoxy-N,2,6-trimethylphenylsulfonamido)- | 2 | 557.3 |
| | ethoxy)-N-(4-phenyl-4-(pyrrolidin-1-yl)cyclohexyl)- | | |
| | acetamide | | |
| 151 | N-(4-Benzyl-4-(pyrrolidin-1-yl)cyclohexyl)-2-((1-(4- | 2 | 597.3 |
| | methoxy-2,6-dimethylphenylsulfonyl)pyrrolidin-2- | | |
| | yl)methoxy)acetamide | | |
| 152 | 2-((1-(4-Methoxy-2,6-dimethylphenylsulfonyl)- | 2 | 613.3 |
| | pyrrolidin-2-yl)methoxy)-N-((4-morpholino-4- | | |
| | phenylcyclohexyl)methyl)acetamide | | |
| 153 | 2-(1-(4-Methoxy-2,6-dimethylphenylsulfonyl)- | 2 | 569.3 |
| | pyrrolidin-3-yloxy)-N-(4-phenyl-4-(pyrrolidin-1- | | |
| | yl)cyclohexyl)acetamide | | |
| 154 | N-((4-Benzyl-4-(4-methylpiperazin-1-yl)cyclohexyl)- | 2 | 654.4 |
| | methyl)-2-((1-(4-methoxy-2,6-dimethylphenyl- | | |
| | sulfonyl)pyrrolidin-2-yl)methoxy)-N-methylacetamide | | |
| 155 | N-(4-Benzyl-4-morpholinocyclohexyl)-2-((1-(4- | 2 | 627.3 |
| | methoxy-2,6-dimethylphenylsulfonyl)piperidin-2- | | |
| | yl)methoxy)acetamide | | |
| 156 | N-(4-Benzyl-4-(pyrrolidin-1-yl)cyclohexyl)-2-((1- | 2 | 641.2 |
| | (2,4,6-trichlorophenylsulfonyl)pyrrolidin-2- | | |
| | yl)methoxy)acetamide | | |
| 157 | 2-((1-(4-Methoxy-2,6-dimethylphenylsulfonyl)- | 2 | 627.3 |
| | piperidin-2-yl)methoxy)-N-((4-morpholino-4- | | |
| | phenylcyclohexyl)methyl)acetamide | | |
| 158 | 2-((1-(4-Methoxy-2,6-dimethylphenylsulfonyl)- | 2 | 613.3 |
| | piperidin-2-yl)methoxy)-N-(4-morpholino-4- | | |
| | phenylcyclohexyl)acetamide | | |
| 159 | N-(4-Benzyl-4-morpholinocyclohexyl)-2-((1-(4- | 2 | 613.3 |
| | methoxy-2,6-dimethylphenylsulfonyl)pyrrolidin-2- | | |
| | yl)methoxy)acetamide | | |
| 160 | 2-((1-(4-Methoxy-2,6-dimethylphenylsulfonyl)- | 2 | 599.3 |
| | pyrrolidin-2-yl)methoxy)-N-(4-morpholino-4- | | |
| | phenylcyclohexyl)acetamide | | |
| 161 | N-Methyl-N-((4-(4-methylpiperazin-1-yl)-4-phenethyl- | 2 | 726.3 |
| | cyclohexyl)methyl)-2-((1-(2,4,6-trichlorophenyl- | | |
| | sulfonyl)piperidin-2-yl)methoxy)acetamide | | |
| 162 | N-((4-Benzyl-4-(4-methylpiperazin-1-yl)- | 2 | 712.2 |
| | cyclohexyl)methyl)-N-methyl-2-((1-(2,4,6- | | |
| | trichlorophenylsulfonyl)piperidin-2- | | |
| | yl)methoxy)acetamide | | |
| 163 | N-(4-Benzyl-4-(pyrrolidin-1-yl)cyclohexyl)-2-((1- | 2 | 655.2 |
| | (2,4,6-trichlorophenylsulfonyl)piperidin-2- | | |
| | yl)methoxy)acetamide | | |
| 164 | N-(4-Morpholino-4-phenylcyclohexyl)-2-((1-(2,4,6- | 2 | 643.1 |
| | trichlorophenylsulfonyl)pyrrolidin-2- | | |
| | yl)methoxy)acetamide | | |
| 165 | 2-(2-(4-Methoxy-N,2,6-trimethylphenylsulfonamido)- | 2 | 642.4 |
| | ethoxy)-N-methyl-N-((4-(4-methylpiperazin-1-yl)-4- | | |
| | phenethylcyclohexyl)methyl)acetamide | | |
| 166 | N-(4-Benzyl-4-(pyrrolidin-1-yl)cyclohexyl)-2-(1-(4- | 2 | 583.3 |
| | methoxy-2,6-dimethylphenylsulfonyl)pyrrolidin-3- | | |
| | yloxy)acetamide | | |
| 167 | 2-(2-(4-Methoxy-N,2,6-trimethylphenylsulfonamido)- | 2 | 573.3 |
| | ethoxy)-N-(4-morpholino-4-phenylcyclohexyl)- | | |
| | acetamide | | |
| 168 | N-((4-Benzyl-4-(4-methylpiperazin-1-yl)cyclohexyl)- | 2 | 698.2 |
| | methyl)-N-methyl-2-((1-(2,4,6-trichlorophenyl- | | |
| | sulfonyl)pyrrolidin-2-yl)methoxy)acetamide | | |
| 169 | N-((4-Benzyl-4-morpholinocyclohexyl)methyl)-2-((1- | 2 | 627.3 |
| | (4-methoxy-2,6-dimethylphenylsulfonyl)pyrrolidin-2- | | |
| | yl)methoxy)acetamide | | |
| 170 | N-(4-Benzyl-4-(pyrrolidin-1-yl)cyclohexyl)-2-(2-(4- | 2 | 571.3 |
| | methoxy-N,2,6-trimethylphenylsulfonamido)ethoxy)- | | |
| | acetamide | | |
| 171 | N-(4-Benzyl-4-morpholinocyclohexyl)-2-(2-(4- | 2 | 587.3 |
| | methoxy-N,2,6-trimethylphenylsulfonamido)ethoxy)- | | |
| | acetamide | | |
| 172 | N-Methyl-N-((4-(4-methylpiperazin-1-yl)-4-phenethyl- | 2 | 712.2 |
| | cyclohexyl)methyl)-2-((1-(2,4,6-trichlorophenyl- | | |
| | sulfonyl)pyrrolidin-2-yl)methoxy)acetamide | | |
| 173 | 2-(1-(4-Methoxy-2,6-dimethylphenylsulfonyl)- | 2 | 599.3 |
| | pyrrolidin-3-yloxy)-N-((4-morpholino-4- | | |
| | phenylcyclohexyl)methyl)acetamide | | |
| 174 | N-((4-Benzyl-4-(4-methylpiperazin-1-yl)cyclohexyl)- | 2 | 640.4 |
| | methyl)-2-(1-(4-methoxy-2,6-dimethyl- | | |
| | phenylsulfonyl)pyrrolidin-3-yloxy)-N-methylacetamide | | |
| 175 | N-((4-Benzyl-4-(4-methylpiperazin-1- | 2 | 698.2 |
| | yl)cyclohexyl)methyl)-N-methyl-2-(1-(2,4,6- | | |
| | trichlorophenylsulfonyl)piperidin-3-yloxy)acetamide | | |
| 176 | 2-(1-(4-Methoxy-2,6-dimethylphenylsulfonyl)piperidin- | 2 | 583.3 |
| | 3-yloxy)-N-(4-phenyl-4-(pyrrolidin-1- | | |
| | yl)cyclohexyl)acetamide | | |
| 177 | 2-(1-(4-Methoxy-2,6-dimethylphenylsulfonyl)- | 2 | 654.4 |
| | pyrrolidin-3-yloxy)-N-methyl-N-((4-(4-methylpiperazin- | | |
| | 1-yl)-4-phenethylcyclohexyl)methyl)acetamide | | |
| 178 | N-(4-Phenyl-4-(pyrrolidin-1-yl)cyclohexyl)-2-(1-(2,4,6- | 2 | 613.1 |
| | trichlorophenylsulfonyl)pyrrolidin-3-yloxy)acetamide | | |
| 179 | 2-(2-(2,4-Dichloro-N-methylphenylsulfonamido)- | 2 | 567.2 |
| | ethoxy)-N-(4-phenyl-4-(pyrrolidin-1-yl)cyclohexyl)- | | |
| | acetamide | | |
| 180 | N-(4-Benzyl-4-(pyrrolidin-1-yl)cyclohexyl)-2-((1-(4- | 2 | 611.3 |
| | methoxy-2,6-dimethylphenylsulfonyl)piperidin-3- | | |
| | yl)methoxy)acetamide | | |
| 181 | N-(4-Benzyl-4-(pyrrolidin-1-yl)cyclohexyl)-2-(1-(4- | 2 | 597.3 |
| | methoxy-2,6-dimethylphenylsulfonyl)piperidin-3- | | |
| | yloxy)acetamide | | |
| 182 | 2-((1-(4-Methoxy-2,6-dimethylphenylsulfonyl)-piperidin- | 2 | 682.4 |
| | 3-yl)methoxy)-N-methyl-N-((4-(4-methylpiperazin- | | |
| | 1-yl)-4-phenethylcyclohexyl)methyl)acetamide | | |
| 183 | N-((4-Benzyl-4-(4-methylpiperazin-1-yl)cyclohexyl)- | 2 | 628.4 |
| | methyl)-2-(2-(4-methoxy-N,2,6-trimethylphenyl- | | |
| | sulfonamido)ethoxy)-N-methylacetamide | | |
| 184 | N-((4-Benzyl-4-(4-methylpiperazin-1-yl)cyclohexyl)- | 2 | 654.4 |
| | methyl)-2-(1-(4-methoxy-2,6-dimethylphenylsulfonyl)- | | |
| | piperidin-3-yloxy)-N-methylacetamide | | |
| 185 | 2-(2-(4-Methoxy-N,2,6-trimethylphenylsulfonamido)- | 2 | 587.3 |
| | ethoxy)-N-((4-morpholino-4-phenylcyclohexyl)- | | |
| | methyl)acetamide | | |
| 186 | N-((4-Benzyl-4-(4-methylpiperazin-1- | 2 | 672.2 |
| | yl)cyclohexyl)methyl)-N-methyl-2-(2-(2,4,6-trichloro- | | |
| | N-methylphenylsulfonamido)ethoxy)acetamide | | |
| 187 | 2-((1-(4-Methoxy-2,6-dimethylphenylsulfonyl)- | 2 | 597.3 |
| | piperidin-3-yl)methoxy)-N-(4-phenyl-4-(pyrrolidin-1- | | |
| | yl)cyclohexyl)acetamide | | |
| 188 | N-((4-Benzyl-4-morpholinocyclohexyl)methyl)-2-(2-(4- | 2 | 601.3 |
| | methoxy-N,2,6-trimethylphenylsulfonamido)ethoxy)- | | |
| | acetamide | | |
| 189 | N-(4-Benzyl-4-(pyrrolidin-1-yl)cyclohexyl)-2-(2-(2,4,6- | 2 | 615.2 |
| | trichloro-N-methylphenylsulfonamido)ethoxy)- | | |
| | acetamide | | |
| 190 | N-(4-Benzyl-4-morpholinocyclohexyl)-2-(1-(4- | 2 | 613.3 |
| | methoxy-2,6-dimethylphenylsulfonyl)piperidin-3- | | |
| | yloxy)acetamide | | |
| 191 | N-(4-Phenyl-4-(pyrrolidin-1-yl)cyclohexyl)-2-(2-(2,4,6- | 2 | 601.1 |
| | trichloro-N-methylphenylsulfonamido)ethoxy)- | | |
| | acetamide | | |
| 192 | N-(4-Phenyl-4-(pyrrolidin-1-yl)cyclohexyl)-2-((1- | 2 | 641.2 |
| | (2,4,6-trichlorophenylsulfonyl)piperidin-3- | | |
| | yl)methoxy)acetamide | | |
| 193 | N-(4-Benzyl-4-(pyrrolidin-1-yl)cyclohexyl)-2-(2-(2,4- | 2 | 581.2 |
| | dichloro-N-methylphenylsulfonamido)ethoxy)- | | |
| | acetamide | | |
| 194 | N-(4-Phenyl-4-(pyrrolidin-1-yl)cyclohexyl)-2-(1-(2,4,6- | 2 | 627.2 |
| | trichlorophenylsulfonyl)piperidin-3-yloxy)acetamide | | |
| 195 | N-Methyl-N-((4-(4-methylpiperazin-1-yl)-4- | 2 | 686.2 |
| | phenethylcyclohexyl)methyl)-2-(2-(2,4,6-trichloro-N- | | |
| | methylphenylsulfonamido)ethoxy)acetamide | | |
| 196 | N-(2-(2-(4-Amino-4-phenylpiperidin-1-yl)-2- | | 489.2 |
| | oxoethoxy)ethyl)-4-methoxy-N,2,6-trimethylbenzene- | | |
| | sulfonamide | | |
| 197 | N-(2-(2-(3-Benzyl-3-(4-methylpiperazin-1- | | 572.3 |
| | yl)pyrrolidin-1-yl)-2-oxoethoxy)ethyl)-4-methoxy- | | |
| | N,2,6-trimethylbenzenesulfonamide | | |
| 198 | N-(4-(Dimethylamino)-4-phenylcyclohexyl)-2-(1- | | 587.1 |
| | (2,4,6-trichlorophenylsulfonyl)pyrrolidin-3-yloxy)- | | |
| | acetamide | | |
| 199 | N-(4-(Dimethylamino)-4-phenylcyclohexyl)-2-(2- | | 575.1 |
| | (2,4,6-trichloro-N-methylphenylsulfonamido)- | | |
| | ethoxy)acetamide | | |
| 200 | (S)-2-((1-(4-Methoxy-2,6-dimethylphenylsulfonyl)- | | 639.4 |
| | piperidin-2-yl)methoxy)-N-methyl-N-(2-(4-phenyl-4- | | |
| | (pyrrolidin-1-yl)cyclohexyl)ethyl)acetamide | | |
| 201 | (S)—N-(2-(4-(Azetidin-1-yl)-4-phenylcyclohexyl)ethyl)- | | 625.3 |
| | 2-((1-(4-methoxy-2,6-dimethylphenylsulfonyl)- | | |
| | piperidin-2-yl)methoxy)-N-methylacetamide | | |
| 202 | 1-(4-(Dimethylamino)-4-phenylpiperidin-1-yl)-2-((1-(4- | | 593.3 |
| | methoxy-2,6-dimethylphenylsulfonyl)piperidin-2- | | |
| | yl)methoxy)ethanone | | |
| 203 | N-(3-(4-(Dimethylamino)-4-phenylcyclohexyl)propyl)- | | 627.4 |
| | 2-((1-(4-methoxy-2,6-dimethylphenylsulfonyl)- | | |
| | piperidin-2-yl)methoxy)-N-methylacetamide | | |
| 204 | N-(3-(4-(3-Fluorophenyl)-4-(pyrrolidin-1-yl)cyclo- | | 671.4 |
| | hexyl)propyl)-2-((1-(4-methoxy-2,6-dimethylphenyl- | | |
| | sulfonyl)piperidin-2-yl)methoxy)-N-methylacetamide | | |
| 205 | N-(3-(4-(Azetidin-1-yl)-4-phenylcyclohexyl)propyl)-2- | | 639.4 |
| | ((1-(4-methoxy-2,6-dimethylphenylsulfonyl)piperidin- | | |
| | 2-yl)methoxy)-N-methylacetamide | | |
| 206 | N-(2-(2-(4-(Dimethylamino)-4-(pyridin-4-yl)piperidin- | | |
| | 1-yl)-2-oxoethoxy)ethyl)-4-methoxy-N,2,6- | | |
| | trimethylbenzenesulfonamide | | |

Further example compounds 210-228 were also prepared via parallel synthesis according to the protocol given below. The correlation between product and reagent, building block and method can be taken from the synthesis matrix.

The crude products from the parallel synthesis were analyzed by HPLC_MS^{[1]} and afterwards purified via reverse phase HPLC-MS^{[2]}. The identification of the products was demonstrated by analytical HPLC-MS^{[1]} measurements.

Parallel synthesis: Protocol for the Synthesis of CC Amides

To a solution of the acid AC (100 μmol) in 1 mL dichlormethane a solution of 1,1′-carbonyldiimidazol (150 μmol) in 1 mL dichlormethane was added and the reaction mixture was stirred at room temperature for 1.5 h. Afterwards a solution of amine AM (150 μmol) and Hünigs base (500 μmol) in 1 mL dichlormethane was added. The mixture was stirred for 18 h at room temperature. The solvent was evaporated under reduced pressure in a vacuum centrifuge (brand: GeneVac). The final purification resulted from HPLC-MS^{[2]}. The final analytics resulted from LC-MS^{[1]}.

### [1] Equipment and Methods for HPLC-MS Analytics:

Parallel synthesis Method: HPLC: Waters Alliance 2795 with PDA Waters 2996; MS: ZQ 2000 MassLynx Single Quadrupol MS Detector; Column: Atlantis dC18 30×2.1 mm, 3 μm; Col. temp.: 40° C., Eluent A: purified water+0.1% formic acid; Eluent B: methanol (gradient grade)+0.1% formic acid; Gradient: 0% B to 100% B in 2.3 min, 100% B for 0.4 min, 100% B to 0% B in 0.01 min, 0% B for 0.8 min; Flow: 1.0 mL/min; Ionisation: ES+, 25V; make up: 100 μL/min 70% methanol+0.2% formic acid; UV: 200-400 nm.

### [2] Equipment and Methods for HPLC-MS Purification:

Prep Pump Waters 2525; Make Up Pump: Waters 515; Auxiliary Detector: Waters DAD 2487; MS Detector: Waters Micromass ZQ; Injector/Fraction Collector: Waters Sample Manager 2767; Gradient: Initial: 60% Water 40% Methanol->12-14.5 min: 0% Water 100% Methanol->14.5-15 min: 60% Water 40% Methanol; Flow: 35 ml/min Column: Macherey-Nagel, C18 Gravity, 100×21 mm, 5μ.

| Example | | | | | [M+] | R.t. |
|---|---|---|---|---|---|---|
| No. | Structure | Name | Acid (S) | Amine (A) | found | [min] |
| 210 | 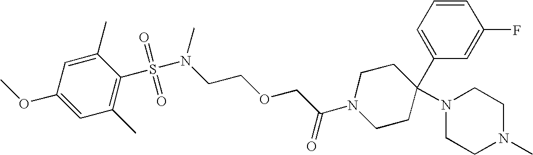 | N-(2-(2-(4-(3-Fluorophenyl)-4-(4- methylpiperazin-1-yl)piperidin-1-yl)- 2-oxoethoxy)ethyl]-4-methoxy- N,2,6-trimethylbenzenesulfonamide | 2-(2-(4-Methoxy-N,2,6- trimethylphenylsulfonamido) ethoxy)acetic acid (AC-01) | 1-(4-(3- Fluorophenyl)piperidin-4- yl)-4-methylpiperazine dihydrochloride (AM-41) | 591.4 | 1.46 |
| 211 | 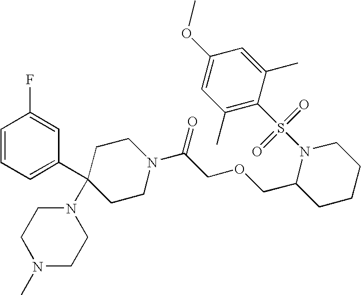 | 1-(4-(3-Fluorophenyl)-4-(4- methylpiperazin-1-yl)piperidin-1-yl)- 2-((1-(4-methoxy-2,6- dimethylphenylsulfonyl)piperidin-2- yl)methoxy)ethanone | 2-((1-(4-Methoxy-2,6- dimethylphenylsulfonyl)pipe- ridin-2- yl)methoxy)acetic acid (AC-09) | 1-(4-(3- Fluorophenyl)piperidin-4- yl)-4-methylpiperazine dihydrochloride (AM-41) | 631.5 | 1.55 |
| 212 | 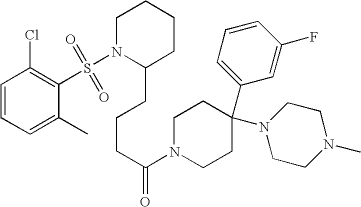 | 4-(1-(2-Chloro-6- methylphenylsulfonyl)piperidin-2- yl)-1-(4-(3-fluorophenyl)-4-(4- methylpiperazin-1-yl)piperidin-1- yl)butan-1-one | 4-(1-(2-Chloro-6- methylphenylsulfonyl)pipe- ridin-2-yl)butanoic acid (AC-30) | 1-(4-(3- Fluorophenyl)piperidin-4- yl)-4-methylpiperazine dihydrochloride (AM-41) | 619.3 | 1.58 |
| 213 | 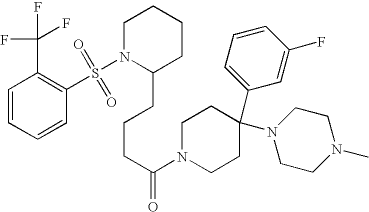 | 1-(4-(3-Fluorophenyl)-4-(4- methylpiperazin-1-yl)piperidin-1-yl)- 4-(1-(2- (trifluoromethyl)phenylsulfonyl)piperi- din-2-yl)butan-1-one | 4-(1-(2- (trifluoromethyl)phenylsulfo- nyl)piperidin-2- yl)butanoic acid (AC-31) | 1-(4-(3- Fluorophenyl)piperidin-4- yl)-4-methylpiperazine dihydrochloride (AM-41) | 639.4 | 1.60 |
| 214 | 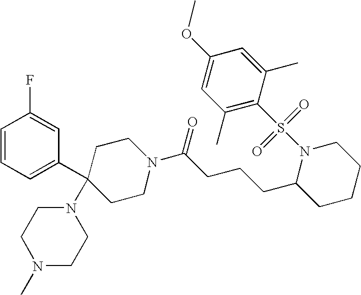 | 1-(4-(3-Fluorophenyl)-4-(4- methylpiperazin-1-yl)piperidin-1-yl)- 4-(1-(4-methoxy-2,6- dimethylphenylsulfonyl)piperidin-2- yl)butan-1-one | 4-(1-(4-Methoxy-2,6- dimethylphenylsulfonyl)pipe- ridin-2-yl)butanoic acid (AC-32) | 1-(4-(3- Fluorophenyl)piperidin-4- yl)-4-methylpiperazine dihydrochloride (AM-41) | 629.4 | 1.61 |
| 215 | 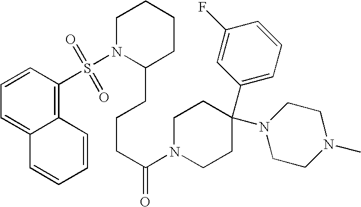 | 1-(4-(3-Fluorophenyl)-4-(4- methylpiperazin-1-yl)piperidin-1-yl)- 4-(1-naphthalen-1- ylsulfonyl)piperidin-2-yl)butan-1-one (CC_06) | 4-(1-(Naphthalen-1- ylsuflonyl)piperidin-2- yl)butanoic acid (AC-33) | 1-(4-(3- Fluorophenyl)piperidin-4- yl)-4-methylpiperazine dihydrochloride (AM-41) | 621.4 | 1.61 |
| 216 | 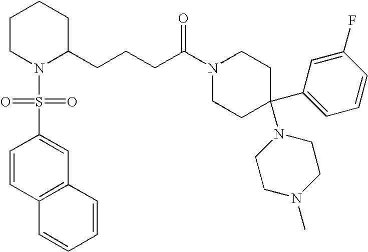 | 1-(4-(3-Fluorophenyl)-4-(4- methylpiperazin-1-yl)piperidin-1-yl)- 4-(1-(naphthalen-2- ylsulfonyl)piperidin-2-yl)butan-1-one | 4-(1-(Naphthalen-2- ylsulfonyl)piperidin-2- yl)butanoic acid (AC-34) | 1-(4-(3- Fluorophenyl)piperidin-4- yl)-4-methylpiperazine dihydrochloride (AM-41) | 621.4 | 1.64 |
| 217 | 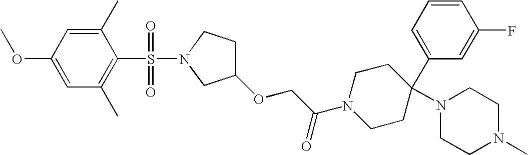 | 1-(4-(3-Fluorophenyl)-4-(4- methylpiperazin-1-yl)piperidin-1-yl)- 2-(1-(4-methoxy-2,6- dimethylphenylsulfonyl)pyrrolidin-3- yloxy)ethanone | 2-(1-(4-Methoxy-2,6- dimethylphenylsulfonyl)py- rrolidin-3-yloxy)acetic acid (AC-17) | 1-(4-(3- Fluorophenyl)piperidin-4- yl)-4-methylpiperazine dihydrochloride (AM-41) | 603.4 | 1.49 |
| 218 | 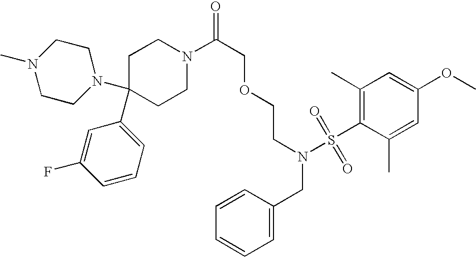 | N-Benzyl-N-(2-(2-(4-(3- fluorophenyl)-4-(4-methylpiperazin- 1-yl)piperidin-1-yl)-2- oxoethoxy)ethyl)-4-methoxy-2,6- dimethylbenzenesulfonamide | 2-(2-(N-Benzyl-4- methoxy-2,6- dimethylphenylsulfonami do)ethoxy)acetic acid (AC-29) | 1-(4-(3- Fluorophenyl)piperidin-4- yl)-4-methylpiperazine dihydrochloride (AM-41) | 667.4 | 1.64 |
| 219 | 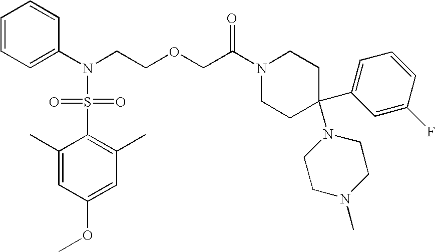 | N-(2-(2-(4-(3-Fluorophenyl)-4-(4- methylpiperazin-1-yl)piperidin-1-yl)- 2-oxoethoxy)ethyl)-4-methoxy-2,6- dimethyl-N- phenylbenzenesulfonamide | 2-(2-(4-Methoxy-2,6- dimethyl-N- phenylphenylsulfonamido) ethoxy)acetic acid (AC- 43) | 1-(4-(3- FLuorophenyl)piperidin-4- yl)-4-methylpiperazine dihydrochloride (AM-41) | 653.4 | 1.59 |
| 220 | 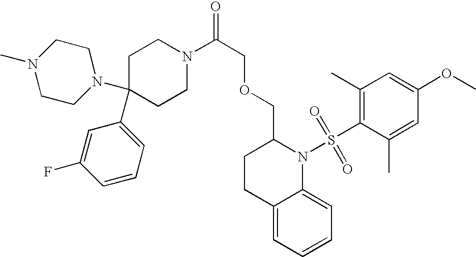 | 1-(4-(3-Fluorophenyl)-4-(4- methylpiperazin-1-yl)piperidin-1-yl)- 2-((1-(4-methoxy-2,6- dimethylphenylsulfonyl)-1,2,3,4- tetrahydroquinolin-2- yl)methoxy)ethanone | 2-((1-(4-Methoxy-2,6- dimethylphenylsulfonyl)- 1,2,3,4- tetrahydroquinolin-2- yl)methoxy)acetic acid (AC-36) | 1-(4-(3- Fluorophenyl)piperidin-4- yl)-4-methylpiperazine dihydrochloride (AM-41) | 679.4 | 1.64 |
| 221 | 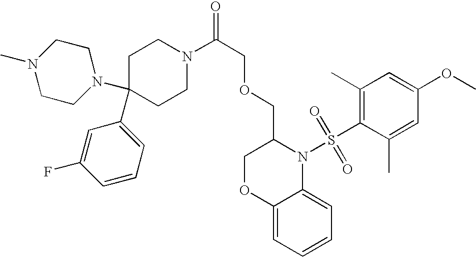 | 1-(4-(3-Fluorophenyl)-4-(4- methylpiperazin-1-yl)piperidin-1-yl)- 2-((4-(4-methoxy-2,6- dimethylphenylsulfonyl)-3,4- dihydro-2H-benzo[b][1,4]oxazin-3- yl)methoxy)ethanone | 2-((4-(4-Methoxy-2,6- dimethylphenylsulfonyl)- 3,4-dihydro-2H- benzo[b][1,4]oxazin-3- yl)methoxy)acetic acid (AC-37) | 1-(4-(3- Fluorophenyl)piperidin-4- yl)-4-methylpiperazine dihydrochoride (AM-41) | 661.4 | 1.63 |
| 222 | 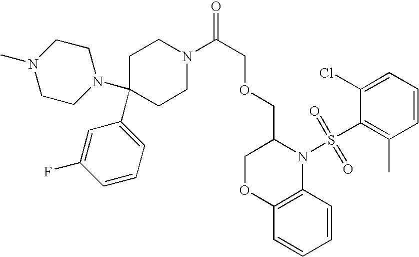 | 2-((4-(2-Chloro-6- methylphenylsulfonyl)-3,4-dihydro- 2H-benzo[b][1,4]oxazin-3- yl)methoxy)-1-(4-(3-fluroophenyl)-4- (4-methylpiperazin-1-yl)piperidin-1- yl)ethanone | 2-((4-(2-Chloro-6- methylphenylsulfonyl)- 3,4-dihydro-2H- benzo[b][1,4]oxazin-3- yl)methoxy)acetic acid (AC-38) | 1-(4-(3- Fluorophenyl)piperidin-4- yl)-4-methylpiperazine dihydrochloride (AM-41) | 671.3 | 1.62 |
| 223 | 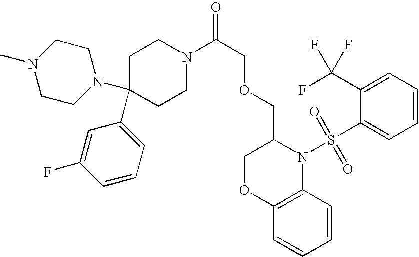 | 1-(4-(3-Fluorophenyl)-4-(4- methylpiperazin-1-yl)piperidin-1-yl)- 2-((4-(2- (trifluoromethyl)phenylsulfonyl)-3,4- dihydro-2H-benzo[b][1,4]oxazin-3- yl)methoxy)ethanone | 2-((4-(2- (Trifluoromethyl)phenylsulfo- nyl)-3,4-dihydro-2H- benzo[b][1,4]oxazin-3- yl)methoxy)acetic acid (AC-39) | 1-(4-(3- Fluorophenyl)piperidin-4- yl)-4-methylpiperazine dihydrochloride (AM-41) | 691.4 | 1.60 |
| 224 | 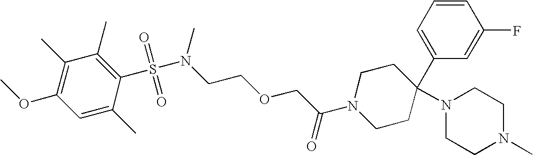 | N-(2-(2-(4-(3-Fluroophenyl)-4-(4- methylpiperaizn-1-yl)piperidin-1-yl)- 2-oxoethoxy)ethyl)-4-methoxy- N,2,3,6- tetramethylbenzenesulfonamide | 2-(2-(4-Methoxy-N,2,3,6- tetramethylphenylsulfona mido)ethoxy)acetic acid (AC-02) | 1-(4-(3- Fluorophenyl)piperidin-4- yl)-4-methylpiperazine dihydrochloride (AM-41) | 605.4 | 1.53 |
| 225 | 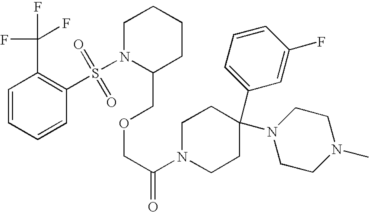 | 1-(4-(3-Fluorophenyl)-4-(4- methylpiperazin-1-yl)piperidin-1-yl)- 2-((1-(2- (trifluoromethyl)phenylsulfonyl)piperi- din-2-yl)methoxy)ethanone | 2-((1-(2- (Trifluoromethyl)phenylsul- fonyl)piperidin-2- yl)methoxy)acetic acid (AC-44) | 1-(4-(3- Fluorophenyl)piperidin-4- yl)-4-methylpiperazine dihydrochloride (AM-41) | 641.3 | 1.53 |
| 226 | 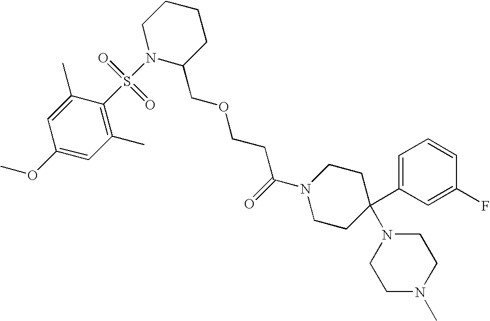 | 1-(4-(3-Fluorophenyl)-4-(4- methylpiperazin-1-yl)piperidin-1-yl)- 3-((1-(4-methoxy-2,6- dimethylphenylsulfonyl)piperidin-2- yl)methoxy)propan-1-one | 3-((1-(4-Methoxy-2,6- dimethylphenylsulfonyl)pipe- ridin-2- yl)methoxy)propenoic acid (AC-40) | 1-(4-(3- Fluorophenyl)piperidin-4- yl)-4-methylpiperazine dihydrochlordie (AM-41) | 645.4 | 1.54 |
| 227 | 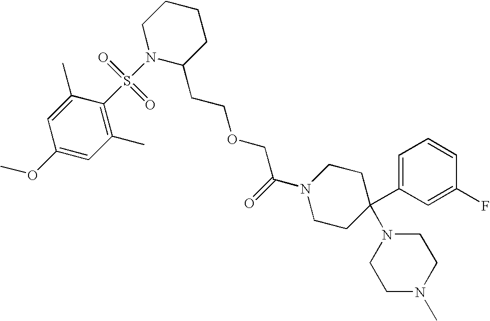 | 1-(4-(3-Fluorophenyl)-4-(4- methylpiperazin-1-yl)piperidin-1-yl)- 2-(2-(1-(4-methoxy-2,6- dimethylphenylsulfonyl)piperidin-2- yl)ethoxy)ethanone | 2-(2-(1-(4-Methoxy-2,6- dimethylphenylsulfonyl)pipe- ridin-2-yl)ethoxy)acetic acid (AC-41) | 1-(4-(3- Fluorophenyl)piperidin-4- yl)-4-methylpiperazine dihydrochloride (AM-41) | 645.3 | 1.52 |
| 228 | 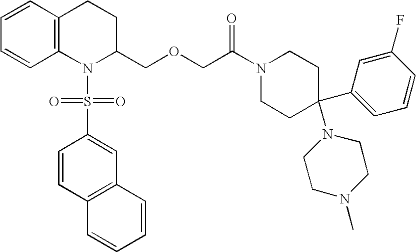 | 1-(4-(3-Fluorophenyl)-4-(4- methylpiperazin-1-yl)piperidin-1-yl)- 2-((1-(naphthalen-2-ylsulfonyl)- 1,2,3,4-tetrahydroquinolin-2- yl)methoxy)ethanone | 2-((1-(Naphthalen-2- ylsulfonyl)-1,2,3,4- tetrahydroquinolin-2- yl)methoxy)acetic acid (AC-35) | 1-(4-(3- Fluorophenyl)piperidin-4- yl)-4-methylpiperazine dihydrochloride (AM-41) | 671.3 | 1.62 |

### Pharmacological Studies

The agonistic and antagonistic action of the compounds according to the invention on the bradykinin 1 receptor (B1R) of the human and rat species were determined as described above.

Antagonists lead to a suppression of the Ca²⁺ inflow. % inhibition compared with the maximum achievable inhibition was calculated. The compounds according to the invention show a good activity on the human and on the rat receptor.

The affinity of the compounds according to the invention for the p opioid receptor was likewise determined as described above.

| | hB1R | rB1R | μ opioid receptor |
|---|---|---|---|
| | [10 μM] | [10 μM] | [1 μM] |
| Example | % inhibition | % inhibition | % inhibition |
| 1 | 96 | 105 | 42 |
| 2 | 48 | 104 | 64 |
| 3 | 98 | 102 | 54 |
| 4 | 43 | 100 | 37 |
| 5 | 104 | 100 | 41 |
| 6 | 100 | 100 | 98 |
| 7 | 103 | 100 | 55 |
| 8 | 73 | 99 | 61 |
| 9 | 104 | 95 | — |
| 10 | 103 | 95 | 50 |
| 11 | 100 | 90 | 43 |
| 12 | 77 | 88 | 37 |
| 13 | 95 | 84 | 60 |
| 14 | 89 | 82 | 40 |
| 15 | 55 | 82 | 15 |
| 16 | 91 | 80 | 30 |
| 17 | 86 | 75 | 18 |
| 18 | 68 | 74 | — |
| 19 | 51 | 73 | 96 |
| 20 | 91 | 69 | 7 |
| 21 | 29 | 67 | 100 |
| 22 | 84 | 66 | 66 |
| 23 | 96 | 66 | 69 |
| 24 | 97 | 65 | 65 |
| 25 | 93 | 63 | 51 |
| 26 | 84 | 56 | 102 |
| 27 | 69 | 51 | 27 |
| 28 | 60 | 48 | 39 |
| 29 | 61 | 46 | 48 |
| 30 | 63 | 39 | 49 |
| 31 | 50 | 29 | 35 |
| 32 | 104 | 102 | 66 |
| 33 | 94 | 101 | 44 |
| 34 | 104 | 101 | 69 |
| 35 | 102 | 101 | 26 |
| 36 | 101 | 101 | 44 |
| 37 | 103 | 101 | 62 |
| 38 | 98 | 101 | 13 |
| 39 | 105 | 101 | 32 |
| 40 | 103 | 101 | 88 |
| 41 | 103 | 101 | — |
| 42 | 105 | 101 | 9 |
| 43 | 102 | 101 | 24 |
| 44 | 102 | 101 | 2 |
| 45 | 104 | 100 | 15 |
| 46 | 104 | 100 | 32 |
| 47 | 102 | 100 | 39 |
| 48 | 102 | 100 | 8 |
| 49 | 103 | 100 | 10 |
| 50 | 102 | 100 | 20 |
| 51 | 102 | 100 | 46 |
| 52 | 104 | 100 | 33 |
| 53 | 103 | 100 | 56 |
| 54 | 103 | 100 | 42 |
| 55 | 99 | 100 | 34 |
| 56 | 101 | 100 | 49 |
| 57 | 100 | 100 | 19 |
| 58 | 103 | 100 | 11 |
| 59 | 101 | 100 | 10 |
| 60 | 103 | 100 | 79 |
| 61 | 49 | 100 | 24 |
| 62 | 103 | 100 | 61 |
| 63 | 102 | 100 | 28 |
| 64 | 84 | 100 | 12 |
| 65 | 104 | 100 | 19 |
| 66 | 102 | 100 | 31 |
| 67 | 101 | 100 | 3 |
| 68 | 103 | 100 | 41 |
| 69 | 102 | 99 | 36 |
| 70 | 104 | 99 | 13 |
| 71 | 92 | 99 | 28 |
| 72 | 103 | 99 | 96 |
| 73 | 104 | 99 | 40 |
| 74 | 102 | 99 | 25 |
| 75 | 103 | 99 | 29 |
| 76 | 98 | 99 | 43 |
| 77 | 100 | 99 | 38 |
| 78 | 101 | 99 | 15 |
| 79 | 102 | 99 | 6 |
| 80 | 86 | 99 | 61 |
| 81 | 104 | 99 | 96 |
| 82 | 99 | 99 | 22 |
| 83 | 103 | 99 | 63 |
| 84 | 104 | 99 | 61 |
| 85 | 99 | 99 | 14 |
| 86 | 101 | 99 | 76 |
| 87 | 99 | 98 | 27 |
| 88 | 101 | 98 | 14 |
| 89 | 92 | 98 | 47 |
| 90 | 97 | 97 | 97 |
| 91 | 98 | 97 | 7 |
| 92 | 101 | 95 | 83 |
| 93 | 98 | 94 | 11 |
| 94 | 64 | 93 | 5 |
| 95 | 99 | 93 | 50 |
| 96 | 100 | 91 | 46 |
| 97 | 102 | 91 | 31 |
| 98 | 72 | 91 | 65 |
| 99 | 100 | 90 | 36 |
| 100 | 51 | 88 | 21 |
| 101 | 99 | 88 | 36 |
| 102 | 97 | 85 | 81 |
| 103 | 73 | 82 | — |
| 104 | 60 | 82 | 26 |
| 105 | 83 | 81 | 55 |
| 106 | 101 | 79 | 78 |
| 107 | 105 | 79 | 99 |
| 108 | 60 | 79 | 8 |
| 109 | 80 | 78 | 94 |
| 110 | 104 | 78 | 54 |
| 111 | 80 | 78 | 100 |
| 112 | 97 | 77 | 19 |
| 113 | 79 | 74 | 74 |
| 114 | 96 | 73 | 40 |
| 115 | 71 | 72 | 9 |
| 116 | 57 | 71 | — |
| 117 | 103 | 70 | 55 |
| 118 | 101 | 69 | 18 |
| 119 | 90 | 69 | 59 |
| 120 | 97 | 67 | 35 |
| 121 | 102 | 66 | 24 |
| 122 | 105 | 66 | 2 |
| 123 | 103 | 60 | 69 |
| 124 | 98 | 58 | 17 |
| 125 | 98 | 56 | 99 |
| 126 | 87 | 55 | 0 |
| 127 | 90 | 54 | 17 |
| 128 | 98 | 53 | 69 |
| 129 | 104 | 52 | 9 |
| 130 | 102 | 49 | 7 |
| 131 | 99 | 47 | 33 |
| 132 | 103 | 43 | 45 |
| 133 | 92 | 42 | 52 |
| 134 | 102 | 38 | 2 |
| 135 | 104 | 37 | 4 |
| 136 | 99 | 32 | 66 |
| 137 | 104 | 30 | 34 |
| 138 | 102 | 28 | — |
| 139 | 105 | 24 | 56 |
| 140 | 103 | 21 | 28 |
| 141 | 104 | 20 | 23 |
| 142 | 92 | 113 | 98 |
| 143 | 103 | 111 | 34 |
| 144 | 103 | 111 | 19 |
| 145 | 92 | 110 | 93 |
| 146 | 64 | 108 | 101 |
| 147 | 94 | 108 | 93 |
| 148 | 102 | 106 | 18 |
| 149 | 103 | 105 | 73 |
| 150 | 102 | 104 | 85 |
| 151 | 90 | 103 | 66 |
| 152 | 84 | 102 | 32 |
| 153 | 97 | 102 | 86 |
| 154 | 95 | 101 | 13 |
| 155 | 97 | 100 | 34 |
| 156 | 76 | 100 | 91 |
| 157 | 76 | 99 | 36 |
| 158 | 87 | 99 | 63 |
| 159 | 45 | 98 | 37 |
| 160 | 93 | 98 | 49 |
| 161 | 101 | 98 | 13 |
| 162 | 102 | 94 | 42 |
| 163 | 84 | 93 | 93 |
| 164 | 74 | 90 | 85 |
| 165 | 103 | 90 | 27 |
| 166 | 92 | 90 | 30 |
| 167 | 100 | 88 | 25 |
| 168 | 88 | 88 | 71 |
| 169 | 41 | 86 | 24 |
| 170 | 104 | 84 | 29 |
| 171 | 75 | 82 | 29 |
| 172 | 98 | 79 | 35 |
| 173 | 64 | 78 | 26 |
| 174 | 99 | 78 | 10 |
| 175 | 97 | 77 | 58 |
| 176 | 89 | 77 | 88 |
| 177 | 103 | 76 | 29 |
| 178 | 53 | 76 | 92 |
| 179 | 41 | 75 | 94 |
| 180 | 71 | 73 | 48 |
| 181 | 78 | 71 | 46 |
| 182 | 106 | 67 | 7 |
| 183 | 102 | 66 | 5 |
| 184 | 93 | 65 | 26 |
| 185 | 93 | 65 | 15 |
| 186 | 97 | 64 | 9 |
| 187 | 95 | 63 | 84 |
| 188 | 95 | 63 | 13 |
| 189 | 65 | 60 | 54 |
| 190 | 90 | 57 | 36 |
| 191 | 90 | 55 | 90 |
| 192 | 99 | 50 | 99 |
| 193 | 93 | 34 | 56 |
| 194 | 96 | 33 | 95 |
| 195 | 100 | 28 | 17 |
| 196 | 103 | 100 | 15 |
| 197 | 98 | 99 | 13 |
| 198 | 63 | 47 | 100 |
| 199 | 76 | 36 | 100 |
| 200 | 99 | 105 | 75 |
| 201 | 100 | 97 | 81 |
| 202 | 100 | 103 | 62 |
| 203 | 99 | 98 | 80 |
| 204 | 99 | 103 | 59 |
| 205 | 100 | 100 | 77 |
| 206 | 100 | 99 | 4 |
| 207 | 100 | 101 | 96 |
| 208 | 100 | 102 | 30 |
| 209 | 100 | 99 | 17 |
| 210 | 100 | 101 | |
| 211 | 100 | 101 | |
| 212 | 100 | 100 | |
| 213 | 100 | 100 | |
| 214 | 99 | 101 | |
| 215 | 100 | 100 | |
| 216 | 100 | 95 | |
| 217 | 100 | 98 | |
| 218 | | 101 | |
| 219 | | 61 | |
| 220 | | 97 | |
| 221 | 99 | 99 | |
| 222 | 97 | 97 | |
| 223 | 84 | 97 | |
| 224 | | 100 | |
| 225 | 100 | 101 | |
| 226 | 100 | 100 | |
| 227 | 100 | 99 | |
| 228 | 100 | 101 | |

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A substituted sulfonamide compound corresponding to formula I 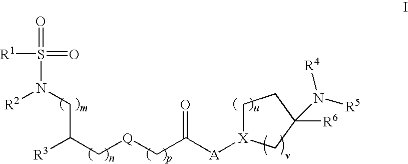
wherein
m represents 0 or 1;
n and p each independently represent 0, 1 or 2;
u and v each independently represent 0, 1, 2, 3 or 4, with the proviso that u+v=1, 2, 3 or 4;
Q represents a single bond, —CH₂— or —O—;
A represents a single bond and X represents N, or
A represents —N(R⁷)—(CH₂)₀₋₅— and X represents CH;
R¹ represents aryl, heteroaryl or an aryl or heteroaryl bonded via a C₁₋₃-alkylene group;
R² represents H, C₁₋₆-alkyl, C₃₋₈-cycloalkyl, aryl or heteroaryl; or denotes a C₃₋₈-cycloalkyl, aryl or heteroaryl bonded via a C₁₋₆-alkylene group, C₂₋₆-alkenylene group or C₂₋₆-alkynylene group;
R³ represents H, C₁₋₆-alkyl, aryl or heteroaryl; or denotes an aryl or heteroaryl bonded via a C₁₋₆-alkylene group, C₂₋₆-alkenylene group or C₂₋₆-alkynylene group; or
R² and R³ together with the —N—(CH₂)ₘ—CH— group joining them form a heterocyclic ring, which can be fused with an aryl or heteroaryl ring,
wherein the heterocyclic ring may be saturated or mono- or polyunsaturated, but not aromatic, is 4-, 5-, 6- or 7-membered, and may contain in addition to the N hetero atom to which R² is bonded, at least one further hetero atom or a hetero atom group selected from the group consisting of N, NR³, O, S, S═O or S(═O)₂; wherein
R⁸ denotes H, C₁₋₆-alkyl, —C(═O)—R⁹, C₃₋₈-cycloalkyl, aryl, heteroaryl or a C₃₋₈-cycloalkyl, aryl or heteroaryl bonded via a C₁₋₃-alkylene group, and
R⁹ denotes C₁₋₆-alkyl, C₃₋₈-cycloalkyl, aryl, heteroaryl or a C₃₋₈-cycloalkyl, aryl or heteroaryl bonded via a C₁₋₃-alkylene group;
R⁴ and R⁵ each independently denote H, C₁₋₆-alkyl, C₂₋₆-alkenyl, C₃₋₈-cycloalkyl, 3- to 8-membered heterocycloalkyl, aryl or heteroaryl or a C₃₋₈-cycloalkyl, 3- to 8-membered heterocycloalkyl, aryl or heteroaryl bonded via a C₁₋₃-alkylene group; or
R⁴ and R⁵ together with the nitrogen atom joining them form an unsubstituted or mono- or polysubstituted heterocyclic ring, which optionally may be fused with a saturated, or mono- or polyunsaturated or aromatic, unsubstituted or mono- or polysubstituted ring system; wherein
the heterocyclic ring may be saturated, or mono- or polyunsaturated, but not aromatic, is 4-, 5-, 6- or 7-membered, and may contain, in addition to the N hetero atom to which R⁴ and R⁵ are bonded, at least one further hetero atom or hetero atom group selected from the group consisting of N, NR¹⁰, O, S, S═O and S(═O)₂, wherein R¹⁰ represents H, C₁₋₆-alkyl, C₃₋₈-cycloalkyl, aryl, or heteroaryl, or an aryl, heteroaryl or C₃₋₈-cycloalkyl bonded via a C₁₋₃-alkylene group; and
the ring system is 4-, 5-, 6- or 7-membered, and may contain at least one hetero atom or hetero atom group selected from the group consisting of N, NR¹¹, O, S, S═O and S(═O)₂, wherein R¹¹ represents H, C₁₋₆-alkyl, C₃₋₈-cycloalkyl, aryl or heteroaryl, or an aryl, heteroaryl or C₃₋₈-cycloalkyl bonded via a C₁₋₃-alkylene group;
R⁶ represents an aryl or heteroaryl, or an aryl or heteroaryl bonded via a C₁₋₆-alkylene group; and
R⁷ represents H, C₁₋₆-alkyl, C₃₋₈-cycloalkyl or a C₃₋₈-cycloalkyl bonded via a C₁₋₃-alkylene group;
wherein
said C₁₋₆-alkyl, C₂₋₆-alkenyl, C₁₋₃-alkylene, C₁₋₆-alkylene, C₂₋₆-alkenylene, C₂₋₆-alkynylene, C₃₋₈-cycloalkyl, heterocycloalkyl, aryl and heteroaryl may in each case be unsubstituted or mono- or poly-substituted by identical or different substituents; and
said C₁₋₆-alkyl, C₂₋₆-alkenyl, C₁₋₃-alkylene, C₁₋₆-alkylene, C₂₋₆-alkenylene and C₂₋₆-alkynylene groups may in each case be branched or unbranched;
or a physiologically acceptable salt thereof.

2. A compound as claimed in claim 1, wherein said compound is in the form of an isolated stereoisomer.

3. A compound as claimed in claim 1, wherein said compound is in the form of a mixture of stereoisomers in any mixing ratio.

4. A compound as claimed in claim 3, wherein said mixture is a racemic mixture.

5. A compound as claimed in claim 1, wherein:
m represents 0 or 1;
n and p each independently represent 0, 1 or 2;
u and v each independently represent 0, 1, 2, 3 or 4, with the proviso that u+v=1, 2, 3 or 4;
Q represents a single bond, —CH₂— or —O—;
A represents a single bond and X represents N, or
A represents —N(R⁷)—(CH₂)₀₋₅— and X represents CH;
R¹ represents aryl or heteroaryl, or an aryl or heteroaryl bonded via a C₁₋₃-alkylene group;
R² represents H, C₁₋₆-alkyl, C₃₋₈-cycloalkyl, aryl or heteroaryl; or denotes a C₃₋₈-cycloalkyl, aryl or heteroaryl bonded via a C₁₋₆-alkylene group, C₂₋₆-alkenylene group or C₂₋₆-alkynylene group; and
R³ represents H, C₁₋₆-alkyl, aryl or heteroaryl; or denotes an aryl or heteroaryl bonded via a C₁₋₆-alkylene group, C₂₋₆-alkenylene group or C₂₋₆-alkynylene group; or
R² and R³ together with the —N—(CH₂)ₘ—CH— group joining them form a heterocyclic ring, which optionally may be fused with an aryl or heteroaryl group;
wherein the heterocyclic ring may be saturated or mono- or polyunsaturated, but not aromatic, is 4-, 5-, 6- or 7-membered, and may contain, in addition to the N hetero atom to which R² is bonded, at least one further hetero atom or a hetero atom group selected from the group consisting of N, NR³, O, S, S═O or S(═O)₂; wherein
R³ denotes H, C₁₋₆-alkyl, —C(═O)—R⁹, C₃₋₈-cycloalkyl, aryl, heteroaryl or a C₃₋₈-cycloalkyl, aryl or heteroaryl bonded via a C₁₋₃-alkylene group, and
R⁹ denotes C₁₋₆-alkyl, C₃₋₈-cycloalkyl, aryl, heteroaryl or a C₃₋₈-cycloalkyl, aryl or heteroaryl bonded via a C₁₋₃-alkylene group;
R⁴ and R⁵ each independently denote H, C₁₋₆-alkyl, C₂₋₆-alkenyl, C₃₋₈-cycloalkyl, 3- to 8-membered heterocycloalkyl, aryl or heteroaryl, or a C₃₋₈-cycloalkyl, 3- to 8-membered heterocycloalkyl, aryl or heteroaryl bonded via a C₁₋₃-alkylene group; or
R⁴ and R⁵ together with the nitrogen atom joining them form an unsubstituted or mono- or polysubstituted heterocyclic ring, which optionally may be fused with a saturated, or mono- or polyunsaturated or aromatic, unsubstituted or mono- or polysubstituted ring system, wherein
the heterocyclic ring is saturated, or mono- or polyunsaturated, but not aromatic, is 4-, 5-, 6- or 7-membered, and may contain, in addition to the N hetero atom to which R⁴ and R⁵ are bonded, at least one further hetero atom or hetero atom group selected from the group consisting of N, NR¹⁰, O, S, S═O and S(═O)₂; wherein R¹⁰ represents H, C₁₋₆-alkyl, C₃₋₈-cycloalkyl, aryl or heteroaryl, or an aryl, heteroaryl or C₃₋₈-cycloalkyl bonded via a C₁₋₃-alkylene group, and
the ring system is 4-, 5-, 6- or 7-membered, and may contain at least one hetero atom or hetero atom group selected from the group consisting of N, NR¹¹, O, S, S═O and S(═O)₂; wherein R¹¹ represents H, C₁₋₆-alkyl, C₃₋₈-cycloalkyl, aryl or heteroaryl, or an aryl, heteroaryl or C₃₋₈-cycloalkyl bonded via a C₁₋₃-alkylene group;
R⁶ represents an aryl or heteroaryl, or an aryl or heteroaryl bonded via a C₁₋₆-alkylene group; and
R⁷ represents H, C₁₋₆-alkyl, C₃₋₈-cycloalkyl or a C₃₋₈-cycloalkyl bonded via a C₁₋₃-alkylene group;
wherein
said C₁₋₆-alkyl, C₂₋₆-alkenyl, C₁₋₃-alkylene, C₁₋₆-alkylene, C₂₋₆-alkenylene, C₂₋₆-alkynylene, C₃₋₈-cycloalkyl, heterocycloalkyl, aryl and heteroaryl may in each case be unsubstituted or mono- or poly-substituted by identical or different substituents; and
said C₁₋₆-alkyl, C₂₋₆-alkenyl, C₁₋₃-alkylene, C₁₋₆-alkylene, C₂₋₆-alkenylene and C₂₋₆-alkynylene may in each case be branched or unbranched;
wherein
a substituted alkyl, alkenyl, alkylene, alkenylene, alkynylene or cycloalkyl is mono- or poly-substituted by identical or different substituents independently selected from the group consisting of F, Cl, Br, I, CN, NH₂, NH—C₁₋₆-alkyl, NH—C₁₋₆-alkylene-OH, C₁₋₆-alkyl, N(C₁₋₆-alkyl)₂, N(C₁₋₆-alkylene-OH)₂, NO₂, SH, S—C₁₋₆-alkyl, S-benzyl, O—C₁₋₆-alkyl, OH, O—C₁₋₆-alkylene-OH, ═O, O-benzyl, C(═O)C₁₋₆-alkyl, CO₂H, CO₂—C₁₋₆-alkyl and benzyl;
a substituted heterocycloalkyl is mono- or poly-substituted by identical or different substituents independently selected from the group consisting of F, Cl, Br, I, —CN, NH₂, NH—C₁₋₆-alkyl, NH—C₁₋₆-alkylene-OH, C₁₋₆-alkyl, N(C₁₋₆-alkyl)₂, N(C₁₋₆-alkylene-OH)₂, pyrrolinyl, piperazinyl, morpholinyl, NO₂, SH, S—C₁₋₆-alkyl, S-benzyl, O—C₁₋₆-alkyl, OH, O—C₁₋₆-alkylene-OH, ═O, O-benzyl, C(═O)C₁₋₆-alkyl, CO₂H, CO₂—C₁₋₆-alkyl and benzyl, or, if an N hetero atom is present, this can be substituted by a C₁₋₆-alkyl, C₃₋₈-cycloalkyl, aryl, heteroaryl or a C₃₋₈-cycloalkyl, aryl or heteroaryl bonded via a C₁₋₃-alkylene group, wherein these alkyl, cycloalkyl, alkylene and aryl and heteroaryl groups can be unsubstituted or mono- or poly-substituted by identical or different substituents;
a substituted aryl or heteroaryl is mono- or poly-substituted by identical or different substituents independently selected from the group consisting of F, Cl, Br, I, CN, NH₂, NH—C₁₋₆-alkyl, NH—C₁₋₆-alkylene-OH, N(C₁₋₆-alkyl)₂, N(C₁₋₆-alkylene-OH)₂, NH-aryl¹, N(aryl¹)₂, N(C₁₋₆-alkyl)aryl¹, pyrrolinyl, piperazinyl, morpholinyl, NO₂, SH, S—C₁₋₆-alkyl, OH, O—C₁₋₆-alkyl, O—C₁₋₆-alkyl-OH, C(═O)C₁₋₆-alkyl, NHSO₂C₁₋₆-alkyl, NHCOC₁₋₆-alkyl, CO₂H, CH₂SO₂-phenyl, CO₂—C₁₋₆-alkyl, OCF₃, CF₃, —O—CH₂—O—, —O—CH₂—CH₂—O—, —O—C(CH₃)₂—CH₂—, unsubstituted C₁₋₆-alkyl, pyrrolidinyl, imidazolyl, piperidinyl, benzyloxy, phenoxy, phenyl, naphthyl, pyridinyl, —C₁₋₃-alkylene-aryl¹, benzyl, thienyl and furyl, wherein aryl¹ represents phenyl, furyl, thienyl or pyridinyl;
a substituted heterocyclic ring is mono- or poly-substituted by identical or different substituents independently selected from the group consisting of F, Cl, Br, I, CN, NH₂, NH—C₁₋₆-alkyl, NH—C₁₋₆-alkylene-OH, C₁₋₆-alkyl, N(C₁₋₆-alkyl)₂, N(C₁₋₆-alkylene-OH)₂, NO₂, SH, S—C₁₋₆-alkyl, S-benzyl, O—C₁₋₆-alkyl, OH, O—C₁₋₆-alkylene-OH, ═O, O-benzyl, C(═O)C₁₋₆-alkyl, CO₂H, CO₂—C₁₋₆-alkyl and benzyl;
a substituted, saturated or at least partly unsaturated ring system which is fused with a heterocyclic ring formed by R⁴ and R⁵ is mono- or poly-substituted by identical or different substituents independently selected from the group consisting of F, Cl, Br, I, CN, NH₂, NH—C₁₋₆-alkyl, NH—C₁₋₆-alkylene-OH, C₁₋₆-alkyl, N(C₁₋₆-alkyl)₂, N(C₁₋₆-alkylene-OH)₂, NO₂, SH, S—C₁₋₆-alkyl, S-benzyl, O—C₁₋₆-alkyl, OH, O—C₁₋₆-alkylene-OH, ═O, O-benzyl, C(═O)C₁₋₆-alkyl, CO₂H, CO₂—C₁₋₆-alkyl and benzyl, and
a substituted aromatic ring system which is fused with the heterocyclic ring formed by R⁴ and R⁵ is substituted as defined above for aryl or heteroaryl.

6. A compound as claimed in claim 1, wherein R¹ represents phenyl, naphthyl, Indolyl, benzofuranyl, benzothiophenyl (benzothienyl); benzoxazolyl, benzoxadiazolyl, pyrrolyl, furanyl, thienyl, pyridinyl, pyridazinyl, pyrimidinyl, pyrazinyl, imidazothiazolyl, carbazolyl, dibenzofuranyl, dibenzothiophenyl (dibenzothienyl), benzyl or 2-phenylethyl, in each case unsubstituted or mono- or poly-substituted by identical or different substituents independently selected from the group consisting of —O—C₁₋₃-alkyl, C₁₋₆-alkyl, —F, —Cl, —Br, —I, —CF₃, —OCF₃, —OH, —SH, phenyl, naphthyl, furyl, thienyl and pyridinyl.

7. A compound as claimed in claim 6, wherein R¹ represents phenyl, naphthyl, benzothiophenyl, benzoxadiazolyl, thiophenyl, pyridinyl, imidazothiazolyl or dibenzofuranyl, in each case unsubstituted or mono- or poly-substituted by identical or different substituents independently selected from the group consisting of —O—C₁₋₃-alkyl, C₁₋₆-alkyl, —F, —Cl, —Br, —I, —CF₃, —OCF₃, —OH, —SH, phenyl, naphthyl, furyl, thienyl and pyridinyl.

8. A compound as claimed in claim 1, wherein R¹ represents phenyl or naphthyl, in each case unsubstituted or mono- or poly-substituted by identical or different substituents independently selected from the group consisting of methyl, methoxy, CF₃, F, Cl and Br.

9. A compound as claimed in claim 1, wherein R² represents H, C₁₋₆-alkyl, C₃₋₆-cycloalkyl or aryl, or a C₃₋₆-cycloalkyl or aryl bonded via a C₁₋₆-alkylene group, C₂₋₆-alkenylene group or C₂₋₆-alkynylene group; wherein said C₁₋₆-alkyl, C₃₋₆-cycloalkyl, C₁₋₆-alkylene, C₂₋₆-alkenylene, C₂₋₆-alkynylene and aryl may in each case be unsubstituted or mono- or poly-substituted by identical or different substituents independently selected from the group consisting of C₁₋₆-alkyl, C₁₋₆-alkyl-O—, F, Cl, Br, I, CF₃, OCF₃, OH and SH.

10. A compound as claimed in claim 1, wherein R² represents H, C₁₋₆-alkyl, cyclopropyl or phenyl, or a phenyl bonded via a C₁₋₆-alkylene group; wherein said phenyl may in each case be unsubstituted or mono- or poly-substituted by identical or different substituents independently selected from the group consisting of methyl, ethyl, n-propyl, iso-propyl, n-butyl, iso-butyl, sec-butyl, tert-butyl, methoxy, F, Cl, Br, I, CF₃, OCF₃ and OH.

11. A compound as claimed in claim 1, wherein R³ represents H, C₁₋₆-alkyl or aryl;
wherein said C₁₋₆-alkyl or aryl may in each case be unsubstituted or mono- or poly-substituted by identical or different substituents independently selected from the group consisting of C₁₋₆-alkyl, C₁₋₆-alkyl-O—, F, Cl, Br, I, CF₃, OCF₃, OH and SH.

12. A compound as claimed in claim 1, wherein R³ represents H or phenyl, wherein said phenyl may in each case be unsubstituted or mono- or poly-substituted by identical or different substituents independently selected from the group consisting of methyl, ethyl, n-propyl, iso-propyl, n-butyl, iso-butyl, sec-butyl, tert-butyl, methoxy, F, Cl, Br, I, CF₃, OCF₃ and OH.

13. A compound as claimed in claim 1, wherein R² and R³ together with the —N—(CH₂)ₘ—CH— group joining them form a 4-, 5-, 6- or 7-membered heterocyclic ring, which optionally may be fused with one or two 6-membered aromatic rings; wherein said heterocyclic ring may be saturated or mono- or polyunsaturated, but not aromatic, and may contain, in addition to the N hetero atom to which R² is bonded, at least one oxygen atom.

14. A compound as claimed in claim 1, wherein:
A represents a single bond, and X represents N; or
A represents —N(R⁷)—, —N(R⁷)—(CH₂)—, N(R⁷)—(CH₂)₂— or N(R⁷)—(CH₂)₃—, and X represents CH.

15. A compound as claimed in claim 1, wherein:
R⁴ and R⁵ each independently represent H, or substituted or unsubstituted C₁₋₆-alkyl; or
the group —NR⁴R⁵ represents a heterocylic ring corresponding to the formula IIa: 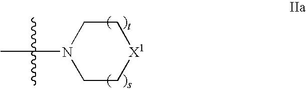
wherein
X¹ represents O, S, NR¹², CH₂ or C(halogen)₂, wherein R¹² represents H, C₁₋₆-alkyl, aryl or heteroaryl, or an aryl, preferably phenyl or naphthyl, bonded via a C₁₋₃-alkylene group; or a heteroaryl bonded via a C₁₋₃-alkylene group; and
s and t each independently represent 0, 1 or 2, with the proviso that s+t=0, 1, 2 or 3,
wherein said C₁₋₆-alkyl, C₁₋₃-alkylene, aryl and heteroaryl may in each case be unsubstituted or mono- or poly-substituted by identical or different substituents.

16. A compound as claimed in claim 15, wherein
halogen denotes F, Cl or Br; and
R¹² represents phenyl or naphthyl or a 5- to 6-membered heteroaryl having 1 or 2 N hetero atoms, or a 5- to 6-membered heteroaryl having 1 or 2 N hetero atoms and bonded via a C₁₋₃-alkylene group.

17. A compound as claimed in claim 1, wherein R⁶ represents phenyl, naphthyl, furyl, thienyl or pyridinyl, or a phenyl, naphthyl, furyl, thienyl or pyridinyl bonded via a C₁₋₃-alkylene group, wherein said phenyl, naphthyl, furyl, thienyl or pyridinyl may in each case be unsubstituted or mono- or poly-substituted by identical or different substituents independently selected from the group consisting of C₁₋₄-alkyl, O—C₁₋₄-alkyl, F, Cl, Br, I, CF₃, OCF₃, OH, —NO₂ and —CN.

18. A compound as claimed in claim 1, wherein R⁷ represents H, methyl, ethyl, n-propyl, iso-propyl, n-butyl, iso-butyl, sec-butyl, tert-butyl, cyclopropyl, cyclobutyl, cyclopropyl or cyclohexyl.

19. A compound as claimed in claim 1, wherein
m represents 0 or 1;
n and p each independently represent 0, 1 or 2;
u and v each independently represent 0, 1, 2, 3 or 4, with the proviso that u+v=1, 2, 3 or 4;
Q represents a single bond, —CH₂— or —O—;
R¹ represents phenyl, naphthyl, indolyl, benzofuranyl, benzothiophenyl, benzoxazolyl, benzoxadiazolyl, pyrrolyl, furanyl, thienyl, pyridinyl, pyridazinyl, pyrimidinyl, pyrazinyl, imidazothiazolyl, carbazolyl, dibenzofuranyl or dibenzothiophenyl, in each case unsubstituted or mono- or poly-substituted with substituents independently selected from the group consisting of —O—C₁₋₃-alkyl, C₁₋₆-alkyl, —F, —Cl, —Br, —I, —CF₃, —OCF₃, —OH, —SH, phenyl, naphthyl, furyl, thienyl and pyridinyl;
R² represents H, C₁₋₄-alkyl, phenyl or benzyl;
R³ represents H, C₁₋₆-alkyl or aryl; or denotes an aryl bonded via a C₁₋₆-alkylene group, wherein the aryl may be unsubstituted or mono- or poly-substituted by identical or different substituents independently selected from the group consisting of C₁₋₆-alkyl, C₁₋₆-alkyl-O—, F, Cl, Br, I, CF₃, OCF₃, OH and SH; or
R² and R³ together with the —N—(CH₂)ₘ—CH— group joining them form a 4-, 5-, 6- or 7-membered heterocyclic ring, which can be fused with one or two 6-membered aromatic ring(s) (benzo group); wherein the heterocyclic ring is saturated or mono- or polyunsaturated, but not aromatic, and can contain, in addition to the N hetero atom to which R² is bonded, at least one oxygen atom;
A represents a single bond and X represents N, or
A represents —N(R⁷)—(CH₂)_{0, 1, 2 or 3}— and X represents CH;
R⁴ and R⁵ each independently represent H or C₁₋₆-alkyl, or
the group —NR⁴R⁵ represents a heterocylic ring corresponding to formula IIa: 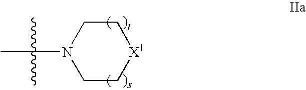
wherein
X¹ represents O, S, NR¹², CH₂ or C(halogen)₂, and R¹² represents H, C₁₋₆-alkyl, phenyl, naphthyl or pyridinyl; and
s and t each independently represent 0, 1 or 2, with the proviso that s+t=0, 1, 2 or 3;
R⁶ represents phenyl, naphthyl, furyl, thienyl or pyridinyl, or a phenyl, naphthyl, furyl, thienyl or pyridinyl bonded via a C₁₋₃-alkylene group, wherein said the phenyl, naphthyl, furyl, thienyl and pyridinyl may in each case be unsubstituted or mono- or poly-substituted by identical or different substituents independently selected from the group consisting of C₁₋₄-alkyl, O—C₁₋₄-alkyl, F, Cl, Br, I, CF₃, OCF₃, OH, —NO₂ and —CN; and
R⁷ represents H, methyl, ethyl, n-propyl, iso-propyl, n-butyl, iso-butyl, sec-butyl, tert-butyl or cyclopropyl.

20. A compound as claimed in claim 19, wherein:
R² represents H, or C₁₋₄-alkyl;
halogen denotes F. Cl or Br, and
if X¹ denotes O, S or NR¹², then s and t preferably each represent 1.

21. A compound as claimed in claim 1, wherein:
m represents 0 or 1;
n and p each independently represent 0, 1 or 2;
u and v each independently represent 0, 1, 2, 3 or 4, with the proviso that u+v=1, 2, 3 or 4;
Q represents a single bond, —CH₂— or —O—;
R¹ represents phenyl or naphthyl, in each case unsubstituted or mono- or poly-substituted by identical or different substituents independently selected from the group consisting of methyl, methoxy, CF₃, F, Cl and Br;
R² represents H, methyl, ethyl, n-propyl, iso-propyl, n-butyl, iso-butyl, sec-butyl, tert-butyl, phenyl or benzyl; and
R³ represents H or phenyl, or
R² and R³ together with the —N—(CH₂)ₘ—CH— group joining them form a 5-, 6- or 7-membered heterocyclic ring, which optionally may be fused with one or two 6-membered aromatic ring(s); wherein said heterocyclic ring may be saturated or mono- or polyunsaturated, but not aromatic, and may contain, in addition to the N hetero atom to which R² is bonded, at least one oxygen atom,
A represents a single bond, and X represents N, or
A represents —N(R⁷)—(CH₂)_{0, 1, 2 or 3}—, and X represents CH;
R⁴ and R⁵ each independently represent H or C₁₋₆-alkyl; or
the group —NR⁴R⁵ represents a heterocylic ring corresponding to the formula IIa: 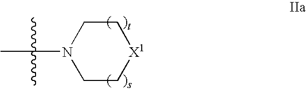
wherein
X¹ represents O, S, NR¹², CH₂ or C(halogen)₂, wherein R¹² represents H; C₁₋₆-alkyl, phenyl, naphthyl or pyridinyl; and
s and t each independently represent 0, 1 or 2, with the proviso that s+t=0, 1, 2 or 3,
R⁶ represents phenyl, naphthyl, furyl, thienyl or pyridinyl, or a phenyl, naphthyl, furyl, thienyl or pyridinyl bonded via a C₁₋₃-alkylene group, wherein said phenyl, naphthyl, furyl, thienyl and pyridinyl may in each case be unsubstituted or mono- or poly-substituted by identical or different substituents independently selected from the group consisting of C₁₋₄-alkyl, O—C₁₋₄-alkyl, F, Cl, Br, I, CF₃, OCF₃, OH, —NO₂ and —CN; and
R⁷ represents H, methyl, ethyl, n-propyl, iso-propyl, n-butyl, iso-butyl, sec-butyl, tert-butyl or cyclopropyl.

22. A compound as claimed in claim 21, wherein
R² represents H, methyl, ethyl, n-propyl, iso-propyl, n-butyl, iso-butyl, sec-butyl or tert-butyl;
halogen preferably denotes F, Cl or Br; and
if X¹ denotes O, S or NR¹², then s and t each represent 1.

23. A compound as claimed in claim 1, wherein:
m represents 0 or 1;
n and p each independently represent 0, 1 or 2;
u and v each independently represent 0, 1, 2, 3 or 4, with the proviso that u+v=1, 2, 3 or 4;
Q represents a single bond, —CH₂— or —O—;
R¹ represents 3,4-dichlorophenyl, 4-methoxyphenyl, 4-methoxy-2,6-dimethylphenyl, 4-methoxy-2,3,6-trimethylphenyl, 2.6-dichlorophenyl, 2,4-dichlorophenyl, 2,4,6-trichlorophenyl, 2-chloro-6-methylphenyl, 2,4,6-trimethylphenyl, 2-(trifluoromethyl)phenyl, 3-(trifluoromethyl)phenyl, 1-naphthyl, 2-naphthyl, 2,4-dichloro-6-methylphenyl or 4-chloro-2,5-dimethylphenyl;
R² represents H, methyl, ethyl, phenyl or benzyl; and
R³ represents H or phenyl; or
R² and R³ together with the —N—(CH₂)ₘ—CH— group joining them form a 5- or 6-membered heterocyclic ring, which can be fused with a 6-membered aromatic ring;
wherein said heterocyclic ring is saturated or mono- or polyunsaturated, but not aromatic, and can contain, in addition to the N hetero atom to which R² is bonded, at least one oxygen atom;
A represents a single bond and X represents N, or
A represents —N(R⁷)—(CH₂)_{0, 1, 2 or 3}— and X represents CH;
R⁴ and R⁵ each independently represent H or methyl, or
R⁴ and R⁵ together with the nitrogen atom joining them form a heterocyclic ring which is selected from the group consisting of: 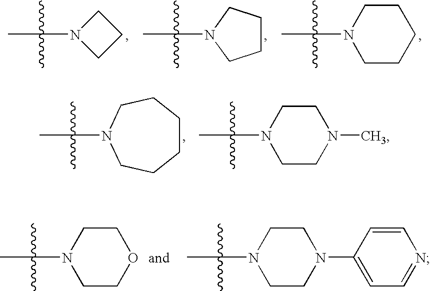
R⁶ represents phenyl or pyridinyl or a phenyl or pyridinyl bonded via —(CH₂)—, —(CH₂)₂— or —(CH₂)₃—, wherein the phenyl or pyridinyl is in each case unsubstituted or mono- or poly-substituted by identical or different substituents independently selected from the group consisting of methyl, ethyl, methoxy, ethoxy, F, Cl, Br, I, CN, CF₃, OCF₃ and OH; and
R⁷ represents H, methyl or cyclopropyl.

24. A compound as claimed in claim 23, wherein:
R¹ represents 3,4-dichlorophenyl, 4-methoxyphenyl, 4-methoxy-2,6-dimethylphenyl, 4-methoxy-2,3,6-trimethylphenyl, 2.6-dichlorophenyl, 2,4-dichlorophenyl, 2,4,6-trichlorophenyl, 2,4,6-trimethylphenyl, 3-(trifluoromethyl)phenyl, 2-naphthyl, 2,4-dichloro-6-methylphenyl or 4-chloro-2,5-dimethylphenyl; and
R² represents H, methyl or ethyl.

25. A compound as claimed in claim 1, wherein n, p and Q in the partial structure: 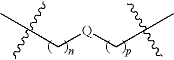
are selected such that said partial structure is selected from the group consisting of a single bond, —(CH₂)—; —(CH₂)₂—; —(CH₂)₃—; —(CH₂)—O—(CH₂)—; —(CH₂)₂—O—(CH₂); —(CH₂)—O—(CH₂)₂; —(CH₂)₂—O—(CH₂)₂; —O—(CH₂) and —(CH₂)—O—.

26. A compound as claimed in claim 1, wherein u and v each independently represent 0, 1, 2 or 3, with the proviso that u+v=2 or 3.

27. A compound as claimed in claim 1, wherein u=1 and v=1, or u=0 and v=2, or u=1 and v=2.

28. A compound as claimed in claim 1, wherein said compound is selected from the group consisting of:
(1) 2-(2-(3,4-dichlorophenylsulfonyl)-1,2,3,4-tetrahydroisoquinolin-1-yl)-N-(4-(dimethylamino)-4-phenethylcyclohexyl)acetamide
(2) N-(4-(dimethylamino)-4-phenethylcyclohexyl)-2-((1-(4-methoxyphenylsulfonyl)piperidin-2-yl)methoxy)acetamide
(3) N-(4-(dimethylamino)-4-phenethylcyclohexyl)-2-(2-(4-methoxyphenylsulfonyl)-1,2,3,4-tetrahydroisoquinolin-1-yl)acetamide
(4) N-(4-(dimethylamino)-4-(2-methylbenzyl)cyclohexyl)-2-((1-(4-methoxyphenylsulfonyl)piperidin-2-yl)methoxy)acetamide
(5) N-(4-(dimethylamino)-4-phenethylcyclohexyl)-2-(1-(4-methoxy-2,6-dimethylphenylsulfonyl)pyrrolidin-3-yloxy)acetamide
(6) N-(4-(dimethylamino)-4-(3-fluorophenyl)cyclohexyl)-2-(2-(4-methoxy-N,2,6-trimethylphenylsulfonamido)ethoxy)acetamide
(7) N-(4-(dimethylamino)-4-phenethylcyclohexyl)-2-(2-(4-methoxy-N,2,6-trimethylphenylsulfonamido)ethoxy)acetamide
(8) N-(4-(dimethylamino)-4-phenethylcyclohexyl)-2-(2-(N-ethyl-4-methoxy-2,3,6-trimethylphenylsulfonamido)ethoxy)acetamide
(9) N-(4-(dimethylamino)-4-(4-fluorobenzyl)cyclohexyl)-2-(2-(4-methoxy-N,2,6-trimethylphenylsulfonamido)ethoxy)acetamide
(10) N-(4-(dimethylamino)-4-(2-methylbenzyl)cyclohexyl)-2-(2-(4-methoxy-N,2,6-trimethylphenylsulfonamido)ethoxy)acetamide
(11) 2-(2-(4-methoxy-N,2,6-trimethylphenylsulfonamido)ethoxy)-N-(4-phenyl-4-(piperidin-1-yl)cyclohexyl)acetamide
(12) 2-(2-(3,4-dichlorophenylsulfonyl)-1,2,3,4-tetrahydroisoquinolin-1-yl)-N-(4-(dimethylamino)-4-(2-methylbenzyl)cyclohexyl)acetamide
(13) 2-(2-(2,6-dichloro-N-methylphenylsulfonamido)ethoxy)-N-(4-(dimethylamino)-4-phenethylcyclohexyl)acetamide
(14) N-(4-(dimethylamino)-4-(2-methylbenzyl)cyclohexyl)-2-(1-(4-methoxy-2,6-dimethylphenylsulfonyl)pyrrolidin-3-yloxy)acetamide
(15) N-(4-benzyl-4-(piperidin-1-yl)cyclohexyl)-2-(2-(3,4-dichlorophenylsulfonyl)-1,2,3,4-tetrahydroisoquinolin-1-yl)acetamide
(16) N-(4-(azepan-1-yl)-4-benzylcyclohexyl)-2-(2-(3,4-dichlorophenylsulfonyl)-1,2,3,4-tetrahydroisoquinolin-1-yl)acetamide
(17) N-(4-benzyl-4-(piperidin-1-yl)cyclohexyl)-2-(2-(4-methoxy-N,2,6-trimethylphenylsulfonamido)ethoxy)acetamide
(18) N-(4-benzyl-4-(piperidin-1-yl)cyclohexyl)-2-(1-(4-methoxy-2,6-dimethylphenylsulfonyl)pyrrolidin-3-yloxy)acetamide
(19) N-(4-(dimethylamino)-4-phenylcyclohexyl)-2-(2-(1-(4-methoxyphenylsulfonyl)piperidin-2-yl)ethoxy)acetamide
(20) N-(4-(azepan-1-yl)-4-benzylcyclohexyl)-2-(2-(4-methoxy-N,2,6-trimethylphenylsulfonamido)ethoxy)acetamide
(21) 2-(2-(2,4-dichloro-N-methylphenylsulfonamido)ethoxy)-N-(4-(dimethylamino)-4-(3-fluorophenyl)cyclohexyl)acetamide
(22) 2-(2-(2,4-dichloro-N-methylphenylsulfonamido)ethoxy)-N-(4-(dimethylamino)-4-phenethylcyclohexyl)acetamide
(23) N-(4-(dimethylamino)-4-phenethylcyclohexyl)-2-(2-(2,4,6-trichloro-N-methylphenylsulfonamido)ethoxy)acetamide
(24) N-(4-(dimethylamino)-4-phenethylcyclohexyl)-2-(2-(4-methoxy-N,2,3,6-tetramethylphenylsulfonamido)ethoxy)acetamide
(25) N-(4-(dimethylamino)-4-phenethylcyclohexyl)-2-(2-(N,2,4,6-tetramethylphenylsulfonamido)ethoxy)acetamide
(26) 2-(2-(3,4-dichlorophenylsulfonyl)-1,2,3,4-tetrahydroisoquinolin-1-yl)-N-(4-(dimethylamino)-4-phenylcyclohexyl)acetamide
(27) 2-(1-(4-methoxy-2,6-dimethylphenylsulfonyl)pyrrolidin-3-yloxy)-N-(4-phenyl-4-(piperidin-1-yl)cyclohexyl)acetamide
(28) 2-(2-(4-methoxy-N,2,3,6-tetramethylphenylsulfonamido)ethoxy)-N-(4-phenyl-4-(piperidin-1-yl)cyclohexyl)acetamide
(29) N-(4-(dimethylamino)-4-phenethylcyclohexyl)-2-(1-(mesitylsulfonyl)pyrrolidin-3-yloxy)acetamide
(30) 2-(2-(2,4-dichloro-N-methylphenylsulfonamido)ethoxy)-N-(4-(dimethylamino)-4-(2-methylbenzyl)cyclohexyl)acetamide
(31) N-(4-(dimethylamino)-4-phenethylcyclohexyl)-2-(2-(N-methyl-3-(trifluoromethyl)phenylsulfonamido)ethoxy)acetamide
(32) 2-((1-(4-methoxy-2,6-dimethylphenylsulfonyl)piperidin-2-yl)methoxy)-N-methyl-N-(3-(4-phenyl-4-(pyrrolidin-1-yl)cyclohexyl)propyl)acetamide
(33) N-methyl-N-((4-phenyl-4-(pyrrolidin-1-yl)cyclohexyl)methyl)-2-(1-(3-(trifluoromethyl)phenylsulfonyl)piperidin-2-yl)acetamide
(34) 2-(2-(4-methoxy-N,2,6-trimethylphenylsulfonamido)ethoxy)-N-methyl-N-(2-(4-phenyl-4-(pyrrolidin-1-yl)cyclohexyl)ethyl)acetamide
(35) 2-(2-(4-methoxy-N,2,6-trimethylphenylsulfonamido)ethoxy)-N-methyl-N-((4-phenyl-4-(pyrrolidin-1-yl)cyclohexyl)methyl)acetamide
(36) 1-(4-benzyl-4-(dimethylamino)piperidin-1-yl)-2-((1-(3,4-dichlorophenylsulfonyl)-1,2,3,4-tetrahydroquinolin-2-yl)methoxy)ethanone
(37) N-methyl-N-(3-(4-phenyl-4-(pyrrolidin-1-yl)cyclohexyl)propyl)-2-(1-(3-(trifluoromethyl)phenylsulfonyl)piperidin-2-yl)acetamide
(38) N-methyl-N-((4-phenethyl-4-(pyrrolidin-1-yl)cyclohexyl)methyl)-2-(1-(3-(trifluoromethyl)phenylsulfonyl)piperidin-2-yl)acetamide
(39) N-(2-(4-benzyl-4-(pyrrolidin-1-yl)cyclohexyl)ethyl)-2-(2-(4-methoxy-N,2,6-trimethylphenylsulfonamido)ethoxy)-N-methylacetamide
(40) N-methyl-3-(naphthalene-2-sulfonamido)-3-phenyl-N-(2-(4-phenyl-4-(pyrrolidin-1-yl)cyclohexyl)ethyl)propanamide
(41) 4-methoxy-N,2,6-trimethyl-N-(2-(2-(4-(4-methylpiperazin-1-yl)-4-phenylpiperidin-1-yl)-2-oxoethoxy)ethyl)benzenesulfonamide
(42) N-(2-(2-(4-(4-fluorophenyl)-4-(4-methylpiperazin-1-yl)piperidin-1-yl)-2-oxoethoxy)ethyl)-4-methoxy-N,2,6-trimethylbenzenesulfonamide
(43) N-methyl-N-(2-(4-phenethyl-4-(pyrrolidin-1-yl)cyclohexyl)ethyl)-2-(1-(3-(trifluoromethyl)phenylsulfonyl)piperidin-2-yl)acetamide
(44) N-(2-(2-(4-(3-fluorophenyl)-4-(4-methylpiperazin-1-yl)piperidin-1-yl)-2-oxoethoxy)ethyl)-4-methoxy-N,2,6-trimethylbenzenesulfonamide
(45) N-((4-benzyl-4-(pyrrolidin-1-yl)cyclohexyl)methyl)-2-(2-(4-methoxy-N,2,6-trimethylphenylsulfonamido)ethoxy)-N-methylacetamide
(46) 2-((1-(4-methoxy-2,6-dimethylphenylsulfonyl)piperidin-2-yl)methoxy)-N-methyl-N-(2-(4-phenethyl-4-(pyrrolidin-1-yl)cyclohexyl)ethyl)acetamide
(47) 2-(2-(4-methoxy-N,2,6-trimethylphenylsulfonamido)ethoxy)-N-methyl-N-((4-phenethyl-4-(pyrrolidin-1-yl)cyclohexyl)methyl)acetamide
(48) N-(2-(2-(4-(dimethylamino)-4-phenethylpiperidin-1-yl)-2-oxoethoxy)ethyl)-4-methoxy-N,2,6-trimethylbenzenesulfonamide
(49) 2-((1-(4-methoxy-2,6-dimethylphenylsulfonyl)piperidin-2-yl)methoxy)-1-(4-(4-methyl piperazin-1-yl)-4-phenylpiperidin-1-yl)ethanone
(50) 1-(4-(dimethylamino)-4-phenethylpiperidin-1-yl)-2-((1-(4-methoxy-2,6-dimethylphenylsulfonyl)piperidin-2-yl)methoxy)ethanone
(51) 2-((1-(4-methoxy-2,6-dimethylphenylsulfonyl)piperidin-2-yl)methoxy)-N-methyl-N-((4-phenethyl-4-(pyrrolidin-1-yl)cyclohexyl)methyl)acetamide
(52) N-(2-(4-(dimethylamino)-4-phenethylcyclohexyl)ethyl)-2-(2-(4-methoxy-N,2,6-trimethylphenylsulfonamido)ethoxy)-N-methylacetamide
(53) N-(2-(4-benzyl-4-(dimethylamino)cyclohexyl)ethyl)-2-(2-(4-methoxy-N,2,6-trimethylphenylsulfonamido)ethoxy)-N-methylacetamide
(54) N-(2-(2-(4-(dimethylamino)-4-phenylpiperidin-1-yl)-2-oxoethoxy)ethyl)-4-methoxy-N,2,6-trimethylbenzenesulfonamide
(55) N-(3-(4-(4-methylpiperazin-1-yl)-4-phenethylpiperidin-1-yl)-3-oxo-1-phenylpropyl)naphthalene-2-sulfonamide
(56) N-(2-(4-benzyl-4-(pyrrolidin-1-yl)cyclohexyl)ethyl)-N-methyl-3-(naphthalene-2-sulfonamido)-3-phenylpropanamide
(57) 1-(4-benzyl-4-(4-methylpiperazin-1-yl)piperidin-1-yl)-2-((1-(4-methoxy-2,6-dimethylphenylsulfonyl)piperidin-2-yl)methoxy)ethanone
(58) 2-(2-(4-methoxy-N,2,6-trimethylphenylsulfonamido)ethoxy)-N-methyl-N-(2-(4-phenethyl-4-(pyrrolidin-1-yl)cyclohexyl)ethyl)acetamide
(59) 2-((1-(4-methoxy-2,6-dimethylphenylsulfonyl)piperidin-2-yl)methoxy)-N-methyl-N-(3-(4-phenethyl-4-(pyrrolidin-1-yl)cyclohexyl)propyl)acetamide
(60) 2-((1-(4-methoxy-2,6-dimethylphenylsulfonyl)piperidin-2-yl)methoxy)-N-methyl-N-(2-(4-phenyl-4-(pyrrolidin-1-yl)cyclohexyl)ethyl)acetamide
(61) N-(2-(4-benzyl-4-(dimethylamino)cyclohexyl)ethyl)-2-((1-(3,4-dichlorophenylsulfonyl)-1,2,3,4-tetrahydroquinolin-2-yl)methoxy)-N-methylacetamide
(62) 2-((1-(4-methoxy-2,6-dimethylphenylsulfonyl)piperidin-2-yl)methoxy)-1-(4-phenyl-4-(4-(pyridin-4-yl)piperazin-1-yl)piperidin-1-yl)ethanone
(63) N-(2-(2-(4-benzyl-4-(dimethylamino)piperidin-1-yl)-2-oxoethoxy)ethyl)-4-methoxy-N,2,6-trimethylbenzenesulfonamide
(64) 1-(4-(4-methylpiperazin-1-yl)-4-phenylpiperidin-1-yl)-2-(1-(3-(trifluoromethyl)phenylsulfonyl)piperidin-2-yl)ethanone
(65) 1-(4-benzyl-4-(dimethylamino)piperidin-1-yl)-2-((1-(4-methoxy-2,6-dimethylphenylsulfonyl)piperidin-2-yl)methoxy)ethanone
(66) 2-((1-(4-methoxy-2,6-dimethylphenylsulfonyl)piperidin-2-yl)methoxy)-N-methyl-N-((4-phenyl-4-(pyrrolidin-1-yl)cyclohexyl)methyl)acetamide
(67) 1-(4-(3-fluorophenyl)-4-(4-methylpiperazin-1-yl)piperidin-1-yl)-2-((1-(4-methoxy-2,6-dimethylphenylsulfonyl)piperidin-2-yl)methoxy)ethanone
(68) N-(2-(4-benzyl-4-(pyrrolidin-1-yl)cyclohexyl)ethyl)-2-((1-(4-methoxy-2,6-dimethylphenylsulfonyl)piperidin-2-yl)methoxy)-N-methylacetamide
(69) 4-methoxy-N,2,6-trimethyl-N-(2-(2-oxo-2-(4-phenyl-4-(4-(pyridin-4-yl)piperazin-1-yl)piperidin-1-yl)ethoxy)ethyl)benzenesulfonamide
(70) 1-(4-(4-fluorophenyl)-4-(4-methylpiperazin-1-yl)piperidin-1-yl)-2-((1-(4-methoxy-2,6-dimethylphenylsulfonyl)piperidin-2-yl)methoxy)ethanone
(71) 1-(4-(dimethylamino)-4-phenethylpiperidin-1-yl)-2-(1-(3-(trifluoromethyl)phenylsulfonyl)piperidin-2-yl)ethanone
(72) N-(2-(4-(dimethylamino)-4-phenylcyclohexyl)ethyl)-2-(2-(4-methoxy-N,2,6-trimethylphenylsulfonamido)ethoxy)-N-methylacetamide
(73) N-(2-(4-(dimethylamino)-4-phenethylcyclohexyl)ethyl)-2-((1-(4-methoxy-2,6-dimethylphenylsulfonyl)piperidin-2-yl)methoxy)-N-methylacetamide
(74) N-(3-(4-benzyl-4-(pyrrolidin-1-yl)cyclohexyl)propyl)-N-methyl-3-(naphthalene-2-sulfonamido)-3-phenylpropanamide
(75) 1-(4-(dimethylamino)-4-phenylpiperidin-1-yl)-2-((1-(4-methoxy-2,6-dimethylphenylsulfonyl)piperidin-2-yl)methoxy)ethanone
(76) N-(2-(4-(dimethylamino)-4-phenethylcyclohexyl)ethyl)-N-methyl-2-(1-(3-(trifluoromethyl)phenylsulfonyl)piperidin-2-yl)acetamide
(77) 2-((1-(4-methoxy-2,6-dimethylphenylsulfonyl)piperidin-2-yl)methoxy)-1-(4-(4-methylpiperazin-1-yl)-4-phenethylpiperidin-1-yl)ethanone
(78) N-((4-benzyl-4-(pyrrolidin-1-yl)cyclohexyl)methyl)-2-((1-(4-methoxy-2,6-dimethylphenylsulfonyl)piperidin-2-yl)methoxy)-N-methylacetamide
(79) N-methyl-3-(naphthalene-2-sulfonamido)-N-(3-(4-phenethyl-4-(pyrrolidin-1-yl)cyclohexyl)propyl)-3-phenylpropanamide
(80) 1-(4-benzyl-4-(dimethylamino)piperidin-1-yl)-3-(1-(4-chloro-2,5-dimethylphenylsulfonyl)piperidin-2-yl)propan-1-one
(81) N-(2-(4-(dimethylamino)-4-phenylcyclohexyl)ethyl)-2-((1-(4-methoxy-2,6-dimethylphenylsulfonyl)piperidin-2-yl)methoxy)-N-methylacetamide
(82) N-(3-(4-(4-methylpiperazin-1-yl)-4-phenylpiperidin-1-yl)-3-oxo-1-phenylpropyl)naphthalene-2-sulfonamide
(83) 2-(2-(4-methoxy-N,2,6-trimethylphenylsulfonamido)ethoxy)-N-methyl-N-(3-(4-phenyl-4-(pyrrolidin-1-yl)cyclohexyl)propyl)acetamide
(84) N-(2-(4-benzyl-4-(dimethylamino)cyclohexyl)ethyl)-2-((1-(4-methoxy-2,6-dimethylphenylsulfonyl)piperidin-2-yl)methoxy)-N-methylacetamide
(85) N-(3-(4-benzyl-4-(pyrrolidin-1-yl)cyclohexyl)propyl)-2-(2-(4-methoxy-N,2,6-trimethylphenylsulfonamido)ethoxy)-N-methylacetamide
(86) N-(2-(4-benzyl-4-(dimethylamino)cyclohexyl)ethyl)-N-methyl-3-(naphthalene-2-sulfonamido)-3-phenylpropanamide
(87) 4-methoxy-N,2,6-trimethyl-N-(2-(2-(4-(4-methylpiperazin-1-yl)-4-phenethylpiperidin-1-yl)-2-oxoethoxy)ethyl)benzenesulfonamide
(88) N-(3-(4-benzyl-4-(pyrrolidin-1-yl)cyclohexyl)propyl)-2-((1-(4-methoxy-2,6-dimethylphenylsulfonyl)piperidin-2-yl)methoxy)-N-methylacetamide
(89) N-methyl-3-(naphthalene-2-sulfonamido)-N-((4-phenethyl-4-(pyrrolidin-1-yl)cyclohexyl)methyl)-3-phenylpropanamide
(90) N-(2-(4-(dimethylamino)-4-phenylcyclohexyl)ethyl)-N-methyl-3-(naphthalene-2-sulfonamido)-3-phenylpropanamide
(91) 2-(2-(4-methoxy-N,2,6-trimethylphenylsulfonamido)ethoxy)-N-methyl-N-(3-(4-phenethyl-4-(pyrrolidin-1-yl)cyclohexyl)propyl)acetamide
(92) N-methyl-3-(naphthalene-2-sulfonamido)-3-phenyl-N-((4-phenyl-4-(pyrrolidin-1-yl)cyclohexyl)methyl)propanamide
(93) N-(3-(4-benzyl-4-(pyrrolidin-1-yl)cyclohexyl)propyl)-N-methyl-2-(1-(3-(trifluoromethyl)phenylsulfonyl)piperidin-2-yl)acetamide
(94) N-((4-benzyl-4-(pyrrolidin-1-yl)cyclohexyl)methyl)-N-methyl-2-(1-(3-(trifluoromethyl)phenylsulfonyl)piperidin-2-yl)acetamide
(95) N-(2-(4-benzyl-4-(dimethylamino)cyclohexyl)ethyl)-N-methyl-2-(1-(3-(trifluoromethyl)phenylsulfonyl)piperidin-2-yl)acetamide
(96) N-(3-(4-(4-fluorophenyl)-4-(4-methylpiperazin-1-yl)piperidin-1-yl)-3-oxo-1-phenylpropyl)naphthalene-2-sulfonamide
(97) 1-(4-benzyl-4-(dimethylamino)piperidin-1-yl)-2-(1-(3-(trifluoromethyl)phenylsulfonyl)piperidin-2-yl)ethanone
(98) N-(3-oxo-1-phenyl-3-(4-phenyl-4-(4-(pyridin-4-yl)piperazin-1-yl)piperidin-1-yl)propyl)naphthalene-2-sulfonamide
(99) 3-(1-(4-chloro-2,5-dimethylphenylsulfonyl)piperidin-2-yl)-1-(4-phenyl-4-(4-(pyridin-4-yl)piperazin-1-yl)piperidin-1-yl)propan-1-one
(100) 2-((1-(3,4-dichlorophenylsulfonyl)-1,2,3,4-tetrahydroquinolin-2-yl)methoxy)-1-(4-phenyl-4-(4-(pyridin-4-yl)piperazin-1-yl)piperidin-1-yl)ethanone
(101) 2-((1-(3,4-dichlorophenylsulfonyl)-1,2,3,4-tetrahydroquinolin-2-yl)methoxy)-N-(2-(4-(dimethylamino)-4-phenethylcyclohexyl)ethyl)-N-methylacetamide
(102) N-(2-(4-(dimethylamino)-4-phenethylcyclohexyl)ethyl)-N-methyl-3-(naphthalene-2-sulfonamido)-3-phenylpropanamide
(103) 2-((1-(3,4-dichlorophenylsulfonyl)-1,2,3,4-tetrahydroquinolin-2-yl)methoxy)-1-(4-(3-fluorophenyl)-4-(4-methylpiperazin-1-yl)piperidin-1-yl)ethanone
(104) N-(2-(4-benzyl-4-(pyrrolidin-1-yl)cyclohexyl)ethyl)-2-((1-(3,4-dichlorophenylsulfonyl)-1,2,3,4-tetrahydroquinolin-2-yl)methoxy)-N-methylacetamide
(105) N-(3-(4-(dimethylamino)-4-phenethylpiperidin-1-yl)-3-oxo-1-phenylpropyl)naphthalene-2-sulfonamide
(106) N-methyl-N-(2-(4-phenyl-4-(pyrrolidin-1-yl)cyclohexyl)ethyl)-2-(1-(3-(trifluoromethyl)phenylsulfonyl)piperidin-2-yl)acetamide
(107) 3-(1-(4-chloro-2,5-dimethylphenylsulfonyl)piperidin-2-yl)-N-(2-(4-(dimethylamino)-4-phenylcyclohexyl)ethyl)-N-methylpropanamide
(108) 1-(4-benzyl-4-(4-methylpiperazin-1-yl)piperidin-1-yl)-2-((1-(3,4-dichlorophenylsulfonyl)-1,2,3,4-tetrahydroquinolin-2-yl)methoxy)ethanone
(109) 2-((1-(3,4-dichlorophenylsulfonyl)-1,2,3,4-tetrahydroquinolin-2-yl)methoxy)-1-(4-(dimethylamino)-4-phenylpiperidin-1-yl)ethanone
(110) 3-(1-(4-chloro-2,5-dimethylphenylsulfonyl)piperidin-2-yl)-1-(4-(4-methylpiperazin-1-yl)-4-phenylpiperidin-1-yl)propan-1-one
(111) 2-((1-(3,4-dichlorophenylsulfonyl)-1,2,3,4-tetrahydroquinolin-2-yl)methoxy)-N-(2-(4-(dimethylamino)-4-phenylcyclohexyl)ethyl)-N-methylacetamide
(112) N-((4-benzyl-4-(pyrrolidin-1-yl)cyclohexyl)methyl)-N-methyl-3-(naphthalene-2-sulfonamido)-3-phenylpropanamide
(113) 1-(4-phenyl-4-(4-(pyridin-4-yl)piperazin-1-yl)piperidin-1-yl)-2-(1-(3-(trifluoromethyl)phenylsulfonyl)piperidin-2-yl)ethanone
(114) N-methyl-3-(naphthalene-2-sulfonamido)-N-(2-(4-phenethyl-4-(pyrrolidin-1-yl)cyclohexyl)ethyl)-3-phenylpropanamide
(115) 2-((1-(3,4-dichlorophenylsulfonyl)-1,2,3,4-tetrahydroquinolin-2-yl)methoxy)-1-(4-(4-methylpiperazin-1-yl)-4-phenylpiperidin-1-yl)ethanone
(116) 2-((1-(3,4-dichlorophenylsulfonyl)-1,2,3,4-tetrahydroquinolin-2-yl)methoxy)-1-(4-(4-methylpiperazin-1-yl)-4-phenethylpiperidin-1-yl)ethanone
(117) N-(2-(4-benzyl-4-(pyrrolidin-1-yl)cyclohexyl)ethyl)-3-(1-(4-chloro-2,5-dimethylphenylsulfonyl)piperidin-2-yl)-N-methylpropanamide
(118) N-(3-(4-benzyl-4-(4-methylpiperazin-1-yl)piperidin-1-yl)-3-oxo-1-phenylpropyl)naphthalene-2-sulfonamide
(119) 1-(4-(4-methylpiperazin-1-yl)-4-phenethylpiperidin-1-yl)-2-(1-(3-(trifluoromethyl)phenylsulfonyl)piperidin-2-yl)ethanone
(120) N-(3-(4-(3-fluorophenyl)-4-(4-methylpiperazin-1-yl)piperidin-1-yl)-3-oxo-1-phenylpropyl)naphthalene-2-sulfonamide
(121) N-(2-(4-benzyl-4-(pyrrolidin-1-yl)cyclohexyl)ethyl)-N-methyl-2-(1-(3-(trifluoromethyl)phenylsulfonyl)piperidin-2-yl)acetamide
(122) 3-(1-(4-chloro-2,5-dimethylphenylsulfonyl)piperidin-2-yl)-1-(4-(4-methylpiperazin-1-yl)-4-phenethylpiperidin-1-yl)propan-1-one
(123) 3-(1-(4-chloro-2,5-dimethylphenylsulfonyl)piperidin-2-yl)-1-(4-(dimethylamino)-4-phenylpiperidin-1-yl)propan-1-one
(124) N-(3-(4-benzyl-4-(pyrrolidin-1-yl)cyclohexyl)propyl)-3-(1-(4-chloro-2,5-dimethylphenylsulfonyl)piperidin-2-yl)-N-methylpropanamide
(125) N-(2-(4-(dimethylamino)-4-phenylcyclohexyl)ethyl)-N-methyl-2-(1-(3-(trifluoromethyl)phenylsulfonyl)piperidin-2-yl)acetamide
(126) 2-((1-(3,4-dichlorophenylsulfonyl)-1,2,3,4-tetrahydroquinolin-2-yl)methoxy)-1-(4-(4-fluorophenyl)-4-(4-methylpiperazin-1-yl)piperidin-1-yl)ethanone
(127) N-((4-benzyl-4-(pyrrolidin-1-yl)cyclohexyl)methyl)-2-((1-(3,4-dichlorophenylsulfonyl)-1,2,3,4-tetrahydroquinolin-2-yl)methoxy)-N-methylacetamide
(128) N-methyl-3-(naphthalene-2-sulfonamido)-3-phenyl-N-(3-(4-phenyl-4-(pyrrolidin-1-yl)cyclohexyl)propyl)propanamide
(129) N-((4-benzyl-4-(pyrrolidin-1-yl)cyclohexyl)methyl)-3-(1-(4-chloro-2,5-dimethylphenylsulfonyl)piperidin-2-yl)-N-methylpropanamide
(130) 1-(4-benzyl-4-(4-methylpiperazin-1-yl)piperidin-1-yl)-3-(1-(4-chloro-2,5-dimethylphenylsulfonyl)piperidin-2-yl)propan-1-one
(131) 3-(1-(4-chloro-2,5-dimethylphenylsulfonyl)piperidin-2-yl)-N-methyl-N-(2-(4-phenethyl-4-(pyrrolidin-1-yl)cyclohexyl)ethyl)propanamide
(132) 3-(1-(4-chloro-2,5-dimethylphenylsulfonyl)piperidin-2-yl)-N-(2-(4-(dimethylamino)-4-phenethylcyclohexyl)ethyl)-N-methylpropanamide
(133) 2-((1-(3,4-dichlorophenylsulfonyl)-1,2,3,4-tetrahydroquinolin-2-yl)methoxy)-N-methyl-N-(3-(4-phenyl-4-(pyrrolidin-1-yl)cyclohexyl)propyl)acetamide
(134) N-methyl-N-(3-(4-phenethyl-4-(pyrrolidin-1-yl)cyclohexyl)propyl)-2-(1-(3-(trifluoromethyl)phenylsulfonyl)piperidin-2-yl)acetamide
(135) 3-(1-(4-chloro-2,5-dimethylphenylsulfonyl)piperidin-2-yl)-1-(4-(3-fluorophenyl)-4-(4-methylpiperazin-1-yl)piperidin-1-yl)propan-1-one
(136) 3-(1-(4-chloro-2,5-dimethylphenylsulfonyl)piperidin-2-yl)-N-methyl-N-(3-(4-phenyl-4-(pyrrolidin-1-yl)cyclohexyl)propyl)propanamide
(137) 3-(1-(4-chloro-2,5-dimethylphenylsulfonyl)piperidin-2-yl)-1-(4-(dimethylamino)-4-phenethylpiperidin-1-yl)propan-1-one
(138) 3-(1-(4-chloro-2,5-dimethylphenylsulfonyl)piperidin-2-yl)-1-(4-(4-fluorophenyl)-4-(4-methylpiperazin-1-yl)piperidin-1-yl)propan-1-one
(139) 3-(1-(4-chloro-2,5-dimethylphenylsulfonyl)piperidin-2-yl)-N-methyl-N-(2-(4-phenyl-4-(pyrrolidin-1-yl)cyclohexyl)ethyl)propanamide
(140) 3-(1-(4-chloro-2,5-dimethylphenylsulfonyl)piperidin-2-yl)-N-methyl-N-((4-phenyl-4-(pyrrolidin-1-yl)cyclohexyl)methyl)propanamide
(141) 3-(1-(4-chloro-2,5-dimethylphenylsulfonyl)piperidin-2-yl)-N-methyl-N-((4-phenethyl-4-(pyrrolidin-1-yl)cyclohexyl)methyl)propanamide
(142) N-(4-phenyl-4-(pyrrolidin-1-yl)cyclohexyl)-2-((1-(2,4,6-trichlorophenylsulfonyl)piperidin-2-yl)methoxy)acetamide
(143) N-((4-benzyl-4-(4-methylpiperazin-1-yl)cyclohexyl)methyl)-2-((1-(4-methoxy-2,6-dimethylphenylsulfonyl)piperidin-2-yl)methoxy)-N-methylacetamide
(144) 2-((1-(4-methoxy-2,6-dimethylphenylsulfonyl)piperidin-2-yl)methoxy)-N-methyl-N-((4-(4-methylpiperazin-1-yl)-4-phenethylcyclohexyl)methyl)acetamide
(145) 2-((1-(4-methoxy-2,6-dimethylphenylsulfonyl)pyrrolidin-2-yl)methoxy)-N-(4-phenyl-4-(pyrrolidin-1-yl)cyclohexyl)acetamide
(146) N-(4-phenyl-4-(pyrrolidin-1-yl)cyclohexyl)-2-((1-(2,4,6-trichlorophenylsulfonyl)pyrrolidin-2-yl)methoxy)acetamide
(147) 2-((1-(4-methoxy-2,6-dimethylphenylsulfonyl)piperidin-2-yl)methoxy)-N-(4-phenyl-4-(pyrrolidin-1-yl)cyclohexyl)acetamide
(148) 2-((1-(4-methoxy-2,6-dimethylphenylsulfonyl)pyrrolidin-2-yl)methoxy)-N-methyl-N-((4-(4-methylpiperazin-1-yl)-4-phenethylcyclohexyl)methyl)acetamide
(149) N-(4-benzyl-4-(pyrrolidin-1-yl)cyclohexyl)-2-((1-(4-methoxy-2,6-dimethylphenylsulfonyl)piperidin-2-yl)methoxy)acetamide
(150) 2-(2-(4-methoxy-N,2,6-trimethylphenylsulfonamido)ethoxy)-N-(4-phenyl-4-(pyrrolidin-1-yl)cyclohexyl)acetamide
(151) N-(4-benzyl-4-(pyrrolidin-1-yl)cyclohexyl)-2-((1-(4-methoxy-2,6-dimethylphenylsulfonyl)pyrrolidin-2-yl)methoxy)acetamide
(152) 2-((1-(4-methoxy-2,6-dimethylphenylsulfonyl)pyrrolidin-2-yl)methoxy)-N-((4-morpholino-4-phenylcyclohexyl)methyl)acetamide
(153) 2-(1-(4-methoxy-2,6-dimethylphenylsulfonyl)pyrrolidin-3-yloxy)-N-(4-phenyl-4-(pyrrolidin-1-yl)cyclohexyl)acetamide
(154) N-((4-benzyl-4-(4-methylpiperazin-1-yl)cyclohexyl)methyl)-2-((1-(4-methoxy-2,6-dimethylphenylsulfonyl)pyrrolidin-2-yl)methoxy)-N-methylacetamide
(155) N-(4-benzyl-4-morpholinocyclohexyl)-2-((1-(4-methoxy-2,6-dimethylphenylsulfonyl)piperidin-2-yl)methoxy)acetamide
(156) N-(4-benzyl-4-(pyrrolidin-1-yl)cyclohexyl)-2-((1-(2,4,6-trichlorophenylsulfonyl)pyrrolidin-2-yl)methoxy)acetamide
(157) 2-((1-(4-methoxy-2,6-dimethylphenylsulfonyl)piperidin-2-yl)methoxy)-N-((4-morpholino-4-phenylcyclohexyl)methyl)acetamide
(158) 2-((1-(4-methoxy-2,6-dimethylphenylsulfonyl)piperidin-2-yl)methoxy)-N-(4-morpholino-4-phenylcyclohexyl)acetamide
(159) N-(4-benzyl-4-morpholinocyclohexyl)-2-((1-(4-methoxy-2,6-dimethylphenylsulfonyl)pyrrolidin-2-yl)methoxy)acetamide
(160) 2-((1-(4-methoxy-2,6-dimethylphenylsulfonyl)pyrrolidin-2-yl)methoxy)-N-(4-morpholino-4-phenylcyclohexyl)acetamide
(161) N-methyl-N-((4-(4-methylpiperazin-1-yl)-4-phenethylcyclohexyl)methyl)-2-((1-(2,4,6-trichlorophenylsulfonyl)piperidin-2-yl)methoxy)acetamide
(162) N-((4-benzyl-4-(4-methylpiperazin-1-yl)cyclohexyl)methyl)-N-methyl-2-((1-(2,4,6-trichlorophenylsulfonyl)piperidin-2-yl)methoxy)acetamide
(163) N-(4-benzyl-4-(pyrrolidin-1-yl)cyclohexyl)-2-((1-(2,4,6-trichlorophenylsulfonyl)piperidin-2-yl)methoxy)acetamide
(164) N-(4-morpholino-4-phenylcyclohexyl)-2-((1-(2,4,6-trichlorophenylsulfonyl)pyrrolidin-2-yl)methoxy)acetamide
(165) 2-(2-(4-methoxy-N,2,6-trimethylphenylsulfonamido)ethoxy)-N-methyl-N-((4-(4-methylpiperazin-1-yl)-4-phenethylcyclohexyl)methyl)acetamide
(166) N-(4-benzyl-4-(pyrrolidin-1-yl)cyclohexyl)-2-(1-(4-methoxy-2,6-dimethylphenylsulfonyl)pyrrolidin-3-yloxy)acetamide
(167) 2-(2-(4-methoxy-N,2,6-trimethylphenylsulfonamido)ethoxy)-N-(4-morpholino-4-phenylcyclohexyl)acetamide
(168) N-((4-benzyl-4-(4-methylpiperazin-1-yl)cyclohexyl)methyl)-N-methyl-2-((1-(2,4,6-trichlorophenylsulfonyl)pyrrolidin-2-yl)methoxy)acetamide
(169) N-((4-benzyl-4-morpholinocyclohexyl)methyl)-2-((1-(4-methoxy-2,6-dimethylphenylsulfonyl)pyrrolidin-2-yl)methoxy)acetamide
(170) N-(4-benzyl-4-(pyrrolidin-1-yl)cyclohexyl)-2-(2-(4-methoxy-N,2,6-trimethylphenylsulfonamido)ethoxy)acetamide
(171) N-(4-benzyl-4-morpholinocyclohexyl)-2-(2-(4-methoxy-N,2,6-trimethylphenylsulfonamido)ethoxy)acetamide
(172) N-methyl-N-((4-(4-methylpiperazin-1-yl)-4-phenethylcyclohexyl)methyl)-2-((1-(2,4,6-trichlorophenylsulfonyl)pyrrolidin-2-yl)methoxy)acetamide
(173) 2-(1-(4-methoxy-2,6-dimethylphenylsulfonyl)pyrrolidin-3-yloxy)-N-((4-morpholino-4-phenylcyclohexyl)methyl)acetamide
(174) N-((4-benzyl-4-(4-methylpiperazin-1-yl)cyclohexyl)methyl)-2-(1-(4-methoxy-2,6-dimethylphenylsulfonyl)pyrrolidin-3-yloxy)-N-methylacetamide
(175) N-((4-benzyl-4-(4-methylpiperazin-1-yl)cyclohexyl)methyl)-N-methyl-2-(1-(2,4,6-trichlorophenylsulfonyl)piperidin-3-yloxy)acetamide
(176) 2-(1-(4-methoxy-2,6-dimethylphenylsulfonyl)piperidin-3-yloxy)-N-(4-phenyl-4-(pyrrolidin-1-yl)cyclohexyl)acetamide
(177) 2-(1-(4-methoxy-2,6-dimethylphenylsulfonyl)pyrrolidin-3-yloxy)-N-methyl-N-((4-(4-methylpiperazin-1-yl)-4-phenethylcyclohexyl)methyl)acetamide
(178) N-(4-phenyl-4-(pyrrolidin-1-yl)cyclohexyl)-2-(1-(2,4,6-trichlorophenylsulfonyl)pyrrolidin-3-yloxy)acetamide
(179) 2-(2-(2,4-dichloro-N-methylphenylsulfonamido)ethoxy)-N-(4-phenyl-4-(pyrrolidin-1-yl)cyclohexyl)acetamide
(180) N-(4-benzyl-4-(pyrrolidin-1-yl)cyclohexyl)-2-((1-(4-methoxy-2,6-dimethylphenylsulfonyl)piperidin-3-yl)methoxy)acetamide
(181) N-(4-benzyl-4-(pyrrolidin-1-yl)cyclohexyl)-2-(1-(4-methoxy-2,6-dimethylphenylsulfonyl)piperidin-3-yloxy)acetamide
(182) 2-((1-(4-methoxy-2,6-dimethylphenylsulfonyl)piperidin-3-yl)methoxy)-N-methyl-N-((4-(4-methylpiperazin-1-yl)-4-phenethylcyclohexyl)methyl)acetamide
(183) N-((4-benzyl-4-(4-methylpiperazin-1-yl)cyclohexyl)methyl)-2-(2-(4-methoxy-N,2,6-trimethylphenylsulfonamido)ethoxy)-N-methylacetamide
(184) N-((4-benzyl-4-(4-methylpiperazin-1-yl)cyclohexyl)methyl)-2-(1-(4-methoxy-2,6-dimethylphenylsulfonyl)piperidin-3-yloxy)-N-methylacetamide
(185) 2-(2-(4-methoxy-N,2,6-trimethylphenylsulfonamido)ethoxy)-N-((4-morpholino-4-phenylcyclohexyl)methyl)acetamide
(186) N-((4-benzyl-4-(4-methylpiperazin-1-yl)cyclohexyl)methyl)-N-methyl-2-(2-(2,4,6-trichloro-N-methylphenylsulfonamido)ethoxy)acetamide
(187) 2-((1-(4-methoxy-2,6-dimethylphenylsulfonyl)piperidin-3-yl)methoxy)-N-(4-phenyl-4-(pyrrolidin-1-yl)cyclohexyl)acetamide
(188) N-((4-benzyl-4-morpholinocyclohexyl)methyl)-2-(2-(4-methoxy-N,2,6-trimethylphenylsulfonamido)ethoxy)acetamide
(189) N-(4-benzyl-4-(pyrrolidin-1-yl)cyclohexyl)-2-(2-(2,4,6-trichloro-N-methylphenylsulfonamido)ethoxy)acetamide
(190) N-(4-benzyl-4-morpholinocyclohexyl)-2-(1-(4-methoxy-2,6-dimethylphenylsulfonyl)piperidin-3-yloxy)acetamide
(191) N-(4-phenyl-4-(pyrrolidin-1-yl)cyclohexyl)-2-(2-(2,4,6-trichloro-N-methylphenylsulfonamido)ethoxy)acetamide
(192) N-(4-phenyl-4-(pyrrolidin-1-yl)cyclohexyl)-2-((1-(2,4,6-trichlorophenylsulfonyl)piperidin-3-yl)methoxy)acetamide
(193) N-(4-benzyl-4-(pyrrolidin-1-yl)cyclohexyl)-2-(2-(2,4-dichloro-N-methylphenylsulfonamido)ethoxy)acetamide
(194) N-(4-phenyl-4-(pyrrolidin-1-yl)cyclohexyl)-2-(1-(2,4,6-trichlorophenylsulfonyl)piperidin-3-yloxy)acetamide
(195) N-methyl-N-((4-(4-methylpiperazin-1-yl)-4-phenethylcyclohexyl)methyl)-2-(2-(2,4,6-trichloro-N-methylphenylsulfonamido)ethoxy)acetamide
(196) N-(2-(2-(4-amino-4-phenylpiperidin-1-yl)-2-oxoethoxy)ethyl)-4-methoxy-N,2,6-trimethylbenzenesulfonamide
(197) N-(2-(2-(3-benzyl-3-(4-methylpiperazin-1-yl)pyrrolidin-1-yl)-2-oxoethoxy)ethyl)-4-methoxy-N,2,6-trimethylbenzenesulfonamide
(198) N-(4-(dimethylamino)-4-phenylcyclohexyl)-2-(1-(2,4,6-trichlorophenylsulfonyl)pyrrolidin-3-yloxy)acetamide
(199) N-(4-(dimethylamino)-4-phenylcyclohexyl)-2-(2-(2,4,6-trichloro-N-methylphenylsulfonamido)ethoxy)acetamide
(200) (S)-2-((1-(4-methoxy-2,6-dimethylphenylsulfonyl)piperidin-2-yl)methoxy)-N-methyl-N-(2-(4-phenyl-4-(pyrrolidin-1-yl)cyclohexyl)ethyl)acetamide
(201) (S)-N-(2-(4-(azetidin-1-yl)-4-phenylcyclohexyl)ethyl)-2-((1-(4-methoxy-2,6-dimethylphenylsulfonyl)piperidin-2-yl)methoxy)-N-methylacetamide
(202) 1-(4-(dimethylamino)-4-phenylpiperidin-1-yl)-2-((1-(4-methoxy-2,6-dimethylphenylsulfonyl)piperidin-2-yl)methoxy)ethanone
(203) N-(3-(4-(dimethylamino)-4-phenylcyclohexyl)propyl)-2-((1-(4-methoxy-2,6-dimethylphenylsulfonyl)piperidin-2-yl)methoxy)-N-methylacetamide
(204) N-(3-(4-(3-fluorophenyl)-4-(pyrrolidin-1-yl)cyclohexyl)propyl)-2-((1-(4-methoxy-2,6-dimethylphenylsulfonyl)piperidin-2-yl)methoxy)-N-methylacetamide
(205) N-(3-(4-(azetidin-1-yl)-4-phenylcyclohexyl)propyl)-2-((1-(4-methoxy-2,6-dimethylphenylsulfonyl)piperidin-2-yl)methoxy)-N-methylacetamide
(206) N-(2-(2-(4-(dimethylamino)-4-(pyridin-4-yl)piperidin-1-yl)-2-oxoethoxy)ethyl)-4-methoxy-N,2,6-trimethylbenzenesulfonamide
(207) N-(2-(4-(Dimethylamino)-4-(pyridin-3-yl)cyclohexyl)ethyl)-2-(2-(4-methoxy-N,2,6-trimethylphenylsulfonamido)ethoxy)-N-methylacetamide
(208) N-(2-(2-(4-(Dimethylamino)-4-(pyridin-3-yl)piperidin-1-yl)-2-oxoethoxy)ethyl)-4-methoxy-N,2,6-trimethylbenzenesulfonamide
(209) 4-Methoxy-N,2,6-trimethyl-N-(2-(2-(4-(methylamino)-4-(pyridin-4-yl)piperidin-1-yl)-2-oxoethoxy)ethyl)benzenesulfonamide
(210) N-(2-(2-(4-(3-Fluorophenyl)-4-(4-methylpiperazin-1-yl)piperidin-1-yl)-2-oxoethoxy)ethyl)-4-methoxy-N,2,6-trimethylbenzenesulfonamide
(211) 1-(4-(3-Fluorophenyl)-4-(4-methylpiperazin-1-yl)piperidin-1-yl)-2-((1-(4-methoxy-2,6-dimethylphenylsulfonyl)piperidin-2-yl)methoxy)ethanone
(212) 4-(1-(2-Chloro-6-methylphenylsulfonyl)piperidin-2-yl)-1-(4-(3-fluorophenyl)-4-(4-methylpiperazin-1-yl)piperidin-1-yl)butan-1-one
(213) 1-(4-(3-Fluorophenyl)-4-(4-methylpiperazin-1-yl)piperidin-1-yl)-4-(1-(2-(trifluoromethyl)phenylsulfonyl)piperidin-2-yl)butan-1-one
(214) 1-(4-(3-Fluorophenyl)-4-(4-methylpiperazin-1-yl)piperidin-1-yl)-4-(1-(4-methoxy-2,6-dimethylphenylsulfonyl)piperidin-2-yl)butan-1-one
(215) 1-(4-(3-Fluorophenyl)-4-(4-methylpiperazin-1-yl)piperidin-1-yl)-4-(1-(naphthalen-1-ylsulfonyl)piperidin-2-yl)butan-1-one
(216) 1-(4-(3-Fluorophenyl)-4-(4-methylpiperazin-1-yl)piperidin-1-yl)-4-(1-(naphthalen-2-ylsulfonyl)piperidin-2-yl)butan-1-one
(217) 1-(4-(3-Fluorophenyl)-4-(4-methylpiperazin-1-yl)piperidin-1-yl)-2-(1-(4-methoxy-2,6-dimethylphenylsulfonyl)pyrrolidin-3-yloxy)ethanone
(218) N-Benzyl-N-(2-(2-(4-(3-fluorophenyl)-4-(4-methylpiperazin-1-yl)piperidin-1-yl)-2-oxoethoxy)ethyl)-4-methoxy-2,6-dimethylbenzenesulfonamide
(219) N-(2-(2-(4-(3-Fluorophenyl)-4-(4-methylpiperazin-1-yl)piperidin-1-yl)-2-oxoethoxy)ethyl)-4-methoxy-2,6-dimethyl-N-phenylbenzenesulfonamide
(220) 1-(4-(3-Fluorophenyl)-4-(4-methylpiperazin-1-yl)piperidin-1-yl)-2-((1-(4-methoxy-2,6-dimethylphenylsulfonyl)-1,2,3,4-tetrahydroquinolin-2-yl)methoxy)ethanone
(221) 1-(4-(3-Fluorophenyl)-4-(4-methylpiperazin-1-yl)piperidin-1-yl)-2-((4-(4-methoxy-2,6-dimethylphenylsulfonyl)-3,4-dihydro-2H-benzo[b][1,4]oxazin-3-yl)methoxy)ethanone
(222) 2-((4-(2-Chloro-6-methylphenylsulfonyl)-3,4-dihydro-2H-benzo[b][1,4]oxazin-3-yl)methoxy)-1-(4-(3-fluorophenyl)-4-(4-methylpiperazin-1-yl)piperidin-1-yl)ethanone
(223) 1-(4-(3-Fluorophenyl)-4-(4-methylpiperazin-1-yl)piperidin-1-yl)-2-((4-(2-(trifluoromethyl)phenylsulfonyl)-3,4-dihydro-2H-benzo[b][1,4]oxazin-3-yl)methoxy)ethanone
(224) N-(2-(2-(4-(3-Fluorophenyl)-4-(4-methylpiperazin-1-yl)piperidin-1-yl)-2-oxoethoxy)ethyl)-4-methoxy-N,2,3,6-tetramethylbenzenesulfonamide
(225) 1-(4-(3-Fluorophenyl)-4-(4-methylpiperazin-1-yl)piperidin-1-yl)-2-((1-(2-(trifluoromethyl)phenylsulfonyl)piperidin-2-yl)methoxy)ethanone
(226) 1-(4-(3-Fluorophenyl)-4-(4-methylpiperazin-1-yl)piperidin-1-yl)-3-((1-(4-methoxy-2,6-dimethylphenylsulfonyl)piperidin-2-yl)methoxy)propan-1-one
(227) 1-(4-(3-Fluorophenyl)-4-(4-methylpiperazin-1-yl)piperidin-1-yl)-2-(2-(1-(4-methoxy-2,6-dimethylphenylsulfonyl)piperidin-2-yl)ethoxy)ethanone
(228) 1-(4-(3-Fluorophenyl)-4-(4-methylpiperazin-1-yl)piperidin-1-yl)-2-((1-(naphthalen-2-ylsulfonyl)-1,2,3,4-tetrahydroquinolin-2-yl)methoxy)ethanone, and
physiologically acceptable salts thereof.

29. A pharmaceutical composition comprising a compound as claimed in claim 1 and at least one pharmaceutically acceptable additive or auxiliary substance.

30. A process for preparing a compound as claimed in claim 1, said process comprising: 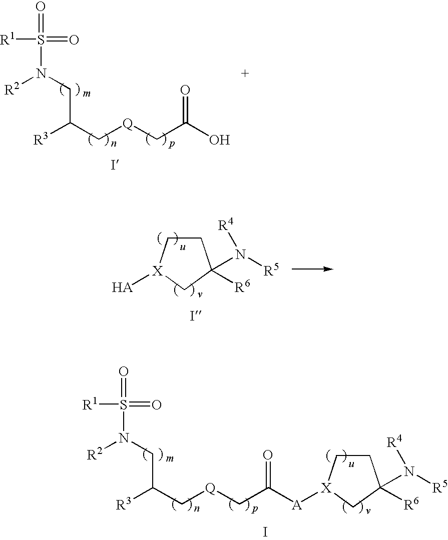
reacting a free amine 1″ and a carboxylic acid 1′ in an amide formation in the presence of a dehydrating agent and optionally an organic base in an organic solvent to yield a compound corresponding to formula I.

31. A method of treating or inhibiting pain in a subject in need thereof, said method comprising administering to said subject a pharmacologically effective amount of a compound as claimed in claim 1.

32. A method as claimed in claim 31, wherein said pain is inflammatory pain, acute pain, visceral pain, neuropathic pain or chronic pain.

33. A method of treating or inhibiting a condition selected from the group consisting of pain, migraine, diabetes, respiratory tract diseases, inflammatory intestinal diseases, neurological diseases, skin inflammations, rheumatic diseases, septic shock, reperfusion syndrome, and obesity, or for inhibiting angiogenesis, in a subject in need thereof, said method comprising administering to said subject a pharmacologically effective amount of a compound as claimed in claim 1.
